(19) United States
(12) Patent Application Publication
(10) Pub. No.: US 2025/0057780 A1
(43) Pub. Date: Feb. 20, 2025

(54) NANOPARTICLES AND METHODS OF PRODUCTION FOR THE ENCAPSULATION OF NUCLEIC ACIDS

(71) Applicant: OPTIMEOS LIFE SCIENCES, INC., Princeton, NJ (US)

(72) Inventors: Robert F. PAGELS, Princeton, NJ (US); Chester E. MARKWALTER, Princeton, NJ (US); Mark ESPOSITO, Princeton, NJ (US)

(73) Assignee: OPTIMEOS LIFE SCIENCES, INC., Princeton, NJ (US)

(21) Appl. No.: 18/723,283

(22) PCT Filed: Dec. 23, 2022
(86) PCT No.: PCT/US2022/053948

§ 371 Date: Jun. 21, 2024

Related U.S. Application Data
(60) Provisional application No. 63/293,497, filed on Dec. 23, 2021.
(60) Provisional application No. 63/314,364, filed on Feb. 25, 2022.
(60) Provisional application No. 63/389,349, filed on Jul. 14, 2022.

(51) Int. Cl. A61K 9/51; A61K 39/00; A61K 39/215; A61K 48/00; C12N 15/88

(52) U.S. Cl. CPC A61K 9/5153; A61K 9/5123; A61K 9/5161; A61K 9/5192; A61K 39/0011; A61K 39/215; A61K 48/0041; C12N 15/88; A61K 2039/53

(57) ABSTRACT

Presented are coated nanoparticles that have a desired in vitro or in vivo effect, compositions of the coated nanoparticles, methods of use of the coated nanoparticles to achieve therapeutic or prophylactic effects in cells, organs, or subjects, and processes for forming an inverse nanoparticle encapsulating a nucleic acid, such as RNA, stabilized by an amphiphilic copolymer and coating the inverse nanoparticle with additional agents to make a water-dispersible coated nanoparticle.

This application claims the benefit of U.S. Provisional Application 63/293,497, filed Dec. 23, 2021, U.S. Provisional Application 63/314,364, filed Feb. 25, 2022, and U.S. Provisional Application 63/389,349, filed Jul. 14, 2022, all of which are hereby incorporated by reference in their entireties herein.

This invention was made with Government support under Award Number 2032023 awarded by the National Science Foundation. The Government has certain rights in the invention.

### FIELD OF THE INVENTION

Embodiments of the invention provide hybrid polymer-lipid nanoparticle compositions encapsulating nucleic acids, a process of nanoparticle production, and methods for use of the compositions for therapeutic or prophylactic effect in cells, organs, tissues, or subjects.

### BACKGROUND OF THE INVENTION

Nucleic acids including RNA and DNA have short half-lives and are rapidly degraded by RNases or DNases respectively and rapidly cleared when administered as soluble species. Nucleic acids can also be recognized and cleared by the immune system. An additional limitation of nucleic acids is that their high water solubility prevents them from permeating cell membranes.

### SUMMARY OF THE INVENTION

U.S. Pat. Nos. 10,231,937 and 11,103,461, U.S. published applications US20200206136, US20210259984, and US20200023332, and International Application Publications WO2020227350 and WO2021046078 are incorporated in their entirety herein and present methods for encapsulating hydrophilic compounds in nanoparticles without charge complexation that instead rely on solubility-driven precipitation of water-soluble therapeutics including nucleic acids. This method of encapsulation is collectively termed “inverse Flash NanoPrecipitation” (iFNP) herein. In an embodiment of the iFNP process, a hydrophilic active agent referred to as the encapsulated agent—such as ribonucleic acid (RNA), deoxyribonucleic acid (DNA), oligonucleotides or other complex polynucleotide structures—and a stabilizing agent, such as a block copolymer, are dissolved in at least one polar solvent stream which is rapidly mixed with at least one non-polar antisolvent stream. Upon mixing, the encapsulated agent precipitates, forming the nanoparticle core. The hydrophilic region(s) of the stabilizing agent stick or adhere to and/or are within the nanoparticle core, and the hydrophobic region(s) of the stabilizing agent face the external non-polar antisolvent phase, for example, the hydrophobic region(s) of the stabilizing agent form a shell around the core, to form an inverse nanoparticle.

In an embodiment, the inverse nanoparticle is transferred into a water-miscible reforming solvent, and a second stabilizing agent is dissolved at a desired ratio. In a second mixing step, the second stabilizing agent sticks to, adheres to, and/or is in contact with the collapsed hydrophobic region of the first stabilizer agent. This forms a coated nanoparticle or coated nanocarrier with a hydrophilic active, such as a nucleic acid, in a hydrophilic core, surrounded by a hydrophobic shell, with a hydrophilic surface of the coating to prevent aggregation in aqueous conditions.

Markwalter et al. (Polymeric Nanocarrier Formulations of Biologics Using Inverse Flash NanoPrecipitation. The AAPS Journal (2020) Vol. 22) report encapsulation efficiency of RNA using this process as 48%; lipid nanoparticle methods may report encapsulation efficiencies greater than 80%.

A process of the invention enhances iFNP formulation properties through the incorporation of lipids; this is surprising and unanticipated.

An embodiment of the invention includes a coated nanoparticle composition including one or more encapsulated agents and a first stabilizing agent, with a surface formed by an additive agent or agents and a second stabilizing agent or agents. Compositions include but are not limited to an encapsulated agent or agents selected from nucleic acid classes including but not limited to RNA, DNA, mRNA (messenger RNA), siRNA (small interfering RNA), microRNA, circular RNA, antisense oligonucleotides, tRNA (transfer RNA), aptamers, DNA origami, or chemically-modified polynucleotides, including nucleic acid species with natural (e.g., from methylation or acetylation) or artificial substitutions, and these can be in circular or linear form or in complex with other molecules. The composition may include a salt, for example, a calcium salt, or a free acid form. The encapsulated agent may be more than one agent such as one or more of nucleic acids, peptides, proteins, or second or more nucleic acids. The encapsulated agent may be multiple nucleic acid sequences. In this text, the terms nanoparticle, nanocarrier, and NC are used interchangeably.

In an embodiment, a composition includes a first stabilizing agent that is a diblock, triblock, or comb copolymer. For example, the hydrophilic block(s) of the first stabilizing agent may be selected from dextran or other polysaccharides, poly(aspartic acid), or poly(glutamic acid). For example, the hydrophobic block(s) may be selected from poly(lactic acid), poly(lactic-co-glycolic acid), or poly(caprolactone).

In an embodiment, a composition includes additive agents selected from lipids, phospholipids, ethyl oleate, POPC (1-palmitoyl-2-oleoyl-sn-glycero-3-phosphocholine), DSPC (1,2-distearoyl-s-glycero-3-phosphocholine), hydrogenated soybean phosphatidylcholine (HSPC), DOPC (1,2-dioleoyl-sn-glycero-3-phosphocholine), DOPE (1,2-dioleoyl-sn-glycero-3-phosphoethanolamine), cationic lipids or cationic ionizable lipids, cholesterol, beta-sitosterol, fatty acid methyl esters, fatty acid ethyl esters, hydrophobic polymers, or cationic or ionizable polymers, or combinations.

Compositions include a second amphiphilic stabilizing agent that is a diblock, triblock, or comb copolymer, or a lipid conjugate, or mixtures. The hydrophilic region of the polymeric or lipid conjugate second stabilizing agent can be selected from poly(ethylene glycol), poly(sarcosine), poly(aspartic acid), poly(glutamic acid), poly(lysine), or poly(arginine), or combinations. The hydrophobic polymer block can be selected from poly(lactic acid), poly(lactic-co-glycolic acid), poly(caprolactone). Compositions include PEG (polyethylene glycol, polyoxyethylene, poly(ethylene oxide)) lipids such as PEG-DMG (1-(monomethoxy-polyethyleneglycol)-2,3-dimyristoylglycerol) or PEG-DSPE (1,2-distearoyl-sn-glycero-3-phosphoethanolamine-N-[amino(polyethylene glycol)-2000]), or PEG-b-PLGA (polyethylene glycol-block-poly(lactic-co-glycolic acid)) or PEG-b-PLA (polyethylene glycol-block-poly(lactic acid)), or mixtures.

A process of the invention is for preparing the coated nanocarrier composition involving forming an inverse nanocarrier using inverse Flash NanoPrecipitation, exchanging the inverse nanocarrier into a reforming solvent along with additive agents and the second stabilizing agent, and assembling the coating using Flash NanoPrecipitation. The coated nanocarrier may then be further processed into a pharmaceutical form.

The inverse nanocarrier is produced by inverse Flash NanoPrecipitation. One or more solvent streams containing the dissolved encapsulated agent or agents and the first stabilizing agent is rapidly micromixed with an antisolvent stream or streams, optionally containing a divalent cation such as calcium. This mixing process drives precipitation of the encapsulated agent or agents which is then stabilized from further growth by the stabilizing agent. The micromixing process is carried out using mixers such as the confined impinging jet mixer or the multi-inlet vortex mixer. A representative solvent would be dimethylsulfoxide (DMSO) and a representative antisolvent would be dichloromethane (DCM).

The inverse nanocarrier is then combined with the additive agent or agents and the second stabilizing agent or agents at a specified or desired composition. A solvent exchange process, such as distillation, is employed to remove the antisolvent and introduce the reforming solvent, for example, acetonitrile.

The inverse nanocarrier and additive agent(s) and second stabilizing agent(s) in the reforming solvent are then rapidly micromixed with an aqueous antisolvent using a mixer as described above. The resulting nanoparticle is a coated nanocarrier. Further processing steps common to the field, such as buffer exchange by ultrafiltration or diafiltration, can be employed to remove residual reforming solvent and prepare the pharmaceutical composition.

In an embodiment of the invention, a nanoparticle overcomes barriers to RNA and DNA delivery by encapsulating the nucleic acid, aiding endocytic internalization, and providing an endosomal escape mechanism for cytosolic delivery. A cationic ionizable lipid can enable both encapsulation, driven by charge complexation with anionic nucleic acids under acidic conditions, and endosomal escape for delivery to the cytosol. An encapsulation process that does not require charge complexation of the nucleic acid may allow for flexibility in nanoparticle design.

Methods of the invention provide for delivering a therapeutic and/or prophylactic to a cell or organ. Delivery of a therapeutic and/or prophylactic to a cell involves administering a coated nanocarrier pharmaceutical composition including the encapsulated agent to a subject, where administration of the composition involves contacting the cell with the composition. Coated nanocarrier compositions and/or pharmaceutical compositions including one or more coated nanocarrier compositions may be administered to any patient or subject, including those patients or subjects that may benefit from a therapeutic effect provided by the delivery of an encapsulated agent or agents to one or more particular cells, tissues, organs, or systems or groups thereof, such as the hepatic system.

The step of contacting a cell, such as a mammalian cell, with a coated nanocarrier may be performed in vivo, ex vivo, in culture, or in vitro. In certain embodiments, an mRNA as the encapsulated agent in a coated nanocarrier may encode a recombinant polypeptide that may replace one or more polypeptides that may be reduced or substantially absent in a cell contacted with the nanoparticle composition. The one or more substantially absent polypeptides may be lacking due to a genetic mutation of the encoding gene or a regulatory pathway thereof. In certain embodiments, one or more nucleic acids including an interfering RNA sequence (e.g., siRNA) may be encapsulated in the coated nanocarrier to provide a method for treatment in vitro and in vivo of a disease or disorder in a mammal by downregulating or silencing the transcription and/or translation of one or more target nucleic acid sequences or genes of interest. In certain embodiments, an mRNA sequence encoding an endonuclease and a small guide RNA may be the encapsulated agents in a coated nanocarrier to provide a method for treatment in vitro and in vivo by gene editing.

In an embodiment of the invention, a nanoparticle includes a core including a more polar region of a first stabilizing amphiphilic copolymer and at least one water soluble agent and a shell including a less polar region of the first stabilizing amphiphilic copolymer, at least one lipid, and a second stabilizing amphiphilic agent. A shell surrounds the core. The shell includes an interior surface and an exterior surface. The interior surface of the shell is in contact with the core. The second stabilizing amphiphilic agent includes a more polar region and a less polar region. The more polar region of the second stabilizing amphiphilic agent is at the exterior surface of the shell. The shell includes the less polar region of the second stabilizing amphiphilic agent. The at least one water soluble agent is not at the exterior surface of the shell. The corona surrounds the shell, and the corona includes the more polar region of the second stabilizing amphiphilic agent. The at least one water soluble agent is not in contact with the more polar region of the second stabilizing amphiphilic agent, and the corona does not include the at least one water soluble agent.

In an embodiment, the at least one water soluble agent is selected from the group consisting of a nucleic acid, a polynucleic acid, ribonucleic acid (RNA), messenger ribonucleic acid (mRNA), self-amplifying messenger ribonucleic acid (saRNA), small interfering ribonucleic acid (siRNA), micro ribonucleic acid (microRNA), circular ribonucleic acid (circular RNA), transfer ribonucleic acid (tRNA), small guide ribonucleic acid (sgRNA), deoxyribonucleic acid (DNA), an antisense oligonucleotide (ASO), a plasmid, an episome, and combinations.

In an embodiment, the at least one water soluble agent includes mRNA encoding SARS-CoV-2 spike protein receptor binding domain (RBD), optionally with pseudo-uridine (C5-glycoside isomer of uridine, 5-ribosyluracil) modification.

The first stabilizing amphiphilic copolymer can be poly(aspartic acid)-block-poly(lactic acid) (PAsp-b-PLA), poly(aspartic acid)-block-poly(lactic-co-glycolic acid) (PAsp-b-PLGA), dextran-poly(lactic acid) (Dex-PLA), or dextran-poly(lactic-co-glycolic acid) (Dex-PLGA). The dextran and poly(aspartic acid) each can have a molecular weight within a range of from about 500 to about 500,000 Da, from about 500 to about 50,000 Da, or from about 750 Da to about 20,000 Da, and the poly(lactic acid) and poly(lactic-co-glycolic acid) each can have a molecular weight within a range of from about 500 to about 500,000 Da, from about 500 to about 50,000 Da, or from about 750 Da to about 20,000 Da.

The first stabilizing amphiphilic copolymer can be poly(glutamic acid)-block-poly(lactic acid) (PGlu-b-PLA), poly(glutamic acid)-block-poly(lactic-co-glycolic acid) (PGlu-b-PLGA), poly(glutamic acid)-block-poly(caprolactone) (PGlu-b-PCL), poly(aspartic acid)-block-poly(caprolactone) (PAsp-b-PCL), or dextran-poly(caprolactone) (Dex-PCL). The dextran, poly(aspartic acid), and poly(glutamic acid) each can have a molecular weight within a range of from about 500 to about 500,000 Da, from about 500 to about 50,000 Da, or from about 750 Da to about 20,000 Da, and the poly(lactic acid), poly(lactic-co-glycolic acid), and poly(caprolactone) each can have a molecular weight within a range of from about 500 to about 500,000 Da, from about 500 to about 50,000 Da, or from about 750 Da to about 20,000 Da.

The at least one lipid can be a phospholipid, a cationic lipid, an anionic lipid, a sterol, a monoglyceride, a triglyceride, a fatty acid methyl ester, a fatty acid ethyl ester, or a combination.

The at least one lipid can be 1,2-distearoyl-s-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1,2-di-O-octadecenyl-sn-glycero-3-phosphocholine (18:0 Diether PC), 1-oleoyl-2-cholesterylhemisuccinoyl-sn-glycero-3-phosphocholine (OchemsPC), 1-hexadecyl-sn-glycero-3-phosphocholine (Cl 6 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), dipalmitoylphosphatidylglycerol (DPPG), palmitoyloleoylphosphatidylethanolamine (POPE), distearoyl-phosphatidyl-ethanolamine (DSPE), dipalmitoyl phosphatidyl ethanolamine (DPPE), dimyristoylphosphoethanolamine (DMPE), 1-stearoyl-2-oleoyl-phosphatidy ethanolamine (SOPE), 1-stearoyl-2-oleoyl-phosphatidylcholine (SOPC), sphingomyelin, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine (LPE), or a combination.

The at least one lipid can include 1-palmitoyl-2-oleoyl-sn-glycero-3-phosphocholine (POPC). The at least one lipid can include a cationic lipid and/or an ionizable cationic lipid. The at least one lipid can be a blend of a cationic lipid, a phospholipid, and a cholesterol or a sterol.

The second stabilizing agent can be 1-(monomethoxy-polyethyleneglycol)-2,3-dimyristoylglycerol (PEG-DMG), pegylated distearoyl-phosphatidyl-ethanolamine (PEG-DSPE), polyethyleneglycol-block-poly(lactic acid) (PEG-b-PLA), polyethyleneglycol-block-poly(lactic-co-glycolic acid) (PEG-b-PLGA), or polyethyleneglycol-block-poly(caprolactone) (PEG-b-PCL). The poly(lactic acid), poly(lactic-co-glycolic acid), and poly(caprolactone) each can have a molecular weight within a range of from about 500 to about 500,000 Da, from about 500 to about 50,000 Da, or from about 750 Da to about 20,000 Da.

The nanoparticle can include at least one hydrophobic polymer. The at least one hydrophobic polymer can be poly(lactic acid) (PLA), poly(lactic-co-glycolic acid) (PLGA), polycaprolactone (PCL), or a combination. The poly(lactic acid), poly(lactic-co-glycolic acid), and poly(caprolactone) each can have a molecular weight within a range of from about 500 to about 500,000 Da, from about 500 to about 50,000 Da, or from about 750 Da to about 20,000 Da.

In an embodiment, the at least one water soluble agent includes mRNA encoding SARS-CoV-2 spike protein receptor binding domain (RBD) and/or mRNA encoding SARS-CoV-2 spike protein receptor binding domain (RBD) with pseudo-uridine (C5-glycoside isomer of uridine, 5-ribosyluracil) modification, the first stabilizing amphiphilic copolymer includes dextran-poly(lactic acid) (Dex-PLA), dextran-poly(lactic-co-glycolic acid) (Dex-PLGA) and/or dextran-poly(caprolactone) (Dex-PCL), the at least one lipid includes a cationic lipid, and the second stabilizing amphiphilic agent includes a polyethylene glycol (PEG) copolymer and/or a polyethylene glycol (PEG) lipid.

In an embodiment, the at least one water soluble agent includes mRNA encoding SARS-CoV-2 spike protein receptor binding domain (RBD) with pseudo-uridine (C5-glycoside isomer of uridine, 5-ribosyluracil) modification, the first stabilizing amphiphilic copolymer includes dextran-poly(lactic-co-glycolic acid) (Dex-PLGA), the at least one lipid includes 8-[(2-hydroxyethyl)[6-oxo-6-(undecyloxy)hexyl]amino]-octanoic acid, 1-octylnonyl ester (SM-102), 1-palmitoyl-2-oleoyl-glycero-3-phosphocholine (POPC), and cholesterol, and the second stabilizing amphiphilic agent includes 1,2-dimyristoyl-rac-glycero-3-methoxypolyethylene glycol-2000 (PEG-DMG).

In an embodiment, a pharmaceutical composition includes a therapeutically effective amount of the nanoparticle and a pharmaceutical acceptable carrier or diluent.

A method of administration to a subject includes administering to the subject a therapeutically effective amount of the nanoparticle.

A method of administration to a cell, includes contacting the cell with the nanoparticle.

The cell can be a mammalian cell or a human cell. The administration to the cell can be performed in vitro.

A method for preventing or treating an infectious disease includes administering a therapeutically effective amount of the nanoparticle to a subject suffering from the infectious disease. The at least one water soluble agent induces production of an antigen associated with the infectious disease by a cell of the subject, and the antigen induces an immune response by the subject to the infectious disease.

The nanoparticle can be used in the prevention or treatment of an infectious disease. The nanoparticle can be used in the manufacture of a medicament for the prevention or treatment of an infectious disease.

The infectious disease can be a virus. The infectious disease can be an adenovirus, Herpes simplex type 1, Herpes simplex type 2; encephalitis virus, papillomavirus, Varicella-zoster virus, Epstein-barr virus, Human cytomegalovirus, Human herpes virus type 8, Human papillomavirus, BK virus, JC virus, Smallpox, polio virus, Hepatitis B virus, Human bocavirus, Parvovirus B19, Human astrovirus, Norwalk virus, coxsackievirus, hepatitis A virus, poliovirus, rhinovirus, Hepatitis C virus, Yellow Fever virus, Dengue virus, West Nile virus, Rubella virus, Hepatitis E virus, Human Immunodeficiency virus (HIV), Guanarito virus, Junin virus, Lassa virus, Machupo virus, Sabia virus, Crimean-Congo hemorrhagic fever virus, Ebola virus, Marburg virus, Measles virus, Mumps virus, Parainfluenza virus, Respiratory syncytial virus, Human metapneumovirus, Hendra virus, Nipah virus, Hepatitis D, Rotavirus, Orbivirus, Coltivirus, Banna virus, Human Enterovirus, Hanta virus, West Nile virus, Japanese encephalitis virus, Vesicular exanthernavirus, or Eastern equine encephalitis. The infectious disease can be a virus, a coronavirus, Middle East Respiratory Syndrome Corona Virus, Severe acute respiratory syndrome virus, SARS-CoV-2, rabies virus, influenza, Zika virus, cytomegalovirus, or Chikungunya virus.

A method for preventing or treating a cancer includes administering a therapeutically effective amount of the nanoparticle to a subject suffering from the cancer. The at least one water soluble agent induces production by a cell of the subject of a tumor antigen associated with cancerous cells of the cancer, and the antigen induces an immune response to the cancer by the subject.

The nanoparticle of claim 1 can be used in the prevention or treatment of a cancer. The nanoparticle can be used in the manufacture of a medicament for the prevention or treatment of a cancer.

A method for preventing or treating a loss-of-function disease includes administering a therapeutically effective amount of the nanoparticle to a subject suffering from the loss-of-function disease. The at least one water soluble agent induces production of a protein that restores the lost function.

The nanoparticle can be used in the prevention or treatment of a loss-of-function disease. The nanoparticle can be used in the manufacture of a medicament for the prevention or treatment of a loss-of-function disease.

The loss-of-function disease can be a urea cycle disorder, N-acetylglutamate synthase (NAGS) deficiency, carbamoyl phosphate synthetase (CPS) deficiency, ornithine transcarbamoylase (OTC) deficiency, Citrullinemia Type 1 (CTLN1), Citrullinemia Type 2 (CTLN2), Argininosuccinic aciduria, Argininemia, or Hyperornithinemia-Hyperammonemia-Homocitrullinuria (H1H) syndrome. The loss-of-function disease can be a polygenic disorder, a monogenic disorder, a polygenic liver disorder, or a monogenic liver disorder.

A method for preventing or treating a disease associated with a premature stop codon includes administering a therapeutically effective amount of the nanoparticle to a subject suffering from the disease associated with the premature stop codon. The at least one water soluble agent includes tRNA, and the tRNA enables translation through a premature stop codon.

The nanoparticle can be used in the prevention or treatment of a disease associated with a premature stop codon. The nanoparticle can be used in the manufacture of a medicament for the prevention or treatment of a disease associated with a premature stop codon.

The disease associated with a premature stop codon can be beta-thalassemia or Charcot-Marie-Tooth disease.

A method for gene editing includes contacting the nanoparticle with a cell, so that a DNA sequence is inserted into a genome of the cell. The at least one water soluble agent includes an endonuclease and/or an mRNA encoding an endonuclease, a small guide RNA (sgRNA), and the DNA sequence. The nanoparticle can be contacted with the cell in vitro. The endonuclease can be selected from the group consisting of a Cas protein, Cas9, and a TALEN.

The nanoparticle can be used in gene editing. The nanoparticle can be used in the manufacture of a medicament for gene editing.

A method for producing a nanoparticle includes dissolving at least one water soluble agent in a first polar process solvent to form a water soluble agent solution, dissolving a first stabilizing amphiphilic copolymer in a second polar process solvent to form a copolymer solution, continuously mixing the water soluble agent solution and the copolymer solution with an antisolvent to form a mixed solution from which nanoparticles assemble to form an inverse nanoparticle dispersion, adding at least one lipid to the inverse nanoparticle dispersion, adding a second stabilizing amphiphilic agent to the inverse nanoparticle dispersion, combining the inverse nanoparticle dispersion with a reforming solvent to form a reforming dispersion, and continuously mixing the reforming dispersion with an aqueous solvent to form the nanoparticle. The first stabilizing amphiphilic copolymer includes at least one region that is more polar and at least one region that is less polar; the second polar process solvent can be the same as or different from the first polar process solvent; the antisolvent is less polar than the first polar process solvent; and the antisolvent is less polar than the second polar process solvent. The nanoparticle includes a core and a shell; the core includes the more polar region of the first stabilizing amphiphilic copolymer and the at least one water soluble agent; and the shell includes the less polar region of the first stabilizing amphiphilic copolymer.

The aqueous solvent can be an aqueous buffer. The second polar process solvent can be the same as the first polar process solvent. The water soluble agent solution and the copolymer solution can be a single mixed solution.

The second polar process solvent can be different from the first polar process solvent.

In an embodiment, the shell includes an interior surface and an exterior surface, the interior surface of the shell is in contact with the core, the second stabilizing amphiphilic agent includes a more polar region and a less polar region, and the more polar region of the second stabilizing amphiphilic agent is at the exterior surface of the shell. The shell includes the less polar region of the second stabilizing amphiphilic agent. The at least one water soluble agent is not at the exterior surface of the shell. A corona surrounds the shell, and the corona includes the more polar region of the second stabilizing amphiphilic agent. The at least one water soluble agent is not in contact with the more polar region of the second stabilizing amphiphilic agent, and the corona does not include the at least one water soluble agent.

The reforming solvent can be acetonitrile.

The at least one water soluble agent can be a nucleic acid, a polynucleic acid, ribonucleic acid (RNA), messenger ribonucleic acid (mRNA), self-amplifying messenger ribonucleic acid (saRNA), small interfering ribonucleic acid (siRNA), micro ribonucleic acid (microRNA), circular ribonucleic acid (circular RNA), transfer ribonucleic acid (tRNA), small guide ribonucleic acid (sgRNA), deoxyribonucleic acid (DNA), an antisense oligonucleotide (ASO), a plasmid, an episome, or a combination.

The at least one water soluble agent can include mRNA encoding SARS-CoV-2 spike protein receptor binding domain (RBD), optionally with pseudo-uridine (C5-glycoside isomer of uridine, 5-ribosyluracil) modification.

The first stabilizing amphiphilic copolymer can be poly(aspartic acid)-block-poly(lactic acid) (PAsp-b-PLA), poly(aspartic acid)-block-poly(lactic-co-glycolic acid) (PAsp-b-PLGA), dextran-poly(lactic acid) (Dex-PLA), dextran-poly(lactic-co-glycolic acid) (Dex-PLGA), poly(glutamic acid)-block-poly(lactic acid) (PGlu-b-PLA), poly(glutamic acid)-block-poly(lactic-co-glycolic acid) (PGlu-b-PLGA), poly(glutamic acid)-block-poly(caprolactone) (PGlu-b-PCL), poly(aspartic acid)-block-poly(caprolactone) (PAsp-b-PCL), or dextran-poly(caprolactone) (Dex-PCL). The dextran, poly(aspartic acid), and poly(glutamic acid) each can have a molecular weight within a range of from about 500 to about 500,000 Da, from about 500 to about 50,000 Da, or from about 750 Da to about 20,000 Da, and the poly(lactic acid), poly(lactic-co-glycolic acid), and poly(caprolactone) each can have a molecular weight within a range of from about 500 to about 500,000 Da, from about 500 to about 50,000 Da, or from about 750 Da to about 20,000 Da.

The first stabilizing amphiphilic copolymer can be poly(glutamic acid)-block-poly(lactic acid) (PGlu-b-PLA) or poly(glutamic acid)-block-poly(lactic-co-glycolic acid) (PGlu-b-PLGA). The poly(glutamic acid) can have a molecular weight within a range of from about 500 to about 500,000 Da, from about 500 to about 50,000 Da, or from about 750 Da to about 20,000 Da, and the poly(lactic acid) and poly(lactic-co-glycolic acid) each can have a molecular weight within a range of from about 500 to about 500,000 Da, from about 500 to about 50,000 Da, or from about 750 Da to about 20,000 Da.

The at least one lipid can be a phospholipid, a cationic lipid, an anionic lipid, a sterol, a monoglyceride, a triglyceride, a fatty acid methyl ester, a fatty acid ethyl ester, or a combination. The at least one lipid can include a cationic lipid and/or 1-palmitoyl-2-oleoyl-sn-glycero-3-phosphocholine (POPC). The at least one lipid can be a blend of a cationic lipid, a phospholipid, and a cholesterol or a sterol.

The second stabilizing amphiphilic agent can be pegylated 1-(monomethoxy-polyethyleneglycol)-2,3-dimyristoylglycerol (PEG-DMG), pegylated distearoyl-phosphatidyl-ethanolamine (PEG-DSPE), polyethyleneglycol-block-poly(lactic acid) (PEG-b-PLA), polyethyleneglycol-block-poly(lactic-co-glycolic acid) (PEG-b-PLGA), or polyethyleneglycol-block-poly(caprolactone) (PEG-b-PCL). The poly(lactic acid), poly(lactic-co-glycolic acid), and poly(caprolactone) each can have a molecular weight within a range of from about 500 to about 500,000 Da, from about 500 to about 50,000 Da, or from about 750 Da to about 20,000 Da.

In an embodiment, the at least one water soluble agent includes mRNA encoding SARS-CoV-2 spike protein receptor binding domain (RBD) and/or mRNA encoding SARS-CoV-2 spike protein receptor binding domain (RBD) with pseudo-uridine (C5-glycoside isomer of uridine, 5-ribosyluracil) modification, the first stabilizing amphiphilic copolymer includes dextran-poly(lactic acid) (Dex-PLA), dextran-poly(lactic-co-glycolic acid) (Dex-PLGA) and/or dextran-poly(caprolactone) (Dex-PCL), the at least one lipid includes a cationic lipid, and the second stabilizing amphiphilic agent includes a polyethylene glycol (PEG) copolymer and/or a polyethylene glycol (PEG) lipid.

In an embodiment, the at least one water soluble agent includes mRNA encoding SARS-CoV-2 spike protein receptor binding domain (RBD) with pseudo-uridine (C5-glycoside isomer of uridine, 5-ribosyluracil) modification, the first stabilizing amphiphilic copolymer includes dextran-poly(lactic-co-glycolic acid) (Dex-PLGA), the at least one lipid includes 8-[(2-hydroxyethyl)[6-oxo-6-(undecyloxy)hexyl]amino]-octanoic acid, 1-octylnonyl ester (SM-102), 1-palmitoyl-2-oleoyl-glycero-3-phosphocholine (POPC), and cholesterol, and the second stabilizing amphiphilic agent includes 1,2-dimyristoyl-rac-glycero-3-methoxypolyethylene glycol-2000 (PEG-DMG).

In an embodiment, the at least one water soluble agent includes a ribonucleic acid (RNA) of which at least one uridine is replaced with a modified uridine. At least 50% of the uridines of the ribonucleic acid (RNA) each can be replaced with a modified uridine, and the modified uridines may be the same or different. All of the uridines of the ribonucleic acid (RNA) can be replaced with modified uridines, and the modified uridines may be the same or different. For example, each modified uridine is independently selected from the group consisting of pseudouridine, 5-methoxyuridine, and N1-methylpseudouridine. For example, each modified uridine is independently selected from the group consisting of pseudouridine and 5-methoxyuridine.

The ribonucleic acid (RNA) can be messenger ribonucleic acid (mRNA), self-amplifying messenger ribonucleic acid (saRNA), small interfering ribonucleic acid (siRNA), micro ribonucleic acid (microRNA), circular ribonucleic acid (circular RNA), transfer ribonucleic acid (tRNA), small guide ribonucleic acid (sgRNA), or an antisense oligonucleotide ribonucleic acid (ASO RNA).

The at least one water soluble agent can be a nucleic acid that is at least partially neutralized with a base. The nucleic acid can be 50% neutralized with a base. The nucleic acid can be fully neutralized with a base. The nucleic acid can be a ribonucleic acid (RNA). The nucleic acid can be messenger ribonucleic acid (mRNA), self-amplifying messenger ribonucleic acid (saRNA), small interfering ribonucleic acid (siRNA), micro ribonucleic acid (microRNA), circular ribonucleic acid (circular RNA), transfer ribonucleic acid (tRNA), small guide ribonucleic acid (sgRNA), or an antisense oligonucleotide ribonucleic acid (ASO RNA). The base can be an amine. The base can be guanidine. The base can be a guanidine derivative or a guanidinium salt. The base can be arginine. The base can be a tertiary amine. The base can be triethylamine or diphenhydramine.

In a method, the at least one water soluble agent includes a ribonucleic acid (RNA) of which at least one uridine is replaced with a modified uridine. At least 50% of the uridines of the ribonucleic acid (RNA) each can be replaced with a modified uridine, and the modified uridines may be the same or different. All of the uridines of the ribonucleic acid (RNA) can be replaced with a modified uridine, and the modified uridines may be the same or different. For example, each modified uridine is independently selected from the group consisting of pseudouridine, 5-methoxyuridine, and N1-methylpseudouridine. For example, each modified uridine is independently selected from the group consisting of pseudouridine and 5-methoxyuridine.

The ribonucleic acid (RNA) can be messenger ribonucleic acid (mRNA), self-amplifying messenger ribonucleic acid (saRNA), small interfering ribonucleic acid (siRNA), micro ribonucleic acid (microRNA), circular ribonucleic acid (circular RNA), transfer ribonucleic acid (tRNA), small guide ribonucleic acid (sgRNA), or an antisense oligonucleotide ribonucleic acid (ASO RNA).

The at least one water soluble agent can be a nucleic acid that is at least partially neutralized with a base prior to dissolving the at least one water soluble agent in the first polar process solvent. The nucleic acid can be 50% neutralized with a base. The nucleic acid can be fully neutralized with a base. The nucleic acid can be a ribonucleic acid (RNA). The nucleic acid can be messenger ribonucleic acid (mRNA), self-amplifying messenger ribonucleic acid (saRNA), small interfering ribonucleic acid (siRNA), micro ribonucleic acid (microRNA), circular ribonucleic acid (circular RNA), transfer ribonucleic acid (tRNA), small guide ribonucleic acid (sgRNA), or an antisense oligonucleotide ribonucleic acid (ASO RNA). The base can be an amine, guanidine, a guanidine derivative, a guanidinium salt, arginine, a tertiary amine, triethylamine, or diphenhydramine.

In a method, the at least one water soluble agent is a salt of a plasmid. The at least one water soluble agent can be a triethylamine (TEA) salt of the plasmid or a guanidine salt of the plasmid. The plasmid can be an episome.

In a method, the first stabilizing amphiphilic copolymer can be a polysaccharide copolymer. The first stabilizing amphiphilic copolymer can be dextran-poly(lactic-co-glycolic acid) (Dex-PLGA). The dextran can have a molecular weight within a range of from about 500 to about 500,000 Da, from about 500 to about 50,000 Da, or from about 750 Da to about 20,000 Da, and the poly(lactic-co-glycolic acid) can have a molecular weight within a range of from about 500 to about 500,000 Da, from about 500 to about 50,000 Da, or from about 750 Da to about 20,000 Da.

In a method, the first stabilizing amphiphilic copolymer can be a polypeptide copolymer. The first stabilizing amphiphilic copolymer can be poly(aspartic acid)-poly(lactic-co-glycolic acid) (PAsp-PLGA). The poly(aspartic acid) can have a molecular weight within a range of from about 500 to about 500,000 Da, from about 500 to about 50,000 Da, or from about 750 Da to about 20,000 Da, and the poly(lactic-co-glycolic acid) can have a molecular weight within a range of from about 500 to about 500,000 Da, from about 500 to about 50,000 Da, or from about 750 Da to about 20,000 Da.

In a method, the at least one lipid can be a cationic lipid, 1-palmitoyl-2-oleoyl-sn-glycero-3-phosphocholine (POPC), cholesterol, or a combination.

In a method, the second amphiphilic stabilizing copolymer can be a pegylated copolymer. The second amphiphilic stabilizing copolymer can be 1-(monomethoxy-polyethyleneglycol)-2,3-dimyristoylglycerol (PEG-DMG).

In a method, the antisolvent includes a metal salt. The metal salt can be MgCl₂, CaCl₂), or ZnCl₂.

### BRIEF DESCRIPTION OF THE DRAWINGS

FIG. 1: Schematic of nanocarrier structure during processing steps (not to scale).

FIG. 2: Expression at 24 hours in 293T cell culture, measured by fluorescence intensity from flow cytometry analysis. The formulation identification (ID) corresponds to the formulation details provided in the Examples section. The bars are for each RNA dose applied in the cell supernatant.

FIG. 3: Expression at (A) 24 hours and (B) 60 hours in 293T cell culture, measured by fluorescence intensity from flow cytometry analysis. The formulation ID corresponds to the formulation details provided in the Examples section. The x axis indicates the RNA dose applied in the cell supernatant.

FIG. 4: Expression at (A) 24 hours and (B) 60 hours in 293T cell culture, measured by fluorescence intensity from flow cytometry analysis. The formulation ID corresponds to the formulation details provided in the Examples section. The x axis indicates the RNA dose applied in the cell supernatant.

FIG. 5A: Flow cytometry histograms for the indicated formulations showing similar expression between an iFNP and lipid nanoparticles (LNP) formulation at 1 μg/mL RNA dose. The control formulation (mRNA-55) without mRNA was significantly less fluorescent.

FIG. 5B: Epifluorescence imaging and brightfield of the formulations after 24 hours incubation with 293T cells.

FIG. 5C: Expression at 24 hours of GFP (green fluorescent protein) in 293T cell culture measured by fluorescence intensity from flow cytometry analysis. The y-axis shows MFI (median fluorescence intensity) of the GFP.

FIG. 6A: Expression in 293T cell culture measured by fluorescence intensity from flow cytometry analysis at 24 hours; the formulation ID corresponds to the formulation details provided in the examples section; the x axis indicates RNA dose applied in the cell supernatant.

FIG. 6B: Expression in HepG2 cell culture, measured by fluorescence intensity from flow cytometry analysis at 24 hours; the formulation ID corresponds to the formulation details provided in the examples section; the x axis indicates RNA dose applied in the cell supernatant.

FIG. 7A: Expression in 293T cell culture, measured by fluorescence intensity from flow cytometry analysis at 24 hours; the formulation ID corresponds to the formulation details provided in the examples section; the x axis indicates RNA dose applied in the cell supernatant. (C) HepG2 data presented as mean instead of median fluorescence intensity.

FIG. 7B: Expression in HepG2 cell culture, measured by fluorescence intensity from flow cytometry analysis at 24 hours; the formulation ID corresponds to the formulation details provided in the examples section; the x axis indicates RNA dose applied in the cell supernatant.

FIG. 7C: HepG2 data presented as mean instead of median fluorescence intensity.

FIG. 8A: Expression in 293T cell culture, measured by fluorescence intensity from flow cytometry analysis at 24 hours; the formulation ID corresponds to the formulation details provided in the examples section; the x axis indicates RNA dose applied in the cell supernatant.

FIG. 8B: HepG2 cell culture, measured by fluorescence intensity from flow cytometry analysis at 24 hours; the formulation ID corresponds to the formulation details provided in the examples section; the x axis indicates RNA dose applied in the cell supernatant.

FIG. 9A: Expression in 293T cell culture, measured by fluorescence intensity from flow cytometry analysis at 24 hours; the formulation ID corresponds to the formulation details provided in the examples section; the x axis indicates RNA dose applied in the cell supernatant.

FIG. 9B: HepG2 cell culture, measured by fluorescence intensity from flow cytometry analysis at 24 hours; the formulation ID corresponds to the formulation details provided in the examples section; the x axis indicates RNA dose applied in the cell supernatant.

FIG. 10A: Expression in 293T, measured by fluorescence intensity from flow cytometry analysis at 24 hours; the formulation ID corresponds to the formulation details provided in the examples section; the x axis indicates RNA dose applied in the cell supernatant.

FIG. 10B: HepG2 cell culture, measured by fluorescence intensity from flow cytometry analysis at 24 hours; the formulation ID corresponds to the formulation details provided in the examples section; the x axis indicates RNA dose applied in the cell supernatant.

FIG. 11A: Expression at 24 hours in 293T cell culture before freezing the coated nanocarrier, measured by fluorescence intensity from flow cytometry analysis; the formulation ID corresponds to the formulation details provided in the Examples section; the bars indicate RNA dose applied in the cell supernatant.

FIG. 11B: Expression at 24 hours in 293T cell culture after freezing the coated nanocarrier, measured by fluorescence intensity from flow cytometry analysis; the formulation ID corresponds to the formulation details provided in the Examples section; the bars indicate RNA dose applied in the cell supernatant.

FIG. 11C: Expression at 24 hours in HepG2 cell culture before freezing the coated nanocarrier, measured by fluorescence intensity from flow cytometry analysis; the formulation ID corresponds to the formulation details provided in the Examples section; the bars indicate RNA dose applied in the cell supernatant.

FIG. 11D: Expression at 24 hours in HepG2 cell culture after freezing the coated nanocarrier, measured by fluorescence intensity from flow cytometry analysis; the formulation ID corresponds to the formulation details provided in the Examples section; the bars indicate RNA dose applied in the cell supernatant.

FIG. 12A: Expression at 24 hours in 293T cell culture after freezing the coated nanocarrier, measured by fluorescence intensity from flow cytometry analysis; the formulation ID corresponds to the formulation details provided in the examples section; the bars indicate RNA dose applied in the cell supernatant.

FIG. 12B: Expression at 24 hours in HepG2 cell culture after freezing the coated nanocarrier, measured by fluorescence intensity from flow cytometry analysis; the formulation ID corresponds to the formulation details provided in the examples section; the bars indicate RNA dose applied in the cell supernatant.

FIG. 13A: Geometric mean of SARS-CoV2 receptor binding domain detected by fluorescent antibody binding in a flow cytometry analysis of 293T cells transfected for 16 hours with either iFNP formulation, LNP formulation, DNA plasmid plus lipofectamine 2000, or no treatment; the dose ranges for the iFNP and LNP mRNA formulations are listed; detection was as described in the text.

FIG. 13B: Flow cytometry distribution data.

FIG. 14A: iFNP antibody titer against SARS-CoV2 receptor binding domain in mouse sera at the indicated timepoint during the immunization protocol; Log(Titer) is the endpoint titer determined by serial dilutions where the endpoint was defined as the luminescence signal two standard deviations above average background for the plate.

FIG. 14B: LNP antibody titer against SARS-CoV2 receptor binding domain in mouse sera at the indicated timepoint during the immunization protocol; Log(Titer) is the endpoint titer determined by serial dilutions where the endpoint was defined as the luminescence signal two standard deviations above average background for the plate.

FIG. 15A Green fluorescent protein (GFP) signal measured by flow cytometry after 24 hour culture in 293T culture; the median value is reported for two mRNA doses in each well.

FIG. 15B: Green fluorescent protein (GFP) signal measured by flow cytometry after 24 hour culture in HepG2 culture; the median value is reported for two mRNA doses in each well.

FIG. 16A: Firefly Luciferase signal after 24 hour incubation of nanoparticles with 293T cells; dose values are of mRNA in the culture media and detection used standard manufacturer recommendations as described in the text.

FIG. 16B: Firefly Luciferase signal after 24 hour incubation of nanoparticles with HepG2 cells; dose values are of mRNA in the culture media and detection used standard manufacturer recommendations as described in the text.

FIG. 17: Firefly Luciferase bioluminescence signal (whole animal total emission, ventral position) after intravenous (IV) injection.

FIG. 18: Firefly Luciferase bioluminescence signal (whole animal total emission, dorsal position) after IV injection of iFNP or LNP formulations.

FIG. 19: Firefly Luciferase bioluminescence signal (whole animal total emission, ventral position) after intramuscular (IM) injection in the hind limb of iFNP or LNP formulations.

FIG. 20: Firefly Luciferase bioluminescence signal (whole animal total emission, dorsal position) after IM injection in the hind limb of iFNP or LNP formulations.

FIG. 21: Firefly Luciferase bioluminescence images (IV—ventral, IM—dorsal) across imaging timepoints for iFNP (formulation—fL3) and LNP nanoparticles. Grey scale images show dark/light gradient splotches with elevated luciferase signal. Darker regions generally correspond to greater signal from luciferase and more concentrated expression of luciferase.

FIG. 22: Firefly Luciferase bioluminescence images at 9 hours post injection for LNP and iFNP (fL2 through fL7) formulations (“No Tx” indicates an untreated mouse as a control). Grey scale images show dark/light gradient splotches with elevated luciferase signal. IV injection produces liver/spleen signal and IM injection produces localized signal (with some liver signal developing with the LNP). Darker regions generally correspond to greater signal from luciferase and more concentrated expression of luciferase.

FIG. 23A: GFP signal measured by flow cytometry after 24 hour incubation in 293T culture; the median value is reported for two mRNA doses in each well.

FIG. 23B: GFP signal measured by flow cytometry after 24 hour incubation in HepG2 culture; the median value is reported for two mRNA doses in each well.

FIG. 24: Percent of GFP-positive 293T cells measured by flow cytometry at 96 and 140 hours post-treatment with iFNP DNA formulations AG1-AG6 at 3 μg/mL. GFP-positivity from iFNP formulated DNA is compared to the same time points after transfection with a Lipofectamine 2000 positive control or mock transfection negative control. The delivered DNA encodes GFP protein.

### DETAILED DESCRIPTION

Embodiments of the invention are discussed in detail below. In describing embodiments, specific terminology is employed for the sake of clarity. However, the invention is not intended to be limited to the specific terminology so selected. A person skilled in the relevant art will recognize that other equivalent parts can be employed and other methods developed without parting from the spirit and scope of the invention. All references cited herein are incorporated by reference as if each had been individually incorporated.

Definitions of several terms follow.

In this specification, solvent or solvent stream are terms used interchangeably to refer to an organic solvent, aqueous buffer, or mixture of the same, often with certain organic or inorganic compounds dissolved. “Stream” often, but not solely, is used in contexts where a continuous mixing or flow process is used or will be used. The properties of the solvent are dictating by the process as defined below.

In this specification, “antisolvent” or “antisolvent stream” are terms used interchangeably to refer to an organic solvent, aqueous buffer, or mixture of the same, often with certain organic or inorganic compounds dissolved. “Stream” often, but not solely, is used in contexts where a continuous mixing or flow process is used or will be used. The properties of the antisolvent are dictated by the process as defined below. In patents and manuscripts related to Flash NanoPrecipitation (FNP) or inverse Flash NanoPrecipitation (iFNP), the term “non-solvent” or “non-process solvent” may be used with the same intended meaning.

All solvents are miscible to some degree in each other. “Miscible” solvents as referred to herein are those that when mixed at the ratios used in the process would produce solutions that have no more than 20% of the volume of the minor phase not dissolved in the majority phase. “Immiscible” solvents as referred to herein are those that when mixed at the volume ratios used in the process produce a second phase with more than 20% of the minor phase not dissolved in the majority phase.

In this specification, the term “soluble” refers to the state of being molecularly dissolved within an indicated solvent or aqueous system. This requires that the solvent solubilize all regions, portions, or groups of the agent, species, polymer, or compound. Species that have precipitated, micellized, or formed collapsed polymer globules are not molecularly dissolved.

In this specification, the terms “species” and “agent” refer to compounds or molecules—organic or inorganic—that are included within the process.

In this specification, the terms “nanoparticles” (“NPs”), “particles”, and “nanocarriers” are used interchangeably, unless a distinction is indicated by the context. Particles in embodiments of the invention that have hydrophilic or more polar cores are at times referred to as “inverse nanocarriers”, to contrast them with nanocarriers that have hydrophobic or less polar cores. However, for the sake of brevity, when the context indicates that particles having hydrophilic or more polar cores according to an embodiment of the invention are being discussed, these may be simply referred to as “particles” or “nanoparticles”. “Coated nanocarriers” refers to “inverse nanocarriers” that have been further processed to have a surface permitting them to be dispersed in water without aggregation. Nanoparticles or nanocarriers typically have hydrodynamic mass average diameters as determined by dynamic light scattering to be between 10 nm and 800 nm.

The term “water-dispersed” refers to the property of being colloidally stable in a water environment such as an aqueous buffer. “Colloidal stability” refers to nanoparticles or other small particles that do not aggregate in the solvent. Colloidal stability may be imparted through a polymer brush that provides a steric barrier to nanoparticle aggregation, because it is solvated by the dispersing liquid, e.g. water.

The term “lipid” refers to a group of organic compounds that include, but are not limited to, esters of fatty acids and are characterized by being insoluble in water, but soluble in many organic solvents. They are usually divided into at least three classes: (1) “simple lipids”, which include fats and oils as well as waxes; (2) “compound lipids”, which include phospholipids and glycolipids; and (3) “derived lipids” or “structural lipids” such as steroids or sterols. A “phospholipid” is a lipid that includes a phosphate moiety and one or more carbon chains, such as unsaturated fatty acid carbon chains. A phospholipid may include one or more multiple (e.g., double or triple) bonds (e.g., one or more unsaturated bonds).

As used herein, “expression” of a nucleic acid sequence refers to the transcription from DNA into mRNA, the translation of an mRNA into a polypeptide or protein, and/or post-translational modification of a polypeptide or protein.

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism. The term “ex vivo” refers to events that occur outside of an organism

As used herein, the term “polypeptide” or “polypeptide of interest” refers to a polymer of amino acid residues typically joined by peptide bonds that can be produced naturally (e.g., isolated or purified) or synthetically. Polypeptides include proteins and enzymes.

As used herein, “targeted cells” refers to any one or more cells of interest. The cells may be found in vitro, in vivo, in situ, or in the tissue or organ of an organism. The organism may be an animal, such as a mammal or a human.

As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

As used herein, the term “immune response” refers to an integrated bodily response to an antigen or a cell expressing an antigen and may refer to a cellular immune response or a cellular as well as a humoral immune response. The immune response may be protective/preventive/prophylactic and/or therapeutic.

A “cellular immune response”, a “cellular response”, a “cellular response against an antigen”, or a similar term is meant to include a cellular response directed to cells expressing an antigen and being characterized by presentation of an antigen with class I or class II MHC. The cellular response relates to cells called T cells or T lymphocytes which act as either “helpers” or “killers”.

The term “antigen” includes any molecule, such as a peptide or protein, which includes at least one epitope that will elicit an immune response and/or against which an immune response is directed. An antigen in the context of an embodiment of the invention may be a molecule which, optionally after processing, induces an immune response, which is may be specific for the antigen or cells expressing the antigen. In particular, an “antigen” relates to a molecule which, optionally after processing, is presented by MHC (major histocompatibility complex) molecules and reacts specifically with T lymphocytes (T cells). An antigen may be a tumor antigen, i.e., a constituent of cancer cells such as a protein or peptide expressed in a cancer cell which may be derived from the cytoplasm, the cell surface or the cell nucleus, in particular those which primarily occur intracellularly or as surface antigens on cancer cells.

Lipid nanoparticles are non-viral formulations for the intracellular delivery of RNA therapeutics such as siRNA and mRNA (Cullis & Hope, Lipid Nanoparticle Systems for Enabling Gene Therapies. Molecular Therapy (2017) 25). Additional methods for RNA encapsulation include polyplexes and liposomes.

### Flash NanoPrecipitation (FNP)

Flash NanoPrecipitation (FNP) is a process that effects (combines) rapid micromixing in a confined geometry of miscible solvent and antisolvent streams to effect high supersaturation of components that are soluble in the solvent and insoluble in the antisolvent. The micromixing of can be effected in various geometries. In FNP high velocity inlet streams cause turbulent mixing that occurs in a central cavity. The time for solvent/antisolvent mixing is more rapid than the precipitation of the components. Although not intended to be limiting, two such geometries have been previously described and analyzed: the Confined Impinging Jet mixer (CIJ) (Johnson, B. K., Prud'homme, R. K. Chemical processing and micromixing in confined impinging jets. AIChE Journal 2003, 49, 2264-2282) and the multi-inlet vortex mixer (MIVM) (Liu, Y., Cheng, C., Liu, Y., Prud'homme, R. K., Fox, R. O. Mixing in a multi-inlet vortex mixer (MIVM) for flash nano-precipitation. Chemical Engineering Science 2008, 63, 2829-2842). These examples are meant to be illustrative rather than limiting or exhaustive.

A stabilization agent is incorporated into the solvent or antisolvent stream. An agent to be incorporated or encapsulated in the nanoparticle is dissolved in the solvent stream. The mixing of the solvent and antisolvent streams produces a composition where the species to be encapsulated is insoluble. The resulting high supersaturation results in rapid precipitation and growth of the nanoparticles. A stabilizing agent in the formulation accumulates on the surface of the nanoparticle and halts growth at a desired size. The process has been described in Process and apparatuses for preparing nanoparticle compositions with amphiphilic copolymers and their use, BK Johnson, R K Prud'homme, U.S. Pat. No. 8,137,699, 2012. It has further been described in the review article by Saad and Prud'homme. (See Saad, W. S. & Prud'homme, R. K., Principles of nanoparticle formation by flash nanoprecipitation. Nano Today 2016, 11 (2), 212-227). These documents and other documents mentioned in this text are incorporated into this application in their entirety. In Flash NanoPrecipitation the encapsulated agents are hydrophobic and the antisolvent (for example, an aqueous antisolvent) is more polar than the solvent.

The Flash NanoPrecipitation process involves a confined mixing volume having one or more solvent streams entering the mixing volume, one or more antisolvent streams entering the mixing volume, and an exit stream (leaving the mixing volume) for the process. The velocity of the inlet streams into the confined mixing volume can be between about 0.01 m/s and 100 m/s, or about 0.1 m/s and 50 m/s, or about 0.1 m/s and 10 m/s. The velocities of the streams may be equal to one another, or they may have different velocities. In the case of unequal velocities, the velocity of the highest velocity stream is the specified velocity.

Inverse Flash NanoPrecipitation (iFNP)

Inverse Flash NanoPrecipitation (iFNP) effects (combines) rapid micromixing in a confined geometry of miscible solvent and antisolvent streams to effect high supersaturation of components that are soluble in the solvent and insoluble in the antisolvent. The micromixing of can be effected in various geometries. In iFNP high velocity inlet streams cause turbulent mixing that occurs in a central cavity. The time for solvent/antisolvent mixing is more rapid than the precipitation of the components. In iFNP the encapsulated agents are hydrophilic, and the antisolvent is more non-polar than the solvent. The inverse Flash NanoPrecipitation process is described in: U.S. Pat. Nos. 10,231,937 and 11,103,461 and applications US20200206136 and US20210259984 and US20200023332 and WO2020227350 and WO2021046078; Pagels & Prud'homme, Polymeric nanoparticles and microparticles for the delivery of peptides, biologics, and soluble therapeutics. J Control Release (2015) vol. 219; Markwalter et al., Polymeric Nanocarrier Formulations of Biologics Using Inverse Flash NanoPrecipitation. The AAPS Journal (2020) Vol. 22; Markwalter et al., Sustained release of peptides and proteins from polymeric nanocarriers produced by inverse Flash NanoPrecipitation. Journal of Controlled Release (2021) Vol. 334. These documents are incorporated into this application in their entirety. In some cases the term “Flash NanoPrecipitation” is used to refer to inverse Flash Nanoprecipitation. However, it should be clear from the encapsulated material, process solvent, and antisolvent whether Flash NanoPrecipitation or inverse Flash NanoPrecipitation is being used.

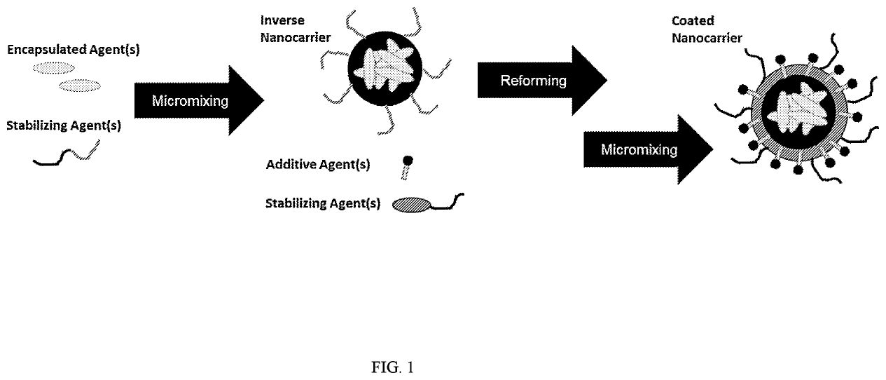

The inverse Flash NanoPrecipitation (iFNP) process can be used to create “inverse” nanocarriers with encapsulated hydrophilic agents, such as water-soluble peptides, proteins, and nucleic acids like RNA, DNA, antisense oligonucleotides, miRNA, siRNA, tRNA, plasmids, and nucleotides (FIG. 1). These inverse nanocarriers have a hydrophobic surface imparted by a first stabilizing agent. The iFNP process can also include processing steps to produce water-dispersible “coated nanocarriers” with a hydrophilic coating imparted by a second stabilizing agent. The additional process steps may include a solvent exchange to remove the non-polar antisolvent and introduce a water-miscible reforming solvent (if the antisolvent is not already water-miscible). Methods of the invention provide for incorporating stabilizing agents and lipid components during this reforming process. The inverse nanocarriers and additional components and stabilizing agents in the reforming solvent are then rapidly micromixed as in FNP using a confined mixing geometry to produce a water-dispersible “coated” nanocarrier. Thus, iFNP can refer to both the process step of producing an inverse nanocarrier and the series of steps required to produce a coated nanocarrier.

The process of producing an inverse nanocarrier by iFNP may utilizes an encapsulated agent, a stabilizing agent, a solvent stream, an antisolvent stream, and, optionally, salts.

### Encapsulated Agent

In some embodiments of the process, the encapsulated agent is, for example, a hydrophilic peptide, protein, or nucleic acid. In an embodiment of the invention the encapsulated agent is a polynucleic acid or nucleic acid such as an oligonucleotide, dinucleotide, ribonucleic acid (RNA), deoxyribonucleic acid (DNA), plasmid DNA, mRNA, hybrids thereof, RNAi-inducing (RNA interference-inducing) agents, short interfering RNA (siRNA), microRNA (miRNA), small hairpin RNA (shRNA), dicer substrate RNA (dsRNA), transfer RNA (tRNA), long non-coding RNA (lncRNA), ribozymes, catalytic DNA, aptamers, vectors, or an antisense oligonucleotide. The nucleic acid can be linear, looped, circular, double stranded, or single stranded. The mRNA may be self-amplifying mRNA. The mRNA may possess native nucleotides or be a modified mRNA containing non-natural nucleotides. An antisense oligonucleotide can possess a modified backbone, or include locked nucleic acids, or be a gapmer. Nucleic acids including different sequences can be co-encapsulated in the process.

A DNA or an mRNA may encode any polypeptide of interest, including any naturally or non-naturally occurring or otherwise modified polypeptide. A polypeptide encoded by an mRNA may be of any size and may have any secondary structure or activity. In some embodiments, a polypeptide encoded by a DNA or an mRNA may have a therapeutic effect when expressed in a cell. The DNA or mRNA may contain coding and non-coding flanking regions such as 5-UTR and 3-UTR (untranslated region).

An siRNA may be capable of selectively knocking down or down regulating expression of a gene of interest. For example, an siRNA could be selected to silence a gene associated with a particular disease, disorder, or condition upon administration to a subject in need thereof of a nanoparticle composition including the siRNA. An siRNA may include a sequence that is complementary to an mRNA sequence that encodes a gene or protein of interest. In some embodiments, the siRNA may be an immunomodulatory siRNA.

In other embodiments, a recombinant polypeptide produced by translation of mRNA may alter the DNA of gene expression in a cell through encoding various endonucleases and accessory factors. Polypeptides including CRISPR (clustered regularly interspaced short palindromic repeats) associated (Cas) proteins, transcription activator-like effector nucleases (TALENs), or other endonucleases may be desirable to change a subject's DNA to treat a disease. For example, an sgRNA-Cas9 complex can affect mRNA translation of cellular genes. In certain embodiments, a therapeutic and/or prophylactic is an sgRNA and/or Cas9 or other Cas protein variant, fragment, or subunit encoded by mRNA.

Nucleic acids and polynucleotides may include one or more naturally occurring components (either as ribonucleic or deoxyribonucleic acid), including any of the canonical nucleotides A (adenosine), G (guanosine), C (cytosine), U (uridine), or T (thymidine). In some embodiments, all or substantially all of the nucleotides including (a) the 5′-UTR, (b) the open reading frame (ORF), (c) the 3′-UTR, (d) the poly A tail, and any combination of (a, b, c, or d above) include naturally occurring canonical nucleotides A (adenosine), G (guanosine), C (cytosine), U (uridine), or T (thymidine). Polynucleotides and nucleic acids may include one or more modified (e.g., altered or alternative) nucleobases, nucleosides, nucleotides, or combinations thereof. The nucleic acids and polynucleotides useful in a nanoparticle composition can include any useful modification or alteration, such as to the nucleobase, the sugar, or the internucleoside linkage (e.g., to a linking phosphate/to a phosphodiester linkage/to the phosphodiester backbone). In certain embodiments, alterations (e.g, one or more alterations) are present in each of the nucleobase, the sugar, and the internucleoside linkage. Alterations according to the present disclosure may be alterations of ribonucleic acids (RNAs) to deoxyribonucleic acids (DNAs), e.g., the substitution of the 2′-OH of the ribofuranosyl ring to 2′-H, threose nucleic acids (TNAs), glycol nucleic acids (GNAs), peptide nucleic acids (PNAs), locked nucleic acids (LNAs), or hybrids thereof. Additional alterations are described herein.

The polynucleotide may contain from about 1% to about 100% alternative nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of a canonical nucleotide (e.g., A, G, U, or C).

Non-canonical or modified bases may include, for example, one or more substitutions or modifications including but not limited to alkyl, aryl, halo, oxo, hydroxyl, alkyloxy, and/or thio substitutions; one or more fused or open rings; oxidation; and/or reduction. In some embodiments, the nucleobase is an alternative uracil. Exemplary nucleobases and nucleosides having an alternative uracil include pseudouridine (y), pyridin-4-one ribonucleoside, 5-aza-uracil, 6-aza-uracil, 2-thio-5-aza-uracil, 2-thio-uracil (s²U), 4-thio-uracil (s⁴U), 4-thio-pseudouridine, 2-thio-pseudouridine, 5-hydroxy-uracil (ho⁵U), 5-aminoallyl-uracil, 5-halo-uracil (e.g., 5-iodo-uracil or 5-bromo-uracil), 3-methyl-uracil (m³U), 5-methoxy-uracil (mo⁵U), uracil 5-oxyacetic acid (cmo⁵U), uracil 5-oxyacetic acid methyl ester (mcmo⁵U), 5-carboxymethyl-uracil (cm⁵U), 1-carboxymethyl-pseudouridine, 5-carboxyhydroxymethyl-uracil (chm⁵U), 5-carboxyhydroxymethyl-uracil methyl ester (mchm⁵U), 5-methoxycarbonylmethyl-uracil (mcm⁵U), 5-methoxycarbonylmethyl-2-thio-uracil (mcm⁵s²U), 5-aminomethyl-2-thio-uracil (nm⁵s²U), 5-methylaminomethyl-uracil (mnm⁵U), 5-methylaminomethyl-2-thio-uracil (mnm⁵s²U), 5-methylaminomethyl-2-seleno-uracil (mnm⁵se²U), 5-carbamoylmethyl-uracil (ncm⁵U), 5-carboxymethylaminomethyl-uracil (cmnm⁵U), 5-carboxymethylaminomethyl-2-thio-uracil (cmnm⁵s²U), 5-propynyl-uracil, 1-propynyl-pseudouracil, 5-taurinomethyl-uracil (xm⁵U), 1-taurinomethyl-pseudouridine, 5-taurinomethyl-2-thio-uracil(xm⁵s²U), 1-taurinomethyl-4-thio-pseudouridine, 5-methyl-uracil (m⁵U, i.e., having the nucleobase deoxythymine), 1-methyl-pseudouridine (mV), 1-ethyl-pseudouridine (EtV), 5-methyl-2-thio-uracil (m⁵s²U), 1-methyl-4-thio-pseudouridine (m′s⁴′|i). 4-thio-1-methyl-pseudouridine, 3-methyl-pseudouridine (m³\|/), 2-thio-1-methyl-pseudouridine, 1-methyl-1-deaza-pseudouridine, 2-thio-1-methyl-1-deaza-pseudouridine, dihydrouracil (D), dihydropseudouridine, 5,6-dihydrouracil, 5-methyl-dihydrouracil (m⁵D), 2-thio-dihydrouracil, 2-thio-dihydropseudouridine, 2-methoxy-uracil, 2-methoxy-4-thio-uracil, 4-methoxy-pseudouridine, 4-methoxy-2-thio-pseudouridine, Nl-methyl-pseudouridine, 3-(3-amino-3-carboxypropyl)uracil (acp³U), 1-methyl-3-(3-amino-3-carboxypropyl)pseudouridine (acp³ y), 5-(isopentenylaminomethyl)uracil (inm⁵U), 5-(isopentenylaminomethyl)-2-thio-uracil (inm⁵s²U), 5,2′-O-dimethyl-uridine (m⁵Um), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), and 5-(isopentenylaminomethyl)-2′-O-methyl-uridine (inm⁵Um), 1-thio-uracil, deoxythymidine, 5-(2-carbomethoxyvinyl)-uracil, 5-(carbamoylhydroxymethyl)-uracil, 5-carbamoylmethyl-2-thio-uracil, 5-carboxymethyl-2-thio-uracil, 5-cyanomethyl-uracil, 5-methoxy-2-thio-uracil, and 5-[3-(1-E-propenylamino)]uracil.

In some embodiments, the nucleobase is an alternative cytosine. Exemplary nucleobases and nucleosides having an alternative cytosine include 5-aza-cytosine, 6-aza-cytosine, pseudoisocytidine, 3-methyl-cytosine (m3C), N4-acetyl-cytosine (ac4C), 5-formyl-cytosine (f5C), N4-methyl-cytosine (m4C), 5-methyl-cytosine (m5C), 5-halo-cytosine (e.g., 5-iodo-cytosine), 5-hydroxymethyl-cytosine (hm5C), 1-methyl-pseudoisocytidine, pyrrolo-cytosine, pyrrolo-pseudoisocytidine, 2-thio-cytosine (s2C), 2-thio-5-methyl-cytosine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytosine, 2-methoxy-5-methyl-cytosine, 4-methoxy-pseudoisocytidine, 4-methoxy-1-methyl-pseudoisocytidine, lysidine (k2C), 5,2′-O-dimethyl-cytidine (m5Cm), N4-acetyl-2′-O-methyl-cytidine (ac4Cm), N4,2′-O-dimethyl-cytidine (m4Cm), 5-formyl-2′-O-methyl-cytidine (f5Cm), N4,N4,2′-O-trimethyl-cytidine (m42Cm), 1-thio-cytosine, 5-hydroxy-cytosine, 5-(3-azidopropyl)-cytosine, and 5-(2-azidoethyl)-cytosine.

In some embodiments, the nucleobase is an alternative adenine. Exemplary nucleobases and nucleosides having an alternative adenine include 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenine (m1A), 2-methyl-adenine (m2A), N6-methyl-adenine (m6A), 2-methylthio-N6-methyl-adenine (ms2m6A), N6-isopentenyl-adenine (i6A), 2-methylthio-N6-isopentenyl-adenine (ms2i6A), N6-(cis-hydroxyisopentenyl)adenine (io6A), 2-methylthio-N6-(cis-hydroxyisopentenyl)adenine (ms2io6A), N6-glycinylcarbamoyl-adenine (g6A), N6-threonylcarbamoyl-adenine (t6A), N6-methyl-N6-threonylcarbamoyl-adenine (m6t6A), 2-methylthio-N6-threonylcarbamoyl-adenine (ms2g6A), N6,N6-dimethyl-adenine (m62A), N6-hydroxynorvalylcarbamoyl-adenine (hn6A), 2-methylthio-N6-hydroxynorvalylcarbamoyl-adenine (ms2hn6A), N6-acetyl-adenine (ac6A), 7-methyl-adenine, 2-methylthio-adenine, 2-methoxy-adenine, N6,2′-O-dimethyl-adenosine (m6Am), N6,N6,2′-O-trimethyl-adenosine (m62Am), 1,2′-O-dimethyl-adenosine (ml Am), 2-amino-N6-methyl-purine, 1-thio-adenine, 8-azido-adenine, N6-(19-amino-pentaoxanonadecyl)-adenine, 2,8-dimethyl-adenine, N6-formyl-adenine, and N6-hydroxymethyl-adenine.

In some embodiments, the nucleobase is an alternative guanine. Exemplary nucleobases and nucleosides having an alternative guanine include inosine (I), 1-methyl-inosine (m11), wyosine (imG), methylwyosine (mimG), 4-demethyl-wyosine (imG-14), isowyosine (imG2), wybutosine (yW), peroxywybutosine (o2yW), hydroxywybutosine (OHyW), undermodified hydroxywybutosine (OHyW*), 7-deaza-guanine, queuosine (Q), epoxyqueuosine (oQ), galactosyl-queuosine (galQ), mannosyl-queuosine (manQ), 7-cyano-7-deaza-guanine (preQO), 7-aminomethyl-7-deaza-guanine (preQl), archaeosine (G+), 7-deaza-8-aza-guanine, 6-thio-guanine, 6-thio-7-deaza-guanine, 6-thio-7-deaza-8-aza-guanine, 7-methyl-guanine (m7G), 6-thio-7-methyl-guanine, 7-methyl-inosine, 6-methoxy-guanine, 1-methyl-guanine (mlG), N2-methyl-guanine (m2G), N2,N2-dimethyl-guanine (m22G), N2,7-dimethyl-guanine (m2,7G), N2, N2,7-dimethyl-guanine (m2,2,7G), 8-oxo-guanine, 7-methyl-8-oxo-guanine, 1-methyl-6-thio-guanine, N2-methyl-6-thio-guanine, N2,N2-dimethyl-6-thio-guanine, N2-methyl-2′-O-methyl-guanosine (m2Gm), N2,N2-dimethyl-2′-O-methyl-guanosine (m22Gm), 1-methyl-2′-O-methyl-guanosine (mlGm), N2,7-dimethyl-2′-O-methyl-guanosine (m2,7Gm), 2′-O-methyl-inosine (Im), 1,2′-O-dimethyl-inosine (mllm), 1-thio-guanine, and O-6-methyl-guanine.

The alternative nucleobase of a nucleotide can be independently a purine, a pyrimidine, or a purine or pyrimidine analog. For example, the nucleobase can be an alternative to adenine, cytosine, guanine, uracil, or hypoxanthine. In some embodiments, the nucleobase can also include, for example, naturally-occurring and synthetic derivatives of a base, including pyrazolo[3,4-d]pyrimidines, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil (pseudouracil), 4-thiouracil, 8-halo (e.g., 8-bromo), 8-amino, 8-thiol, 8-thioalkyl, 8-hydroxy and other 8-substituted adenines and guanines, 5-halo, e.g., 5-bromo, 5-trifluoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo[3,4-d]pyrimidine, imidazo[1,5-a]1,3,5 triazinones, 9-deazapurines, imidazo[4,5-d]pyrazines, thiazolo[4,5-d]pyrimidines, pyrazin-2-ones, 1,2,4-triazine, pyridazine; or 1,3,5-triazine. When the nucleotides are depicted using the shorthand A, G, C, T, or U, each letter refers to the representative base and/or derivatives thereof, e.g., A includes adenine or adenine analogs, e.g., 7-deaza adenine.

Generally, RNA includes the sugar group ribose, which is a 5-membered ring having an oxygen. Exemplary, non-limiting alternative nucleotides include replacement of the oxygen in ribose (e.g., with S (sulfur), Se (selenium), or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino (that also has a phosphoramidate backbone)); multicyclic forms (e.g., tricyclo and“unlocked” forms, such as glycol nucleic acid (GNA) (e.g., R-GNA or S-GNA, where ribose is replaced by glycol units attached to phosphodiester bonds), threose nucleic acid (TNA, where ribose is replacef with a-L-threofuranosyl-(3′ 2′)), and peptide nucleic acid (PNA, where 2-amino-ethyl-glycine linkages replace the ribose and phosphodiester backbone).

Alternative nucleotides can be altered on the internucleoside linkage (e.g., phosphate backbone). Herein, in the context of the polynucleotide backbone, the phrases “phosphate” and “phosphodiester” are used interchangeably. Backbone phosphate groups can be altered by replacing one or more of the oxygen atoms with a different substituent. The alternative nucleotides can include the wholesale replacement of an unaltered phosphate moiety with another internucleoside linkage as described herein. Examples of alternative phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be altered by the replacement of a linking oxygen with nitrogen (bridged phosphoramidates), sulfur (bridged phosphorothioates), and carbon (bridged methylene-phosphonates).

The replacement of one or more of the oxygen atoms at the alpha position of the phosphate moiety (e.g., a-thio phosphate) can be provided to confer stability (such as against exonucleases and endonucleases) to RNA and DNA through the unnatural phosphorothioate backbone linkages. Phosphorothioate DNA and RNA have increased nuclease resistance and subsequently a longer half-life in a cellular environment.

Polynucleotides may contain an internal ribosome entry site (IRES). An IRES may act as the sole ribosome binding site, or may serve as one of multiple ribosome binding sites of an mRNA. A polynucleotide containing more than one functional ribosome binding site may encode several peptides or polypeptides that are translated independently by the ribosomes (e.g., multicistronic mRNA). When polynucleotides are provided with an IRES, further optionally provided is a second translatable region. Examples of IRES sequences that can be used according to the present disclosure include without limitation, those from picomaviruses (e.g., FMDV), pest viruses (CFFV), polio viruses (PV), encephalomyocarditis viruses (ECMV), foot-and-mouth disease viruses (FMDV), hepatitis C viruses (HCV), classical swine fever viruses (CSFV), murine leukemia virus (MILV), simian immune deficiency viruses (SIV), and/or cricket paralysis viruses (CrPV).

A polynucleotide (e.g., an mRNA) may include a 5′-cap structure. The 5′-cap structure of a polynucleotide is involved in nuclear export and increasing polynucleotide stability and binds the mRNA Cap Binding Protein (CBP), which is responsible for polynucleotide stability in the cell and translation competency through the association of CBP with poly-A binding protein to form the mature cyclic mRNA species. The cap further assists the removal of 5′-proximal introns removal during mRNA splicing.

A polynucleotide or nucleic acid (e.g., an mRNA) may include a polyA sequence and/or polyadenylation signal. A polyA sequence may be formed entirely or mostly of adenine nucleotides or analogs or derivatives thereof. A polyA sequence may be a tail located adjacent to a 3′ untranslated region of a nucleic acid.

The encapsulated agent may be an interfering RNA. The term “interfering RNA” or “RNAi” or “interfering RNA sequence” refers to single-stranded RNA (e.g., mature miRNA) or double-stranded RNA (i.e., duplex RNA such as siRNA, aiRNA (asymmetric interfering RNA), or pre-miRNA) that is capable of reducing or inhibiting the expression of a target gene or sequence (e.g., by mediating the degradation or inhibiting the translation of mRNAs which are complementary to the interfering RNA sequence) when the interfering RNA is in the same cell as the target gene or sequence. Interfering RNA thus refers to the single-stranded RNA that is complementary to a target mRNA sequence or to the double-stranded RNA formed by two complementary strands or by a single, self-complementary strand. Interfering RNA may have substantial or complete identity to the target gene or sequence, or may include a region of mismatch (i.e., a mismatch motif). The sequence of the interfering RNA can correspond to the full-length target gene, or a subsequence thereof.

Interfering RNA includes “small-interfering RNA” or “siRNA,” e.g., interfering RNA of about 15-60, 15-50, or 15-40 (duplex) nucleotides in length, for example, about 15-30, 15-25, or 19-25 (duplex) nucleotides in length, for example, about 20-24, 21-22, or 21-23 (duplex) nucleotides in length (e.g., each complementary sequence of the double-stranded siRNA is 15-60, 15-50, 15-40, 15-30, 15-25, or 19-25 nucleotides in length, for example, about 20-24, 21-22, or 21-23 nucleotides in length, and the double-stranded siRNA is about 15-60, 15-50, 15-40, 15-30, 15-25, or 19-25 base pairs in length, for example about 18-22, 19-20, or 19-21 base pairs in length). siRNA duplexes may include 3′ overhangs of about 1 to about 4 nucleotides or about 2 to about 3 nucleotides and 5′ phosphate termini. Examples of siRNA include, without limitation, a double-stranded polynucleotide molecule assembled from two separate stranded molecules, wherein one strand is the sense strand and the other is the complementary antisense strand; a double-stranded polynucleotide molecule assembled from a single stranded molecule, where the sense and antisense regions are linked by a nucleic acid-based or non-nucleic acid-based linker; a double-stranded polynucleotide molecule with a hairpin secondary structure having self-complementary sense and antisense regions; and a circular single-stranded polynucleotide molecule with two or more loop structures and a stem having self-complementary sense and antisense regions, where the circular polynucleotide can be processed in vivo or in vitro to generate an active double-stranded siRNA molecule.

In one embodiment, the nucleic acid is an antisense oligonucleotide directed to a target gene or sequence of interest. The terms “antisense oligonucleotide” or “antisense” include oligonucleotides such as single strands of DNA or RNA that are complementary to a chosen polynucleotide sequence. Antisense RNA oligonucleotides prevent the translation of complementary RNA strands by binding to the RNA. Antisense DNA oligonucleotides can be used to target a specific, complementary (coding or non-coding) RNA. If binding occurs, this DNA/RNA hybrid can be degraded by the enzyme RNase H. In a particular embodiment, antisense oligonucleotides include from about 10 to about 60 nucleotides, for example, from about 15 to about 30 nucleotides. The term also encompasses antisense oligonucleotides that may not be exactly complementary to the desired target gene. Thus, an embodiment of the invention can be utilized in instances where non-target specific-activities are found with antisense, or where an antisense sequence containing one or more mismatches with the target sequence is for a particular use.

As described above, the encapsulated agent can be a polynucleic acid or nucleic acid such as an oligonucleotide, dinucleotide, ribonucleic acid (RNA), deoxyribonucleic acid (DNA), plasmid DNA, mRNA, hybrids thereof, RNAi-inducing agents, short interfering RNA (siRNA), microRNA (miRNA), small hairpin RNA (shRNA), dicer substrate RNA (dsRNA), transfer RNA (tRNA), ribozymes, catalytic DNA, aptamers, vectors, or an antisense oligonucleotide. The encapsulated agent can be a single species or combinations of multiple types of oligonucleotides or oligonucleotides with multiple sequences. The encapsulated agent or agents can include non-nucleic acid species including hydrophilic small molecules, peptides, or proteins. For example, a protein encapsulated agent could be a Cas endonuclease. Non-nucleic acid species that are co-encapsulated with nucleotide-based encapsulated agents may be included because they have a therapeutic benefit, or they may aid in the encapsulation, delivery, or expression of the nucleotide-based encapsulated agent.

### Stabilizing Agent

The stabilizing agents suitable to iFNP have been described in U.S. Pat. Nos. 10,231,937 and 11,103,461, US published applications US20200206136, US20210259984, and US20200023332, and published international applications WO2020227350 and WO2021046078. The stabilizing agent used in iFNP is also referred to here as a first stabilizing agent or first stabilizing polymer. The agent can be a copolymer of a more polar block coupled with a more nonpolar (or less polar) block. The term “block” may be interpreted as either a distinct domain with a single molecular composition, or it may mean a region of the polymer chain that has regions that are predominantly more polar and other regions that are less polar. The polarity may be imparted by the monomers forming the polymer backbone or grafted pendant groups or chains attached to the main polymer backbone. For example, the copolymer may be amphiphilic (the more nonpolar block is not water soluble), however, this is not a requirement and copolymers may be fully water soluble or fully non-water soluble, as long as solubilities of the blocks differ significantly enough in the antisolvent to enable surface stabilization driven by assembly of the stabilizing agent on the nanoparticle surface. The copolymer should self-assemble in the antisolvent, with the more polar blocks precipitating and the more nonpolar blocks remaining soluble. When used in the iFNP process to make particles, the more polar blocks go to the core of the particle, and the more nonpolar blocks form a sterically protective shell or brush. The sterically protective shell prevents particle aggregation and prevents release of encapsulated material during subsequent processing steps.

Nanoparticles formed by the disclosed process can be formed with graft, block, or random copolymers. For example, these copolymers can have a molecular weight between about 1000 g/mole and about 1,000,000 g/mole, or between about 3000 g/mole and about 25,000 g/mole, or at least about 2000 g/mole.

The copolymers are formed of repeat units or blocks that have different solubility characteristics. For example, these repeat units can be in groups of at least two forming a block of a given character. Depending on the method of synthesis, these blocks could be of all the same repeat unit or contain different repeat units dispersed throughout the block, but still yielding blocks of the copolymer with polar and more non-polar portions. These blocks can be arranged into a series of two blocks (diblock) or three blocks (triblock), or more (multiblock), forming the backbone of a block copolymer. In addition, the polymer chain can have chemical moieties covalently attached or grafted to the backbone. Such polymers are graft polymers. Block units making up the copolymer can occur in regular intervals or they can occur randomly making a random copolymer. In addition, grafted side chains can occur at regular intervals along the polymer backbone or randomly making a randomly grafted copolymer. In graft polymers, polar blocks may be grafted on a non-polar polymer. Alternatively, non-polar blocks may be grafted on a more polar polymer chain. In graft copolymers, the length of a grafted moiety can vary. For example, the grafted segments can be equivalent to 2 to 22 ethylene units in length. The grafted hydrophobic groups which create at least one less polar region of the copolymer may include tocopherol, tocopherol derivatives, lipids, alcohols with carbon numbers from 12 to 40, cholesterols, other sterols, unsaturated and/or hydrogenated fatty acids, salts, esters or amides thereof, fatty acids, mono-, di- or triglycerides, waxes, ceramides, cholesterol derivatives, or combinations. The grafting of the polymer backbone can be useful to enhance solvation or nanoparticle stabilization properties.

The copolymer used in the compositions and methods of an embodiment of the invention may be formed of blocks of at least two repeat units or with a minimum contour length equivalent to at least 25 ethylene units. Contour lengths are the linear sum of the polymer backbone, the molecular dimensions of which can be approximated using the Polymer Handbook, 4th Edition, eds. J. Brandrup, E. H. Immergut, and E. A. Grulke, assoc. ed. A. Abe, D. R. Bloch, 1999, New York, John Wiley & Sons, which is hereby incorporated by reference in its entirety.

Examples of suitable nonpolar (or hydrophobic or less polar) blocks in a copolymer include but are not limited to the following: acrylates including methyl acrylate, ethyl acrylate, propyl acrylate, n-butyl acrylate (BA), isobutyl acrylate, 2-ethyl acrylate, and t-butyl acrylate; methacrylates including ethyl methacrylate, n-butyl methacrylate, and isobutyl methacrylate; acrylonitriles; methacrylonitrile; vinyls including vinyl acetate, vinylversatate, vinylpropionate, vinylformamide, vinylacetamide, vinylpyridines, vinyl phenols, and vinyllimidazole; aminoalkyls including aminoalkylacrylates, aminoalkylsmethacrylates, and aminoalkyl(meth)acrylamides; styrenes; cellulose acetate phthalate, cellulose acetate succinate, hydroxypropylmethylcellulose phthalate, poly(D,L-lactide), poly(D,L-lactide-co-glycolide), poly(glycolide), poly(hydroxybutyrate), poly(alkylcarbonate) and poly(orthoesters), polyesters, poly(hydroxyvaleric acid), polydioxanone, poly(ethylene terephthalate), poly(malic acid), poly(tartronic acid), polyanhydrides, polyphosphazenes, poly(amino acids), lactic acid, poly(lactic acid) (PLA), caprolactone, poly(caprolactone) (PCL), glycolic acid, poly(glycolic acid), and their copolymers, e.g., poly(lactic-co-glycolic acid) (PLGA); hydrophobic peptide-based polymers and copolymers based on poly(L-amino acids), poly(ethylene-vinyl acetate) (“EVA”) copolymers, silicone rubber, polyethylene, polypropylene, polydienes (polybutadiene, polyisoprene, and hydrogenated forms of these polymers), maleic anhydride copolymers of vinyl methylether and other vinyl ethers, polyamides (nylon 6,6), polyurethane, poly(ester urethanes), poly(ether urethanes), and poly(esterurea). Hydrophobically modified sugar polymers, such as HPMC (hydroxypropyl methylcellulose), acetalated dextran, or acetylated dextran can be used. For example, polymeric blocks can include poly(ethylenevinyl acetate), poly(D,L-lactic acid) oligomers and polymers, poly(L-lactic acid) oligomers and polymers, poly(glycolic acid), copolymers of lactic acid and glycolic acid, poly(caprolactone), poly(valerolactone), polyanhydrides, copolymers of poly(caprolactone) or poly(lactic acid), or poly(propylene sulfide). For example, for non-biologically related applications polymeric blocks can include, for example, polystyrene, polyacrylates, and butadienes.

Natural products with sufficient hydrophobicity to act as the non-polar portion of the polymer include the following: hydrophobic vitamins (for example vitamin E, vitamin K, and vitamin A), carotenoids, and retinols (for example, beta carotene, astaxanthin, trans and cis retinal, retinoic acid, folic acid, dihydrofolate, retinylacetate, retinyl palmintate), cholecalciferol, calcitriol, hydroxycholecalciferol, ergocalciferol, alpha-tocopherol, alpha-tocopherol acetate, alphatocopherol nicotinate, estradiol, lipids, alcohols with carbon numbers from 12 to 40, cholesterols, other sterols, unsaturated and/or hydrogenated fatty acids, salts, esters or amides thereof, fatty acids, mono-, di- or triglycerids, waxes, ceramides, cholesterol derivatives, or mixtures thereof. For example, a natural product is vitamin E which can be readily obtained as a vitamin E succinate, which facilitates functionalization to amines and hydroxyls on the active species.

Examples of suitable polar (or hydrophilic or more polar) blocks in an amphiphilic polymer that is a block copolymer include, but are not limited to the following: carboxylic acids including acrylic acid, methacrylic acid, itaconic acid, and maleic acid; polyoxyethylenes or polyethylene oxide; polyacrylamides and copolymers thereof with dimethyl-aminoethyl-methacrylate, diallyl-dimethyl-ammonium chloride, vinylbenzyl trimethylammonium chloride, acrylic acid, methacrylic acid, 2-acryamideo-2-methylpropane sulfonic acid and styrene sulfonate, polyvinyl pyrrolidone, starches and starch derivatives, dextran and dextran derivatives; polypeptides, such as polylysines, polyarginines, polyaspartic acids, polyglutamic acids; poly hyaluronic acids, alginic acids, polylactides, polyethyleneimines, polyionenes, polyacrylic acids, and polyiminocarboxylates, gelatin, and unsaturated ethylenic mono or dicarboxylic acids. To prepare anionic copolymers, acrylic acid, methacrylic acid, and/or poly aspartic acid or glutamic acid polymers can be used. To produce cationic copolymers, DMAEMA (dimethyl aminoethyl methacrylate), polyvinyl pyridine (PVP), and/or dimethyl aminoethyl acrylamide (DMAMAM) can be used. Polar blocks of the stabilizing polymer may be non-charged, cationic, cationizable, anionic, or anionizable, or combinations of these. A listing of suitable polar, water soluble, polymers can be found in Handbook of Water-Soluble Gums and Resins, R. Davidson, McGraw-Hill (1980), which is hereby incorporated by reference in its entirety.

The nonpolar polymers and polar polymers each may be selected to have a molecular weight or be within a range of molecular weights. For example, and not to be construed as limiting, dextran, poly(aspartic acid), and poly(glutamic acid) polymers (alone or as a component of a copolymer) may have molecular weights within a range of from about 500 to about 500,000 Da, from about 500 to about 50,000 Da, or from about 750 Da to about 20,000 Da. For example, and not to be construed as limiting, PLA, PLGA, and PCL polymers (alone or as a component of a copolymer) may have molecular weights within a range of from about 500 to about 500,000 Da, from about 500 to about 50,000 Da, or from about 750 Da to about 20,000 Da.

The lists above of nonpolar polymers and polar polymers should not be considered exclusive of one another. Copolymers of two polymers given in a single list may have sufficient differences in solubilities in a given antisolvent to be used in this process. As an illustrative example, poly(ethylene oxide) and poly(acrylic acid) are both given in the list of polar polymers. However, poly(ethylene oxide) is soluble in chloroform and acetone, while poly(acrylic acid) is not. Therefore, copolymers of poly(ethylene oxide) and poly(acrylic acid) may be used in this process with chloroform or acetone as the antisolvent.

Stabilizing agent polymers can be linear polymers including one or more polar blocks and one or more nonpolar blocks. For example, the nonpolar block(s) can be poly(lactic acid) (PLA), poly(lactic-co-glycolic acid) (PLGA), polycaprolactone (PCL), a polyester, a poly(ortho ester), poly[(carboxyphenoxy)propane-sebacic acid], a polyphosphoester, a polyester amide, a polyurethane, a polyvinyl acrylate, a poly(amino acid), or a hydrophobically modified polysaccharide. For example, the polar block(s) can be a poly(amino acid) such as poly(glutamic acid) (PGlu), poly(aspartic acid) (PAsp), poly(lysine), poly(arginine), poly(serine), poly(threonine), poly(glutamine), poly(asparagine), poly(cysteine), or combinations or modifications of these. For example, the polar block(s) can be a polysaccharide such as cellulose, dextran, maltodextrin, dextrin, dextran sulfate, dextrin sulfate, hyaluronic acid, pectins, amylopectin, amylose, pullulan, xylan, carrageenan, chitin, chitosan, starch, or combinations or modifications of these. For example, the stabilizing polymer can be PAsp-b-PLA, PAsp-b-PLGA, PAsp-b-PCL, PAsp-b-PLA-b-PAsp, PAsp-b-PLGA-b-PAsp, poly(ethylene glycol)-b-PLA-b-PAsp, poly(ethylene glycol)-b-PLGA-b-PAsp, poly(ethylene glycol)-b-PCL-b-PAsp, PGlu-b-PLA, PGlu-b-PLGA, PGlu-b-PCL, PGlu-b-PLA-b-PGlu, PGlu-b-PLGA-b-PGlu, poly(ethylene glycol)-b-PLA-b-PGlu, poly(ethylene glycol)-b-PLGA-b-PGlu, poly(ethylene glycol)-b-PCL-b-PGlu, or other combinations thereof. For example, the stabilizing polymer can be a polysaccharide-b-PLGA, a polysaccharide-b-PLA, a polysaccharide-b-PCL, or a higher order block copolymer composed of polysaccharide and PLA, PCL, and/or PLGA blocks.

Stabilizing agents that are block copolymers include poly(styrene)-b-poly(acrylic acid), poly(lactic acid)-b-poly(aspartic acid), poly(lactic acid-co-glycolic acid)-b-poly(aspartic acid), poly(ethylene glycol)-b-poly(lactic acid)-b-poly(aspartic acid), poly(aspartic acid)-b-poly(lactic acid-co-glycolic acid)-b-poly(aspartic acid), dextran-b-poly(lactic acid), and dextran-b-poly(lactic-co-glycolic acid).

In an embodiment of the invention the stabilizing polymer is a comb polymer of or including a hydrophilic backbone and hydrophobic branches or grafts. In an embodiment of the invention the backbone of the comb polymer can be branched, like a dextran or a poly(aspartic acid) produced through condensation polymerization. In an embodiment of the invention the backbone of the comb polymer can be linear, like cellulose. In an embodiment of the invention, the hydrophilic backbone of the comb polymer is a polysaccharide. For example, the backbone can be cellulose, dextran, maltodextrin, dextrin, dextran sulfate, dextrin sulfate, hyaluronic acid, pectins, amylopectin, amylose, pullulan, xylan, carrageenan, chitin, chitosan, starch, or combinations or modifications of these. In an embodiment of the invention the hydrophilic backbone of the comb polymer is a poly(amino acid) such as poly(glutamic acid), poly(aspartic acid), poly(lysine), poly(arginine), poly(serine), poly(threonine), poly(glutamine), poly(asparagine), poly(cysteine), or combinations or modifications of these. In an embodiment of the invention the backbone is polyvinyl alcohol. In an embodiment of the invention the nonpolar side chains are poly(lactic acid) (PLA), poly(lactic-co-glycolic acid) (PLGA), polycaprolactone (PCL), a polyester, a poly(ortho ester), poly[(carboxyphenoxy)propane-sebacic acid], a polyphosphoester, a polyester amide, a polyurethane, a polyvinyl acrylate, a poly(amino acid), or a hydrophobically modified polysaccharide. The comb polymer may have between 20, 30, 40, 50, 60, 70, or 80 wt % and 70, 80, 90, 95 wt % nonpolar side chains. The backbone of the comb polymer can be between 500 Da, 1000 Da, 2500 Da, 5000 Da, 10000 Da, or 20000 Da and 20000 Da, 50000 Da, 100000 Da, or 500000 Da. Each nonpolar side chain or graft can be between 100, 500, 1000, 5000 Da, 10000 Da, or 20000 Da and 20000 Da, 50000 Da, 100000 Da, or 500000 Da. The comb polymer may have between 20, 30, 40, 50, 60, 70, or 80 wt % and 70, 80, 90, 95 wt % nonpolar side chains. Someone skilled in the art will recognize that the number of nonpolar side chains per polar backbone that will result in the proper wt % of nonpolar groups will depend on the molecular weights of each species. For example, the stabilizing polymer can be a dextran-graft-PLGA, dextran-graft-PLA, dextran-graft-PCL, or another polysaccharide with PLA, PCL, or PLGA grafted on. For example, the stabilizing comb polymer can be PAsp-graft-PLA, PAsp-graft-PLGA, PAsp-graft-PCL or another poly(amino acid) with PLA, PLGA, or PCL grafted on.

Random copolymers include hydroxypropyl cellulose, methyl cellulose, ethyl methyl cellulose, hydroxypropylmethylcellulose, carboxymethyl cellulose, or a combination of these. A cellulosic polymer can include hydroxypropyl, hydroxyethyl, hydroxymethyl, succinate, and/or acetate substitution(s). In another embodiment of the invention poly(meth)acrylate-based random copolymers are used as stabilizers. Eudragit polymers produced by Evonik Industries are one commercialized version of these. However, embodiments of the invention are not limited to the aforementioned random copolymers. Stabilizing random copolymers may have molecular weights ranging from about 0.1 kDa, 1 kDa, 10 kDa, 100 kDa, or 1000 kDa to about 5 kDa, 50 kDa, 100 kDa, 1000 kDa or greater.

The first stabilizing agent may be a blend of different stabilizing agents.

### Salts

Other non-liquid compounds that aid in the solvent quality of the streams may be added and are also considered part of the solvent or antisolvent. For example, a surfactant, a salt, or a cosolvent may be added to a solvent and considered part of the solvent. These excipient compounds may or may not be in the coated nanocarrier, depending on the requirements of the final product.

Patent application WO2021046078 describes suitable salt forms of the nucleic acid or polynucleic acid. The protonated (free acid) form may be used, or a form of the nucleic acid that is partially (1%, 5%, 10%, 15%, 25%, 50%, 75%, 99% of acid groups neutralized) or fully neutralized with sodium hydroxide, lithium hydroxide, Tris (tris(hydroxymethyl)aminomethane), triethylamine, ammonia, cesium hydroxide, potassium hydroxide, choline, thiamine, pyroxidine, urea, guanidine, diphenhydramine, a primary amine, a secondary amine, a quaternary amine, or a tertiary amine may be used.

Any method known to the field may be used to modify the salt form of the nucleic acid. The nucleic acid may be neutralized in situ in the solvent stream. The nucleic acid may be neutralized in a salt exchange process and then purified and isolated prior to dissolution in the solvent. The nucleic acid salt form may be modified after in vitro transcription by buffer selection.

Crosslinking of acidic residues within a stabilizing copolymer, such as poly(aspartic acid) or poly(acrylic acid) or poly(glutamic acid), have been described (U.S. Pat. Nos. 10,231,937 and 11,103,461 and Pagels, R. F.; Prud'homme, R. K., Polymeric nanoparticles and microparticles for the delivery of peptides, biologics, and soluble therapeutics. J Control Release 2015, vol. 219, 519-535, these documents are hereby incorporated herein in their entirety). Suitable crosslinkers include but are not limited to salt forms of calcium, iron, zinc, manganese, divalent cations, and trivalent cations. Polyvalent amines, such as spermine, tetraethylene pentaamine, protamine, poly(lysine), or other organic amines may be used.

For example, the nanoparticle can be crosslinked during assembly of the nanoparticle. For example, the nanoparticle can be crosslinked after assembly of the nanoparticle. The crosslinking can be covalent crosslinking. For example, the crosslinking can be disulfide crosslinking. For example, the crosslinking can occur through “Click Chemistry”. The crosslinking can involve cleavable ester linkage of the types described in U.S. application Ser. No. 13/969,449, Particulate Constructs for Release of Active Agents, Lawrence Mayer, et al. The crosslinking can be non-covalent. For example, the crosslinking can be ionic, chelation, acid-base, or hydrogen bonding crosslinking.

A crosslinking agent can be added to crosslink the copolymer. For example, the crosslinking agent can be added to crosslink groups of the copolymer having anionic functionality or character. For example, the crosslinking agent can be an alkaline earth halide, a magnesium halide, magnesium chloride, a calcium halide, calcium chloride, a transition metal halide, an iron halide, iron(III) chloride, spermine, or combinations. For example, the crosslinking agent can be a metal acetate, an alkaline earth acetate, a transition metal acetate, calcium acetate, or combinations. For example, the crosslinking agent can be chromium(III) acetate, or another chromium (III) salt. For example, the crosslinking agent can be a metal nitrate, an alkaline earth nitrate, a transition metal nitrate, calcium nitrate, zinc nitrate, iron nitrate, or combinations. Other bio-compatible multi-cationic water-soluble agents may be used as crosslinking agents, for example, to crosslink anionic sections of the copolymer. For example, the water-soluble agent can include tobramycin and the tobramycin can crosslink the copolymer. One example is tetraethylene pentamine. Ammonia or another chemical with basic character can be added to promote ionic interactions between the cationic crosslinker and the hydrophilic groups of the copolymer, if they have anionic functionality.

Alternatively, the shell of the nanoparticle may be crosslinked to enhance nanoparticle stability. For example, the end groups or other groups of the hydrophobic (non-polar) polymer chains or blocks can be crosslinked. For example, the crosslinking can be ionic or covalent. For example two or more hydrophobic polymer chains may be crosslinked directly or through the addition of a multifunctional crosslinker. For example, a hydrophobic polymer shell that contains groups with functionality “X” (on the chain end or throughout the chain) that is reactive to functionality “Y” may be crosslinked using a crosslinker that has two or more “Y” functionality groups. For example, the X and Y functional groups can be thiols and acrylates, thiols and maleimides, azides and alkynes, amines and acids, or vice versa. Functional groups X and Y can be groups that are reactive through “click chemistry.” Added catalysts or other agents may be needed or used to induce the crosslinking of the shell.

The crosslinking agent may also be used to neutralize charge on the nucleic acid. In the absence of an acidic residue on the stabilizing copolymer, the crosslinking agent may still be included in the process.

### Solvent

Suitable antisolvents have been described in U.S. Pat. Nos. 10,231,937 and 11,103,461. Examples of polar process solvents include, but are not limited to, water, alcohols, acetone, acetonitrile, glycol ethers, dimethyl sulfoxide (DMSO), dimethylformamide, N-methyl-2-pyrrolidone, dihydrolevoglucosenone, glycofurol, and mixtures thereof. In an embodiment, the encapsulated agent is dissolved in a process solvent or mixture. The stabilizing agent is dissolved in the same process solvent or mixture, or may be dissolved in a different process solvent. The solutions of encapsulated agent and stabilizing agent may be combined to form a single solution or may be maintained separately until the mixing process. The polar process solvent can be heated or pressurized or both to facilitate dissolution of the encapsulated agent and the stabilizing agent, depending on the dissolution characteristics. Selection of the polar process solvent is informed by the solubility characteristics of the encapsulated agent and the stabilizing agent. The polar process solvent containing the stabilizing agent is chosen such that the stabilizing agent is molecularly dissolved. This means that the process solvent solubilizes all parts of the stabilizing agent. The process solvent containing the encapsulated agent, if present, is also chosen such that material is molecularly dissolved. These process solvents may be, but are not required to be, the same. In some cases, both the stabilizing agent and the encapsulated agent may be dissolved in a single solution of the process solvent.

The concentration of the encapsulated agent may be within an order of magnitude of the concentration of the stabilizing agent. If the concentration of the encapsulated agent is much lower than the concentration of the stabilizing agent, then the final drug loading may be low (small). If the concentration of the encapsulated active is much higher than the concentration of the stabilizing agent, then the inverse nanoparticles may not be sufficiently stabilized to prevent aggregation. In an embodiment, encapsulated agents that are poorly soluble in the antisolvent are coated, encapsulated, or confined as a core component and sterically stabilized by stabilizing agent.

A stabilizing agent can be dissolved in a polar process solvent at a concentration of at least 0.01% by weight; the concentration of stabilizing agent can be at least 0.1% by weight to form a first process solution. In an embodiment, the stabilizing agent can be dissolved in the polar process solvent at a concentration in a range of from about 0.01 wt %, 0.1 wt %, 0.5 wt %, 1 wt %, 2 wt %, 5 wt %, 10 wt %, or 20 wt % to about 0.2 wt %, 0.5 wt %, 1 wt %, 2 wt %, 5 wt %, 10 wt %, 20 wt %, or 40 wt %. A person of skill in the art will appreciate that a factor such as the economics of a process can constrain a lower bound of concentration, and that factors such as the viscosity of the process solution or the solubility limit of the copolymer in the polar process solvent can constrain an upper bound of concentration. For example, if the viscosity of the first process solution is much greater than that of the antisolvent, mixing of the first process solution with the antisolvent may be inhibited. A person of skill in the art will appreciate that factors such as the molecular weight of the copolymer and the composition of the copolymer can affect the maximum concentration that can be attained in the polymer solution before the viscosity becomes too high (large).

In an embodiment, the encapsulated agent can be dissolved in the polar process solvent at a concentration in a range of from about 0.01 wt %, 0.1 wt %, 0.5 wt %, 1 wt %, 2 wt %, 5 wt %, 10 wt %, or 20 wt % to about 0.2 wt %, 0.5 wt %, 1 wt %, 2 wt %, 5 wt %, 10 wt %, 20 wt %, or 40 wt %. A person of skill in the art will appreciate that a factor such as the economics of a process can constrain a lower bound of concentration, and that factors such as the viscosity of the process solution or the solubility limit in the polar process solvent can constrain an upper bound of concentration.

In an embodiment of the process, the solvent is DMSO (dimethylsulfoxide). In an alternative embodiment, the DMSO is in a mixture with water. For example, the water content in the DMSO and water mixture may be 0.5 volume percent (% vol), 1% vol, 2.5% vol, 5% vol, 7.5% vol, 10% vol, 15% vol, 20% vol, or 25% vol.

### Antisolvent

In an embodiment of the invention, suitable antisolvents are more non-polar than the process solvent and have been described in U.S. Pat. Nos. 10,231,937 and 11,103,461. Examples include chloroform, dichloromethane, alcohols, alkanes such as hexane, tetrahydrofuran (THF), toluene, ethyl acetate, methyl acetate, acetonitrile, acetone, or combinations. Selection of the antisolvent is driven by the solubility characteristics of the stabilizing agent and the encapsulated agent. The encapsulated agent becomes supersaturated under the mixing conditions and precipitates from solution. Additionally, the antisolvent is selected such that the dissimilar solubility characteristics of regions or portions of the stabilizing agent are manifested, and the more polar portions of the copolymer can no longer exist in the soluble state, so that the stabilizing agent assembles onto the encapsulated agent and precipitates and produces a stable “inverse” nanocarrier. It is important to note that process solvents of one system may work well as the antisolvent in another system; thus, the listing above of an example as a process solvent should not be considered to exclude its use as an antisolvent, and the listing above of an example as an antisolvent should not be considered to exclude its use as a process solvent.

The antisolvent is chosen such that the more polar sections of the stabilizing agent rapidly precipitate while the more non-polar components of the stabilizing agent remain solubilized. Thus, the stabilizing agent can self-assemble into the desired nanoparticle form in the antisolvent. The antisolvent is chosen such that the encapsulated agent rapidly precipitates in the final mixture. In some cases the process solvent and antisolvent can be fully miscible at the final composition. In some cases, no more than 20 volume percent of the process solvent may phase separate in the final composition.

In an embodiment, the crosslinking agent or salt can be incorporated into the antisolvent. In some embodiments, the crosslinking agent can be dissolved first in a secondary antisolvent. For example, a solution in methanol could be produced and then added to the dichloromethane antisolvent. The secondary antisolvent can be 1 vol %, 2 vol %, 5 vol %, 10 vol %, 20 vol % or higher (greater) within the antisolvent composition. The concentration of the crosslinking agent is selected to be a desired charge equivalent with respect to the total acid residue composition of the encapsulated agent and the stabilizing agent. The charge ratio is a molar ratio of the positive charge from the crosslinking agent to the negative charge from all acid residues. The crosslinking charge may be 0.1, or 0.2, or 0.25, or 0.3, or 0.5, or 0.6, or 0.7, or 0.75, or 0.9, or 1, or 1.2, or 1.5, or 2, or higher (greater) with respect to the acid charge.

### Mixing

The intense micromixing of the process solution and the antisolvent can be effected in various geometries. The high velocity inlet streams cause turbulent flow and mixing that occurs in a central cavity. The time for process solvent/antisolvent mixing is more rapid than the assembly time of the nanoparticles. While not meant to be limiting, two such geometries have been previously described and analyzed: The Confined Impinging Jet mixer (CIJ) (Johnson, B. K., Prud'homme, R. K. Chemical processing and micromixing in confined impinging jets. AIChE Journal 2003, 49, 2264-2282) and the multi-inlet vortex mixer (MIVM) (Liu, Y., Cheng, C., Liu, Y., Prud'homme, R. K., Fox, R. O. Mixing in a multi-inlet vortex mixer (MIVM) for flash nano-precipitation. Chemical Engineering Science 2008, 63, 2829-2842). These examples are meant to be illustrative rather than limiting or exhaustive.

The fast mixing and high energy dissipation involved in the iFNP process provide mixing timescales that are shorter than the timescale for nucleation and growth of particles, which leads to the formation of nanoparticles with active agent loading contents and size distributions not provided by other technologies. When forming the nanoparticles via inverse Flash NanoPrecipitation (iFNP), mixing occurs fast enough to allow high supersaturation levels, for example, as high as 10,000, of all components to be reached prior to the onset of aggregation. The supersaturation level is the ratio of the actual concentration of a material, for example, an encapsulated agent, in a solvent to the saturation concentration of that material in that solvent. For example, the supersaturation levels can be at least about 1, 3, 10, 30, 100, 300, 1000, or 3000 and can be at most about 3, 10, 30, 100, 300, 1000, 3000, 10,000, 30,000, or 100,000. The timescales of aggregation of the encapsulated agent active material and copolymer self-assembly may be balanced. Therefore, the hydrophilic active material and polymers may precipitate simultaneously, and overcome the limitations of low encapsulated agent incorporations and aggregation found with other techniques based on slow solvent exchange (e.g., dialysis). The inverse Flash NanoPrecipitation process may be insensitive to the chemical specificity of the components, so that it is a broadly applicable nanoparticle formation technique.

In an embodiment, the encapsulated agent and stabilizing agent are dissolved in a single process solvent stream or solution. This stream is then rapidly mixed with an antisolvent in a mixer such that the time for solvent/antisolvent mixing is more rapid than the assembly time of the nanoparticles, for example, as described in WO 2017/112828 A1, WO 2015/200054, and U.S. Pat. No. 8,137,699. A stream is synonymous with a solution phase used as the input to the mixing process. The single process solvent stream may alternatively be mixed against more than one antisolvent stream. The flow rates of the solvent and antisolvent streams may be the same or different. The flow rates may be varied to further tune the solvent/antisolvent composition in the central cavity of a mixer. For example, the composition in the central cavity of the mixer can be 50 vol % solvent or less, or 40 vol % solvent or less, or 25 vol % solvent or less, or 10 vol % solvent or less. In an embodiment, the crosslinking agent, if included, may be dissolved in all antisolvent inlet streams. In an embodiment, the crosslinking agent, if included, may be dissolved in a single antisolvent stream or in multiple antisolvent streams.

In an embodiment, the encapsulated agent and stabilizing agent are dissolved in separate process solvent streams. The process solvent used to dissolve the stabilizing agent and the process solvent used to dissolve the encapsulated agent may be, but are not required to be, the same. For example, the encapsulated agent can be dissolved in a first polar process solvent to form an encapsulated agent solution, and the stabilizing agent can be dissolved in a second polar process solvent to form a stabilizing agent solution. The encapsulated agent and stabilizing agent solutions, are mixed, e.g., simultaneously mixed, with the antisolvent to form a mixed solution. The first polar process solvent and the second polar process solvent can be miscible, or they can be completely miscible (i.e., so that another phase is not formed) at the volumetric ratios at which they are mixed. The first polar process solvent and the antisolvent can be miscible, or they can be completely miscible (i.e., so that another phase is not formed) at the volumetric ratios at which they are mixed. The second polar process solvent and the antisolvent can be miscible, or they can be completely miscible (i.e., so that another phase is not formed) at the volumetric ratios at which they are mixed.

A person skilled in the art will recognize that all solvents are miscible to some degree in each other. Miscible solvents are used in the initial nanoparticle precipitation process. “Miscible” solvents as referred to herein are those that when mixed at the ratios used in the process would produce solutions that have no more than 20% of the volume of the minor phase (e.g., a polar process solvent) not dissolved in the majority phase. Completely miscible solvents as referred to herein are those that when mixed at the ratios used in the nanoparticle formation process, the microparticle process, or another process would produce solutions with no phase separation. “Immiscible” solvents as referred to herein are those that when mixed at the ratios used in the process would produce solutions that have 20% or more of the volume of the minor phase not dissolved in the majority phase.

Nanoparticles can be produced from copolymers that are dissolved in a process solvent with no hydrophilic active material added.

Additional antisolvent may be added after the micromixing process to adjust the final composition of the solvent mixture as desired. The additional antisolvent may be added by inline mixing or it may already be present in the collection vessel for the outlet of the mixer. The final composition of the mixture may be adjusted so that the process solvent content is 25 vol % or less, or 20 vol % or less, or 15 vol % or less, or 10 vol % or less, or 5 vol % or less. This may be referred to as the quench or the collection bath or the dilution bath.

The size of the resulting nanoparticles from this process can be controlled by controlling the mixing velocity used to create them, the total mass concentration of the encapsulated agent and the stabilizing agent in the process solvent, the process solvent(s) and antisolvent(s), the ratio of the encapsulated agent and the stabilizing agent, and the supersaturation of the encapsulated agent and the stabilizing agent upon mixing with the antisolvent.

In a method of the invention, particles can be made that have sizes in the range of 15 nm to 10500 nm, sizes in the range of 20 nm to 6000 nm, sizes in the range of 20 nm to 1000 nm, sizes in the range of 35 nm to 400 nm, or sizes in the range of 40 nm to 300 nm. Sizes can be determined by dynamic light scattering. For example, particles can be made that have sizes of at least about 15 nm, 20 nm, 35 nm, 40 nm, 50 nm, 100 nm, 200 nm, 300 nm, 400 nm, 600 nm, 900 nm, 1000 nm, 2000 nm, 4000 nm, or 6000 nm, and have sizes of at most about 20 nm, 35 nm, 40 nm, 50 nm, 100 nm, 200 nm, 300 nm, 400 nm, 600 nm, 900 nm, 1000 nm, 2000 nm, 4000 nm, 6000 nm, or 10500 nm. Sizes reported and cited herein are the intensity average reported values as determined by the Malvern Nanosizer deconvolution program for particles smaller than 2000 nm, and determined by scanning electron microscopy. Other intensity weighted deconvolution methods can be used to determine sizes of the nanoparticles.

### Extraction of Inverse Nanocarriers

After inverse nanoparticle formation, additional processing steps can be carried out to generate a desirable formulation. Residual DMSO or other process solvent can be removed using an extraction if the nanoparticles are dispersed in a water immiscible antisolvent. The composition of the aqueous solution can be modified to promote stability of the polymer stabilizer. For example, the aqueous stream may contain 150 mM sodium chloride to tune the osmolarity. A sugar, a polyethylene glycol (PEG), or other osmolyte may be used to achieve a similar effect. The pH can be adjusted to limit stabilizer solubility. Organic acids like acetic acid or citric acid can be used, as can mineral acids such as hydrochloric acid. Similarly, bases such as ammonium hydroxide and sodium hydroxide can be added. Buffer systems such as borate or carboxylate or others may be used. The extraction can be carried out for 15 minutes or longer using standard protocols acceptable to the processing scale. In an embodiment, the aqueous phase along with the interface is sent to waste, and the organic phase containing the inverse nanoparticles is retained for further processing. Standard extraction methods known to the art can be used in this process. This is described in Markwalter, C. E. et al., Polymeric Nanocarrier Formulations of Biologics Using Inverse Flash NanoPrecipitation. The AAPS Journal (2020) Vol. 22, No. 2, p. 18 (doi: 10.1208/s12248-019-0405-z), which is hereby incorporated by reference in its entirety.

### Additive Agent and Second Stabilizing Agent Incorporation

In an embodiment of the invention, additive agents are added to the inverse nanocarrier and then processed such that they assemble onto the surface of the inverse nanocarrier. The additive agent assembly is solubility driven and is not controlled by charge complexation as is the case for lipid nanoparticles. Without being bound by theory, the additive agents (for example, hydrophobic or amphiphilic species) can be assembled onto the surface during sequential solvent exchange processes—first to a reforming solvent and then to an aqueous system. The exchange into a reforming solvent can be achieved by distillation or dialysis or other techniques known to the field. The exchange to an aqueous system can subsequently be achieved using Flash NanoPrecipitation techniques for rapidly mixing an organic stream containing components to be assembled with an aqueous antisolvent.

It is surprising that the incorporation of additive agents, such as lipids, had a positive effect on the encapsulation efficiency of encapsulated agents. In the examples below, improvements in encapsulation efficiency and therapeutic effect are demonstrated using a process and compositions reported herein.

The additive agent and second stabilizing agent may be added to the inverse nanocarrier dispersion in a pure form or as a solution in which these are dissolved in a suitable organic solvent. A suitable organic solvent is one in which the additive agent is soluble (can be molecularly dissolved), but does not solubilize the first stabilizing agent from the surface of the inverse nanoparticle. For example, organic solvents such as chloroform, dichloromethane, ethanol, tetrahydrofuran, acetonitrile or methanol may be suitable solvents for adding the agent. The suitable solvent may be the same as the solvent in which the inverse nanoparticles are dispersed. The above list is representative and is not exhaustive. One skilled in the art would recognize that the particular agent and inverse nanocarrier composition can guide or determine solvent choices. The additive agent may be directly dissolved into the inverse nanocarrier dispersion without being first dissolved in an organic solution.

In an embodiment of the invention, the agent or agents include fatty acids, lipids, phospholipids, fatty acid methyl esters, fatty acid ethyl esters, sterols, cholesterol, organic acids, oils, cationic ionizable lipids, cationic lipids, multivalent cationic lipids, sphingosines, sphingolipids, branched tail lipids, lipidoids, anionic lipids, glycosylated lipids, pH sensitive lipids, lipids containing modified headgroups, biotinylated lipids, ceramides, diactylene lipids, adjuvants, sphingomyelin, prostaglandins, eicosanoids, glycerides, glycosylated diacyl glycerols, triglycerides, monoglycerides, diglycerides, oxygenated fatty acids, prenols, cholic acids, bile acids, taurocholic acids, carnitine lipids, oxysterols, cholesteryl esters, glycosylated sterols, hydrophobic small molecules, and/or bioactive small molecules. The second stabilizing agent may include PEG-modified lipids, PEG-containing block copolymers, PEG-b-PLA, PEG-b-PLGA, PEG-b-PCL, dextran-modified lipids, dextran-containing block copolymers, poly(sarcosine)-modified lipids, poly(sarcosine)-containing block copolymers, poly(sarcosine)-b-PLA, poly(sarcosine)-b-PLGA, and/or poly(sarcosine)-b-PCL. The stabilizing agent has a water-soluble (or hydrophilic) region and a hydrophobic region.

The agent or agents may be a mixture of components and is not limited to single component addition. Ratios of these agents to each other and to the inverse nanocarrier are defined below. The additive agent mixture can be between a phospholipid and a cholesterol, 30-[N—(N′,N′-dimethylaminoethane)-carbamoyl]cholesterol (DC-Chol), a second lipid, a cationic lipid, a cationic ionizable lipid, an anionic lipid, ethyl oleate, beta-sitosterol, dioleoylphosphatidylethanolamine (DOPE), a fatty acid, a fatty acid ester, a polymer, a polyester, a poly(lactic-co-glycolic acid), or hydrophobic organic compound of known pharmaceutical effect. This list is not intended to be exhaustive and other additive agents of suitable properties can be selected as well.

An embodiment of the invention employs additive agents that are a lipid blend of a cationic ionizable lipid, phospholipid, and structural lipid such as a sterol. The cationic ionizable lipid may be one known to the field. The cationic ionizable lipid may be permanently ionized, that is, it may be a quaternary amine bearing a permanent charge to form a cationic lipid. Such lipid blends have been widely described in publications and patents such as U.S. Pat. Nos. 8,058,069, 9,364,435, U.S. Pat. No. 10,221,127, WO2018170306A1, and WO2020061367A1. This listing of patents and applications is not intended to be exhaustive, but rather to provide representative examples of suitable lipid blends.

Phospholipids useful in the compositions and methods may be selected from the non-limiting group consisting of 1,2-distearoyl-s-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (Cl 6 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(l-glycerol) sodium salt (DOPG), dipalmitoylphosphatidylglycerol (DPPG), palmitoyloleoylphosphatidylethanolamine (POPE), distearoyl-phosphatidyl-ethanolamine (DSPE), dipalmitoyl phosphatidyl ethanolamine (DPPE), dimyristoylphosphoethanolamine (DMPE), 1-stearoyl-2-oleoyl-phosphatidy ethanolamine (SOPE), 1-stearoyl-2-oleoyl-phosphatidylcholine (SOPC), sphingomyelin, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine (LPE), and mixtures thereof. In some embodiments, a nanoparticle composition includes POPC. In certain embodiments, a nanoparticle composition includes DOPE. In certain embodiments, a nanoparticle composition includes DOPC. In some embodiments, a nanoparticle composition includes both POPC and DOPE.

The additive agent or agents may be selected from anionic lipids. The term “anionic lipid” refers to any lipid that is negatively charged at physiological pH. These lipids include, but are not limited to, phosphatidylglycerols, cardiolipins, diacylphosphatidylserines, diacylphosphatidic acids, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids.

A cationic lipid or cationic ionizable lipid may be selected from any list known in the field. See, for example, U.S. Pat. No. 10,221,127, International Application Publications WO2018170306A1 and WO2020061367A1, U.S. Patent Publication Nos. 20060083780, 20060240554, and 20210101875, U.S. Pat. Nos. 5,208,036, 5,264,618, 5,279,833, 5,283,185, 5,753,613, and 5,785,992, WO2019246203A1, and U.S. Pat. Nos. 10,562,849, 10,888,626, and 5,885,613. The term “cationic lipid” refers to any of a number of lipid species that carry a net positive charge at a selected pH, such as physiological pH (e.g., pH of about 7.0). In some cases, the cationic lipids include a protonatable tertiary amine (e.g., pH titratable) head group, alkyl chains, ether or ester linkages between the head group and alkyl chains, and 0 to 3 double bonds. In other cases, the cationic lipid contains branched lipid tails formed of saturated alkyl chains, such as those reported in WO2020061367A1. Other cationic lipid variations include thiourea and squaramide moieties at the head group. Commonly used cationic lipids are summarized in the literature (Hou, Zaks, Langer, and Dong. Nature Reviews Materials, 6, 1078-1094, 2021. Kauffman, Webber, and Anderson. Journal of Controlled Release, 240, 227-234, 2016) and include Dlin-MC3-DMA, SM-102, ALC-0315, ePC, C12-200, cKK-E12, OF-Deg-Lin, A2-iso5-2DC18, 306Oᵢ₁₀, 503O₁₃, BAME-O16B, TT3, and FTT5. Cationic lipids can be multivalent, such as MVL5 and GL67.

Additional cationic lipids include: 1,2-dilinoleyloxy-N,N-dimethylaminopropane (DLinDMA), 1,2-dilinolenyloxy-N,N-dimethylaminopropane (DLenDMA), 2,2-dilinoleyl-4-(2-dimethylaminoethyl)-[1,3]-dioxolane (Dlin-KC2-DMA; “XTC2”), 2,2-dilinoleyl-4-(3-dimethylaminopropyl)-[1,3]-dioxolane (Dlin-KC3-DMA), 2,2-dilinoleyl-4-(4-dimethylaminobutyl)-[1,3]-dioxolane (Dlin-KC4-DMA), 2,2-dilinoleyl-5-dimethylaminomethyl-[1,3]-dioxane (Dlin-K6-DMA), 2,2-dilinoleyl-4-N-methylpepiazino-[1,3]-dioxolane (Dlin-K-MPZ), 2,2-dilinoleyl-4-dimethylaminomethyl-[1,3]-dioxolane (Dlin-K-DMA), 1,2-dilinoleylcarbamoyloxy-3-dimethylaminopropane (Dlin-C-DAP), 1,2-dilinoleyoxy-3-(dimethylamino)acetoxypropane (Dlin-DAC), 1,2-dilinoleyoxy-3-morpholinopropane (Dlin-MA), 1,2-dilinoleoyl-3-dimethylaminopropane (DLinDAP), 1,2-dilinoleylthio-3-dimethylaminopropane (Dlin-S-DMA), 1-linoleoyl-2-linoleyloxy-3-dimethylaminopropane (Dlin-2-DMAP), 1,2-dilinoleyloxy-3-trimethylaminopropane chloride salt (Dlin-TMA), 1,2-dilinoleoyl-3-trimethylaminopropane chloride salt (Dlin-TAP), 1,2-dilinoleyloxy-3-(N-methylpiperazino)propane (Dlin-MPZ), 3-(N,N-dilinoleylamino)-1,2-propanediol (DlinAP), 3-(N,N-dioleylamino)-1,2-propanedio (DOAP), 1,2-dilinoleyloxo-3-(2-N,N-dimethylamino)ethoxypropane (Dlin-EG-DMA), N,N-dioleyl-N,N-dimethylammonium chloride (DODAC), 1,2-dioleyloxy-N,N-dimethylaminopropane (DODMA), 1,2-distearyloxy-N,N-dimethylaminopropane (DSDMA), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy)propyl)-N,N,N-trimethylammonium chloride (DOTAP), 3-(N—(N′,N′-dimethylaminoethane)-carbamoyl)cholesterol (DC-Chol), N-(1,2-dimyristyloxyprop-3-yl)-N,N-dimethyl-N-hydroxyethyl ammonium bromide (DMRIE), 2,3-dioleyloxy-N-[2(spermine-carboxamido)ethyl]-N,N-dimethyl-1-propanaminiumtrifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy]-3-dimethyl-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-N,N′-dilinoleylcarbamyl-3-dimethylaminopropane (DLincarbDAP), or mixtures thereof. In certain embodiments, the cationic lipid is DLinDMA, Dlin-KC2-DMA (“XTC2”), 4-(dimethylamino)-butanoic acid, (10Z,13Z)-1-(9Z,12Z)-9,12-octadecadien-1-yl-10,13-nonadecadien-1-yl ester (Dlin-MC3-DMA) or mixtures thereof. In addition to these, a cationic lipid may also be a lipid including a cyclic amine group.

The additive agent or agents may include a polymer. This may include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycobde), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, poly orthoesters, poly(ester amides), polyamides, lipocationic polymers such as disclosed in U.S. Pat. No. 9,801,944, poly(ester ethers), polycarbonates, polyalkylenes such as polyethylene and polypropylene, polyalkylene terephthalates such as poly(ethylene terephthalate), polyvinyl alcohols (PVA), polyvinyl ethers, polyvinyl esters such as poly (vinyl acetate), polyvinyl halides such as poly(vinyl chloride) (PVC), polyvinylpyrrolidone (PVP), polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, polyoxamines, poly(ortho)esters, poly(butyric acid), poly(valeric acid), poly(lactide-co-caprolactone), trimethylene carbonate, poly (7V-acryloyl morpholine) (PacM), poly(2-methyl-2-oxazoline) (PMOX), poly(2-ethyl-2-oxazoline) (PEOZ), and polyglycerol.

The additive agent or agents can include structural lipids selected from, but not limited to, sterols, cholesterol, fecosterol, sitosterol, ergosterol, campesterol, stigmasterol, brassicasterol, tomatidine, tomatine, ursolic acid, alpha-tocopherol, and mixtures thereof. Cholesterol derivatives may be selected, such as cholestanol, cholestanone, cholestenone, coprostanol, cholesteryl-2′-hydroxyethyl ether, and/or cholesteryl-4′-hydroxybutyl ether. In some embodiments, the structural lipid is cholesterol. In some embodiments, the structural lipid includes cholesterol and a corticosteroid (such as prednisolone, dexamethasone, prednisone, and hydrocortisone), or a combination thereof.

In some embodiments, a nanoparticle composition that includes one or more lipids described herein may also include one or more lipidated adjuvants, e.g., Glucopyranosyl Lipid Adjuvant (GLA) and Pam3CSK4.

The second stabilizing agent can be a copolymer of a hydrophilic block coupled with a hydrophobic block, for example, the second stabilizing agent can be a second stabilizing amphiphilic agent or second stabilizing amphiphilic copolymer. Inverse nanocarriers coated by the disclosed process can be coated with graft, block, or random amphiphilic copolymers. These amphiphilic polymers can have a molecular weight of between about 1000 g/mole and about 50,000 g/mole, between about 3000 g/mole and about 25,000 g/mole, or at least about 2000 g/mole.

Examples of suitable hydrophobic blocks in an amphiphilic polymer that is a block copolymer include, but are not limited to the following: acrylates including methyl acrylate, ethyl acrylate, propyl acrylate, n-butyl acrylate (BA), isobutyl acrylate, 2-ethyl acrylate, and t-butyl acrylate; methacrylates including ethyl methacrylate, n-butyl methacrylate, and isobutyl methacrylate; acrylonitriles; methacrylonitrile; vinyls including vinyl acetate, vinylversatate, vinylpropionate, vinylformamide, vinylacetamide, vinylpyridines, vinyl phenols and vinylimidazole; aminoalkyls including aminoalkylacrylates, aminoalkylmethacrylates, and aminoalkyl(meth)acrylamides; styrenes; cellulose acetate phthalate, cellulose acetate succinate, hydroxypropylmethylcellulose phthalate, poly(D,L lactide), poly (D,L-lactide-co-glycolide), poly(glycolide), poly(hydroxybutyrate), poly(alkylcarbonate) and poly(orthoesters), polyesters, poly(hydroxyvaleric acid), polydioxanone, poly(ethylene terephthalate), poly(malic acid), poly(tartronic acid), polyanhydrides, polyphosphazenes, poly(amino acids), and their copolymers; hydrophobic peptide-based polymers and copolymers based on poly(L-amino acids), poly(ethylene-vinyl acetate) (“EVA”) copolymers, silicone rubber, polyethylene, polypropylene, polydienes (polybutadiene, polyisoprene, and hydrogenated forms of these polymers), maleic anhydride copolymers of vinyl methylether and other vinyl ethers, polyamides (nylon 6,6), polyurethane, poly(ester urethanes), poly(ether urethanes), and poly(esterurea). For example, polymeric blocks include poly(ethylenevinyl acetate), poly(D,L-lactic acid) oligomers and polymers, poly(L-lactic acid) oligomers and polymers, poly(glycolic acid), copolymers of lactic acid and glycolic acid, poly(caprolactone), poly(valerolactone), polyanhydrides, copolymers of poly(caprolactone) or poly(lactic acid).

Natural products with sufficient hydrophobicity to act as the hydrophobic portion of the amphiphilic polymer include, for example, hydrophobic vitamins (for example, vitamin E, vitamin K, and vitamin A), carotenoids, sterols, cholesterols, and retinols (for example beta carotene, astaxanthin, trans- and cis-retinal, retinoic acid, folic acid, dihydrofolate, retinylacetate, retinyl palmintate), cholecalciferol, calcitriol, hydroxycholecalciferol, ergocalciferol, alpha-tocopherol, alpha-tocopherol acetate, alpha-tocopherol nicotinate, and estradiol.

Examples of suitable hydrophilic blocks in an amphiphilic polymer include but are not limited to the following: carboxylic acids including acrylic acid, methacrylic acid, itaconic acid, and maleic acid; polyoxyethylenes or polyethylene oxide; polyacrylamides and copolymers thereof with dimethylaminoethylmethacrylate, diallyldimethylammonium chloride, vinylbenzyltrimethylammonium chloride, acrylic acid, methacrylic acid, 2-acrylamido-2-methylpropane sulfonic acid and styrene sulfonate, polyvinyl pyrrolidone, starches and starch derivatives, dextran and dextran derivatives; polypeptides, such as poly(lysine), poly(arginine), poly(glutamic acid), poly(sarcosine); polyhyaluronic acids, alginic acids, polylactides, polyethyleneimines, polyionenes, polyacrylic acids, and polyiminocarboxylates, poly(ethylene glycol), gelatin, and unsaturated ethylenic mono or dicarboxylic acids. For example, the hydrophilic blocks can be of poly(ethylene glycol). To produce cationic amphiphilic polymers DMAEMA (dimethylaminoethylmethacrylate), polyvinyl pyridine (PVP), chitosan, poly(lysine), poly(ethylenimine) or dimethylaminoethylacrylamide (DMAMAM) can be used. A representative list of suitable cationic polymers may be found in the literature, such as Kauffman, Webber, and Anderson. Journal of Controlled Release, 240, 227-234 (2016), and a further listing of suitable hydrophilic polymers can be found in Handbook of Water-Soluble Gums and Resins, R. Davidson, McGraw-Hill (1980); these documents are hereby incorporated by reference herein in their entirety.

The polymer blocks can be diblock, triblock, or multiblock repeats. For example, the amphiphilic polymer can be polystyrene-block-poly(ethylene glycol) (PS-b-PEG), poly(lactic acid)-block-poly(ethylene glycol) (PLA-b-PEG), poly(caprolactone)-block-poly(ethylene glycol) (PCL-b-PEG), poly(lactic-co-glycolic acid)-block-poly(ethylene glycol) (PLGA-b-PEG), or tri-block forms of the diblock copolymers listed above. Furthermore, triblock copolymers such as poly(ethylene oxide)-block-poly(propylene oxide)-block-poly(ethylene oxide) (PEO-b-PPO-b-PEO) or poly(ethylene oxide)-block-poly(butylene oxide)-block-poly(ethylene oxide) (PEO-b-PBO-b-PEO) may be used.

In an amphiphilic polymer that is a graft copolymer, the length of a grafted moiety can vary. For example, the grafted segments can be alkyl chains of 4 to 22 carbons or equivalent to 2 to 11 ethylene units in length. Grafted groups may also include lipids, phospholipids or cholesterol. The grafting of the polymer backbone can be useful to enhance solvation or nanoparticle stabilization properties. Suitable chemical moieties grafted to the block unit of the copolymer include alkyl chains containing species such as amides, imides, phenyl, carboxy, aldehyde, or alcohol groups.

In some embodiments, the second stabilizing agent is a conjugated lipid. Examples of suitable conjugated lipids include, but are not limited to, PEG-lipid conjugates, dextran-lipid conjugates, cationic-polymer-lipid conjugates (CPLs), sarcosine-lipid conjugates, and mixtures thereof. In certain embodiments, the particles include either a PEG-lipid conjugate together with a CPL. In certain embodiments, the PEG lipid is selected from a PEG-modified phosphatidylethanolamine, a PEG-modified phosphatidic acid, a PEG-modified ceramide, a PEG-modified dialkylamine, a PEG-modified diacylglycerol, and/or a PEG-modified dialkylglycerol. For example, some embodiments include a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), a pegylated ceramide (PEG-cer), or a PEG dialkoxypropylcarbamate such as ω-methoxy(polyethoxy)ethyl-N-(2,3-di(tetradecanoxy)propyl)carbamate or 2,3-di(tetradecanoxy)propyl-N-(ω-methoxy(polyethoxy)ethyl)carbamate, or 1,2-distearoyl-sn-glycero-3-phosphoethanolamine-N-[amino(polyethylene glycol)-2000](PEG-DPSE).

The second stabilizing agent may be a blend of different stabilizing agents. For example, two block copolymers may be used at a desired ratio. For example, a PEG-lipid and a block copolymer may be used at a desired ratio.

The additive agent or agents is added at a desired mass or mole ratio with respect to the encapsulated agent mass (for example, the mRNA mass). For the purposes of this text, the encapsulated agent mass is the amount of encapsulated agent in inverse nanocarrier after the iFNP assembly step. (This accounts for process hold-up in the mixer.) When multiple additive agents are added, they are also added at a defined mass or mole ratio to each other. Simple screening experiments may be employed to identify suitable compositions of the encapsulated agent, first stabilizing agent, additive agent(s), and second stabilizing agent(s). For a given inverse nanocarrier composition, the additive agent or agents may be added at a mass ratio selected from the range of 1:0.01 to 1:100 (encapsulated agent to additive agent or agents). The mass ratio may be selected from the range of 1:0.25 to 1:25. The mass ratio may be selected from the range of 1:0.5 to 1:15.

When two additive agents are added, they may be at a defined ratio with respect to one another. That ratio may be selected from the range of 1:0.01 to 1:100. The ratio may be selected from the range of 1:0.1 to 1:10. For example, the additive agents may be a lipid and a polyester. In some instances, the additive agent may be two lipids or a lipid and a sterol. The ratio may be selected based on a known mechanism or synergistic ratio. For example, cholesterol and a lipid may be employed at a ratio that mimics mammalian cell membrane compositions. On a molar basis, the cholesterol ratio with the lipid can be 20-50 mol % or 25-40 mol % or 30-40 mol %. These examples are intended to be illustrative and not limiting.

Similarly, multiple additive agents may be added. The total mass of the additive agents may follow the ranges outlined above. The additive agents may be added at a mass ratio with respect to the encapsulated agent of 1:0.1 to 1:100. The ratio of the multiple additive agents may be selected empirically through direct screening experiments. The ratio may be selected based on a known mechanism or synergistic ratio. For example, the multiple additive agents may include a cationic ionizable lipid, a phospholipid, and a structural lipid such as cholesterol. In considering the molar composition of these three additive agents, the cationic ionizable lipid may be included at a mole ratio of 0% to 75%, the phospholipid may be included at a ratio of 0% to 50%, and the structural lipid may be included at a ratio of 0% to 50%. The cationic ionizable lipid may be included at a mole ratio of 20% to 60%, the phospholipid may be included at a ratio of 0% to 40%, and the structural lipid may be included at a ratio of 10% to 50%. In the particular instance where the second stabilizing agent is a PEG-lipid, the PEG-lipid mole ratio may also be taken into account. For example, the cationic ionizable lipid may be included at a mole ratio of 0% to 75%, the phospholipid may be included at a ratio of 0% to 50%, the structural lipid may be included at a ratio of 0% to 50%, and the PEG-lipid may be included at a ratio of 0.5% to 10%. The cationic ionizable lipid may be included at a mole ratio of 20% to 60%, the phospholipid may be included at a ratio of 5% to 25%, the structural lipid may be included at a ratio of 15% to 50%, and the PEG-lipid may be included at a ratio of 1% to 8%. For example, the cationic ionizable lipid may be included at a ratio from 45% to 60%, the phospholipid may be included at a ratio from 5% to 10%, the structural lipid may be included at a ratio from 30% to 40%, and the PEG-lipid may be included at a ratio from 2% to 8%. In an embodiment, a PEG block copolymer such as PEG-b-PLA may be used instead of a PEG-lipid.

In an embodiment where cationic agents such as cationic ionizable lipids are co-formulated with a nucleic acid species, an “N:P ratio”, which is the molar ratio of nitrogen groups on the cationic species to the phosphate groups on the nucleic acid species, can be reported. In the disclosed process, when a cationic ionizable lipid species is included, the cationic species may be included at an N:P ratio of 0.1:1 to 10:1. The N:P ratio may be selected from the range of 1:1 to 6:1. This ratio is a further refining of the overall composition ranges defined on a mass basis above. The specific value may be selected on an empirical basis through activity-based screening assays suitable to the particular application.

After addition of the agent or agents, the dispersion of the inverse nanocarrier and the agent or agents may be aged for a specified period of time. The age time may be 5 minutes or more. The age time may be 15 minutes or more. The age time may be 1 hour or more. The age time may be 24 hours or more. One skilled in the art will recognize that stability considerations, including degradation profiles of the nucleic acid, may dictate age conditions. The age process may be carried out under controlled temperature conditions. The dispersion of inverse nanocarrier and agent or agents may be aged at 37° C. or higher. The dispersion of inverse nanocarrier and agent or agents may be aged at 20° C. or higher. The dispersion of inverse nanocarrier and agent or agents may be aged at 2-8° C. or higher. The dispersion of inverse nanocarrier and agent or agents may be aged at −20° C. or higher. The dispersion of inverse nanocarrier and agent or agents may be stirred or agitated (using any technique known to the field, including magnetic stir bars, stirred tanks using impellers, overhead impellers of any suitable design including retreat curve impellers or chevron impellers) or may be stored static without agitation.

In general, the second stabilizing agent(s) are added prior to solvent exchange to the reforming solvent. Agents that are fully soluble in the reforming solvent (not partially or fully insoluble and therefore not assembled onto the inverse nanocarrier) may be added after the solvent exchange but before aqueous mixing to form the coated nanocarrier.

The second stabilizing agent or agents is added at a desired mass or mole ratio with respect to the encapsulated agent mass (for example, the mRNA mass). For the purposes of this text, the encapsulated agent mass is the amount of encapsulated agent in inverse nanocarrier after the iFNP assembly step. (This accounts for process hold-up in the mixer.) When multiple second stabilizing agents are added, they are also added at a defined mass or mole ratio to each other. Simple screening experiments may be employed to identify suitable compositions of the encapsulated agent, first stabilizing agent, additive agent(s), and second stabilizing agent(s). For a given inverse nanocarrier composition, the second stabilizing agent or agents may be added at a mass ratio selected from the range of 1:0.01 to 1:100 (encapsulated agent to second stabilizing agent(s)). In an embodiment, the mass ratio may be selected from the range of 1:0.25 to 1:25. In an embodiment, the mass ratio may be selected from the range of 1:0.5 to 1:15.

When two stabilizing agents are added, they may be at a defined ratio with respect to one another. That ratio may be selected from the range of 1:0.01 to 1:100. The ratio may be selected from the range of 1:0.1 to 1:10. The ratio may be selected based on a known mechanism or synergistic ratio or based upon standard screening studies.

### Solvent Exchange

The solvent exchange for iFNP can serve to transfer the inverse nanocarrier into a water-miscible organic solvent referred to as the reforming solvent, in preparation for the coating process. In the disclosed process, the solvent exchange serves an additional purpose of driving the assembly of the additive agent onto the inverse nanocarrier surface prior to the coating step. In the examples provided below using POPC or POPC blends as the additive agent(s), process performance (as defined by high encapsulation efficiency and low burst release) were adversely impacted when the additive agent(s) were added after addition of the reforming solvent. In general, the second stabilizing agent may be molecularly dissolved (both hydrophobic and hydrophilic portions remain soluble in the reforming solvent) or it may assemble like the additive agent during solvent exchange to the reforming solvent. This role of the reforming solvent was unexpected.

The reforming solvent should be selected such that the hydrophobic block or region of the first stabilizing agent remains soluble during solvent exchange. The reforming solvent should be selected, so that the additive agent does not exhibit bulk precipitation upon addition of the reforming solvent to the inverse nanocarrier dispersion in the iFNP antisolvent; however, bulk precipitation of the additive agent upon addition of the reforming solvent may occur in the absence of an inverse nanocarrier dispersion. For additive agents with amphiphilic features, the reforming solvent can be selected such that the hydrophilic feature is poorly soluble but the hydrophobic feature is soluble. When the additive agent or agents includes phospholipids, lipids, or cationic lipids, representative but non-limiting reforming solvents include acetonitrile, propionitrile, acetone, or tetrahydrofuran.

The solvent exchange from antisolvent to reforming solvent can be achieved by any method known to the field. For example, distillation or dialysis can be employed. If used, the distillation may be carried out using standard techniques known to the field. If the solvent exchange is achieved through a distillation method, the reforming solvent can have a higher boiling point than the antisolvent, or an azeotrope composition that allows for removal of the antisolvent by evaporation. The distillation may be completed in a put-and-take fashion (concentrating and then adding additional reforming solvent before repeating the process). The distillation may be run as a constant volume distillation (concentrating to a desired volume and then continually adding reforming solvent at a rate approximately matching the evaporation rate of the solvent mixture).

In an embodiment of the invention, the dispersion of inverse nanocarriers, additive agent(s), and second stabilizing agent in the antisolvent is first concentrated to a desired concentration before adding a first portion of the reforming solvent as required by the selected distillation method. As a non-limiting example, given an approximately 1 mg RNA batch size in dichloromethane (DCM), the antisolvent, the volume was reduced from around 4.5 mL to around 2 mL by distillation. Then, 8 mL of acetonitrile, the reforming solvent, was added. The mixture was then concentrated to about 1.5 mL and 8 mL of acetonitrile was added again. This was repeated until acetonitrile had been added 3 times. The dispersion in acetonitrile was then concentrated to a target range. A practitioner of ordinary skill in the art will recognize that the volumes and associated concentration ranges listed here may be adjusted to accommodate process constraints such as minimum or maximum volume constraints.

The distillation should proceed until the residual antisolvent content has been reduced such that the solution can be homogeneously mixed with the aqueous antisolvent in the coating step. That is, the distillation should proceed, so that the residual antisolvent does not form a second liquid phase.

One skilled in the art will recognize that distillation unit operations can be conducted with a range of operating temperatures, pressures, and total mass concentrations of species in the solvents. Considerations such as process throughput, economics, vessel volumes, time constraints, cooling and heating capabilities, and encapsulated agent stability may dictate these decisions. Generally speaking, the temperature and pressure should be selected to afford distillative conditions identifiable from vapor-liquid equilibrium data for the contemplated solvent mixture. Temperature may be selected in the range from 5° C. to 50° C., or 15° C. to 40° C. or 25° C. to 35° C. Pressure may be selected in the range from 10 torr to 400 torr, or 50 torr to 250 torr. As the distillation progresses and the solvent composition changes, the temperature and pressure may also be adjusted as required. The total mass concentration of the encapsulated agent, first stabilizing agent, additive agent(s) and second stabilizing agent can be in the range from 0.1 mg/mL to 200 mg/mL or from 0.5 mg/mL to 40 mg/mL.

### Formation of Coated Nanocarriers—the Coating Step Mixing Process

In an embodiment of the invention, the inverse nanocarrier with additive agent and second stabilizing agent is then processed into an aqueous environment. This process is referred to as the “coating step” because it drives assembly of the second stabilizing agent on the nanocarrier surface and ensures the formation of a hydrophobic layer around the encapsulated agent. The method of coating the nanocarriers is as previously described by Johnson et al., termed “Flash NanoPrecipitation” (FNP), Johnson, B. K., et al., AIChE Journal (2003) 49:2264-2282 and U.S. Pat. No. 8,137,699, which are incorporated herein by reference in their entirety. This mixing technique was also used in the formation of the inverse nanocarrier. Solvent quality is rapidly reduced by micromixing against water or an aqueous buffer or mixture wherein the time of mixing is faster than the aggregation of the nanoparticles and balances with the timescale of stabilizing agent self-assembly.

While not meant to be limiting, two such geometries are the Confined Impinging Jet mixer (CIJ) and the multi-inlet vortex mixer (MIVM). These examples are meant to be illustrative rather than limiting or exhaustive and are mentioned above.

The vortex mixer consists of a confined volume chamber where one solvent stream containing reforming solvent with inverse nanocarrier, additives, and stabilizing agent, is mixed at high velocity with one or more solvent streams containing water or an aqueous buffer. When coating the nanoparticles via Flash NanoPrecipitation, mixing occurs fast enough to allow high supersaturation levels of all components to be reached prior to the onset of inverse nanocarrier aggregation. The Flash NanoPrecipitation process may be insensitive to the chemical specificity of the components, so that it is a broadly applicable nanoparticle coating technique. The confined impinging jet mixer is a similar mixing geometry to the vortex mixer but with only one inlet stream for the reforming solvent and one for the aqueous stream.

In an embodiment, the coating formed by the second stabilizing agent can have an inner region and an outer region. The inner region can include hydrophobic or less polar region(s) of the second stabilizing agent, and the outer region can include hydrophilic or more polar region(s) of the second stabilizing agent.

In an embodiment, the coated nanocarrier includes a nanoparticle with multiple regions. The core contains the encapsulated agent, such as a water soluble agent, for example, a nucleic acid, and hydrophilic or more polar region(s) of the first stabilizing agent, for example, a first stabilizing amphiphilic copolymer, such as region(s) including dextran. Around this core, i.e., surrounding the core, is a shell including hydrophobic or less polar region(s) of the first stabilizing agent, for example, a first stabilizing amphiphilic copolymer, such as region(s) including PLGA, additive agent(s) (such as a lipid, phospholipid, or lipid blends), and hydrophobic or more polar region(s) of a second stabilizing agent, for example, a second stabilizing amphiphilic agent, such as a second stabilizing amphiphilic copolymer (also termed a coating stabilizer or coating polymer). The shell can have an interior surface and an exterior surface, and the interior surface can be in contact with the core. The second stabilizing amphiphilic agent can include hydrophilic or more polar region(s) and hydrophobic or less polar region(s). The hydrophilic or more polar region(s) of the second stabilizing amphiphilic agent can be at the exterior surface of the shell, for example, as a corona on the exterior surface of the shell, which extends beyond the exterior surface of the shell, away from a center of the coated nanocarrier or nanoparticle, for example, towards an environment that surrounds the coated nanocarrier or nanoparticle. The corona can be around the shell, i.e., the corona can surround the shell. That is, the hydrophilic or more polar region(s) of the second stabilizing amphiphilic agent can be in contact with the environment that is outside of the shell and outside of the nanoparticle as a whole. The hydrophobic or less polar region(s) of the second stabilizing amphiphilic agent can be within the shell. For example, the hydrophobic or less polar region(s) of the first stabilizing amphiphilic copolymer and the hydrophobic or less polar region(s) of the second stabilizing amphiphilic agent can be in contact with each other. For example, the surface of the coated nanocarrier can include the hydrophilic or less polar region(s) of the second stabilizing agent, for example, poly(ethylene glycol) (PEG).

For example, the shell can include non-polar, less polar, or hydrophobic agents or regions of agents, including of the first stabilizing agent, the additive agent, and of the second stabilizing agent.

In a method of the invention, nanoparticles can be made that have sizes in the range of 15 nm to 10500 nm, sizes in the range of 20 nm to 6000 nm, sizes in the range of 20 nm to 1000 nm, sizes in the range of 35 nm to 400 nm, or sizes in the range of 40 nm to 300 nm. Sizes can be determined by dynamic light scattering. For example, particles can be made that have sizes of at least about 15 nm, 20 nm, 35 nm, 40 nm, 50 nm, 100 nm, 200 nm, 300 nm, 400 nm, 600 nm, 900 nm, 1000 nm, 2000 nm, 4000 nm, or 6000 nm and have sizes of at most about 20 nm, 35 nm, 40 nm, 50 nm, 100 nm, 200 nm, 300 nm, 400 nm, 600 nm, 900 nm, 1000 nm, 2000 nm, 4000 nm, 6000 nm, or 10500 nm. Sizes reported and cited herein are determined by dynamic light scattering using the manufacturer-installed deconvolution programs.

A nanoparticle composition may be relatively homogenous. A polydispersity index may be used to indicate the homogeneity of a nanoparticle composition, e.g., the particle size distribution of the nanoparticle composition. A small (e.g., less than 0.3) polydispersity index generally indicates a narrow particle size distribution. A nanoparticle composition may have a polydispersity index of from about 0 to about 0.3, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.29, or 0.30 or be within a range between two of these values. In some embodiments, the polydispersity index of a nanoparticle composition may be from about 0.10 to about 0.20.

The zeta potential may describe the surface charge of a nanoparticle. Compositions can be selected to provide different nanocarrier zeta potentials. In some embodiments, the zeta potential of a nanoparticle composition may be from about −30 mV to about +30 mV, from about −20 mV to about +20 mV, from about −12 mV to about +10 mV, from about −12 mV to about +5 mV, from about −12 mV to about 0 mV, from about −12 mV to about −5 mV, from about −10 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

Nanoparticles that are mixtures of polymers and lipids are reviewed by Hadinoto, Sundaresan, Cheow, European Journal of Pharmaceutics and Biopharmaceutics (2013) 85, 427-443, which is hereby incorporated by reference herein in its entirety. The existing methods all rely on charge complexation to encapsulate the RNA, as with lipid nanoparticles, and employ different emulsification or nanoprecipitation techniques to form a polymer core. For example, the process discussed herein is distinct in that it employs sequential nanoprecipitation steps to apply sequential layers to the nanoparticle.

### Formation of Coated Nanocarriers—Stream Composition

The concentrations, stabilizing agent, reforming solvent, and aqueous buffer (antisolvent) used in the coating process may be optimized such that individual inverse nanocarriers are coated. The number of mixer inlet streams may be governed by the mixer geometry. The multi-inlet vortex mixer generally contains four streams, whereas the confined impinging jet mixer generally contains two streams. The volumetric ratios of these streams are selected as described above for inverse Flash NanoPrecipitation (iFNP) and Flash NanoPrecipitation (FNP) to ensure suitable mixing speed and intensity. The aqueous content after mixing can be selected to be 50 vol % or more with the reforming solvent. The aqueous content after mixing can be 75 vol % or more, or 90 vol % or more. Further, an additional aqueous dilution after mixing can be employed to adjust the residual reforming solvent content to produce a stable nanocarrier dispersion.

The aqueous composition can be deionized water, or a suitable buffer such as phosphate-buffered saline, or 4-(2-hydroxyethyl)-1-piperazineethanesulfonic acid (HEPES) buffer, acetate buffer, citrate buffer, or another organic or inorganic buffer controlling the pH in the range of from 2 to 8, or from 4 to 7.5, such as those in the list of Good's buffers. These representative buffers are not meant to be limiting. The aqueous composition in the mixer can be deionized water, but the additional aqueous dilution after mixing can be a suitable buffer. The aqueous stream may be unbuffered but contain species to tune the osmolarity of the aqueous system. Osmolytes can be chosen, such as sugars, PEG oligomers, salts, or amino acids.

The concentration of species in the reforming solvent can be selected to achieve the desired outcome. One skilled in the art will recognize that a concentration may be selected that is uneconomical because it is too dilute. Alternatively, concentrations may be selected that are too high and can result in inverse nanocarrier aggregation, for example, as the concentration approaches the regime where, during coating, inverse nanocarrier collisions happen faster than the second stabilizing agent can reach the surface or where the stabilizing agent is in a polymer overlap regime. Typical concentration ranges that are suitable for coating include from 0.5 mg/mL to 50 mg/mL, or from 1 mg/mL to 20 mg/mL, or from 5 mg/mL to 15 mg/mL.

When a vortex mixer that has 4 inlet streams is used there are additional inlet stream composition possibilities. For example, one stream can be the reforming solvent, two streams can be the aqueous streams, and one stream can include a second reforming solvent or solvent mixture. For example, the second reforming solvent can include the second stabilizing agent when that agent has not been added directly to the inverse nanocarrier dispersion at another point in the process. This method allows for the use of stabilizing agents that do not have suitable solubility profiles in the desired reforming solvent.

### Post-Processing and Preparation of Pharmaceutical Form—Buffer Exchange

The coated nanocarrier may be further processed to a pharmaceutical form useful for treating a patient or subject in need thereof. These methods of preparing a pharmaceutical form are known to the field. For example, the coated nanocarrier can be prepared with a pharmaceutically acceptable carrier (e.g., physiological saline or phosphate buffer) selected in accordance with the route of administration and standard pharmaceutical practice. Normal buffered saline (e.g., 135-150 mM NaCl) can be employed as the pharmaceutically-acceptable carrier. Other suitable carriers include, e.g., water, buffered water, 0.4% saline, 0.3% glycine, and the like, including glycoproteins for enhanced stability, such as albumin, lipoprotein, globulin, etc. These are examples and are not meant to limiting what can be used. As used herein, “carrier” includes any and all solvents, dispersion media, vehicles, coatings, diluents, antibacterial and antifungal agents, isotonic and absorption delaying agents, buffers, carrier solutions, and the like. Carrier is distinct from the term “nanocarrier”. For example, the phrase “pharmaceutically-acceptable” refers to molecular entities and compositions that do not produce an undesireable allergic or similar unacceptable reaction when administered to a human.

The pharmaceutically-acceptable carrier is generally added following coated nanocarrier formation. The residual reforming solvent can be removed to an acceptable level as determined according to ICH (International Council for Harmonisation of Technical Requirements for Pharmaceuticals for Human Use) guidelines. Any suitable method known in the art may be used for reforming solvent removal and carrier introduction. For example, suitable methods include but are not limited to dialysis, ultrafiltration, tangential flow filtration, diafiltration, and centrifugal ultrafiltration.

### Post-Processing and Preparation of Pharmaceutical Form—Excipients

Coated nanocarriers may include any substance useful in pharmaceutical compositions. For example, the composition may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. A number of pharmaceutically acceptable excipients are known in the art.

Examples of diluents may include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and/or combinations thereof. Granulating and dispersing agents may be selected from the non-limiting list consisting of potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked polyvinyl pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Surface active agents and/or emulsifiers may include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN®20], polyoxyethylene sorbitan [TWEEN® 60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g. polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g. CREMOPHOR®), polyoxyethylene ethers, (e.g. polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER® 188, cetrimonium bromide, cetylpyridinium chloride, benzalkonium chloride, docusate sodium, and/or combinations thereof.

A binding agent may be starch (e.g. cornstarch and starch paste); gelatin; sugars (e.g., sucrose, glucose, dextrose, dextrin, molasses, lactose, lactitol, mannitol); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; poly methacrylates; waxes; water; alcohol; and combinations thereof, or any other suitable binding agent.

Examples of preservatives may include, but are not limited to, antioxidants, chelating agents, antimicrobial preservatives, antifungal preservatives, alcohol preservatives, acidic preservatives, and/or other preservatives. Examples of antioxidants include, but are not limited to, alpha tocopherol, ascorbic acid, acorbyl palmitate, butylated hydroxyanisole, butylated hydroxy toluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxy ethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxy benzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxy toluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN®II, NEOLONE™, KATHON™, and/or EUXYL®.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levubnate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof. Lubricating agents may selected from the non-limiting group consisting of magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, and combinations thereof.

Examples of oils include, but are not limited to, almond, apricot kernel, avocado, babassu, bergamot, black current seed, borage, cade, chamomile, canola, caraway, camauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropylmyristate, jojoba, kukui nut, lavandin, lavender, lemon, macademia nut, mallow, mango seed, meadowfoam seed, mink, nutmeg, olive, orange, palm, palm kernel, peach kernel, peanut, poppy seed, pumpkin seed, rapeseed, rice bran, rosemary, safflower, sandalwood, sea buckthorn, sesame, shea butter, silicone, soybean, sunflower, tea tree, thistle, vetiver, walnut, and wheat germ oils as well as butyl stearate, caprylic triglyceride, capric triglyceride, cyclomethicone, diethyl sebacate, dimethicone 360, simethicone, isopropyl myristate, mineral oil, octyldodecanol, oleyl alcohol, silicone oil, and/or combinations thereof.

Excipients may be included as cryoprotectants or lyoprotectants. Examples of excipients are glucose, sucrose, trehalose, lactose, mannitol, sorbitol, aerosil, maltose, fructose, dextran, glycerol, poly(vinyl pyrrolidone), poly(vinyl alcohol), glycine, cyclodextrins, hydroxypropyl-beta-cyclodextrin, gelatine, poly(ethylene glycol), alanine, sodium chloride, citrate, histidine, and starch derivatives.

### Post-Processing and Preparation of Pharmaceutical Form—Freezing or Lyophilization

The pharmaceutical form may be prepared in a liquid or solid form. Liquid forms may be stored at a suitable temperature. For example, the liquid form may be stored at room temperature, or at 2-8° C. The dispersion may be stored as a frozen liquid. For example, the coated nanocarrier dispersion can be stored at −12° C. or less, or at −20° C. or less, or at −80° C. or less. The coated nanocarrier pharmaceutical form may be prepared as a lyophilized or spray dried powder. Methods known to the field may be employed to lyophilize or spray dry the coated nanocarrier pharmaceutical form, including the addition of cryoprotectant or lyoprotectant excipients described above.

### Compositions

Compositions may include an encapsulated agent or agents selected from nucleic acid classes including but not limited to RNA, DNA, mRNA, siRNA, microRNA, circular RNA, antisense oligonucleotides, tRNA, or plasmids. In an embodiment, the composition may include a salt, for example, a calcium salt. In an embodiment, the encapsulated agent or agents may be a nucleic acid and a peptide, protein, or second nucleic acid, or combinations thereof. The encapsulated agent may be multiple nucleic acid sequences.

Compositions may include a first stabilizing agent that is a diblock, triblock, or comb copolymer. The hydrophilic block can be selected from dextran, poly(aspartic acid), and poly(glutamic acid). The hydrophobic block can be selected from poly(lactic acid), poly(lactic-co-glycolic acid), and poly(caprolactone).

Compositions may include additive agents selected from lipids, phospholipids, ethyl oleate, POPC, DSPC, HSPC, DOPC, DOPE, cationic lipids or cationic ionizable lipids, cholesterol, beta-sitosterol, fatty acid methyl esters, fatty acid ethyl esters, hydrophobic polymers, cationic or ionizable polymers, and combinations.

Compositions include a second stabilizing agent that is a diblock, triblock, or comb copolymer, or a lipid conjugate, or mixtures. The hydrophilic polymer block or the hydrophilic lipid conjugate can be selected from poly(ethylene glycol), poly(sarcosine), poly(aspartic acid), poly(glutamic acid), poly(lysine), or poly(arginine), and combinations. The hydrophobic polymer block can be selected from poly(lactic acid), poly(lactic-co-glycolic acid), ad poly(caprolactone). Compositions include PEG lipids such as PEG-DMG or PEG-DSPE, or PEG-b-PLGA or PEG-b-PLA, or mixtures.

### Methods of Use

The present application provides compositions and methods of delivering a therapeutic and/or prophylactic agent to a cell, tissue, or organ by administering a coated nanocarrier pharmaceutical composition of said agent to a subject in need thereof. Delivery of a therapeutic and/or prophylactic agent to a subject may involve administering a coated nanocarrier pharmaceutical composition including the encapsulated agent, where administration of the composition involves contacting a cell membrane with the composition. Coated nanocarrier compositions and/or pharmaceutical compositions including one or more coated nanocarrier compositions may be administered to any patient or subject, including those patients or subjects that may benefit from a therapeutic effect provided by the delivery of a therapeutic and/or prophylactic to one or more particular cells, tissues, organs, or systems or groups thereof, such as the hepatic system. Although the descriptions provided herein include those of compositions that are suitable for administration to humans or materials derived from humans, it will be understood by the skilled artisan that such compositions are may be suitable for administration to another living system, including animals or animal-derived materials. Modification of compositions suitable for administration to humans in order to render the compositions suitable for administration to various animals or animal-derived materials may be done, and an ordinarily skilled veterinary pharmacologist may be able to design and/or perform such modification with ordinary, if any, experimentation. Subjects to which administration of the compositions is contemplated include, but are not limited to, humans, other primates, and other animals, including commercially relevant animals such as cattle, pigs, horses, sheep, cats, dogs, rodents, mice, chickens, and/or rats. Subjects to which administration of the compositions is contemplated also include materials derived from humans or other animals, such as cells, tissues, and organs.

Coated nanocarrier pharmaceutical forms may be prepared for administration by routes that include, without limitation, oral, topical, transdermal, inhalation, parenteral, intraocular, subretinal, sublingual, mucosal, buccal, rectal, vaginal, and intranasal. The term parenteral as used herein includes subcutaneous injections, intravenous, intramuscular or other direct injections into tissues or organs, intradermal, intrathecal, intraperitoneal, intraarterial, intracisternal, intracerebral, or intratumoral injection or infusion techniques. Pharmaceutical compositions of an embodiment of the invention are formulated so as to allow the active ingredients contained therein to be bioavailable upon administration of the composition to a patient. Compositions for administration to a subject or patient may take the form of one or more dosage units, where for example, a tablet, or other dosage form, may be a single dosage unit, and a container of a compound of an embodiment of the invention in aerosol form may hold a plurality of dosage units. Actual methods of preparing such dosage forms may be known or apparent, to those skilled in this art.

Injectable preparations, for example, sterile injectable aqueous or oleaginous suspensions may be formulated according to the known art using suitable dispersing agents, wetting agents, and/or suspending agents. Compositions for rectal or vaginal administration are typically suppositories which can be prepared by mixing compositions with suitable non-irritating excipients such as cocoa butter, polyethylene glycol, and/or a suppository wax which are solid at ambient temperature but liquid at body temperature and therefore melt in the rectum or vaginal cavity and release the active ingredient. Solid dosage forms for oral administration include capsules, tablets, pills, films, powders, and granules. Dosage forms for topical and/or transdermal administration of a composition may include ointments, pastes, creams, lotions, gels, powders, solutions, sprays, inhalants, and/or patches. Additionally, the present disclosure contemplates the use of transdermal patches, which often have the added advantage of providing controlled delivery of a compound to the body. Such dosage forms may be prepared, for example, by dissolving and/or dispensing the compound in the proper medium. Suitable devices for use in delivering intradermal pharmaceutical compositions described herein include short needle devices such as those described in U.S. Pat. Nos. 4,886,499; 5, 190,521; 5,328,483; 5,527,288; 4,270,537; 5,015,235; 5, 141,496; and 5,417,662. Pharmaceutical compositions formulated for pulmonary or intranasal delivery may provide an active ingredient in the form of droplets of a solution and/or suspension or as a dry powder.

In general, the step of contacting a cell with a coated nanocarrier may be performed in vivo, ex vivo, in culture, or in vitro. The amount of coated nanocarrier contacted with a cell, and/or the amount of encapsulated agent therein, may depend on the type of cell or tissue being contacted, the means of administration, the physiochemical characteristics, and other factors.

In certain embodiments, an mRNA included in a coated nanocarrier may encode a natural or recombinant polypeptide, such as a protein, that may replace one or more polypeptides that may be reduced or substantially absent in a cell contacted with the nanoparticle composition. The one or more substantially absent polypeptides may be lacking due to a genetic mutation of the encoding gene or a regulatory pathway thereof. The one or more substantially absent polypeptides may be enzymes with partially or wholly absent activity. Alternatively, a recombinant polypeptide produced by translation of the mRNA may antagonize the activity of an endogenous protein present in, on the surface of, or secreted from the cell. An antagonistic recombinant polypeptide may be desirable to combat deleterious effects caused by activities of the endogenous protein, such as altered activities or localization caused by mutation. In other embodiments, a recombinant polypeptide produced by translation of the mRNA may alter the DNA of gene expression in a cell through encoding various endonucleases and accessory factors. Polypeptides including Cas proteins, TALENs, and other endonucleases may be desirable to change a subject's DNA to treat a disease. In other embodiments, a recombinant polypeptide produced by translation of the mRNA in a cell may encode for a polypeptide not normally found in that cell. Such polypeptides may include but are not limited to transcription factors, insulin, antibodies, hormones, cytokines, complement factors, clotting factors, and growth factors and may be used to treat various diseases.

In certain embodiments, the coated nanocarrier composition with an encapsulated agent including of one or more nucleic acids forming an interfering RNA sequence (e.g., siRNA) or an antisense oligonucleotide sequence provides a method for treatment in vitro and/or in vivo of a disease or disorder in a mammal or animal subject by downregulating or silencing the transcription and/or translation of one or more target nucleic acid sequences or genes of interest.

In certain embodiments, the coated nanocarrier composition with an encapsulated agent including one or more nucleic acids forming a non-coding ribnonucleic acid (RNA) sequence (e.g., transfer RNA (tRNA), microRNA (miRNA), small interfering RNA (siRNA), Piwi-interacting RNA (piRNA), small nucleolar RNA (snoRNA), messenger RNA (mRNA), or long non-coding RNA acid (lncRNA)) provides a method for treatment in vitro and/or in vivo of a disease or disorder in an animal by regulating the transcription and/or translation of one or more target nucleic acid sequences or genes of interest.

In certain embodiments, the coated nanocarrier composition with an encapsulated agent including one or more nucleic acids forming a DNA sequence (e.g. DNA aptamers or donor sequences for homologous repair) provides a method for treatment in vitro and/or in vivo of a disease or disorder in an animal by regulating the sequence, transcription and/or translation of one or more target nucleic acid sequences or genes of interest.

In certain embodiments, a DNA included in a coated nanocarrier may encode a natural or recombinant RNA sequence, such as an mRNA, an siRNA, or another RNA species, whether natural or unnatural. In some embodiments, the utility of DNA included in a coated nanocarrier is subsumed by the same utility as RNA. In other embodiments, DNA included in a coated nanocarrier may contain or encode for additional elements that promote retention within the cell as an episomal complex or may promote integration into the chromosomal DNA. In some embodiments, coated nanocarrier-DNA complexes may be used to treat various diseases through both acute and extended expression. In some embodiments, DNA included in a nanocarrier may be used as a non-viral gene therapy with applications to various diseases.

### Method of Use: Treating Diseases and Disorders

Coated nanocarrier compositions may be useful for treating a disease, disorder, or condition. For example, such compositions may be useful in treating a disease, disorder, or condition characterized by missing or aberrant protein or polypeptide activity. For example, a nanoparticle composition including an mRNA encoding a missing or aberrant polypeptide may be administered or delivered to a cell. Subsequent translation of the mRNA may produce the polypeptide, thereby reducing or eliminating an issue caused by the absence of or aberrant activity caused by the polypeptide. Because translation may occur rapidly, the methods and compositions may also be useful in the treatment of acute diseases, disorders, or conditions such as sepsis, stroke, hyperammonemia, and myocardial infarction or chronic diseases. Diseases, disorders, and/or conditions characterized by dysfunctional or aberrant protein or polypeptide activity for which a composition may be administered include, but are not limited to, rare diseases, infectious diseases (as both vaccines and therapeutics), cancer, proliferative, and hyperproliferative diseases, fibrotic diseases, genetic diseases (e.g., cystic fibrosis), autoimmune diseases, diabetes, neurodegenerative diseases, neurologic, cardio- and reno-vascular diseases, endocrine, neuroendocrine, hepatobiliary, ophthalmologic, musculoskeletal, gastrointestinal, reproductive, genitourinary, lymphatic, dermatologic, hematologic, and metabolic diseases.

For example, a nanoparticle composition including a DNA or an mRNA may treat monogenic disorders of the liver such as urea cycle disorders by replacing substantially reduced or absent proteins. Example of such in-born errors in metabolism include NAGS (N-acetylglutamate synthase) deficiency, CPS (carbamoyl phosphate synthetase) deficiency, OTC (ornithine transcarbamoylase) deficiency, Citrullinemia Type 1, Citrullinemia Type 2, Argininosuccinic aciduria, Argininemia, or HHH (Hyperornithinemia-Hyperammonemia-Homocitrullinuria) syndrome. Administration of a nanoparticle composition including a DNA or an mRNA of a native or recombinant polypeptide encoding for NAGS, CPS1, OTC1, ASS1, SLC25A13, ASL1, ARG1, SLC25A15 or ORNT1 may treat a urea cycle disorder in a subject in need thereof.

A nanoparticle composition including a DNA or an mRNA may treat other monogenic or polygenic disorders including, but not limited to, lysosomal storage disorders such as Fabry disease, acidurias such as methylmalonic aciduria, and Wilson's disease where substantially reduced or absent levels of liver enzymes results in the disease. Additionally, a nanoparticle composition including a DNA or an mRNA that is translated in the liver may produce a polypeptide or protein that is absent in other tissues. This method may treat a disease by elevating plasma levels of therapeutic proteins including but not limited to insulin in Type 1 diabetes or Type 2 diabetes, clotting factors in hemophilia, or protease inhibitors in alpha 1 antitrypsin deficiency.

In addition, a nanoparticle composition including a DNA or an mRNA may treat diseases including, but not limited to, neurological disorders, musculoskeletal disorders, hematologic disorders, methyl malonic acidemia, tyrosinemia, Wilson disease, hemophilia A, hemophilia B, hereditary fructose intolerance, glutaric aciduria, chronic granulomatous disease, fatty acid oxidation disorders, MTP (mitochondrial trifunctional protein) deficiency, propionic acidemia, maple syrup urine disease, alpha-1 antitrypsin inhibitor deficiency, intermittent porphyria, galactosemia, hyperoxaluria, Fabry, ADA (adenosine deaminase) deficiency, cystic fibrosis, familial-hypercholesterolemia, hemophilia, Duchenne muscular dystrophy, Becker muscular dystrophy, Rett syndrome, fragile-X syndrome, sarcosinemia, ataxias, dystrophies, Fanconi anemia, sickle-cell anemia, Gaucher's disease, Hunter syndrome, X-linked SCID (severe combined immunodeficiency), and the like. These provided diseases are intended to be exemplary and non-limiting.

A nanoparticle composition including a DNA or an mRNA may treat cancer or proliferative disorders by encoding for growth suppressing or apoptotic proteins. Delivery of natural or recombinant TP53, p21INK4a, Rb, APC, SMAD2, SMAD3, BAX or other DNAs/mRNAs may be therapeutic in various cancers or fibrotic diseases such as colorectal cancers or hepatocellular cancers.

A nanoparticle composition including a DNA or an mRNA may treat an autoimmune or immune-mediated inflammatory disorder by encoding for an autoantigen or other peptides or proteins involved generating an autoimmune or deleterious immune response. Such autoantigens include, but are not limited to, any that are known to play a role in any of various autoimmune or immune-mediated inflammatory disorders, for example multiple sclerosis, rheumatoid arthritis, systemic lupus erythematosis, scleroderma, psoriasis, dermatomyositis, pemphigus vulgaris, inflammatory bowel disease, intersitial lung disease. In another embodiment, the nanoparticle composition includes a DNA or an mRNA co-encapsulated with other compounds that are capable of modulating immune responses, including, but not limited to immunomodulatory drugs, biologics or other molecules.

In another embodiment, the disorder treated involves one of the polypeptides or proteins listed below. These are exemplary and are not limiting as a selection: ABCA4; ABCD3; ACADM; AGL; AGT; ALDH4A1; ALPL; AMPD1; APOA2; AVSD1; BRCD2; C1QA; C1QB; C1QG; C8A; C8B; CACNA1S; CCV; CD3Z; CDC2L1; CHML; CHS1; CIAS1; CLCNKB; CMD1A; CMH2; CMM; COL11A1; COL8A2; COL9A2; CPT2; CRB1; CSE; CSF3R; CTPA; CTSK; DBT; DIO1; DISC1; DPYD; EKV; ENO1; ENO1P; EPB41; EPHX1; F13B; F5; FCGR2A; FCGR2B; FCGR3A; FCHL; FH; FMO3; FMO4; FUCA1; FY; GALE; GBA; GFND; GJA8; GJB3; GLC3B; HF1; HMGCL; HPC1; HRD; HRPT2; HSD3B2; HSPG2; KCNQ4; KCS; KIF1B; LAMB3; LAMC2; LGMD1B; LMNA; LOR; MCKD1; MCL1; MPZ; MTHFR; MTR; MUTYH; MYOC; NB; NCF2; NEM1; NPHS2; NPPA; NRAS; NTRK1; OPTA2; PBX1; PCHC; PGD; PHA2A; PHGDH; PKLR; PKP1; PLA2G2A; PLOD; PPOX; PPTO; PRCC; PRG4; PSEN2; PTOS1; REN; RFX5; RHD; RMD1; RPE65; SCCD; SERPINC1; SJS1; SLC19A2; SLC2A1; SPG23; SPTA1; TAL1; TNFSF6; TNNT2; TPM3; TSHB; UMPK; UOX; UROD; USH2A; VMGLOM; VWS; WS2B; ABCB11; ABCG5; ABCG8; ACADL; ACP1; AGXT; AHHR; ALMS1; ALPP; ALS2; APOB; BDE; BDMR; BJS; BMPR2; CHRNA1; CMCWTD; CNGA3; COL3A1; COLAA3; COL4A4; COL6A3; CPS1; CRYGA; CRYGEP1; CYP1B1; CYP27A1; DBI; DES; DYSF; EDAR; EFEMPI; EIF2AK3; ERCC3; FSHR; GINGF; GLC1B; GPD2; GYPC; HADHA; HADHB; HOXD13; HPE2; IGKC; IHH; IRS1; ITGA6; KHK; KYNU; LCT; LHCGR; LSFC; MSH2; MSH6; NEB; NMTC; NPHP1; PAFAHIP1; PAX3; PAX8; PMS1; PNKD; PPH1; PROC; REG1A; SAG; SFTPB; SLC11A1; SLC3A1; SOS1; SPG4; SRD5A2; TCL4; TGFA; TMD; TPO; UGT1A@; UV24; WSS; XDH; ZAP70; ZFHX1B; ACAA1; AGS1; AGTR1; AHSG; AMT; ARMET; BBS3; BCHE; BCPM; BTD; CASR; CCR2; CCR5; CDL1; CMT2B; COL7A1; CP; CPO; CRV; CTNNB1; DEM; ETM1; FANCD2; FIH; FOXL2; GBE1; GLB1; GLCLC; GNAI2; GNAT1; GP9; GPX1; HGD; HRG; ITIH1; KNG; LPP; LRS1; MCCC1; MDS1; MHS4; MITF; MLH1; MYL3; MYMY; OPA1; P2RY12; PBXP1; PCCB; POU1F1; PPARG; PROS1; PTHR1; RCA1; RHO; SCA7; SCLC1; SCN5A; SI; SLC25A20; SLC2A2; TF; TGFBR2; THPO; THRB; TKT; TM4SF1; TRH; UMPS; UQCRC1; USH3A; VHL; WS2A; XPC; ZNF35; ADH1B; ADH1C; AFP; AGA; AIH2; ALB; ASMD; BFHD; CNGA1; CRBM; DCK; DSPP; DTDP2; ELONG; ENAM; ETFDH; EVC; F11; FABP2; FGA; FGB; FGFR3; FGG; FSHMD1A; GC; GNPTA; GNRHR; GYPA; HCA; HCL2; HD; HTN3; HVBS6; IDUA; IF; JPD; KIT; KLKB1; LQT4; MANBA; MLLT2; MSX1; MTP; NR3C2; PBT; PDE6B; PEE1; PITX2; PKD2; QDPR; SGCB; SLC25A4; SNCA; SOD3; STATH; TAPVR1; TYS; WBS2; WFS1; WHCR; ADAMTS2; ADRB2; AMCN; AP3B1; APC; ARSB; B4GALT7; BHR1; C6; C7; CCAL2; CKN1; CMDJ; CRHBP; CSF1R; DHFR; DIAPH1; DTR; EOS; EPD; ERVR; F12; FBN2; GDNF; GHR; GLRA1; GM2A; HEXB; HSD17B4; ITGA2; KFS; LGMDLA; LOX; LTC4S; MAN2A1; MCC; MCCC2; MSH3; MSX2; NR3C1; PCSK1; PDE6A; PFBI; RASA1; SCZD1; SDHA; SGCD; SLC22A5; SLC26A2; SLC6A3; SM1; SMA@; SMN1; SMN2; SPINK5; TCOF1; TELAB1; TGFB1; ALDH5A1; ARG1; AS; ASSP2; BCKDHB; BF; C2; C4A; CDKN1A; COL10A1; COL11A2; CYP21A2; DYX2; EJM1; ELOVL4; EPM2A; ESR1; EYA4; F13A1; FANCE; GCLC; GJA1; GLYS1; GMPR; GSE; HCR; HFE; HLA-A; HLA-DPB1; HLA-DRA; HPFH; ICS1; IDDM1; IFNGR1; IGAD1; IGF2R; ISCW; LAMA2; LAP; LCA5; LPA; MCDR1; MOCS1; MUT; MYB; NEU1; NKS1; NYS2; OA3; ODDD; OFCO; PARK2; PBCA; PBCRA1; PDB1; PEX3; PEX6; PEX7; PKHD1; PLA2G7; PLG; POLH; PPAC; PSORS1; PUJO; RCD1; RDS; RHAG; RP14; RUNX2; RWS; SCA1; SCZD3; SIASD; SOD2; ST8; TAP1; TAP2; TFAP2B; TNDM; TNF; TPBG; TPMT; TULP1; WISP3; AASS; ABCB1; ABCB4; ACHE; AQP1; ASL; ASNS; AUTS1; BPGM; BRAF; C7orf2; CACNA2D1; CCM1; CD36; CFTR; CHORDOMA; CLCN1; CMH6; CMT2D; COL1A2; CRS; CYMD; DFNA5; DLD; DYT11; EEC1; ELN; ETV1; FKBP6; GCK; GHRHR; GHS; GLI3; GPDS1; GUSB; HLXB9; HOXA13; HPFH2; HRX; IAB; IMMP2L; KCNH2; LAMBI; LEP; MET; NCF1; NM; OGDH; OPNISW; PEX1; PGAM2; PMS2; PON1; PPP1R3A; PRSS1; PTC; PTPN12; RP10; RP9; SERPINE1; SGCE; SHFM1; SHH; SLC26A3; SLC26A4; SLOS; SMAD1; TBXAS1; TWIST; ZWS1; ACHM3; ADRB3; ANK1; CA1; CA2; CCAL1; CLN8; CMT4A; CNGB3; COH1; CPP; CRH; CYP1IB1; CYP11B2; DECR1; DPYS; DURS1; EBS1; ECA1; EGI; EXT1; EYA1; FGFR1; GNRH1; GSR; GULOP; HR; KCNQ3; KFM; KWE; LGCR; LPL; MCPH1; MOS; MYC; NAT1; NAT2; NBS1; PLAT; PLEC1; PRKDC; PXMP3; RP1; SCZD6; SFTPC; SGM1; SPG5A; STAR; TG; TRPS1; TTPA; VMD1; WRN; ABCA1; ABL1; ABO; ADAMTS13; AK1; ALAD; ALDH1A1; ALDOB; AMBP; AMCD1; ASS; BDMF; BSCL; C5; CDKN2A; CHAC; CLA1; CMD1B; COL5A1; CRAT; DBH; DNAI1; DYS; DYT1; ENG; FANCC; FBP1; FCMD; FRDA; GALT; GLDC; GNE; GSM1; GSN; HSD17B3; HSN1; IBM2; INVS; JBTS1; LALL; LCCS1; LCCS; LGMD2H; LMX1B; MLLT3; MROS; MSSE; NOTCHI; ORM1; PAPPA; PIP5K1B; PTCH; PTGS1; RLN1; RLN2; RMRP; ROR2; RPD1; SARDH; SPTLC1; STOM; TDFA; TEK; TMC1; TRIM32; TSC1; TYRP1; XPA; CACNB2; COL17A1; CUBN; CXCL12; CYP17; CYP2C19; CYP2C9; EGR2; EMX2; ERCC6; FGFR2; HK1; HPS1; IL2RA; LGI1; LIPA; MAT1A; MBL2; MKI67; MXI1; NODAL; OAT; OATL3; PAX2; PCBD; PEO1; PHYH; PNLIP; PSAP; PTEN; RBP4; RDPA; RET; SFTPA1; SFTPD; SHFM3; SIAL; THC2; TLX1; TNFRSF6; UFS; UROS; AA; ABCC8; ACAT1; ALX4; AMPD3; ANC; APOAL; APOA4; APOC3; ATM; BSCL2; BWS; CALCA; CAT; CCND1; CD3E; CD3G; CD59; CDKNLC; CLN2; CNTF; CPT1A; CTSC; DDB1; DDB2; DHCR7; DLAT; DRD4; ECB2; ED4; EVR1; EXT2; F2; FSHB; FTH1; G6PT1; G6PT2; GIF; HBB; HBBP1; HBD; HBE1; HBG1; HBG2; HMBS; HND; HOMG2; HRAS; HVBS1; IDDM2; IGER; INS; JBS; KCNJ11; KCNJ1; KCNQ1; LDHA; LRP5; MEN1; MLL; MYBPC3; MYO7A; NNO1; OPPG; OPTB1; PAX6; PC; PDX1; PGL2; PGR; PORC; PTH; PTS; PVRL1; PYGM; RAG1; RAG2; ROM1; RRAS2; SAA1; SCA5; SCZD2; SDHD; SERPINGI; SMPD1; TCIRG1; TCL2; TECTA; TH; TREH; TSG101; TYR; USH1C; VMD2; VRNI; WT1; WT2; ZNF145; A2M; AAAS; ACADS; ACLS; ACVRL1; ALDH2; AMHR2; AOM; AQP2; ATD; ATP2A2; BDC; CIR; CD4; CDK4; CNA1; COL2A1; CYP27B1; DRPLA; ENUR2; FEOM1; FGF23; FPF; GNB3; GNS; HAL; HBP1; HMGA2; HMN2; HPD; IGF1; KCNA1; KERA; KRAS2; KRT1; KRT2A; KRT3; KRT4; KRT5; KRT6A; KRT6B; KRTHB6; LDHB; LYZ; MGCT; MPE; MVK; MYL2; OAP; PAH; PPKB; PRB3; PTPN11; PXR1; RLS; RSN; SAS; SAX1; SCA2; SCNN1A; SMAL; SPPM; SPSMA; TBX3; TBX5; TCF1; TPI1; TSC3; ULR; VDR; VWF; ATP7B; BRCA2; BRCD1; CLN5; CPB2; ED2; EDNRB; ENUR1; ERCC5; F10; F7; GJB2; GJB6; IPF1; MBS1; MCOR; NYS4; PCCA; RB1; RHOK; SCZD7; SGCG; SLC10A2; SLC25A15; STARP1; ZNF198; ACHM1; ARVD1; BCH; CTAA1; DAD1; DFNB5; EML1; GALC; GCH1; IBGC1; IGH@; IGHC group; IGHG1; IGHM; IGHR; IV; LTBP2; MCOP; MJD; MNG1; MPD1; MPS3C; MYH6; MYH7; NP; NPC2; PABPN1; PSEN1; PYGL; RPGRIP1; SERPINA1; SERPINA3; SERPINA6; SLC7A7; SPG3A; SPTB; TCL1A; TGM1; TITF1; TMIP; TRA@; TSHR; USHLA; VP; ACCPN; AHO2; ANCR; B2M; BBS4; BLM; CAPN3; CDAN1; CDAN3; CLN6; CMH3; CYP19; CYP1A1; CYP1A2; DYX1; EPB42; ETFA; EYCL3; FAH; FBN1; FES; HCVS; HEXA; IVD; LCS1; LIPC; MYO5A; OCA2; OTSC1; PWCR; RLBP1; SLC12A1; SPG6; TPM1; UBE3A; WMS; ABCC6; ALDOA; APRT; ATP2A1; BBS2; CARD15; CATM; CDH1; CETP; CHST6; CLN3; CREBBP; CTH; CTM; CYBA; CYLD; DHS; DNASE1; DPEP1; ERCC4; FANCA; GALNS; GAN; HAGH; HBA1; HBA2; HBHR; HBQ1; HBZ; HBZP; HP; HSD11B2; IL4R; LIPB; MC1R; MEFV; MHC2TA; MLYCD; MMVP1; PHKB; PHKG2; PKD1; PKDTS; PMM2; PXE; SALL1; SCA4; SCNN1B; SCNN1G; SLC12A3; TAT; TSC2; VDI; WT3; ABR; ACACA; ACADVL; ACE; ALDH3A2; APOH; ASPA; AXIN2; BCL5; BHD; BLMH; BRCA1; CACD; CCA1; CCZS; CHRNB1; CHRNE; CMT1A; COL1A1; CORD5; CTNS; EPX; ERBB2; G6PC; GAA; GALK1; GCGR; GFAP; GH1; GH2; GP1BA; GPSC; GUCY2D; ITGA2B; ITGB3; ITGB4; KRT10; KRT12; KRT13; KRT14; KRT14L1; KRT14L2; KRT14L3; KRT16; KRT16L1; KRT16L2; KRT17; KRT9; MAPT; MDB; MDCR; MGI; MHS2; MKS1; MPO; MYO15A; NAGLU; NAPB; NF1; NME1; P4HB; PAFAHIB1; PECAM1; PEX12; PHB; PMP22; PRKAR1A; PRKCA; PRKWNK4; PRP8; PRPF8; PTLAH; RARA; RCV1; RMSA1; RP17; RSS; SCN4A; SERPINF2; SGCA; SGSH; SHBG; SLC2A4; SLC4A1; SLC6A4; SMCR; SOST; SOX9; SSTR2; SYM1; SYNS1; TCF2; THRA; TIMP2; TOC; TOP2A; TP53; TRIM37; VBCH; ATP8B1; BCL2; CNSN; CORD1; CYB5; DCC; F5F8D; FECH; FEO; LAMA3; LCFS2; MADH4; MAFD1; MC2R; MCL; MYP2; NPC1; SPPK; TGFBRE; TGIF; TTR; AD2; AMH; APOC2; APOE; ATHS; BAX; BCKDHA; BCL3; BFIC; C3; CACNA1A; CCO; CEACAM5; COMP; CRX; DBA; DDU; DFNA4; DLL3; DM1; DMWD; E11S; ELA2; EPOR; ERCC2; ETFB; EXT3; EYCL1; FTL; FUT1; FUT2; FUT6; GAMT; GCDH; GPI; GUSM; HB1; HCL1; HHC2; HHC3; ICAM3; INSR; JAK3; KLK3; LDLR; LHB; LIG1; LOH19CR1; LYL1; MAN2B1; MCOLN1; MDRV; MLLT1; NOTCH3; NPHS1; OFC3; OPA3; PEPD; PRPF31; PRTN3; PRX; PSG1; PVR; RYR1; SLC5A5; SLC7A9; STK11; TBXA2R; TGFB1; TNNI3; TYROBP; ADA; AHCY; AVP; CDAN2; CDPD1; CHED1; CHED2; CHRNA4; CST3; EDN3; EEGV1; FTLL1; GDF5; GNAS; GSS; HNF4A; JAG1; KCNQ2; MKKS; NBIA1; PCK1; PI3; PPCD; PPGB; PRNP; THBD; TOP1; AIRE; APP; CBS; COL6A1; COL6A2; CSTB; DCR; DSCR1; FPDMM; HLCS; HPE1; ITGB2; KCNE1; KNO; PRSS7; RUNX1; SOD1; TAM; ADSL; ARSA; BCR; CECR; CHEK2; COMT; CRYBB2; CSF2RB; CTHM; CYP2D6; CYP2D7P1; DGCR; DIA1; EWSR1; GGT1; MGCR; MN1; NAGA; NE2; OGS2; PDGFB; PPARA; PRODH; SC02; SCZD4; SERPIND1; SLC5A1; SOX10; TCN2; TIMP3; TST; VCF; ABCD1; ACTL1; ADFN; AGMX2; AHDS; AIC; AIED; AIH3; ALAS2; AMCD; AMELX; ANOP1; AR; ARAF1; ARSC2; ARSE; ARTS; ARX; ASAT; ASSP5; ATP7A; ATRX; AVPR2; BFLS; BGN; BTK; BZX; C1HR; CACNA1F; CALB3; CBBM; CCT; CDR1; CFNS; CGF1; CHM; CHR39c; CIDX; CLA2; CLCN5; CLS; CMTX2; CMTX3; CND; COD1; COD2; COL4A5; COL4A6; CPX; CVD1; CYBB; DCX; DFN2; DFN4; DFN6; DHOF; DIAPH2; DKC1; DMD; DSS; DYT3; EBM; EBP; ED1; ELK1; EMD; EVR2; F8; F9; FCP1; FDPSL5; FGD1; FGS1; FMR1; FMR2; G6PD; GABRA3; GATA1; GDI1; GDXY; GJB1; GK; GLA; GPC3; GRPR; GTD; GUST; HMS1; HPRT1; HPT; HTC2; HTR2c; HYR; IDS; IHG1; IL2RG; INDX; IP1; IP2; JMS; KAL1; KFSD; L1CAM; LAMP2; MAA; MAFD2; MAOA; MAOB; MCF2; MCS; MEAX; MECP2; MF4; MGC1; MIC5; MID1; MLLT7; MLS; MRSD; MRX14; MRX1; MRX20; MRX2; MRX3; MRX40; MRXA; MSD; MTM1; MYCL2; MYP1; NDP; NHS; NPHL1; NROB1; NSX; NYS1; NYX; OAl; OASD; OCRL; ODT1; OFD1; OPA2; OPD1; OPEM; OPN1LW; OPN1MW; OTC; P3; PDHA1; PDR; PFC; PFKFB1; PGK1; PGK1P1; PGS; PHEX; PHKA1; PHKA2; PHP; PIGA; PLP1; POF1; POLA; POU3F4; PPMX; PRD; PRPS1; PRPS2; PRS; RCCP2; RENBP; RENS1; RP2; RP6; RPGR; RPS4X; RPS6KA3; RS1; S11; SDYS; SEDL; SERPINA7; SH2D1A; SHFM2; SLC25A5; SMAX2; SRPX; SRS; STS; SYN1; SYP; TAF1; TAZ; TBX22; TDD; TFE3; THAS; THC; TIMM8A; TIM1; TKCR; TNFSF5; UBE1; UBE2A; WAS; WSN; WTS; WWS; XIC; XIST; XK; XM; XS; ZFX; ZIC3; ZNF261; ZNF41; ZNF6; AMELY; ASSP6; AZF1; AZF2; DAZ; GCY; RPS4Y; SMCY; SRY; ZFY; ABAT; AEZ; AFA; AFD1; ASAH1; ASD1; ASMT; CCAT; CECR9; CEPA; CLA3; CLN4; CSF2RA; CTS1; DF; DIH1; DWS; DYT2; DYT4; EBR3; ECT; EEF1A1L14; EYCL2; FANCB; GCSH; GCSL; GIP; GTS; HHG; HMI; HOAC; HOKPP2; HRPT1; HSD3B3; HTC1; HV1S; ICHQ; ICR1; ICR5; IL3RA; KAL2; KMS; KRT18; KSS; LCAT; LHON; LIMM; MANBB; MCPH2; MEB; MELAS; MIC2; MPFD; MS; MSS; MTATP6; MTCO1; MTC03; MTCYB; MTND1; MTND2; MTND4; MTND5; MTND6; MTRNR1; MTRNR2; MTTE; MTTG; MTTI; MTTK; MTTL1; MTTL2; MTTN; MTTP; MTTS1; NAMSD; OCD1; OPD2; PCK2; PCLD; PCOS1; PFKM; PKD3; PRCA1; PRO1; PROP1; RBS; RFXAP; RP; SHOX; SLC25A6; SPG5B; STO; SUOX; THM; or TTD.

In other examples, a nanoparticle composition including an mRNA encoding an endonuclease, DNA-binding, or RNA-binding factor and further including one or more small guide RNAs (sgRNAs) can be used for various diseases or conditions. For example, mRNA encoding Cas9 and a guide RNA may be used to treat various inherited diseases such as sickle cell anemia or beta-thalassemia. Alternatively, mRNA encoding Cas13d and a guide RNA may be useful in treating RNA repeat disorders such as Huntington's Disease or ALS (amyotrophic lateral sclerosis).

In other examples, a nanoparticle composition including an endonuclease, DNA-binding or RNA-binding factor and further including one or more small guide RNAs can be used for various diseases or conditions. For example, Cas9 and a guide RNA (also referred to as a ribonucleoprotein) may be used to treat various inherited diseases such as sickle cell anemia or beta-thalassemia.

A nanoparticle composition including an siRNA or miRNA encapsulated agent or agents can be used to downregulate or silence the translation (i.e., expression) of a gene of interest. Genes of interest include, but are not limited to, genes associated with viral infection and survival, genes associated with metabolic diseases and disorders (e.g., liver diseases and disorders), genes associated with tumorigenesis and cell transformation (e.g., cancer), angiogenic genes, immunomodulator genes such as those associated with inflammatory and autoimmune responses, ligand receptor genes, and genes associated with neurodegenerative disorders.

A nanoparticle composition including a non-coding RNA such as a tRNA may be used in various disorders resulting from dysregulation or loss of tRNA synthesis including but not limited to Charcot-Marie-Tooth disease, or diseases associated with premature stop codons such as beta-thalassemia. In addition, said tRNA constructs may be engineered with unnatural amino acids to treat various diseases such as cancer.

In another embodiment, a coated nanocarrier composition can be used to induce an immune response directed against one or more tumor-associated antigens or cells, such as cancer cells, expressing one or more tumor-associated antigens. An embodiment of the invention envisions the use of coding or non-coding DNAs or RNAs that lead to expression of tumor-associated antigens (also termed “antigen” herein) as the encapsulated agent in the coated nanocarrier. These antigens may include a sequence essentially corresponding to or being identical to the sequence of a tumor-associated antigen or one or more fragments thereof. In a method of the invention, a coated nanocarrier composition encapsulating an mRNA encoding for antigen is capable of inducing an antigen-specific immune response in a subject including administering to the subject an effective amount to produce an antigen-specific immune response. In some embodiments, the antigen specific immune response includes a T cell response. In some embodiments, the antigen specific immune response includes a B cell response. In some embodiments, the method of producing an antigen-specific immune response involves a single administration of the vaccine. In some embodiments, the method further includes administering one or more booster doses of the vaccine. In some embodiments, the vaccine is administered to the subject by intradermal or intramuscular injection.

In some embodiments, a disease-associated antigen is a tumor antigen. In this embodiment, the coated nanocarrier compositions described herein may be useful in treating cancer or cancer metastasis. Examples for tumor antigens that may be useful in an embodiment of the invention are p53, ART-4, BAGE, beta-catenin/m, Bcr-abL CAMEL, CAP-1, CASP-8, CDC27/m, CDK4/m, CEA, the cell surface proteins of the claudin family, such as CLAUDIN-6, CLAUDIN-18.2 and CLAUDIN-12, c-MYC, CT, Cyp-B, DAM, ELF2M, ETV6-AML1, G250, GAGE, GnT-V, Gap 100, HAGE, HER-2/neu, HPV-E7, HPV-E6, HAST-2, hTERT (or hTRT), LAGE, LDLR/FUT, MAGE-A, for example, MAGE-A1, MAGE-A2, MAGE-A3, MAGE-A4, MAGE-A5, MAGE-A6, MAGE-A7, MAGE-A8, MAGE-A9, MAGE-A10, MAGE-A11, or MAGE-A12, MAGE-B, MAGE-C, MART-1/Melan-A, MC1R, Myosin/m, MUC1, MUM-1, -2,-3, NA88-A, NF1, NY-ESO-1, NY-BR-1, p190 minor BCR-abL, Pml/RARa, PRAME, proteinase 3, PSA, PSM, RAGE, RU1 or RU2, SAGE, SART-1 or SART-3, SCGB3A2, SCP1, SCP2, SCP3, SSX, SURVIVIN, TEL/AML1, TPI/m, TRP-1, TRP-2, TRP-2/INT2, TPTE and WT, for example, WT-1.

In some embodiments, a coated nanoparticle pharmaceutical composition described herein may also include one or more adjuvants, including but not limited to, Glucopyranosyl Lipid Adjuvant (GLA), CpG oligodeoxynucleotides (e.g., Class A or B), poly(I:C), or aluminum hydroxide.

In some embodiments, the encapsulating agent is a DNA or an mRNA encoding for one or more antigens or other proteins derived from an infectious agent. Such infectious agents include any microorganism capable of causing disease in humans or other mammals. In one embodiment, the infectious agent is a strain of virus selected from adenovirus; Herpes simplex, type 1; Herpes simplex, type 2; encephalitis virus, papillomavirus, Varicella-zoster virus; Epstein-barr virus; Human cytomegalovirus; Human herpes virus, type 8; Human papillomavirus; BK virus; JC virus; Smallpox; polio virus; Hepatitis B virus; Human bocavirus; Parvovirus B19; Human astrovirus; Norwalk virus; coxsackievirus; hepatitis A virus; poliovirus; rhinovirus; Severe acute respiratory syndrome virus; SARS-CoV-2; Hepatitis C virus; Yellow Fever virus; Dengue virus; West Nile virus; Rubella virus; Hepatitis E virus; Human Immunodeficiency virus (HIV); Influenza virus; Guanarito virus; Junin virus; Lassa virus; Machupo virus; Sabia virus; Crimean-Congo hemorrhagic fever virus; Ebola virus; Marburg virus; Measles virus; Mumps virus; Parainfluenza virus; Respiratory syncytial virus; Human metapneumovirus; Hendra virus; Nipah virus; Rabies virus; Hepatitis D; Rotavirus; Orbivirus; Coltivirus; Banna virus; Human Enterovirus; Hanta virus; West Nile virus; Middle East Respiratory Syndrome Corona Virus; coronavirus; Japanese encephalitis virus; Vesicular exanthernavirus; Eastern equine encephalitis, Zika virus, and/or Chikungunya virus.

In some embodiments, the virus is a strain of Influenza A or Influenza B or combinations thereof. In some embodiments, the strain of Influenza A or Influenza B is associated with birds, pigs, horses, dogs, humans, or non-human primates. In some embodiments, the antigenic polypeptide encodes a hemagglutinin protein or fragment thereof.

In some embodiments, the infectious agent is a strain of bacteria, such as Tuberculosis (Mycobacterium tuberculosis), clindamycin-resistant Clostridium difficile, fluoroquinolone-resistant Clostridium difficile, methicillin-resistant Staphylococcus aureus (MRSA), multidrug-resistant Enterococcus faecalis, multidrug-resistant Enterococcus faecium, multidrug-resistant Pseudomonas aeruginosa, multidrug-resistant Acinetobacter baumannii, and vancomycin-resistant Staphylococcus aureus (VRSA). In some embodiments, the bacteria is Clostridium difficile.

In some embodiments, the encapsulated agent is multivalent. In some embodiments, the open reading frame of the one or more encapsulated agent (e.g. mRNA sequences) encodes 2 or more antigenic polypeptides. In some embodiments, the open reading frame of the one or more encapsulated agents (e.g., mRNA sequences) encode at least 10, 15, 20, or 50 antigenic polypeptides.

Mammalian cells to be contacted by the composition may include, but are not limited to, hepatocytes, epithelial cells, hematopoietic cells, monocyte cells, endothelial cells, lung cells, alveolar cells, type I alveolar cells, type II alveolar cells, bone cells, stem cells, mesenchymal cells, neural cells, cardiac cells, adipocytes, vascular smooth muscle cells, cardiomyocytes, skeletal muscle cells, lymphoid cells, beta cells, pituitary cells, synovial lining cells, ovarian cells, testicular cells, fibroblasts, B cells, T cells, reticulocytes, leukocytes, granulocytes, and tumor or cancer cells. The target cell may be an antigen presenting cell, a dendritic cell, a macrophage, a spleen cell, a lung cell, a liver sinusoidal cell, or Claudius' cell, Hensen cell, Merkel cell, Müller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, zymogenic cell, kidney cell, or glandular cell.

A person of ordinary skill in the art may employ standard methods to determine the appropriate dosing range and dosage levels for a coated nanocarrier pharmaceutical composition. For example, coated nanocarrier compositions in accordance with the present disclosure may be administered in vivo at dosage levels sufficient to deliver from about 0.0001 mg/kg to about 100 mg/kg, from about 0.001 mg/kg to about 50 mg/kg, from about 0.005 mg/kg to about 10 mg/kg, from about 0.01 mg/kg to about 10 mg/kg, from about 0.05 mg/kg to about 10 mg/kg, from about 0.1 mg/kg to about 10 mg/kg, from about 1 mg/kg to about 10 mg/kg, from about 2 mg/kg to about 10 mg/kg, from about 5 mg/kg to about 10 mg/kg, from about 0.0001 mg/kg to about 5 mg/kg, from about 0.001 mg/kg to about 5 mg/kg, from about 0.005 mg/kg to about 5 mg/kg, from about 0.01 mg/kg to about 5 mg/kg, from about 0.05 mg/kg to about 5 mg/kg, from about 0.1 mg/kg to about 5 mg/kg, or from about 1 mg/kg to about 5 mg/kg, where a dose of 1 mg/kg (mpk) provides 1 mg of an encapsulated agent per 1 kg of subject body weight.

Coated nanocarrier compositions including one or more encapsulated agents may be used in combination with one or more other therapeutic, prophylactic, diagnostic, or imaging agents. The phrase “in combination with” is not intended to imply that the agents must be administered at the same time and/or formulated for delivery together, although these methods of delivery are within the scope of this application. For example, one or more nanoparticle compositions including one or more different therapeutic and/or prophylactics may be administered in combination. Compositions can be administered concurrently with, prior to, or subsequent to, one or more other desired therapeutics or medical procedures. For example, each agent can be administered at a dose and/or on a time schedule determined for that agent.

### EXAMPLES

Note about Naming of First Stabilizing Agents Used in Examples

As described above, the iFNP process uses a first stabilizing agent (e.g., a first stabilizing amphiphilic copolymer). In subsequent examples, stabilizing agents used in the iFNP process are composed of either a poly(aspartic acid) (PAsp) or dextran (Dex) hydrophilic (more polar) region or regions, and a poly(lactic acid) (PLA) or poly(lactic-co-glycolic acid) (PLGA) hydrophobic (less polar) region or regions. The PAsp-b-PLA or PAsp-b-PLGA polymers are diblock copolymers in which the PAsp block was polymerized off of a PLA or PLGA macroinitiator. The dextran-PLGA (or Dex-PLGA) polymers are comb polymers in which PLGA was grafted onto a dextran backbone such that each dextran chain has one or more PLGA chains grafted onto it. The short-form names used for these stabilizing polymers as well as a description of each polymer is provided below in Table 1. The polymers presented in this table are not meant to reflect the full range of stabilizing agent compositions that can be used in this process. For example, poly(glutamic acid) (PGlu) can be used instead of or together with PAsp and/or dextran to form a hydrophilic (more polar) region of a first stabilizing amphiphilic copolymer. For example, polycaprolactone (PCL) can be used instead of or together with PLA and/or PLGA to form a hydrophobic (less polar) region of a first stabilizing amphiphilic copolymer.

In the following Examples, encapsulation efficiency (EE) is the amount of RNA inside the nanoparticles divided by the total amount in solution. Alternatively, EE can be calculated as 100% minus the amount of free (unencapsulated) RNA in solution divided by the total amount of RNA in solution.

Burst release is the fraction of RNA that releases (is no longer encapsulated within a nanocarrier) within 15 minutes. The released RNA is separated from the nanocarrier using an ultrafilter. (Burst release=[RNA outside of the nanocarrier]/[total RNA inside and outside nanocarrier]).

RNA loading is the mass fraction of RNA within the nanocarrier. (RNA Loading=[RNA mass]/[RNA+first stabilizing agent+additive agents+second stabilizing agent mass]).

The terms RNAse protection and Protection are used interchangeably. Protection is the fraction of total RNA in the nanocarrier that is not degraded by an RNAse enzyme added to the dispersion as measured by the RiboGreen assay protocol. (Protection=[non-degraded RNA]/[total RNA]).

TABLE 1
Summary of polymers used as stabilizing agents in the subsequent examples.

| Polymer Name | Description |
|---|---|
| 503-Asp | PAsp-b-PLGA diblock copolymer, 2 kDa PAsp block, 16 kDa PLGA |
| | block with a 50:50 L:G (lactic acid unit:glycolic acid unit) ratio |
| 753-Asp | PAsp-b-PLGA diblock copolymer, 2 kDa PAsp block, 16 kDa PLGA |
| | block with a 75:25 L:G ratio |
| 203-Asp | PAsp-b-PLA diblock copolymer, 2 kDa PAsp block, 16 kDa PLA block |
| 503-80 | Dex-PLGA comb polymer, 10 kDa dextran backbone, 16 kDa PLGA |
| | (50:50 L:G ratio) chains grafted on, final polymer is 80 wt % PLGA |
| 502-70 | Dex-PLGA comb polymer, 10 kDa dextran backbone, 6.3 kDa PLGA |
| | (50:50 L:G ratio) chains grafted on, final polymer is 70 wt % PLGA |
| 502-75 | Dex-PLGA comb polymer, 10 kDa dextran backbone, 6.3 kDa PLGA |
| | (50:50 L:G ratio) chains grafted on, final polymer is 75 wt % PLGA |

### Example 1: Unanticipated Impacts of Lipid and Solvent Selection on RNA Encapsulation

RNA (˜5 kDa in size with unmodified bases) in the triethylamine salt form was dissolved in 0.5 mL of DMSO at 2.5 mg/mL (triethylamine (TEA) salt basis) with a block copolymer stabilizer (2.5 mg/mL) as indicated in Table 2. The naming system for the stabilizing polymers is given at the beginning of the Examples section in Table 1. The DMSO contained 5 vol % deionized water. This solution was rapidly mixed with an antisolvent stream of DCM in a multi-inlet vortex mixer. The DCM (dichloromethane) entered the mixer in three separate streams of 0.5 mL volume. CaCl₂) at 1 charge equivalent relative to acid groups in the formulation was included in one DCM stream. This mixing step formed an inverse nanocarrier with RNA encapsulated. The RNA content, accounting for mixer hold-up and losses was about 0.75 mg (free acid basis). The inverse nanocarrier was then coated with PLA-b-PEG (2.5 mg) after a brine extraction and solvent exchange into a water-miscible reforming solvent (THF (tetrahydrofuran)). The inclusion of 5 mg of a bulking polymer (PLGA or PLA) prior to solvent exchange, salt change of the block copolymer stabilizer, and additional crosslinker afforded EE values less than 45% (formulation IDs 1-3).

RNA release from the PEG-coated nanocarrier was measured by separating the nanocarriers from soluble RNA using a 300 kDa molecular-weight-cut-off ultrafilter. RNA content of the crude nanoparticles and the ultrafilter flowthrough was then measured spectroscopically by absorbance at 260 nm. As a screening metric, the PEG-coated nanocarriers were diluted to 150 mM NaCl (“brine”). Burst release from the nanocarrier was measured after 15 minutes of incubation in this media. Formulations ID 1, 2, and 3 all had burst release of >(greater than) 50%.

Unexpectedly, addition of the lipid POPC in the antisolvent stream during the iFNP step resulted in EE values ranging from 57%-71% and burst release values less than 32% (formulations 4-7). Continued improvement in EE was seen on increasing the POPC amount from 1.95 mg to 3.9 (2×POPC). The following examples demonstrate that POPC can be included after the iFNP step with similar effect.

Surprisingly, a specific and enhanced effect on process performance was observed if acetonitrile (MeCN) was substituted for THE as the reforming solvent. Formulations 8 and 9 exhibited EE values >75% and burst release <20% using this substitution. However, addition of POPC after the solvent exchange into MeCN resulted in EE and burst release values on-par with THE results (53% EE and 37% burst, not included in Table 2).

TABLE 2
Formulation components and analysis summary

| | | | | Bulk | Reforming | | Burst |
|---|---|---|---|---|---|---|---|
| ID | iFNP Stabilizer | Crosslink | Lipid | Polymer | Solvent | EE | Release |
| 1 | 203-Asp | CaCl₂ | None | PLGA | THF | 38% | 53% |
| 2 | TEA + 203-Asp | CaCl₂ | None | PLA | THF | 43% | 61% |
| 3 | TEA + 203-Asp | 2 eq CaCl₂ | None | PLA | THF | 38% | 78% |
| 4 | 203-Asp | CaCl₂ | POPC | PLA | THF | 66% | 28% |
| 5 | 203-Asp | CaCl₂ | 2x POPC | PLA | THF | 65% | 21 |
| 6 | 503-Asp | CaCl₂ | POPC | PLA | THF | 57% | 31% |
| 7 | 503-Asp | CaCl₂ | 2x POPC | PLA | THF | 66% | 23% |
| 8 | 503-Asp | CaCl₂ | POPC | PLA | MeCN | 76% | 17% |
| 9 | 503-Asp | CaCl₂ | 2x POPC | PLA | MeCN | 82% | 17% |

To confirm that composition alone did not afford the process metric enhancements, the same formulation compositions were prepared using a modified process. RNA (˜5 kDa in size with unmodified bases) in the triethylamine salt form was dissolved in 0.5 mL of DMSO at 2.5 mg/mL with a block copolymer stabilizer (5 mg/mL) as indicated in Table 3. The DMSO contained 5 vol % deionized water. This solution was rapidly mixed with an antisolvent stream of either THE or MeCN in a confined impinging jet (CIJ) mixer. The antisolvent stream contained CaCl₂) at 1 charge equivalent, and, optionally, POPC at two different amounts (1.95 or 3.9 mg). After the solvent and antisolvent streams were mixed, 2.5 mg of PLA-b-PEG and 5 mg of PLGA homopolymer were added to the inverse nanoparticles. The mixed stream was not diluted further before the coating step, achieved by CIJ mixing with an equal volume of water. The coated nanocarrier dispersion was then diluted 10× with additional water.

The EE and burst release values are reported in Table 3 and indicate that the composition alone was not responsible for the high EE and low burst values achieved through the use of POPC and acetonitrile in the iFNP process.

TABLE 3
Formulation summary for modified antisolvent process

| | | | | | Burst |
|---|---|---|---|---|---|
| BCP | Crosslink | Antisolvent | Lipid | EE | Release |
| 503-Asp | CaCl₂ | THF | None | 45% | 68% |
| 503-Asp | CaCl₂ | THF | POPC | 47% | 23% |
| 503-Asp | CaCl₂ | THF | 2 × POPC | 47% | 45% |
| 503-Asp | CaCl₂ | MeCN | None | 29% | 89% |
| 503-Asp | CaCl₂ | MeCN | POPC | 54% | 6% |
| 503-Asp | CaCl₂ | MeCN | 2 × POPC | 53% | 33% |

### Example 2: Variations of the Modified iFNP Process Using POPC and Acetonitrile

Variations on the process employing POPC additive and acetonitrile (MeCN) for the reforming solvent were carried out. RNA (˜5 kDa in size with unmodified bases) in the triethylamine salt form was dissolved in 0.5 mL of DMSO at 2.5 mg/mL with a block copolymer stabilizer (5 mg/mL) as indicated in Table 4. The DMSO contained the indicated volume percent of deionized water. This solution was rapidly mixed with an antisolvent stream of DCM in a multi-inlet vortex mixer (MIVM) or confined impinging jet (CIJ) mixer. For the MIVM, the DCM entered the mixer in three separate streams of 0.5 mL volume. For the CIJ, a single stream of 0.5 mL DCM was used. CaCl₂) or Ca(NO₃)₂ at 1 charge equivalent (except where noted) was included in one DCM stream. Additional DCM was added to the mixer outlet to bring the DMSO content to 10 vol % or less. This produces an inverse nanocarrier dispersion in DCM with about 0.75 mg of RNA (free acid basis, accounting for mixer apparatus hold-up and losses).

Samples were then aged for ˜18 hours except where noted in Table 4 and then extracted with a volume of 150 mM NaCl in water (generally about 25-75% of the total inverse nanocarrier dispersion volume) for about 30 minutes. The DCM phase was isolated and the lipid or lipids, hydrophobic additive, and PEG-based coating stabilizer (PLA-b-PEG or vitamin E TPGS (tocopheryl polyethylene glycol succinate)) were added (the coating stabilizer is also termed the second stabilizing amphiphilic agent).

The solvent exchange to the reforming solvent, acetonitrile (MeCN), was carried out as follows. MeCN was added at an equal volume to the DCM and then the dispersion was concentrated by rotary evaporation. A put-and-take distillation involving two additions of MeCN was carried out. For a 0.5 mL DMSO input volume, these additions totaled 8 mL and the final concentration volume was ˜1 mL. The temperature used was 35° C. and the vacuum set point was varied as required to achieve distillation.

The inverse nanocarrier dispersion in acetonitrile was then rapidly mixed against an equal volume of deionized water (except where noted in Table 4) in a CIJ mixer, and a collection bath of additional deionized water was used to reduce the MeCN volume content to less than 10 vol %. This process produced the PEG-coated nanocarrier. Encapsulation efficiency (EE) was determined by separating free RNA from the nanocarrier using a 300 kDa molecular-weight-cut-off ultrafilter. The coated nanocarriers were further diluted, generally about 4-fold in water prior to analysis. RNA content in filtered and unfiltered samples was then measured spectroscopically by absorbance at 260 nm. As a screening metric, the PEG-coated nanocarriers dispersed in water had 600 mM aqueous NaCl added to result in the PEG-coated nanocarriers being dispersed in 150 mM aqueous NaCl (“brine”). Burst release from the nanocarrier was measured after 15 minutes of incubation in this media in the same manner as EE was determined.

Table 5 reports the EE and burst release data for the process variations. Table 6 reports the size as determined by dynamic light scattering (DLS) analysis for select formulations. These data demonstrate that process variations can be employed to produce formulations with high EE (>80%) and low burst (<10%). In contrast to previous observations with iFNP, the inclusion of a suitable lipid, such as POPC, enables high EEs that are further enhanced through the use of acetonitrile (MeCN) as the reforming solvent. At the compositions tested, DSPC and DOPE did not result in process metrics as favorable as POPC.

TABLE 4
Process variation summary. The iFNP stabilizers are described in the text. Water content
refers to the composition of the DMSO stream in iFNP. The two mixers are a CIJ (confined
impinging jet mixer) and a MIVM (multi-inlet vortex mixer). The hydrophobic additive
and coating stabilizer are incorporated during the FNP coating step.

| | iFNP | Water | iFNP | | Hydrophobic | Coating | |
|---|---|---|---|---|---|---|---|
| ID | Stabilizer | (vol %) | Mixer | Lipid | Additive | Stabilizer | Process Note |
| 1 | 503-Asp | 5 | MIVM | POPC, 3.9 mg | PLA, 5 mg | PLA-PEG, 2.5 mg | Zinc rather than |
| | | | | | | | Calcium |
| 2 | 503-80 | 10 | MIVM | POPC, 3.9 mg | PLA, 5 mg | PLA-PEG, 2.5 mg | |
| 3 | 503-Asp | 5 | CIJ | POPC, 3.9 mg | PLA, 5 mg | PLA-PEG, 2.5 mg | |
| 4 | 503-Asp | 12.5 | MIVM | POPC, 3.9 mg | PLA, 5 mg | PLA-PEG, 2.5 mg | |
| 5 | 503-Asp | 5 | MIVM | POPC, 3.9 mg | PLA, 5 mg | PLA-PEG, 2.5 mg | 2 eq of Ca |
| 6 | 503-Asp | 5 | CIJ | POPC, 3.2 mg | none | PLA-PEG, 2.5 mg | |
| 7 | 753-Asp | 5 | CIJ | POPC, 3.2 mg | none | PLA-PEG, 2.5 mg | |
| 8 | 203-Asp | 5 | CIJ | POPC, 3.2 mg | none | PLA-PEG, 2.5 mg | |
| 9 | 503-80 | 10 | CIJ | POPC, 3.2 mg | none | PLA-PEG, 2.5 mg | |
| 10 | 503-Asp | 5 | CIJ | POPC, 2.4 mg | none | PLA-PEG, 2.5 mg | |
| 11 | 503-Asp | 5 | CIJ | POPC, 1.6 mg | none | PLA-PEG, 2.5 mg | |
| 12 | 503-Asp | 5 | CIJ | POPC, 0.8 mg | none | PLA-PEG, 2.5 mg | |
| 13 | 753-Asp | 5 | CIJ | POPC, 2.5 mg | Cholesterol, | PLA-PEG, 2.5 mg | |
| | | | | | 0.45 mg | | |
| 14 | 753-Asp | 5 | MIVM | POPC, 2.5 mg | PLGA, 5 mg | PLA-PEG, 2.5 mg | |
| 15 | 753-Asp | 5 | CIJ | POPC, 2.5 mg | none | PLA-PEG, 2.5 mg | |
| 16 | 753-Asp | 5 | CIJ | POPC, 1.5 mg | none | PLA-PEG, 2.5 mg | |
| 17 | 753-Asp | 5 | CIJ | POPC, 2.5 mg | Cholesterol, | TPGS, 2.5 mg | Vitamin E-TPGS |
| | | | | | 0.45 mg | | |
| 18 | 753-Asp | 5 | CIJ | POPC, 2.5 mg | none | PLA-PEG, 2.5 mg | |
| 19 | 753-Asp | 5 | MIVM | POPC, 2.5 mg | PLGA, 5 mg | PLA-PEG, 2.5 mg | |
| 20 | 753-Asp | 5 | MIVM | POPC-Ca, 2.5 | PLGA, 5 mg | PLA-PEG, 2.5 mg | No age before |
| | | | | mg | | | extraction |
| 21 | 753-Asp | 5 | CIJ | POPC, 2.75 mg | none | PLA-PEG, 2.5 mg | |
| 22 | 753-Asp | 5 | CIJ | POPC, 3.6 mg | PLGA, 5 mg | PLA-PEG, 2.5 mg | |
| 23 | 753-Asp | 5 | MIVM | POPC, 2.5 mg | PLGA, 5 mg | PLA-PEG, 2.5 mg | No age before |
| | | | | | | | extraction |
| 24 | 753-Asp | 5 | CIJ | DSPC, 2.6 mg | PLGA, 5 mg | PLA-PEG, 2.5 mg | |
| 25 | 753-Asp | 5 | MIVM | POPC-Ca, 2.5 | PLGA, 5 mg | PLA-PEG, 2.5 mg | |
| | | | | mg | | | |
| 26 | 753-Asp | 5 | CIJ | DOPE, 2.5 mg | PLGA, 5 mg | PLA-PEG, 2.5 mg | |
| 27 | 753-Asp | 5 | CIJ | POPC, 2 mg | none | PLA-PEG, 2.5 mg | |
| | | | | DOPE, 0.5 mg | | | |
| 28 | 753-Asp | 5 | MIVM | POPC, 2.5 mg | PLGA, 5 mg | PLA-PEG, 2.5 mg | 150 mM NaCl |
| | | | | | | | antisolvent |
| 29 | 753-Asp | 5 | CIJ | POPC, 2.5 mg | none | PLA-PEG, 2.5 mg | POPC after |
| | | | | | | | MeCn |

TABLE 5
Encapsulation efficiency (EE) and burst release
in 150 mM NaCl corresponding to formulations
in Table 4. The analysis is described in the text.

| ID | EE | Burst Release |
|---|---|---|
| 1 | 49% | 59% |
| 2 | 86% | 14% |
| 3 | 82% | 11% |
| 4 | 83% | 16% |
| 5 | 73% | 17% |
| 6 | 73% | 14% |
| 7 | 80% | 13% |
| 8 | 74% | 9% |
| 9 | 66% | 13% |
| 10 | 73% | 21% |
| 11 | 71% | 16% |
| 12 | 55% | 32% |
| 13 | 88% | 15% |
| 14 | 82% | 9% |
| 15 | 82% | 15% |
| 16 | 82% | 9% |
| 17 | 79% | 45% |
| 18 | 77% | 17% |
| 19 | 75% | 22% |
| 20 | 73% | 12% |
| 21 | 66% | 29% |
| 22 | 65% | 23% |
| 23 | 59% | 16% |
| 24 | 59% | 54% |
| 25 | 52% | 26% |
| 26 | 49% | 55% |
| 27 | 18% | 46% |
| 28 | 62% | 0% |
| 29 | 53% | 37% |

TABLE 6
DLS size analysis
of select formulations

| ID | NP Size (nm) |
|---|---|
| 25 | 152 |
| 19 | 150.8 |
| 28 | 106.2 |
| 23 | 164.6 |

### Example 3: Removal of the Extraction Step in the Modified iFNP Process

RNA (˜5 kDa in size with unmodified bases) in the triethylamine salt form was dissolved in 0.5 mL of DMSO at 2.5 mg/mL with a block copolymer stabilizer (5 mg/mL) as indicated in Table 7. The stabilizing agent naming scheme is consistent with Table 1. The DMSO solution contained the indicated volume percent of deionized water. This solution was rapidly mixed with an antisolvent stream of DCM in a multi-inlet vortex mixer (MIVM) or confined impinging jet (CIJ) mixer. For the MIVM, the DCM entered the mixer in three separate streams of 0.5 mL volume. For the CIJ, a single stream of 0.5 mL DCM was used. CaCl₂) at 1 charge equivalent to the total acid content of the formulation was included in one DCM stream. No additional DCM was included in the collection container. Therefore, the MIVM formulation produces an inverse nanocarrier dispersion in DCM with 25 vol % DMSO and the CIJ formulation produces an inverse nanocarrier in 50% DCM150% DMSO. Surprisingly, the produced inverse nanocarriers were stable in this environment and stable to further processing. The RNA content after accounting for mixer apparatus hold-up and losses was about 0.75 mg (free acid basis).

Samples were aged between 45 min and 18 hrs, as noted in Table 7, and no extraction to remove DMSO was performed on the samples. After inverse nanocarrier formation, the lipid or lipids, hydrophobic additive and PEG-b-PLA coating stabilizer (except where noted) were added directly to the dispersion.

The solvent exchange to the reforming solvent, acetonitrile, was carried out as follows. Acetonitrile (MeCN) was added at an equal volume to the DCM and then the dispersion was concentrated by rotary evaporation. A put-and-take distillation involving two additions of MeCN were carried out. For a 0.5 mL DMSO input volume, these additions were 8 mL in total and the concentration volume was 1 mL. The temperature used was 35° C. and the vacuum set point was varied as required to achieve distillation. Surprisingly, the inverse nanocarrier (NC) dispersion was stable during the solvent exchange even in the presence of higher DMSO concentrations.

The inverse NC dispersion in acetonitrile was then rapidly mixed against an equal volume of deionized water (except where noted in Table 7) in a CIJ mixer, and a collection bath of additional deionized water was used to reduce the MeCN volume content to less than 10 vol %. This process produced the PEG-coated nanocarrier. Encapsulation efficiency (EE) was determined by separating free RNA from the nanocarrier using a 300 kDa molecular-weight-cut-off ultrafilter. The nanocarriers were further diluted, generally about 4-fold in water prior to analysis. RNA content in filtered and unfiltered samples was then measured spectroscopically by absorbance at 260 nm. As a screening metric, the PEG-coated nanocarriers dispersed in water had 600 mM aqueous NaCl added to result in the PEG-coated nanocarriers being dispersed in 150 mM aqueous NaCl (“brine”). Burst release from the nanocarrier was measured after 15 minutes of incubation in this media in the same manner as EE was determined. These data are summarized in Table 7.

Table 7: Summary of formulation parameter variation for the process without an extraction. The iFNP stabilizers are described in the text. Water content refers to the composition of the DMSO stream in iFNP. The two mixers are a CJ (confined impinging jet mixer) and a MIVM (multi-inlet vortex mixer). EE is encapsulation efficiency, measured spectroscopically. Burst release is described in the text. (n.t.=not tested)

TABLE 7
Summary of formulation parameter variation for the process without an extraction.
The iFNP stabilizers are described in the text. Water content refers to the composition
of the DMSO stream in iFNP. The two mixers are a CIJ (confined impinging jet
mixer) and a MIVM (multi-inlet vortex mixer). EE is encapsulation efficiency,
measured spectroscopically. Burst release is described in the text.

| | iFNP | Water | iFNP | | Hydrophobic | | | Burst |
|---|---|---|---|---|---|---|---|---|
| ID | Stabilizer | (vol %) | Mixer | Lipid | Additive | Process Note | EE | Release |
| 1 | 503-Asp | 5 | CIJ | POPC, | 203, 5 mg | Aged 18 hr | 75% | 12% |
| | | | | 3.9 mg | | | | |
| 2 | 753-Asp | 5 | CIJ | POPC, | none | Aged 45 min | 79% | 9% |
| | | | | 1.6 mg | | | | |
| 3 | 503-Asp | 5 | CIJ | POPC, | 203, 5 mg | Aged 45 min | 76% | 3% |
| | | | | 3.2 mg | | | | |
| 4 | 503-Asp | 5 | CIJ | POPC, | none | Aged 45 min | 71% | 16% |
| | | | | 1.6 mg | | | | |
| 5 | 503-80 | 10 | MIVM | POPC, | none | Aged 45 min | 20% | 14% |
| | | | | 1.6 mg | | | | |
| 6 | 503-Asp | 5 | CIJ | POPC, | none | PEG-Lipid | 43% | 47% |
| | | | | 1.6 mg | | replaces PEG- | | |
| | | | | | | PLA, aged 45 | | |
| | | | | | | min | | |
| 7 | 503-80 | 10 | MIVM | POPC, | 203, 5 mg | Aged 45 min | 57% | n.t. |
| | | | | 1.6 mg | | | | |
| 8 | 503-Asp | 5 | MIVM | POPC, | 203, 5 mg | Aged 45 min | 39% | n.t. |
| | | | | 1.6 mg | | | | |
| 9 | 503-Asp | 20 | MIVM | POPC, | 203, 5 mg | Aged 45 min | 37% | n.t. |
| | | | | 1.6 mg | | | | |
| 10 | 503-Asp | 5 | CIJ | POPC, | 203, 5 mg | Aged 45 min | 73% | n.t. |
| | | | | 1.6 mg | | | | |
| 11 | 753-Asp | 5 | CIJ | POPC, | 203, 5 mg | Aged 45 min | 81% | n.t. |
| | | | | 1.6 mg | | | | |
| 12 | 503-Asp | 5 | MIVM | POPC, | 203, 5 mg | Concentrate | 45% | n.t. |
| | | | | 1.6 mg | | to 1 mL | | |
| | | | | | | immediately | | |
| | | | | | | after iFNP, | | |
| | | | | | | age 1 hr | | |
| 13 | 503-80 | 10 | MIVM | POPC, | 203, 5 mg | Aged | 22% | n.t. |
| | | | | 1.6 mg | | overnight | | |
| 14 | 503-Asp | 5 | MIVM | POPC, | 203, 5 mg | Aged | 47% | n.t. |
| | | | | 1.6 mg | | overnight | | |
| 15 | 503-Asp | 5 | CIJ | POPC, | oleic acid, 5 mg | Aged 1 hr | 79% | n.t. |
| | | | | 1.6 mg | | | | |
| 16 | 503-Asp | 5 | CIJ | POPC, | ethyloleate, 5 mg | Aged 1 hr | 68% | n.t. |
| | | | | 1.6 mg | | | | |
| 17 | 503-Asp | 5 | CIJ | POPC, | glyceryl | Unstable | n.t. | n.t. |
| | | | | 1.6 mg | trioleate, 5 mg | | | |
| 18 | 503-Asp | 5 | CIJ | POPC, | glyceryl | Unstable in | n.t. | n.t. |
| | | | | 1.6 mg | tripalmitate 5 mg | MeCN | | |

(n.t. = not tested)

These data demonstrate that it is possible to remove the extraction process, optimize the formulation for high EE, and include alternative hydrophobic additives and PEG coatings other than polymers.

### Example 4: Use of Lipid Blends and Ionizable Lipids in the Modified iFNP Process

RNA (˜5 kDa in size with unmodified bases) in the triethylamine salt form was dissolved in 0.5 mL of DMSO at 2.5 mg/mL with a Dex-PLGA polymer (503-80) stabilizer (2.5 mg/mL). The DMSO contained 10 vol % of deionized water. This solution was rapidly mixed with an antisolvent stream of DCM in a multi-inlet vortex mixer (MIVM). The DCM entered the mixer in three separate streams of 0.5 mL volume. Ca(NO₃)₂ at the charge equivalent (relative to the total acid content of the formulation) noted in Table 8 was included in one DCM stream from a concentrated solution in methanol. The outlet stream from the mixer was collected in a container with additional DCM to bring the DMSO content to 10 vol % or less. The RNA content after accounting for mixer apparatus hold-up losses was 0.75 mg (free acid basis).

After inverse nanocarrier formation, 2.85 mg of the cationic ionizable lipid referred to as Dlin-DMA (CAS No. 871258-12-7) was added to the dispersion and aged for the time indicated in Table 8. Then, 0.84 mg of POPC and 1.46 mg of cholesterol were added to the dispersion and aged an additional 15 minutes. Finally, a PEG stabilizer was added as indicated in Table 8: either 2 mg of the lipid-based PEG-DMG (Cas No.: 1397695-86-1) or 1.5 mg of the polymeric PEG-b-PLA were added as noted in Table 8.

The solvent exchange to the reforming solvent, acetonitrile (MeCN), was carried out as follows. The DCM dispersion was concentrated to about 1 mL and then 4 mL of MeCN was added with stirring. The MeCN dispersion was then concentrated to about 0.75 mL and an additional 4 mL of MeCN was added. The dispersion was again concentrated to 0.5 mL. The inverse NC dispersion in acetonitrile was then mixed against an equal volume of aqueous buffer (either deionized (DI) water, phosphate buffered saline (PBS), or a citrate buffer (citrate)) as noted in Table 8 using a CIJ mixer. A collection bath of additional buffer was used to reduce the MeCN volume content to less than 10 vol %. This process produced the PEG-coated nanocarrier. Encapsulation efficiency (EE) was determined by separating free RNA from the nanocarrier using a 300 kDa molecular-weight-cut-off ultrafilter. The coated nanocarriers were further diluted, generally about 4-fold in water prior to analysis. RNA content in filtered and unfiltered samples was then measured using standard methods for RiboGreen.

TABLE 8
Parameter variations for Dlin-containing formulations. Age time
is the delay before adding lipids or extracting. Ca Eq is the equivalents
of calcium with respect to the acid residues in the formulation
(RNA phosphates). Coating stabilizer is what was used in the FNP
coating step to create a stabilized surface on the coated nanoparticle.
EE is encapsulation efficiency, measured spectroscopically. Size
was measured by DLS using a cumulants fit.

| | Age | | | | | | |
|---|---|---|---|---|---|---|---|
| | Time | | Coating | Coating | EE | Size | |
| ID | (hr) | Ca Eq | Stabilizer | Antisolvent | (%) | (nm) | PDI |
| 1 | 18 | 1 | PEG-DMG | DI Water | 96.3 | 173 | 0.38 |
| 2 | 0.25 | 1 | PEG-DMG | DI Water | 96.3 | 166 | 0.44 |
| 3 | 0.25 | 1 | PEG-DMG | PBS | N.D. | 120 | — |
| 4 | 0.25 | 0.5 | PEG-DMG | DI Water | 96.7 | 128 | 0.33 |
| 5 | 0.25 | 0.5 | PEG-DMG | Citrate | 97.1 | 178 | 0.33 |
| 6 | 0.25 | 0.25 | PEG-DMG | DI Water | 96.3 | 214 | 0.35 |
| 7 | 0.25 | 0.25 | PEG-PLA | DI Water | 96.7 | 143 | 0.18 |

(N.D. = not determined)

These data demonstrate that ionizable lipids and lipid blends can be successfully incorporated into the process. Notable process improvements were observed with the use of a blend, including EE values >95% that were not dependent on process variations. Size control was demonstrated with coated nanocarrier sizes that were suitable for sterile filtration. Further, the use of PEG anchored by PLA rather than a lipid was demonstrated with this process. The high polydispersity index (PDI) observed by DLS reflects a small micelle population in addition to the coated nanocarrier population.

### Example 5: Formulation Screening with Model mRNA Using Modified iFNP Process

RNA (>200 base pairs in size with unmodified bases, Qiagen Carrier RNA) in the triethylamine salt form was dissolved in 0.5 mL of DMSO at 2.5 mg/mL. The block copolymer stabilizer 753-Asp was included to afford the RNA loading indicated in Table 9. The DMSO stream included 10 vol % deionized (DI) water unless noted. This solution was rapidly mixed with an antisolvent stream of DCM in a multi-inlet vortex mixer (MIVM). Table 9 notes a few instances where a different antisolvent or the CIJ mixer was employed. For the MIVM, the DCM entered the mixer in three separate streams of 0.5 mL volume. For the CIJ, a single stream of 0.5 mL DCM was used. Ca(NO₃)₂ at 1 charge equivalent, unless noted, to the total acid content of the formulation was included in one DCM stream. No additional DCM was included in the collection container except where noted in the table. This additional DCM is referred to as the “iFNP quench”. Therefore, the MIVM formulation produces an inverse nanocarrier dispersion in DCM with 25 vol % DMSO.

Samples were aged and extracted with 150 mM brine according to the data in Table 9. The extraction was carried out as described in Example 2. After inverse nanocarrier formation, the lipid or lipids, hydrophobic additive, and PEG-b-PLA coating stabilizer (except where noted) were added directly to the dispersion. The solvent exchange to the reforming solvent, acetonitrile, was carried out as described in Example 4.

The inverse NC dispersion in acetonitrile was then mixed against an equal volume of deionized water in a CIJ mixer, and a collection bath of additional deionized water was used to reduce the MeCN volume content to less than 10 vol %. Where the use of a separate PEG-b-PLA block copolymer (BCP) coating stream is noted in Table 9, a MIVM was employed with one inlet containing the inverse nanocarrier dispersion, one inlet containing the coating stabilizer, and two inlets containing the antisolvent (deionized water or 10 mM HEPES buffer, pH 7). This process produced the PEG-coated nanocarrier. Encapsulation efficiency (EE) was determined by separating free RNA from the nanocarrier using a 300 kDa molecular-weight-cut-off ultrafilter. The nanocarriers were diluted, generally about 4-fold, in water prior to this analysis. RNA content in filtered and unfiltered samples was then measured spectroscopically by absorbance at 260 nm. In some instances, DLS size data were obtained using standard techniques. These data are summarized in Table 9.

TABLE 9
Process screening with model mRNA in the modified iFNP process. RNA loading is calculated
from the RNA and block copolymer masses used in iFNP. Post iFNP age time is the delay before
adding lipids or extracting. Coating stabilizer is what was used in the FNP coating step
to create a stabilized surface on the coated nanoparticle. EE is encapsulation efficiency,
measured spectroscopically. Size was measured by DLS using a cumulants fit.

| | RNA | Post iFNP | | | Coating | | EE | Size |
|---|---|---|---|---|---|---|---|---|
| ID | Loading | Age Time | | Lipid | Stabilizer | Process Note | (%) | (nm) |
| 1 | 21% | 18 | hr | POPC, 2.5 mg | PLA-PEG, | CIJ mixer used, iFNP | 84.2 | — |
| | | | | | 2.5 mg | quench, 1.3 eq Ca, | | |
| | | | | | | extraction | | |
| 2 | 21% | 18 | hr | POPC, 2.5 mg | PLA-PEG, | Chloroform used, iFNP | 86.3 | — |
| | | | | | 2.5 mg | quench, 1.3 eq Ca, | | |
| | | | | | | extraction | | |
| 3 | 33% | 18 | hr | POPC, 1.5 mg | PLA-PEG, | iFNP quench, extraction | 90.5 | — |
| | | | | | 2.5 mg | | | |
| 4 | 33% | 18 | hr | POPC, 1.5 mg | PLA-PEG, | | 89.8 | — |
| | | | | | 2.5 mg | | | |
| 5 | 33% | 1 | hr | POPC, 1.5 mg | PLA-PEG, | | 82.8 | 110 |
| | | | | | 2.5 mg | | | |
| 6 | 33% | 1 | hr | POPC, 1.5 mg | PLA-PEG, | 2.5 mg of PLGA added | 88.8 | 130 |
| | | | | | 2.5 mg | | | |
| 7 | 33% | 1 | hr | POPC, 1.5 mg | PLA-PEG, | | 86.8 | 92 |
| | | | | | 2.5 mg | | | |
| 8 | 33% | 18 | hr | POPC, 1.5 mg | PLA-PEG, | Extraction, separate BCP | 74 | 131 |
| | | | | | 2 mg | coating stream | | |
| | | | | | PLA-Pasp, | | | |
| | | | | | 0.5 mg | | | |
| 9 | 33% | 1 | hr | POPC, 1.5 mg | PLA-PEG, | | 80.3 | — |
| | | | | | 2.5 mg | | | |
| 10 | 33% | 1 | hr | POPC, 1.5 mg | PLA-PEG, | Add DCM quench after | 76.4 | — |
| | | | | | 2 mg | age before extraction. | | |
| | | | | | PLA-Pasp, | Separate BCP coating | | |
| | | | | | 0.5 mg | stream | | |
| 11 | 33% | 18 | hr | POPC, 1.5 mg | PLA-PEG, | | 88.8 | — |
| | | | | | 2.5 mg | | | |
| 12 | 33% | 18 | hr | POPC, 1.5 mg | PLA-PEG, | | 86.9 | — |
| | | | | DOPE, 0.15 mg | 2.5 mg | | | |
| 13 | 33% | 18 | hr | POPC, 1.5 mg | PLA-Pasp, | Add DCM quench after | 90.2 | 166 |
| | | | | | 2.5 mg | age before extraction. | | |
| | | | | | | Separate BCP coating | | |
| | | | | | | stream with 10 mM | | |
| | | | | | | HEPES antisolvent. | | |
| 14 | 33% | 18 | hr | POPC, 1.5 mg | PLA-PEG, | MeCN before POPC | 76.4 | 189 |
| | | | | DOPE, 0.15 mg | 2.5 mg | | | |
| 15 | 33% | 18 | hr | POPC, 1.1 mg | PLA-PEG, | MeCN before POPC | 80.8 | 203 |
| | | | | DOPE, 0.55 mg | 2.5 mg | | | |
| 16 | 33% | 18 | hr | POPC, 1.1 mg | PLA-PEG, | MeCN before POPC | 77.9 | 180 |
| | | | | | 2.5 mg | | | |
| 17 | 40% | 18 | hr | POPC, 1.1 mg | PLA-PEG, | MeCN before POPC | 75.5 | 155 |
| | | | | | 2.5 mg | | | |
| 18 | 33% | 1 | hr | POPC, 1.5 mg | PLA-PEG, | | 87.4 | 140 |
| | | | | Chol, 0.27 mg | 2.5 mg | | | |
| 19 | 33% | 1 | hr | POPC, 1.5 mg | PLA-PEG, | | 93.6 | 118 |
| | | | | DOPE, 0.27 mg | 2.5 mg | | | |
| 20 | 33% | 1 | hr | POPC, 1.5 mg | PLA-PEG, | | 87.6 | 152 |
| | | | | DC-Chol, 0.27 | 2.5 mg | | | |
| | | | | mg | | | | |
| 21 | 40% | 18 | hr | POPC, 1.3 mg | PLA-PEG, | 7.5% water in DMSO | 90.5 | 193 |
| | | | | | 2.1 mg | | | |
| 22 | 40% | 18 | hr | POPC, 0.85 mg | PLA-PEG, | 7.5% water in DMSO | 93.4 | 221 |
| | | | | | 2 mg | | | |
| 23 | 33% | 18 | hr | POPC, 1.5 mg | PEG-DMG, | Add DCM quench after | 88.2 | — |
| | | | | | 1 mg | age before extraction | | |
| 24 | 33% | 1 | hr | POPC, 1.5 mg | PEG-DMG, | | 91.4 | 160 |
| | | | | | 2 mg | | | |
| 25 | 33% | 1 | hr | POPC, 1.5 mg | PEG-DMG, | | 93.6 | 297 |
| | | | | DODAP, 0.27 | 1 mg | | | |
| | | | | mg | | | | |
| 26 | 33% | 1 | hr | POPC, 1.5 mg | PEG-DMG, | | 93.2 | 283 |
| | | | | DODAP, 0.75 | 1 mg | | | |
| | | | | mg | | | | |
| 27 | 33% | 1 | hr | DOPC, 1.5 mg | PEG-DMG, | | 91.3 | 137 |
| | | | | | 2 mg | | | |

These results show broadly applicable conditions using different lipids, different coatings, and varied iFNP compositions.

### Example 6: Screening Using an Enzyme Protection Assay Based Upon RNAse Digestion

RNA (>200 base pairs in size with unmodified bases, Qiagen Carrier RNA) in the triethylamine salt form was dissolved in 0.5 mL of DMSO at 2.5 mg/mL. A Dex-PLGA stabilizing polymer (503-80) was included to afford the RNA loading indicated in Table 10, which also notes a few formulations that used alternative block copolymer stabilizers. The DMSO stream included 10 vol % deionized water. This solution was rapidly mixed with an antisolvent stream of DCM in a multi-inlet vortex mixer (MIVM). The DCM entered the mixer in three separate streams of 0.5 mL volume. Ca(NO₃)₂ at 1 charge equivalent, unless noted, to the total acid content of the formulation was included in one DCM stream. Additional DCM, referred to as the “iFNP quench,” was included in the collection vial so that DMSO content after collection was <10 vol %. The RNA content, after accounting for mixer apparatus hold-up and losses, was about 0.75 mg (free acid basis).

Samples were aged 1-2 hours and were not extracted prior to lipid addition and solvent exchange, unless noted in Table 10. The lipid(s), hydrophobic additive(s) and coating stabilizer(s) (as noted in Table 10) were added directly to the dispersion. Generally, any ionizable components were added first, followed by POPC and cholesterol, followed by the coating stabilizer. The coating stabilizer was generally 2-3 mg for the input concentrations used in this example. The solvent exchange to the reforming solvent, acetonitrile, was carried out as described in Example 4.

The inverse NC dispersion in acetonitrile was then mixed against an equal volume of deionized water in a CIJ mixer, and a collection bath of additional deionized water was used to reduce the MeCN volume content to less than 10 vol %. This process produced the PEG-coated nanocarrier. The nanocarriers were then buffer-exchanged by ultrafiltration into deionized water (removing residual acetonitrile) or PBS where noted in Table 10.

DLS size data were obtained using standard techniques. The nanocarriers were also evaluated for RNAse A protection of the encapsulated RNA. This assay was conducted as follows. 50 μL of nanocarriers (˜200 μg/mL RNA) in PBS were mixed with 100 μL of RNAse A in deionized water (20 g/mL). After 1 hour incubation at 37° C., 100 μL of proteinase K (1 mg/mL) plus DTT (dithiothreitol) (5 mM) were added. This digestion continued for 30 minutes at 55° C. A negative control was conducted using deionized water without the enzymes. RNA content was then measured by dissolving the nanoparticles in Triton X-100 (a nonionic surfactant having a polyethylene oxide chain) (1% in Tris-EDTA, pH 8) for 15-30 minutes at 55° C. Exposed RNA is degraded, resulting in no signal in a RiboGreen concentration measurement while protected RNA is detected by RiboGreen. Each assay included a positive control of soluble RNA. Additional controls confirmed that the proteinase K degradation conditions were sufficient to abolish RNAse activity. This RiboGreen assay does not detect single strand breaks but an average extent of degradation. The protection value is calculated as [RNA signal with RNAse]/[RNA signal without RNAse]*100. These data are summarized in Table 10.

As a positive control, standard lipid nanoparticles were produced using methods known in the field. Charge ratios of 5× or 6× (lipid:RNA) were used. The lipid nanoparticles were assembled in an MIVM using one ethanolic stream containing the lipid components and three aqueous streams (citrate buffer, pH 4) containing the RNA. The lipid nanoparticles were then dialyzed overnight into phosphate buffer saline (pH 7.3) and characterized for size and RNAse protection. Known lipid formulations for DODAP (1,2-dioleoyl-3-dimethylammonium propane), DC-Cholesterol (DC-Chol) (30-[N—(N′,N′-dimethylaminoethane)-carbamoyl]cholesterol hydrochloride), and Dlin-DMA (1,2-dilinoleyloxy-n,n-dimethyl-3-aminopropane) were used. However, the charge ratio (lipid:RNA) was significantly higher for the lipid nanoparticle than used for iFNP. Lipid nanoparticles were generally 70-100 nm in size with RNAse protection in the 90-100% range.

To confirm that the RNAse protection enhancements seen for certain lipid compositions were due to iFNP structure rather than charge complexation of RNA, control experiments characterized RNAse protection when RNA was added to the aqueous phase during the coating step. In these experiments, the iFNP/coating process was run with all the same components, but RNA was not included in the formation of the inverse nanocarrier. The inverse nanocarriers behaved the same throughout processing. The RNA, with the amount adjusted for hold-up losses in the mixer during inverse nanocarrier assembly, was dissolved in the aqueous stream used as the antisolvent in the coating process. The nanocarriers were visually similar to those produced with RNA encapsulated. The nanocarriers were then analyzed using the RNAse protection assay described above, after buffer exchange into PBS. Ionizable lipids (Dlin-DMA, DODAP) exhibited protection percentages equal to controls where soluble RNA is added to the digestion assay (˜5-10% protection). This indicates that the elevated protection (>60%) seen relied on the iFNP process rather than charge complexation. For DC-Chol, which bears a permanent charge, the control exhibited 100% protection, indicating that soluble RNA not encapsulated within the iFNP nanocarrier was complexed by DC-Chol.

TABLE 10
Process parameter variations with RNAse A enzymatic degradation assay. RNA loading is calculated
from the RNA and block copolymer masses used in iFNP. Lipids use standard naming. DC-Chol is
DC-cholesterol (CAS: 166023-21-8), Dlin is Dlin-DMA (CAS: 871258-12-7), Chol is cholesterol.
The charge ratio refers to the stoichiometry of any charge ionizable lipid in the formulation
and the phosphate charges of the RNA. Coating stabilizer is what was used in the FNP coating
step to create a stabilized surface on the coated nanoparticle. Protection refers to the RNAse
A degradation protection as described in the text. Size was measured by DLS using a cumulants fit.

| | RNA | Lipid | Charge | Coating | | Protection | Size |
|---|---|---|---|---|---|---|---|
| ID | Loading | Composition | Ratio | Stabilizer | Process Notes | (%) | (nm) |
| 1 | 33% | POPC (1.5 mg) | — | PLA-PEG | 753-Asp | 32% | 262 |
| | | | | | No quench | | |
| 2 | 33% | POPC (1.5 mg) | — | PEG-DMG | 753-Asp | 12% | 392 |
| | | | | | No quench | | |
| 3 | 33% | POPC/Ethyl Oleate | — | PLA-PEG | 753-Asp | 30% | 244 |
| | | (1.5/1.5 mg) | | | No quench | | |
| 4 | 33% | POPC/DODAP | 0.65 | PLA-PEG | 753-Asp | 21% | 206 |
| | | (0.75/0.75 mg) | | | No quench | | |
| 5 | 33% | POPC (1.5 mg) | — | PEG-DMG | 502-70 | — | 157 |
| | | | | | Aged overnight | | |
| 6 | 33% | DODAP | 2 | PEG-DMG | | 2% | 402 |
| 7 | 33% | DODAP/POPC/Chol | 2 | PEG-DMG | Lipid Ratio = | 69% | 90 |
| | | | | | 45:23:24:7.7 | | |
| 8 | 33% | DODAP | 2 | PEG-DMG | 0.5 eq Ca. | 3% | 200 |
| 9 | 33% | DODAP/POPC/Chol | 2 | PEG-DMG | 0.5 eq Ca. Lipid | 49% | 154 |
| | | | | | Ratio = | | |
| | | | | | 45:23:24:7.7 | | |
| 10 | 33% | DODAP | 3 | PEG-DMG | 0.5 eq Ca | 2% | 554 |
| 11 | 33% | DODAP | 3 | PEG-DMG | 0.5 eq Ca. Coating | 1% | 596 |
| | | | | | antisolvent = | | |
| | | | | | acetate buffer | | |
| | | | | | (pH 4) | | |
| 12 | 33% | DODAP/POPC/Chol | 2 | PEG-DMG | Aged overnight. | 82% | 104 |
| | | | | | Lipid ratio = | | |
| | | | | | 43:12:38:7 | | |
| 13 | 33% | DODAP/POPC/Chol | 2 | PLA-PEG | Aged overnight. | 64% | 253 |
| | | | | | Lipid ratio = | | |
| | | | | | 43:12:38:7 | | |
| 14 | 33% | DC-Chol/POPC | 2 | PEG-DMG | Aged overnight. | 97% | 108 |
| | | | | | Lipid ratio = | | |
| | | | | | 46.3:46.3:7.3 | | |
| 15 | 33% | DC-Chol/POPC | 2 | PLA-PEG | Aged overnight. | 98% | — |
| | | | | | Lipid ratio = | | |
| | | | | | 46.3:46.3:7.3 | | |
| 16 | 33% | DODAP/POPC/Chol | 2 | PEG-DMG | Lipid ratio = | 97% | 85 |
| | | | | | 43:12:38:7 | | |
| 17 | 33% | DODAP/POPC/Chol | 2 | PLA-PEG | Lipid ratio = | 60% | 333 |
| | | | | | 43:12:38:7 | | |
| 18 | 50% | DC-Chol/POPC | 2 | PEG-DMG | Lipid ratio = | 101% | 120 |
| | | | | | 46.3:46.3:7.3 | | |
| 19 | 50% | DC-Chol/POPC | 2 | PLA-PEG | Lipid ratio = | 103% | 348 |
| | | | | | 46.3:46.3:7.3 | | |
| 20 | 33% | Dlin/POPC/Chol | 2 | PEG-DMG | Lipid ratio: | 58% | 147 |
| | | | | | 44.1:11:37.5:7.4 | | |
| 21 | 33% | Dlin/POPC/Chol | 2 | PLA-PEG | Lipid ratio: | 76% | 185 |
| | | | | | 44.1:11:37.5:7.4 | | |
| 22 | 50% | Dlin/POPC/Chol | 2 | PEG-DMG | Lipid ratio: | 56% | 211 |
| | | | | | 44.1:11:37.5:7.4 | | |
| 23 | 50% | Dlin/POPC/Chol | 2 | PLA-PEG | Lipid ratio: | 81% | 213 |
| | | | | | 44.1:11:37.5:7.4 | | |
| 24 | 50% | POPC (1.5 mg) | — | PLA-PEG | Wash into PBS | 27% | 74 |
| 25 | 50% | POPC/Chol | — | PLA-PEG | Wash into PBS | 31% | 117 |
| | | (1.5/0.38 mg) | | | | | |
| 26 | 50% | — | — | PLA-PEG | Wash into PBS | 9% | 283 |
| 29 | 50% | DOPC/POPC/Chol | — | PEG-DMG | Lipid ratio = | 23% | 371 |
| | | | | | 43:12:38:7 | | |
| | | | | | PBS wash | | |

### Example 7: Formulation Screening Using SM-102 Cationic Lipid

RNA (>200 base pairs in size with unmodified bases, Qiagen Carrier RNA) in the triethylamine salt form was dissolved in 0.5 mL of DMSO at 2.5 mg/mL. (In one instance, where noted in Table 11, the triethylamine salt was recovered from solution by standard LiCl/Ethanol precipitation and then used in the process.) A Dex-PLGA stabilizing polymer (503-80, unless otherwise noted in Table 11) was added to the DMSO stream at 2.5 mg/mL. The DMSO stream included 10 vol % deionized water. This solution was rapidly mixed with an antisolvent stream of DCM in a multi-inlet vortex mixer (MIVM). The DCM entered the mixer in three separate streams of 0.5 mL volume. Ca(NO₃)₂ at a specified charge ratio (Table 11) with respect to the total acid content of the formulation was included in one DCM stream. Additional DCM, referred to as the “iFNP quench,” was included in the collection vial so that DMSO content after collection was <10 vol %. This afforded RNA encapsulated in inverse nanocarriers with a total content of about 0.75 mg (free acid basis, accounting for mixer apparatus hold-up and losses).

Samples were not extracted prior to lipid addition and solvent exchange. The dispersion was aged 10 minutes unless noted in Table 11. A cationic lipid, SM-102 (heptadecan-9-yl 8-((2-hydroxyethyl)(6-oxo-6-(undecyloxy)hexyl)amino)octanoate) (CAS #2089251-47-6), was added to the dispersion. Then, POPC, cholesterol, and PEG-DMG were added. Stock solutions were in chloroform or ethanol (no differences were observed). SM-102 was included at a charge ratio of about 1.9. The mole ratio of lipid components was about 47% SM-102, 10% POPC, 37% cholesterol, and 6% PEG-DMG. The solvent exchange to the reforming solvent, acetonitrile, was carried out as described in Example 4, but using three additions of acetonitrile instead of two.

The inverse NC dispersion in acetonitrile was then mixed against an equal volume of deionized water in a CIJ mixer, and a collection bath of additional deionized water was used to reduce the MeCN volume content to less than 10 vol %. This process produced the PEG-coated nanocarrier. The nanocarriers were then buffer-exchanged by ultrafiltration into PBS. Some formulations, where noted, were diluted with an equal volume of buffer prior to ultrafiltration. The same buffer was then used for the buffer exchange stage of ultrafiltration. Buffers tested were 150 mM NaCl, PBS, and 25 mM acetate buffer (pH 4).

DLS size data was measured using standard techniques. The nanocarriers were also evaluated for RNAse A protection of the encapsulated RNA. This assay was conducted as follows. 50 μL of nanocarriers (˜200 μg/mL RNA) in PBS were mixed with 100 μL of RNAse A in deionized water (20 μg/mL). After 1 hour incubation at 37° C., 100 μL of proteinase K (1 mg/mL) plus DTT (5 mM) were added. This digestion continued for 30 minutes at 55° C. A negative control was conducted using deionized water without the enzymes. RNA content was then measured by dissolving the nanoparticles in Triton X-100 (1% in Tris-EDTA, pH 8) for 15-30 minutes at 55° C. Exposed RNA is degraded, resulting in no signal in a RiboGreen concentration measurement while protected RNA is detected by RiboGreen. Each assay included a positive control of soluble RNA. Additional controls confirmed that the proteinase K degradation conditions were sufficient to abolish RNAse activity. This RiboGreen assay does not detect single strand breaks but an average extent of degradation. The protection value is calculated as [RNA signal with RNAse]/[RNA signal without RNAse]*100. These data are summarized in Table 11.

TABLE 11
Process parameter variations with SM-102 formulations. Calcium
equivalents are on a charge basis with respect to the phosphate
groups in the RNA. The charge ratio refers to the stoichiometry
of the ionizable lipid in the formulation and the phosphate
charges of the RNA. Protection refers to the RNAse A degradation
assay as described in the text. Size was measured by DLS
using a cumulants fit.

| | | | | Protec- | | |
|---|---|---|---|---|---|---|
| | Ca | Charge | | tion | Size | |
| ID | (eq) | Ratio | Process Notes | (%) | (nm) | PDI |
| 1 | 0.5 | 1.9 | | 101% | 120 | 0.20 |
| 2 | 0.5 | 1.9 | 1.5 hr age pre-lipid | 90% | 129 | 0.20 |
| 3 | 1 | 1.9 | 0.25M Urea in iFNP | 77% | 132 | 0.15 |
| 4 | 0.5 | 1.9 | | 92% | 115 | 0.24 |
| 5 | 0.5 | 1.9 | 2 hr age pre-lipid | 87% | 109 | 0.20 |
| 6 | 0.25 | 1.9 | | 70% | 134 | 0.28 |
| 7 | 0.25 | 1.9 | 2 hr age pre-lipid | 65% | 147 | 0.30 |
| 8 | 0.5 | 1.9 | Age 1 hr pre-lipid | 87% | 129 | 0.25 |
| 9 | 0.5 | 0 | Age 1 hr pre-lipid. Replace | 8% | 124 | 0.34 |
| | | | SM-102 with POPC | | | |
| 10 | 0.5 | 1.9 | Age 1 hr pre-lipid | 88% | 126 | 0.21 |
| 11 | 0.5 | 1.9 | Use 502-75 Dex-PLGA, | 71% | 112 | 0.34 |
| | | | age 1 hr pre-lipid | | | |
| 12 | 0.5 | 1.9 | Use 502-75 Dex-PLGA. | 64% | 103 | 0.32 |
| | | | Use EtOH stocks of lipids | | | |
| 13 | 0.5 | 1.9 | Dilute with PBS before | 81% | 112 | 0.16 |
| | | | ultrafiltration | | | |
| 14 | 0.5 | 1.9 | Dilute with 150 mM NaCl | 87% | 113 | 0.16 |
| | | | before ultrafiltration. Wash | | | |
| | | | with same | | | |
| 15 | 0.5 | 1.9 | Dilute with 25 mM Acetate | 106% | 126 | 0.19 |
| | | | buffer (pH 4) before | | | |
| | | | ultrafiltration. Wash with | | | |
| | | | same | | | |
| 16 | 0.5 | 1.9 | Dilute with MQ (deionized | 78% | 112 | 0.25 |
| | | | water) before ultrafiltration. | | | |
| | | | Wash with PBS | | | |
| 17 | 0.5 | 1.9 | Add PEG-DMG at end of | 84% | 134 | 0.24 |
| | | | solvent exchange. Dilute | | | |
| | | | with MQ and wash with | | | |
| | | | PBS | | | |
| 18 | 0.5 | 1.9 | LiCl/EtOH precipitation for | 52% | 100 | 0.38 |
| | | | RNA recovery as input to | | | |
| | | | process | | | |
| 19 | 0.5 | 1.9 | | 93% | 108 | 0.21 |

### Example 8: Cell Transfection Studies

The ability for these formulations to transfect cells was evaluated using an mRNA sequence encoding green fluorescent protein (GFP) (Dasher GFP mRNA from Aldevron). Blends of mRNA encoding GFP and carrier RNA in the TEA salt form were prepared (ratio as noted in Table 12). The RNA was mixed with a block copolymer in DMSO containing 10 vol % deionized water, unless noted in Table 12. The stabilizing polymer was Dex-PLGA (503-80), unless otherwise stated in Table 12 under the process note column. RNA was added to the DMSO stream at 2.5 mg/mL and the polymer stabilizer was added at the concentration necessary to produce the loading noted in Table 12. This solution was rapidly mixed with an antisolvent stream of DCM in a multi-inlet vortex mixer (MIVM). The DCM entered the mixer in three separate streams of 0.5 mL volume each. Ca(NO₃)₂ at a 1 charge equivalent ratio (except where noted in Table 12) with respect to the total acid content of the formulation was included in one DCM stream. Additional DCM, referred to as the “iFNP quench,” was included in the collection vial so that DMSO content after collection was <10 vol %. The RNA content in the resulting inverse nanocarrier was 0.75 mg, accounting for hold-up losses to the mixer apparatus.

Samples were not extracted prior to lipid addition, except where noted in Table 12. Prior to the solvent exchange process, a cationic lipid, generally SM-102 (CAS #2089251-47-6) but sometimes DODAP (CAS #127512-29-2) or Dlin-MC3-DMA ((6Z,9Z,28Z,31Z)-heptatriaconta-6,9,28,31-tetraen-19-yl 4-(dimethylamino)butanoate) (CAS #1224606-06-7), was added to the dispersion. Then, other components as noted in Table 12 were added. Stock solutions were in chloroform or ethanol. The solvent exchange to the reforming solvent, acetonitrile, was carried out as described in Example 7.

The inverse NC dispersion in acetonitrile was then mixed against an equal volume of deionized water in a CIJ mixer (except where noted), and a collection bath of additional deionized water was used to reduce the MeCN volume content to less than 10 vol %. This process produced the PEG-coated nanocarrier. The nanocarriers were then buffer-exchanged by ultrafiltration into deionized water or PBS. Some formulations, where noted, were diluted with an equal volume of buffer prior to ultrafiltration. The same buffer was then used for the buffer exchange stage of ultrafiltration. Buffers used were 150 mM NaCl, PBS, and 25 mM acetate buffer (pH 4), and isotonic HEPES/NaCl.

DLS size data were obtained using standard techniques. The nanocarriers were also evaluated for RNAse A protection of the encapsulated RNA. This assay was conducted as follows. 50 μL of nanocarriers (˜200 μg/mL RNA) in PBS were mixed with 100 μL of RNAse A in deionized water (20 μg/mL). After 1 hour incubation at 37° C., 100 μL of proteinase K (1 mg/mL) plus dithiothreitol (DTT) (5 mM) were added. This digestion continued for 30 minutes at 55° C. A negative control was conducted using deionized water without the enzymes. RNA content was then measured by dissolving the nanoparticles in Triton X-100 (1% in Tris-EDTA, pH 8) for 15-30 minutes at 55° C. Exposed RNA is degraded, resulting in no signal in a RiboGreen concentration measurement while protected RNA is detected by RiboGreen. Each assay included a positive control of soluble RNA. Additional controls confirmed that the proteinase K degradation conditions were sufficient to abolish RNAse activity. This RiboGreen assay does not detect single strand breaks but an average extent of degradation. The protection value is calculated as [RNA signal with RNAse]/[RNA signal without RNAse]*100. These data are summarized in Table 13.

As a positive control, standard lipid nanoparticles were produced using methods known in the field. Charge ratios of 5× or 6× (lipid:RNA) were used. The lipid nanoparticles were assembled in an MIVM using one ethanolic stream containing the lipid components and three aqueous streams (citrate buffer, pH 4) containing the RNA. The lipid nanoparticles were then dialyzed overnight into phosphate buffer saline (PBS, pH 7.3) and characterized for size and RNAse protection. Lipid formulations for DODAP, DC-Cholesterol (DC-Chol), Dlin-DMA, and SM-102 used standard compositions. The charge ratio (lipid:RNA) was significantly higher for the lipid nanoparticle than used for iFNP. Lipid nanoparticles are characterized in Table 13.

TABLE 12
Parameter variations of mRNA formulations. The mRNA % represents the fraction
of total RNA content that is GFP mRNA rather than carrier RNA. The iFNP loading
is the percent of nanoparticle mass that is RNA at the iFNP step. The coating
polymer is the stabilizer on the surface of the nanoparticle after processing
into water. Process notes capture changes from the baseline noted in the text.

| | | mRNA | iFNP | | Coating | |
|---|---|---|---|---|---|---|
| Name | Process | (%) | Loading | Lipids | Polymer | Process Notes |
| mRNA- | iFNP | 10 | 33% | 1.6 mg POPC | 2.5 mg PLA- | 753-Pasp, POPC, |
| 001 | | | | | b-PEG | PLA-PEG |
| mRNA- | iFNP | 10 | 25% | 1.6 mg POPC | 2.5 mg PLA- | 753-Pasp (more), |
| 002 | | | | | b-PEG | POPC, PLA-PEG |
| mRNA- | iFNP | 10 | 33% | 1.6 mg POPC, | 2.5 mg PLA- | 753-Pasp, POPC, |
| 003 | | | | 0.29 mg | b-PEG | cholesterol, PLA- |
| | | | | cholesterol | | PEG |
| mRNA- | iFNP | 10 | 33% | 1.6 mg POPC | 0.5 mg PEG- | 753-Pasp, POPC, |
| 004 | | | | | DMG | PEG-lipid |
| mRNA- | iFNP | 10 | 33% | 1.6 mg POPC | 2.5 mg PLA- | mRNA-001 replicate |
| 005 | | | | | b-PEG | |
| mRNA- | iFNP | 10 | 33% | 1.6 mg POPC | 2.5 mg PLA- | mRNA-001, but |
| 006 | | | | | b-PEG | CaCl2 in DMSO |
| | | | | | | stream |
| mRNA- | iFNP | 0 | 33% | 1.5 mg POPC | 2.5 mg 753- | 753-PAsp in iFNP. |
| 007 | | | | | PAsp | Extraction process |
| mRNA- | iFNP | 0 | 33% | 1.5 mg POPC | 1 mg PEG- | 753-PAsp in iFNP. |
| 008 | | | | | DMG | Extraction process |
| mRNA- | iFNP | 0 | 33% | 1.5 mg POPC | 2.5 mg PEG- | 753-PAsp in iFNP. |
| 009 | | | | | PLA | Extraction process |
| mRNA- | iFNP | 10 | 33% | 1.5 mg POPC | 2.5 mg 753- | 753-PAsp in iFNP. |
| 010 | | | | | PAsp | Extraction process |
| mRNA- | iFNP | 10 | 33% | 1.5 mg POPC | 1 mg PEG- | 753-PAsp in iFNP. |
| 011 | | | | | DMG | Extraction process |
| mRNA- | iFNP | 10 | 33% | 1.5 mg POPC | 2.5 mg PEG- | 753-PAsp in iFNP. |
| 012 | | | | | PLA | Extraction process |
| mRNA- | iFNP | 10 | 33% | 1.5 mg POPC | 4 mg PEG- | 753-PAsp in iFNP. |
| 013 | | | | | PLA | Extraction process |
| mRNA- | iFNP | 10 | 33% | 1.5 mg POPC | 2 mg PEG- | 753-PAsp in iFNP. |
| 014 | | | | 0.27 mg Chol | DMG | |
| mRNA- | iFNP | 10 | 33% | 1.5 mg POPC | 2 mg PEG- | 753-PAsp in iFNP. |
| 015 | | | | 0.27 mg DC- | DMG | |
| | | | | Chol | | |
| mRNA- | iFNP | 10 | 33% | 1 mg POPC | 2 mg PEG- | 753-PAsp in iFNP. |
| 016 | | | | 0.27 mg DC- | DMG | |
| | | | | Chol | | |
| mRNA- | iFNP | 10 | 33% | 1.5 mg POPC | 2 mg PEG- | 753-PAsp in iFNP. |
| 017 | | | | 0.27 mg DOPE | DMG | |
| mRNA- | iFNP | 10 | 33% | 0.75 mg POPC | 2 mg PEG- | 753-PAsp in iFNP. |
| 018 | | | | 0.75 mg | DMG | |
| | | | | DODAP | | |
| mRNA- | iFNP | 10 | 33% | 0.75 mg POPC | 2 mg PEG- | 753-PAsp in iFNP. |
| 019 | | | | 0.75 mg | DMG | |
| | | | | DODAP | | |
| | | | | 0.27 mg Chol | | |
| mRNA- | iFNP | 10 | 33% | 0.75 mg POPC | 2 mg PEG- | 753-PAsp in iFNP. |
| 020 | | | | 0.75 mg | DMG | Add DODAP at end |
| | | | | DODAP | | of solvent exchange |
| | | | | 0.27 mg Chol | | |
| mRNA- | iFNP | 10 | 33% | 0.75 mg POPC | 2 mg PEG- | 753-PAsp in iFNP. |
| 021 | | | | 0.75 mg | DMG | Add DODAP at end |
| | | | | DODAP | | of solvent exchange. |
| | | | | 0.27 mg Chol | | Antisolvent coating is |
| | | | | | | acetate buffer (pH 4) |
| mRNA- | iFNP | 10 | 33% | 0.75 mg POPC | 2 mg PEG- | 753-PAsp in iFNP. |
| 022 | | | | 0.75 mg | DMG | Add Acetic Acid - |
| | | | | DODAP | | DODAP (1:1 ratio) at |
| | | | | 0.27 mg Chol | | end of solvent |
| | | | | | | exchange |
| mRNA- | iFNP | 10 | 33% | 1.7 mg POPC | 2 mg PEG- | 2x charge ratio of |
| 023 | | | | 2.87 mg | DMG | DODAP |
| | | | | DODAP | | |
| | | | | 1 mg Chol | | |
| mRNA- | iFNP | 10 | 33% | 1.7 mg DSPC | 2 mg PEG- | 2x charge ratio of |
| 024 | | | | 2.87 mg | DMG | DODAP |
| | | | | DODAP | | |
| | | | | 1 mg Chol | | |
| mRNA- | iFNP | 10 | 33% | 1.7 mg POPC | 2 mg PEG- | 1x charge ratio of |
| 025 | | | | 1.44 mg | DMG | DODAP |
| | | | | DODAP | | |
| | | | | 1 mg Chol | | |
| mRNA- | iFNP | 0 | 33% | 1.7 mg POPC | 2 mg PEG- | 2x charge ratio of |
| 026 | | | | 2.87 mg | DMG | DODAP |
| | | | | DODAP | | |
| | | | | 1 mg Chol | | |
| mRNA- | LNP | 10 | — | — | — | 5x DODAP LNP |
| 027 | | | | | | |
| mRNA- | LNP | 10 | — | — | — | 2x DODAP LNP |
| 028 | | | | | | |
| mRNA- | LNP | 0 | — | — | — | 5x DODAP LNP |
| 029 | | | | | | (25:20:45:10) |
| mRNA- | LNP | 100 | — | — | — | 5x DODAP LNP |
| 030 | | | | | | (25:20:45:10) |
| mRNA- | LNP | 10 | — | — | — | 5x DODAP LNP |
| 031 | | | | | | (25:20:45:10) |
| mRNA- | LNP | 10 | — | — | — | 5x DODAP LNP |
| 032 | | | | | | (25:20:45:10) DOPE |
| | | | | | | instead of DSPC |
| mRNA- | LNP | 10 | — | — | — | 6x DC-Chol LNP |
| 033 | | | | | | (45:45:10 with DSPC |
| | | | | | | and PEG-DMG) |
| mRNA- | LNP | 0 | — | — | — | 6x Dlin-DMA LNP |
| 034 | | | | | | (50:10:38.5:1.5) |
| mRNA- | LNP | 100 | — | — | — | 6x Dlin-DMA LNP |
| 035 | | | | | | (50:10:38.5:1.5) |
| mRNA- | LNP | 100 | — | — | — | 6x Dlin-DMA LNP |
| 036 | | | | | | (50:10:38.5:1.5) sub |
| | | | | | | DOPE for DSPC |
| mRNA- | iFNP | 10 | 50% | 0.84 mg POPC | 2 mg PEG- | 2x charge ratio of |
| 037 | | | | 2.85 mg Dlin- | DMG | Dlin-DMA |
| | | | | DMA | | |
| | | | | 1.46 mg Chol | | |
| mRNA- | iFNP | 10 | 50% | 0.84 mg DOPE | 2 mg PEG- | 2x charge ratio of |
| 038 | | | | 2.85 mg Dlin- | DMG | Dlin-DMA |
| | | | | DMA | | |
| | | | | 1.46 mg Chol | | |
| mRNA- | iFNP | 10 | 50% | 0.84 mg DOPC | 2 mg PEG- | 2x charge ratio of |
| 039 | | | | 2.85 mg Dlin- | DMG | Dlin-DMA |
| | | | | DMA | | |
| | | | | 1.46 mg Chol | | |
| mRNA- | iFNP | 10 | 50% | 0.42 mg POPC | 2 mg PEG- | 1x charge ratio of |
| 040 | | | | 1.42 mg Dlin- | DMG | Dlin-DMA |
| | | | | DMA | | |
| | | | | 0.73 mg Chol | | |
| mRNA- | LNP | 10 | — | — | — | 6x Dlin-DMA LNP |
| 041 | | | | | | (50:10:38.5:1.5) sub |
| | | | | | | DOPE for DSPC |
| mRNA- | LNP | 10 | — | — | — | 6x Dlin-DMA LNP |
| 042 | | | | | | (50:10:38.5:1.5) sub |
| | | | | | | POPC for DSPC |
| mRNA- | LNP | 10 | — | — | — | 6x Dlin-DMA LNP |
| 043 | | | | | | (50:10:38.5:1.5) sub |
| | | | | | | POPC for DSPC |
| mRNA- | LNP | 10 | — | — | — | 2x Dlin-DMA LNP |
| 044 | | | | | | (50:10:38.5:1.5) sub |
| | | | | | | POPC for DSPC |
| mRNA- | iFNP | 10 | 50% | 0.84 mg POPC | 2 mg PEG- | 2x charge ratio of |
| 045 | | | | 2.85 mg Dlin- | DMG | Dlin-DMA |
| | | | | DMA | | |
| | | | | 1.46 mg Chol | | |
| mRNA- | iFNP | 10 | 50% | 0.84 mg POPC | 3 mg PEG- | 2x charge ratio of |
| 046 | | | | 2.85 mg Dlin- | PLA | Dlin-DMA |
| | | | | DMA | | |
| | | | | 1.46 mg Chol | | |
| mRNA- | iFNP | 10 | 50% | 1 mg POPC | 1.5 mg PEG- | 3x charge ratio of |
| 047 | | | | 4.25 mg Dlin- | DMG | Dlin-DMA |
| | | | | DMA | | |
| | | | | 1.97 mg Chol | | |
| mRNA- | iFNP | 0 | 50% | 0.84 mg POPC | 2 mg PEG- | 2x charge ratio of |
| 048 | | | | 2.85 mg Dlin- | DMG | Dlin-DMA |
| | | | | DMA | | |
| | | | | 1.46 mg Chol | | |
| mRNA- | iFNP | 10 | 50% | 3 mg SM-102 | 1.5 mg PEG- | 1.9x charge ratio of |
| 050 | | | | 1.3 mg Chol | DMG | SM-102 |
| | | | | 0.7 mg POPC | | |
| mRNA- | LNP | 10 | — | — | — | 6x SM-102 LNP |
| 051 | | | | | | (50:10:38.5:1.5) sub |
| | | | | | | POPC for DSPC |
| mRNA- | iFNP | 10 | 50% | 3 mg SM-102 | 1.5 mg PEG- | 1.9x charge ratio of |
| 054 | | | | 1.3 mg Chol | DMG | SM-102 |
| | | | | 0.7 mg POPC | | |
| mRNA- | LNP | 10 | — | — | — | 6x SM-102 LNP |
| 056 | | | | | | (50:10:38.5:1.5) sub |
| | | | | | | POPC for DSPC |
| mRNA- | iFNP | 10 | 50% | 3 mg SM-102 | 1.5 mg PEG- | 0.5 eq Ca, 12% MQ |
| 57 | | | | 1.3 mg Chol | DMG | in iFNP. 1.9x charge |
| | | | | 0.7 mg POPC | | ratio of SM-102 |
| mRNA- | iFNP | 10 | 50% | 1.65 mg SM- | 1.5 mg PEG- | 0.5 eq Ca, 12% MQ |
| 58 | | | | 102 | DMG | in iFNP. 1.1x charge |
| | | | | 0.715 mg Chol | | ratio of SM-102 |
| | | | | 0.385 mg | | |
| | | | | POPC | | |
| mRNA- | iFNP | 10 | 50% | 3 mg SM-102 | 1.5 mg PEG- | 0.5 eq Ca, 12% MQ |
| 59 | | | | 2 mg Ethyl | DMG | in iFNP. 1.9x charge |
| | | | | Oleate | | ratio of SM-102 |
| mRNA- | LNP | 10 | — | — | — | 6x SM-102 LNP |
| 60 | | | | | | (50:10:38.5:1.5) sub |
| | | | | | | POPC for DSPC |
| mRNA- | LNP | 10 | — | — | — | 2x SM-102 LNP |
| 61 | | | | | | (50:10:38.5:1.5) sub |
| | | | | | | POPC for DSPC |
| x-1 | iFNP | 10 | 50% | 3 mg SM-102 | 1.5 mg PEG- | 0.5 eq Ca, 10% MQ |
| | | | | 1.3 mg Chol | DMG | in iFNP. 1.9x charge |
| | | | | 0.7 mg POPC | | ratio of SM-102 |
| x-2 | iFNP | 10 | 50% | 3 mg SM-102 | 2.2 mg PEG- | 0.5 eq Ca, 10% MQ |
| | | | | 1.3 mg Chol | PLA | in iFNP. 1.9x charge |
| | | | | 0.7 mg POPC | | ratio of SM-102 |
| mRNA- | iFNP | 100 | 50% | 3 mg SM-102 | 1.5 mg PEG- | 0.5 eq of Ca, 1.9x |
| 62 | | | | 1.3 mg Chol | DMG | charge ratio of SM- |
| | | | | 0.7 mg POPC | | 102 |
| mRNA- | iFNP | 100 | 50% | 3 mg SM-102 | 1.5 mg PEG- | 0.5 eq of Ca, another |
| 63 | | | | 1.3 mg Chol | DMG | 0.5 eq of Ca added |
| | | | | 0.7 mg POPC | | after iFNP. 1.9x |
| | | | | | | charge ratio of SM- |
| | | | | | | 102 |
| mRNA- | iFNP | 100 | 50% | 3 mg SM-102 | 2.2 mg PEG- | 0.5 eq of Ca, 1.9x |
| 64 | | | | 1.3 mg Chol | DMG | charge ratio of SM- |
| | | | | | | 102 |
| mRNA- | LNP | 100 | — | — | — | 6x SM-102 LNP |
| 65 | | | | | | (50:10:38.5:1.5) |
| mRNA- | LNP | 100 | — | — | — | 2x SM-102 LNP |
| 66 | | | | | | (50:10:38.5:1.5) |
| mRNA- | iFNP | 0 | 50% | 3 mg SM-102 | 1.5 mg PEG- | 0.5 eq of Ca. 1.9x |
| 67 | | | | 1.3 mg Chol | DMG | charge ratio of SM- |
| | | | | 0.7 mg POPC | | 102 |
| mRNA- | LNP | 100 | — | — | — | 6x SM-102 LNP |
| 69 | | | | | | (50:10:38.5:1.5) |
| mRNA- | iFNP | 100 | 50% | 3 mg SM-102 | 1.5 mg PEG- | 0.5 eq of Ca. 1.9x |
| 70 | | | | 1.3 mg Chol | DMG | charge ratio of SM- |
| | | | | 0.7 mg POPC | | 102. Dilute with 150 |
| | | | | | | mM NaCl before |
| | | | | | | ultrafiltration with |
| | | | | | | PBS |
| x-3 | iFNP | 25 | 50% | 3 mg SM-102 | 1.5 mg PEG- | 0.5 eq of Ca. 1.9x |
| | | | | 1.3 mg Chol | DMG | charge ratio of SM- |
| | | | | 0.7 mg POPC | | 102. Dilute with 150 |
| | | | | | | mM NaCl before |
| | | | | | | ultrafiltration with |
| | | | | | | 150 mM NaCl |
| x-4 | iFNP | 25 | 50% | 3 mg SM-102 | 1.5 mg PEG- | 0.5 eq of Ca. 1.9x |
| | | | | 1.3 mg Chol | DMG | charge ratio of SM- |
| | | | | 0.7 mg POPC | | 102. Quench bath of |
| | | | | | | coating is 150 mM |
| | | | | | | NaCl. Dilute with |
| | | | | | | 150 mM NaCl before |
| | | | | | | ultrafiltration with |
| | | | | | | 150 mM NaCl |
| x-5 | iFNP | 25 | 50% | 3 mg SM-102 | 1.5 mg PEG- | 0.5 eq of Ca. 1.9x |
| | | | | 1.3 mg Chol | DMG | charge ratio of SM- |
| | | | | 0.7 mg POPC | | 102. Use MIVM for |
| | | | | | | the coating with 3 |
| | | | | | | deionized water |
| | | | | | | streams and 1 |
| | | | | | | acetonitrile stream. |
| | | | | | | Dilute with 150 mM |
| | | | | | | NaCl before |
| | | | | | | ultrafiltration with |
| | | | | | | 150 mM NaCl |
| x-6 | iFNP | 25 | 50% | 3 mg SM-102 | 1.5 mg PEG- | 2 eq of Ca. 1.9x |
| | | | | 1.3 mg Chol | DMG | charge ratio of SM- |
| | | | | 0.7 mg POPC | | 102. Dilute with 150 |
| | | | | | | mM NaCl before |
| | | | | | | ultrafiltration with |
| | | | | | | 150 mM NaCl |
| mRNA- | iFNP | 100 | 50% | 3 mg SM-102 | 1.5 mg PEG- | Add 0.25M urea to |
| 71 | | | | 1.3 mg Chol | DMG | DMSO input. 0.5 eq |
| | | | | 0.7 mg POPC | | of Ca. 1.9x charge |
| | | | | | | ratio of SM-102. |
| | | | | | | Dilute with 150 mM |
| | | | | | | NaCl before |
| | | | | | | ultrafiltration with |
| | | | | | | PBS |
| mRNA- | iFNP | 100 | 50% | 3 mg SM-102 | 1.5 mg PEG- | Add 0.25M urea to |
| 72 | | | | 1.3 mg Chol | DMG | DMSO input. 0.5 eq |
| | | | | 0.7 mg POPC | | of Ca. 1.9x charge |
| | | | | | | ratio of SM-102. |
| | | | | | | Dilute with 25 mM |
| | | | | | | acetate buffer before |
| | | | | | | ultrafiltration with |
| | | | | | | PBS |
| mRNA- | iFNP | 100 | 33% | 3 mg SM-102 | 1.5 mg PEG- | 1.9x charge ratio of |
| 73 | | | | 1.3 mg Chol | DMG | SM-102. Dilute with |
| | | | | 0.7 mg POPC | | 150 mM NaCl buffer |
| | | | | | | before ultrafiltration |
| | | | | | | with PBS |
| mRNA- | iFNP | 100 | 33% | 2.7 mg Dlin- | 1.5 mg PEG- | 1.9x charge ratio of |
| 74 | | | | MC3 | DMG | Dlin-MC3-DMA |
| | | | | 1.3 mg Chol | | |
| | | | | 0.7 mg POPC | | |
| mRNA- | LNP | 100 | — | — | — | 6x SM-102 LNP |
| 75 | | | | | | (50:10:38.5:1.5) |
| mRNA- | LNP | 100 | — | — | — | 6x SM-102 LNP |
| 76 | | | | | | (50:10:38.5:1.5) |
| mRNA- | iFNP | 100 | 33% | 3 mg SM-102 | 1.5 mg PEG- | 0.75 eq of Ca. 1.9x |
| 77 | | | | 1.3 mg Chol | DMG | charge ratio of SM- |
| | | | | 0.7 mg POPC | | 102. Dilute with 150 |
| | | | | | | mM NaCl before |
| | | | | | | ultrafiltration with |
| | | | | | | isotonic HEPES |
| | | | | | | buffer |
| mRNA- | iFNP | 100 | 33% | 3 mg SM-102 | 1.5 mg PEG- | 0.75 eq of Ca. 1.9x |
| 78 | | | | 1.3 mg Chol | DMG | charge ratio of SM- |
| | | | | 0.7 mg POPC | | 102. Dilute with 25 |
| | | | | | | mM acetate buffer |
| | | | | | | (pH 4) before |
| | | | | | | ultrafiltration with |
| | | | | | | PBS |
| mRNA- | iFNP | 100 | 33% | 3.1 mg SM-102 | 1.5 mg PEG- | iFNP solvent stream |
| 79 | | | | 1.3 mg Chol | DMG | was DMSO with 5% |
| | | | | 0.7 mg POPC | | 90 mM NaCl in |
| | | | | | | water, 1 eq Ca., 2x |
| | | | | | | charge ratio of SM- |
| | | | | | | 102, coated in water, |
| | | | | | | diluted and then |
| | | | | | | washed in PBS |
| mRNA- | iFNP | 100 | 33% | 3.1 mg SM-102 | 1.125 mg | iFNP solvent stream |
| 80 | | | | 1.3 mg Chol | PEG-DMG, | was DMSO with 5% |
| | | | | 0.7 mg POPC | 0.55 mg PLA- | 90 mM NaCl in |
| | | | | | b-PEG | water, 1 eq Ca., 2x |
| | | | | | | charge ratio of SM- |
| | | | | | | 102, coated in water, |
| | | | | | | diluted and then |
| | | | | | | washed in PBS |

Nanoparticles were added to adherent cell cultures with a starting confluence of 30-50% and were incubated for 24 hours at standard cell culture conditions. Cells were washed and resuspended in PBS+500 ng/mL DAPI (2-(4-carbamimidoylphenyl)-1H-indole-6-carboximidamide) followed by analysis using the Attune NxT flow cytometer (ThermoFisher). Singlet cells were gated using SSC-A×SSC-H, and live cells were gated from the DAPI-negative population. Fluorescent intensities for GFP were captured on the BL-1 channel without compensation. Cell cultures were HEK-293T and HepG2.

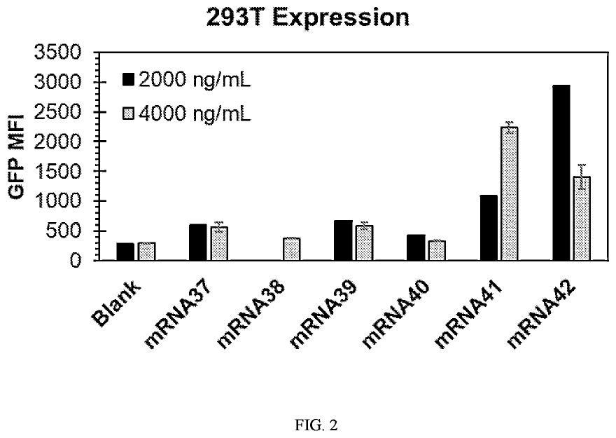

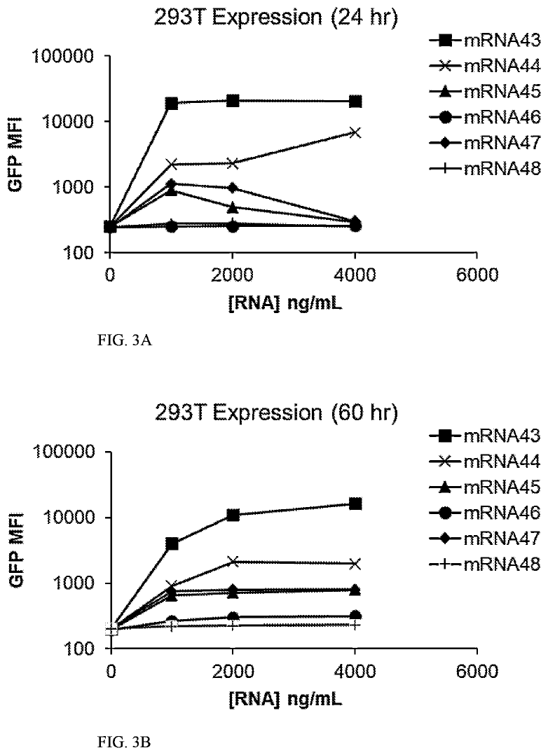

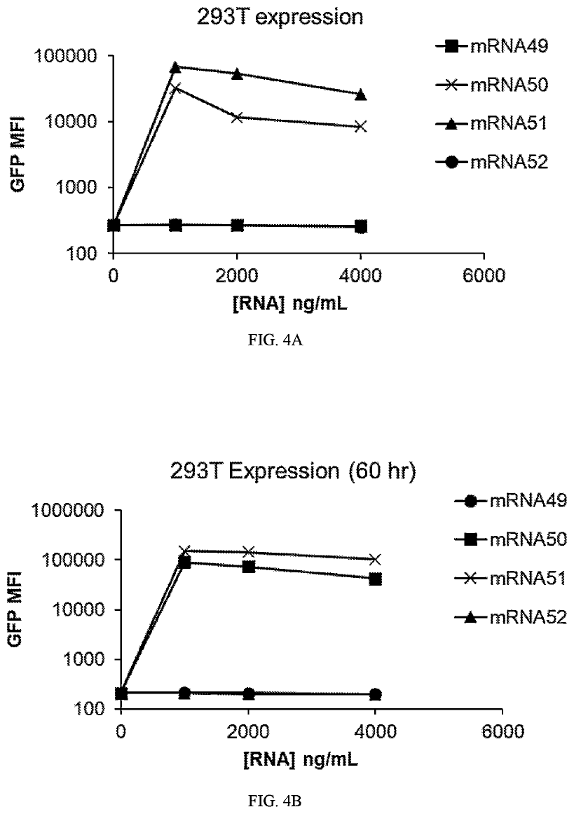

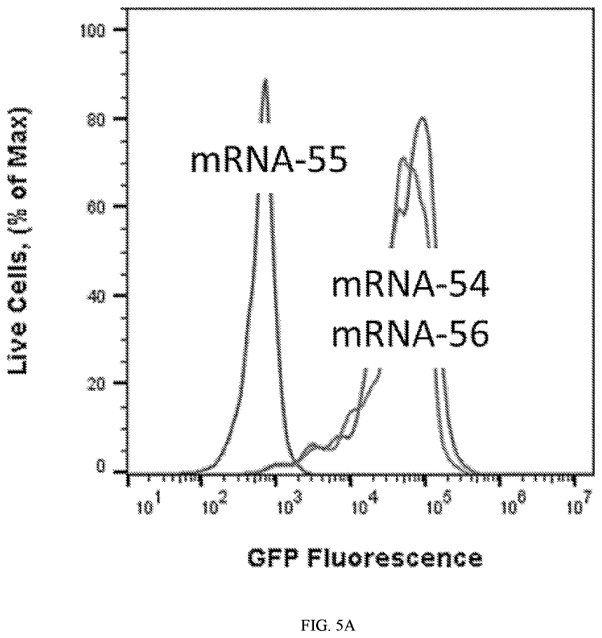

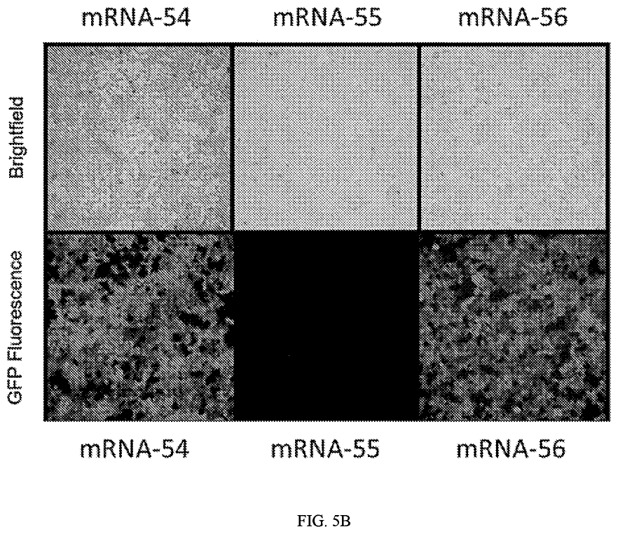

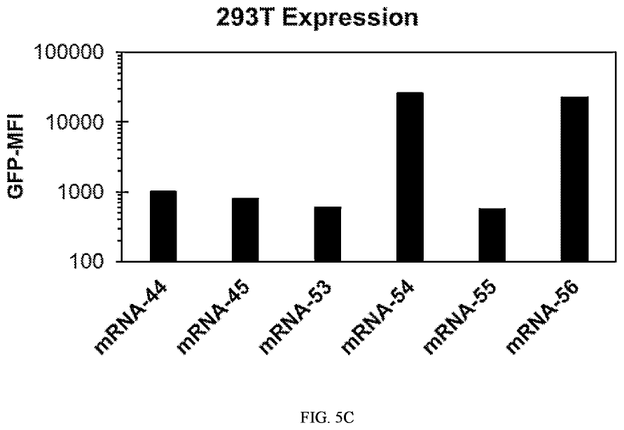

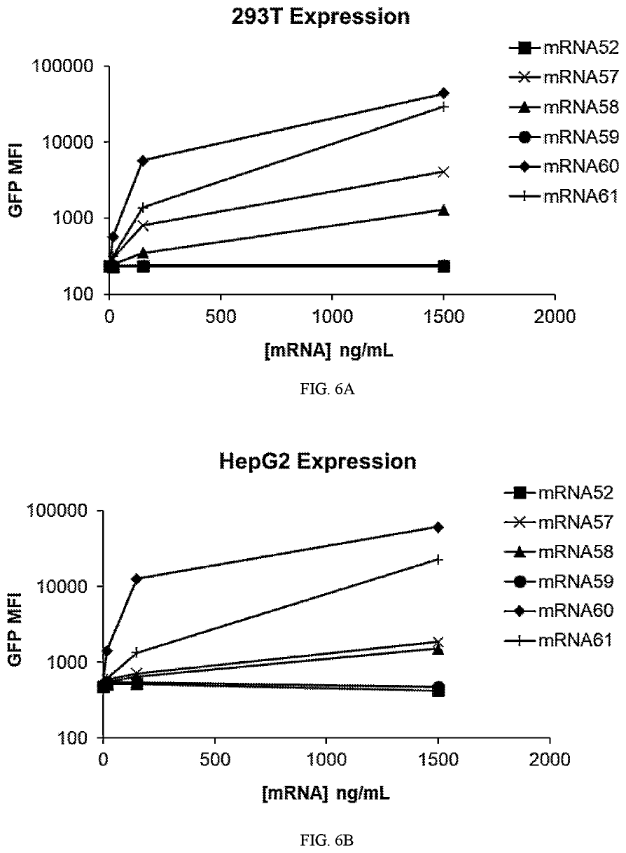

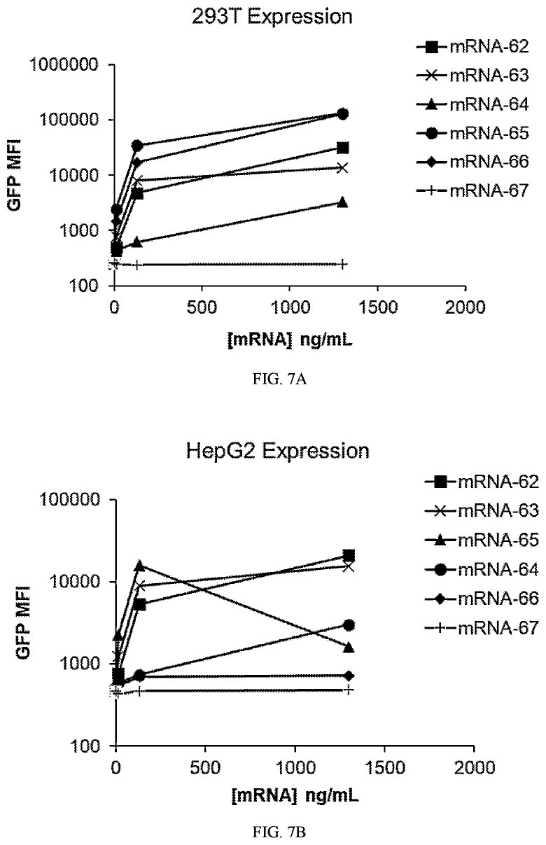

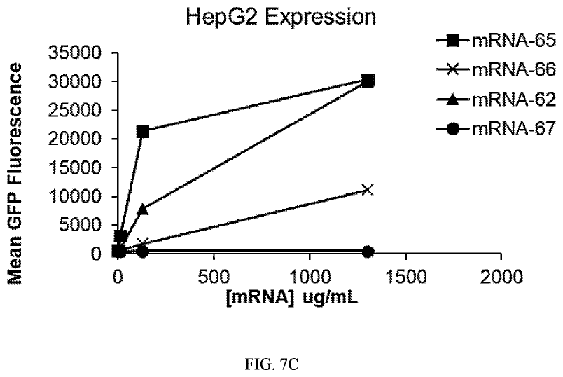

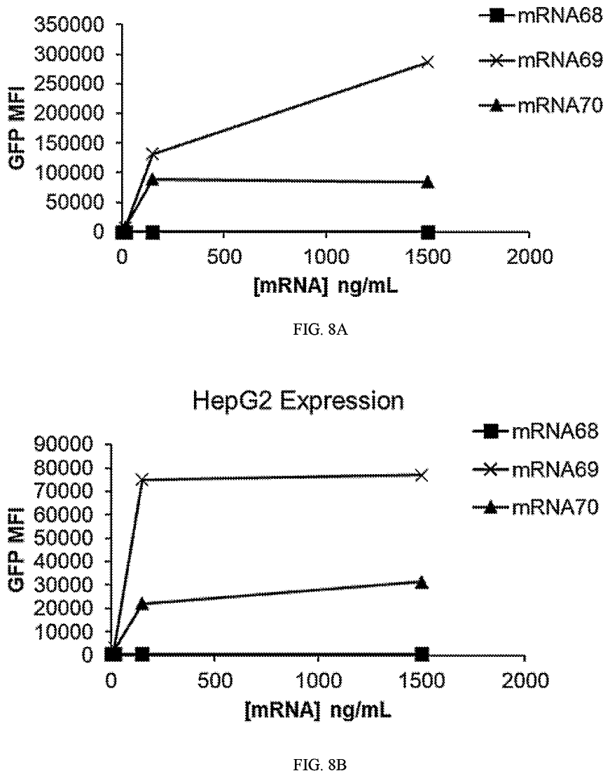

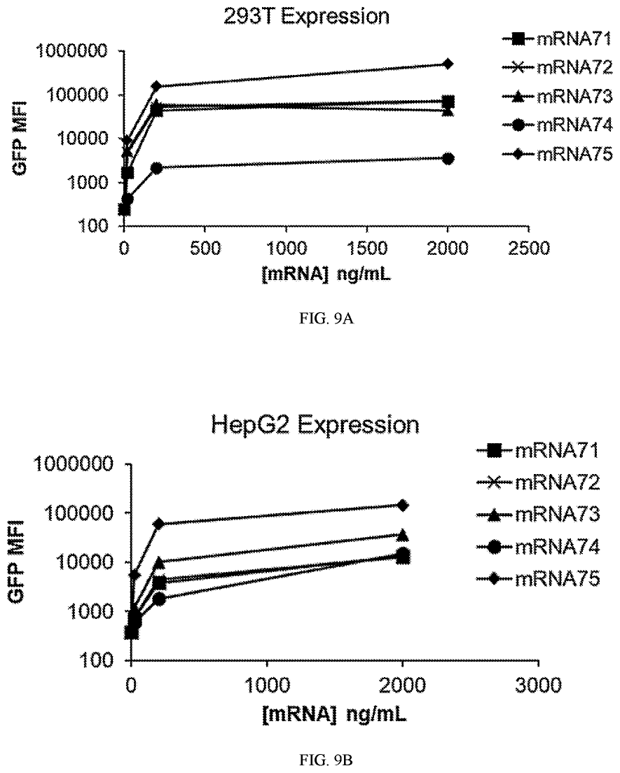

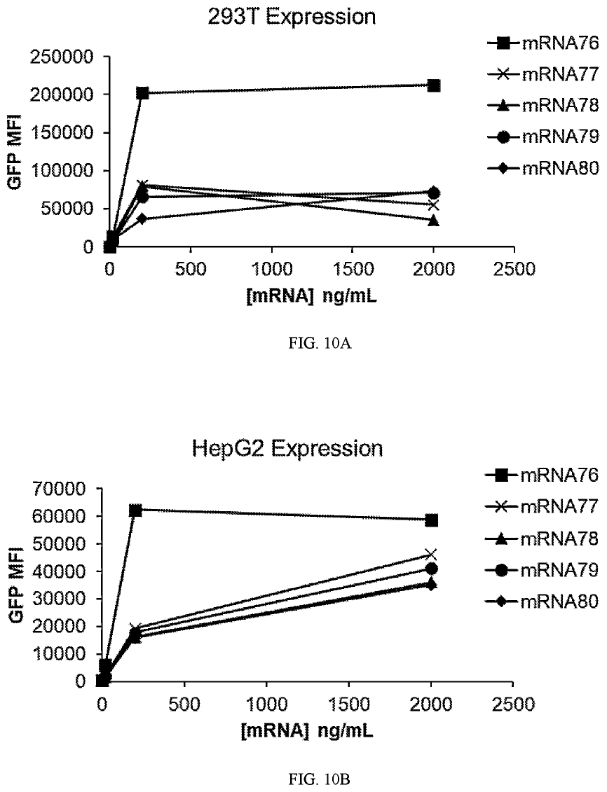

The characterization results of each formulation are summarized in Table 13. As a summarizing value, the GFP signal is reported in Table 13. This is calculated as [nanoparticle signal−negative control]−[positive control−negative control] at an mRNA dose of 100-200 ng/mL in the 293T cell line. The positive control is the standard lipid nanoparticle run in that cohort. The negative control is an iFNP formulation containing only carrier RNA (0% mRNA). Dose curves were produced for most formulations and are summarized in FIGS. 2, 3A, 3B, 4A, 4B, 6A, 6B, 7A, 7B, 7C, 8A, 8B, 9A, 9B, 10A, and 10B. FIG. 5A shows flow cytometry histograms for the indicated formulations showing equivalent similar expression between an iFNP and lipid nanoparticles (LNP) formulation at 1 μg/mL RNA dose. The control formulation (mRNA-55) without mRNA was significantly less fluorescent; the control formulation, mRNA-55, was made with the same steps and conditions and with the same components as mRNA-54, but with no mRNA. FIG. 5B shows epifluorescence imaging and brightfield of the formulations after 24 hours incubation with 293T cells. FIG. 5C shows expression at 24 hours of GFP in 293T cell culture measured by fluorescence intensity from flow cytometry analysis.

TABLE 13
Nanoparticle characterization results. Size and PDI
are calculated by DLS. RNAse protection and
GFP signal calculations are summarized in the text. “pos”
indicates a sample that served as the
positive control for a cohort of formulations.

| | | | | RNAse | |
|---|---|---|---|---|---|
| | Size | | Zeta | Protection | GFP |
| Name | (nm) | PDI | (mV) | (%) | Signal |
| mRNA- | 255 | 0.35 | −9 | nt | 0.000 |
| 001 | | | | | |
| mRNA- | 490 | 0.45 | nt | nt | 0.000 |
| 002 | | | | | |
| mRNA- | 395 | 0.38 | nt | nt | 0.000 |
| 003 | | | | | |
| mRNA- | 795 | 0.60 | nt | nt | 0.000 |
| 004 | | | | | |
| mRNA- | 270 | 0.45 | nt | nt | nt |
| 005 | | | | | |
| mRNA- | 207 | 0.43 | nt | nt | nt |
| 006 | | | | | |
| mRNA- | 116 | 0.30 | −31 | nt | 0.001 |
| 007 | | | | | |
| mRNA- | 171 | 0.20 | −12 | nt | 0.000 |
| 008 | | | | | |
| mRNA- | 145 | 0.27 | −13 | nt | 0.001 |
| 009 | | | | | |
| mRNA- | 172 | 0.30 | −29 | nt | 0.000 |
| 010 | | | | | |
| mRNA- | 148 | 0.17 | −11 | 20.5% | 0.000 |
| 011 | | | | | |
| mRNA- | 176 | 0.41 | −9 | nt | 0.001 |
| 012 | | | | | |
| mRNA- | 138 | 0.38 | −11 | nt | 0.001 |
| 013 | | | | | |
| mRNA- | 120 | 0.29 | −9 | nt | 0.000 |
| 014 | | | | | |
| mRNA- | 258 | 0.33 | −8 | nt | 0.000 |
| 015 | | | | | |
| mRNA- | 309 | 0.42 | −13 | nt | 0.000 |
| 016 | | | | | |
| mRNA- | 159 | 0.30 | −10 | nt | 0.000 |
| 017 | | | | | |
| mRNA- | 383 | 0.34 | −9 | nt | 0.000 |
| 018 | | | | | |
| mRNA- | 207 | 0.28 | −7 | 32.3% | 0.000 |
| 019 | | | | | |
| mRNA- | 151 | 0.18 | −7.1 | 43% | nt |
| 020 | | | | | |
| mRNA- | 198 | 0.23 | −7.7 | 62% | nt |
| 021 | | | | | |
| mRNA- | 218 | 0.27 | −7.3 | 53% | nt |
| 022 | | | | | |
| mRNA- | 163 | 0.28 | −4.8 | 57% | 0.000 |
| 023 | | | | | |
| mRNA- | 119 | 0.36 | −10.5 | 50% | 0.000 |
| 024 | | | | | |
| mRNA- | 168 | 0.25 | −4.6 | 70% | 0.000 |
| 025 | | | | | |
| mRNA- | 96 | 0.15 | −2.1 | 69% | 0.000 |
| 026 | | | | | |
| mRNA- | 67 | 0.17 | −1.8 | 100% | 0.000 |
| 027 | | | | | |
| mRNA- | 769 | 0.84 | −0.9 | 70% | 0.000 |
| 028 | | | | | |
| mRNA- | 84 | 0.21 | nt | 79% | −0.001 |
| 029 | | | | | |
| mRNA- | 54 | 0.23 | nt | 27% | −0.001 |
| 030 | | | | | |
| mRNA- | 76 | 0.18 | nt | 75% | −0.001 |
| 031 | | | | | |
| mRNA- | 55 | nt | nt | 25% | −0.001 |
| 032 | | | | | |
| mRNA- | 51 | nt | nt | 27% | 0.004 |
| 033 | | | | | |
| mRNA- | 92 | 0.24 | −0.4 | nt | 0.005 |
| 034 | | | | | |
| mRNA- | 84 | 0.34 | 8.0 | nt | pos |
| 035 | | | | | |
| mRNA- | 105 | 0.18 | 11.6 | nt | 8.36 |
| 036 | | | | | |
| mRNA- | 167 | 0.18 | −10.5 | 53% | 0.234 |
| 037 | | | | | |
| mRNA- | 196 | 0.30 | −11.2 | 56% | 0.037 |
| 038 | | | | | |
| mRNA- | 16 | 0.16 | −9.1 | 52% | 0.26 |
| 039 | | | | | |
| mRNA- | 171 | 0.23 | −10.1 | 39% | 0.028 |
| 040 | | | | | |
| mRNA- | 94 | 0.10 | −1.7 | 91% | pos |
| 041 | | | | | |
| mRNA- | 110 | 0.11 | 0.8 | 88% | pos |
| 042 | | | | | |
| mRNA- | 147 | 0.13 | −0.4 | 112% | pos |
| 043 | | | | | |
| mRNA- | 127 | 0.12 | −15.7 | 96% | 0.105 |
| 044 | | | | | |
| mRNA- | 365 | 0.46 | −14.2 | 58% | 0.034 |
| 045 | | | | | |
| mRNA- | 409 | 0.37 | −2.1 | 81% | 0.000 |
| 046 | | | | | |
| mRNA- | 115 | 0.57 | −9.7 | 70% | 0.047 |
| 047 | | | | | |
| mRNA- | 200 | 0.23 | −9.4 | 59% | 0.002 |
| 048 | | | | | |
| mRNA- | 183 | 0.17 | −5.2 | 77% | 0.476 |
| 050 | | | | | |
| mRNA- | 102 | 0.17 | 2 | 91% | pos |
| 051 | | | | | |
| mRNA- | 205 | nt | nt | 63% | 1.140 |
| 054 | | | | | |
| mRNA- | 76 | nt | nt | 99% | pos |
| 056 | | | | | |
| mRNA- | 245 | 0.24 | −11.8 | 52% | 0.09 |
| 57 | | | | | |
| mRNA- | 215 | 0.21 | −15.4 | 14% | 0.02 |
| 58 | | | | | |
| mRNA- | 166 | 0.17 | −14.7 | 7% | 0 |
| 59 | | | | | |
| mRNA- | 102 | 0.10 | 1.6 | 99% | pos |
| 60 | | | | | |
| mRNA- | 197 | 0.15 | −1.3 | 8% | 0.67 |
| 61 | | | | | |
| x-1 | 228 | 0.30 | nt | 65% | nt |
| x-2 | 209 | 0.22 | nt | 79% | nt |
| mRNA- | 341 | | nt | 17% | 0.24 |
| 62 | | | | | |
| mRNA- | 312 | 0.44 | nt | 19% | 0.1 |
| 63 | | | | | |
| mRNA- | 300 | 0.38 | nt | 12% | 0.02 |
| 64 | | | | | |
| mRNA- | 84 | 0.24 | nt | 29% | pos |
| 65 | | | | | |
| mRNA- | 98 | 0.32 | nt | 27% | 0.97 |
| 66 | | | | | |
| mRNA- | 115 | 0.24 | nt | 92% | 0 |
| 67 | | | | | |
| mRNA- | 99 | 0.20 | −2 | 49% | pos |
| 69 | | | | | |
| mRNA- | 164 | 0.16 | −14 | 16% | 0.67 |
| 70 | | | | | |
| x-3 | 113 | 0.34 | nt | 81% | nt |
| x-4 | 122 | 0.24 | nt | 76% | nt |
| x-5 | 118 | 0.19 | nt | 69% | nt |
| x-6 | 184 | 0.37 | nt | 67% | nt |
| mRNA- | 279 | 0.24 | −11.4 | 18% | 0.28 |
| 71 | | | | | |
| mRNA- | 228 | 0.23 | −12.8 | 18% | 0.33 |
| 72 | | | | | |
| mRNA- | 177 | 0.28 | −11.1 | 19% | 0.39 |
| 73 | | | | | |
| mRNA- | 147 | 0.13 | −5.51 | 14% | 0.01 |
| 74 | | | | | |
| mRNA- | — | — | nt | 27% | pos |
| 75 | | | | | |
| mRNA- | 77 | 0.22 | −3.88 | 31% | pos |
| 76 | | | | | |
| mRNA- | 167 | 0.27 | −6.47 | 27% | 0.4 |
| 77 | | | | | |
| mRNA- | 162 | 0.37 | −7 | 30% | 0.39 |
| 78 | | | | | |
| mRNA- | 193 | 0.16 | −24.7 | nt | 0.33 |
| 79 | | | | | |
| mRNA- | 167 | 0.21 | −20.3 | nt | 0.18 |
| 80 | | | | | |

### Example 9: Alternative Buffer Exchange Processing of GFP mRNA Formulations

mRNA encoding GFP was mixed with a Dex-PLGA (503-80) in DMSO containing 10 vol % deionized water. The RNA in the TEA salt form was present at 2.5 mg/mL (mass including the TEA counterion) and the polymer stabilizer was present at 5 mg/mL. This solution (0.5 mL) was rapidly mixed with an antisolvent stream of DCM in a multi-inlet vortex mixer (MIVM). The DCM entered the mixer in three separate streams of 0.5 mL volume. CaCl₂) at a 0.75 charge equivalent ratio with respect to the total acid content of the formulation was included in one DCM stream. Additional DCM, referred to as the “iFNP quench,” was included in the collection vial so that DMSO content after collection was <10 vol %. Accounting for hold-up in the mixer apparatus, the mRNA mass was 0.75 mg (free acid basis) and the Dex-PLGA mass was 1.875 mg.

Prior to the solvent exchange process, a cationic lipid, SM-102 (CAS #2089251-47-6), was added to the dispersion (3 mg). After 5 minutes age time, POPC (0.7 mg) and cholesterol (1.3 mg) were added. After an additional 5 minutes age time, PEG-DMG (1.5 mg) was added. These components were all added in ethanol. They were in a 47:10:37:6 (SM-102:POPC cholesterol:PEG-DMG) mol ratio with respect to each other. The SM-102 was added at approximately 1.9 charge equivalents with respect to the RNA acid groups. The DCM solution was concentrated to rotovap about 4-fold to 1-1.5 mL. Then acetonitrile (8 mL) was slowly added to the vial. The dispersion was concentrated to 1-1.5 mL at 70 torr with the heat bath set to 35° C. Then acetonitrile (8 mL) was added to the vial. The dispersion was concentrated to 1-1.5 mL at 65 torr with the heat bath set to 35° C. Then acetonitrile (8 mL) was added to the vial. The dispersion was concentrated to 1 mL at 60 torr with the heat bath set to 35° C.

The inverse NC dispersion in acetonitrile was then rapidly mixed against an equal volume of deionized water in a CIJ mixer, and a collection bath of additional deionized water (sample A) or phosphate-buffered saline (sample B) was used to reduce the MeCN volume content to less than 10 vol %. This process produced the PEG-coated nanocarrier.

The formulations were processed into a final buffer as follows. For sample A1, the nanocarriers were dialyzed in 500 volume equivalents excess of PBS at 2-8° C. for 18 hours. The nanocarriers were then concentrated by ultrafiltration on a 50 kDa molecular-weight-cut-off filter to the desired RNA concentration. For sample A2, the nanocarriers were diluted with an equal volume of PBS and stored at 2-8° C. for 18 hours. The nanocarriers were then concentrated by ultrafiltration on a 50 kDa molecular-weight-cut-off filter. An equal volume of PBS as the total nanocarrier input volume was added to the filter and the concentration step was repeated. A second PBS addition and concentration to the desired RNA range afforded the nanocarrier in the final buffer. For A3, the nanocarriers were not diluted but stored at 2-8° C. for 18 hours without stirring. The nanocarriers were then concentrated by ultrafiltration on a 50 kDa molecular-weight-cut-off filter. An equal volume of deionized water as the total nanocarrier input volume was added to the filter and the concentration step was repeated. A second water addition and concentration to the desired RNA range afforded the nanocarriers in concentrated form. Sample B1, which had been prepared with a collection bath that was PBS rather than deionized water, was dialyzed in 500 volume equivalents excess of PBS at 2-8° C. for 18 hours. The nanocarriers were then concentrated by ultrafiltration on a 50 kDa molecular-weight-cut-off filter to the desired RNA concentration. For sample B2, the nanocarriers were stored at 2-8° C. for 18 hours. The nanocarriers were then concentrated by ultrafiltration on a 50 kDa molecular-weight-cut-off filter. PBS at 2 times the total nanocarrier input volume was added to the filter and the concentration step was repeated. A second PBS addition and concentration to the desired RNA range afforded the nanocarriers in the final buffer.

DLS size data were obtained using standard techniques. The nanocarriers were also evaluated for RNAse A protection of the encapsulated RNA. This assay was conducted as follows. 50 μL of nanocarriers (˜200 μg/mL RNA) in PBS were mixed with 100 μL of RNAse A in deionized water (20 g/mL). After 1 hour incubation at 37° C., 100 μL of proteinase K (1 mg/mL) plus DTT (5 mM) was added. This digestion continued for 30 minutes at 55° C. A negative control was conducted using deionized water without the enzymes. RNA content was then measured by dissolving the nanoparticles in Triton X-100 (1% in Tris-EDTA, pH 8) for 15-30 minutes at 55° C. Exposed RNA is degraded, resulting in no signal in a RiboGreen concentration measurement, whereas protected RNA is detected by RiboGreen. Each assay included a positive control of soluble RNA. Additional controls confirmed that the proteinase K degradation conditions were sufficient to abolish RNAse activity. This RiboGreen assay does not detect single strand breaks but an average extent of degradation. The protection value is calculated as [RNA signal with RNAse]/[RNA signal without RNAse]*100. Encapsulation efficiency (EE) was measured by the standard RiboGreen protocol and not by ultrafiltration, as was done in previous Examples. In short, a nanocarrier dispersion was diluted in either tris(hydroxymethyl)aminomethane (Tris) buffer or Tris/EDTA/Triton x-100 (1%) buffer and incubated for 30 minutes at 37° C. The nanocarrier mixture was added to Tris buffer containing a 200× dilution of the RiboGreen dye stock and the fluorescence signal was collected at 525 nm, with excitation at 485 nm. Standard curves of mRNA prepared in Tris or Tris/EDTA/Triton buffer were used to calculate the total RNA detected in each buffer. The Tris buffer allows for detection of free RNA and the Tris/EDTA/Triton buffer measures total RNA content. These data are summarized in Table 14.

TABLE 14
Characterization of mRNA formulations by process variations

| | | | | | | RNAse |
|---|---|---|---|---|---|---|
| | Bath | Buffer | Size | | EE | Protection |
| ID | Composition | Exchange | (nm) | PDI | (%) | (%) |
| A1 | MQ | Dialysis | 130 | 0.2 | 45% | 23% |
| A2 | | PBS | 137 | 0.21 | 34% | 26% |
| A3 | | MQ | 153 | 0.16 | 31% | 27% |
| B1 | PBS | Dialysis | 90 | 0.24 | 38% | 18% |
| B2 | | PBS | 93 | 0.2 | 38% | 19% |

The stability of the nanocarrier formulation to rapid freezing was assessed using cryoprotectants of sucrose or trehalose added at a final composition of 5 wt %. Rapid freezing was achieved using a dry ice bath. The nanocarrier size was assessed after the frozen composition was thawed again. The results are summarized in Table 15.

TABLE 15
Nanocarrier size change on freeze/thaw processing

| | Input | Output Size | Output Size |
|---|---|---|---|
| | Size | (nm) | (nm) |
| | (nm) | Trehalose | Sucrose |
| A1 | 130 | 145 | 168 |
| B1 | 90 | 103 | 111 |

### Example 10: Co-Encapsulation with Small Molecule Co-Cores

The impact of incorporating small molecule co-cores was tested with the encapsulation of either of carrier RNA (>200 base pairs in size with unmodified bases, Qiagen Carrier RNA) or GFP-encoding mRNA (Dasher GFP mRNA from Aldeveron) in the TEA salt form (Table 16). The solvent stream was prepared by dissolving RNA and a Dex-PLGA stabilizer (503-80) in DMSO containing 10 vol % deionized water. For formulations with no co-core, the solvent stream contained 2.5 mg/mL RNA in the TEA salt form and either 2.5 or 5 mg/mL Dex-PLGA, as given in Table 16. For formulations with a choline co-core, 1.65 mg/mL of choline was included in the solvent stream. For formulations with a trehalose co-core, 5 mg/mL of trehalose was included in the solvent stream. This solution (0.5 mL) was rapidly mixed with three antisolvent streams of DCM in a multi-inlet vortex mixer (MIVM). The DCM entered the mixer in three separate streams of 0.5 mL volume each. CaCl₂) at 0.5 or 0.75 charge equivalent ratio with respect to the acid content of the RNA was included in one DCM stream. Additional DCM, referred to as the “iFNP quench,” was included in the collection vial so that DMSO content after collection was <10 vol %. Accounting for mixer hold-up losses, the RNA content in the inverse nanocarriers was 0.75 mg (free acid basis).

Samples were not extracted prior to lipid addition. Prior to the solvent exchange process, 3.1 mg (2 charge equivalents with respect to the acid groups in the RNA) of SM-102 (CAS #2089251-47-6) was added to the dispersion, followed by 0.7 mg of POPC, 1.3 mg of cholesterol, and 1.5 mg of PEG-DMG. The solvent exchange to the reforming solvent, acetonitrile, was carried out as described in Example 8.

The inverse nanocarrier dispersion in acetonitrile was then mixed against an equal volume of deionized water in a CIJ mixer and collected in a bath of either additional deionized water or PBS, as noted in Table 16, such that the final MeCN volume content was less than 10 vol %. This process produced the PEG-coated nanocarrier. If the bath was deionized water, then the nanocarrier dispersions was diluted further with an equal volume of PBS after coating.

TABLE 16
Parameter variations of mRNA formulations.
The Dex-PLGA concentration is the
concentration of the polymer in the iFNP solvent
stream. The calcium equivalents is the charge
equivalent with respect to the RNA.

| | | Dex-PLGA | | | |
|---|---|---|---|---|---|
| | RNA | concen- | eq. | | Coating |
| ID | Type | tration | CaCl2 | co-core | bath |
| 1 | carrier | 2.5 mg/ml | 0.5 | choline | deionized |
| | RNA | | | | water |
| 2 | carrier | 2.5 mg/ml | 0.5 | trehalose | deionized |
| | RNA | | | | water |
| mRNA- | mRNA | 5 mg/ml | 0.75 | choline | deionized |
| 84 | | | | | water |
| mRNA- | mRNA | 5 mg/ml | 0.75 | trehalose | deionized |
| 85 | | | | | water |
| mRNA- | mRNA | 2.5 mg/ml | 0.5 | n/a | deionized |
| 89 | | | | | water |
| mRNA- | mRNA | 2.5 mg/ml | 0.5 | n/a | PBS |
| 90 | | | | | |
| mRNA- | mRNA | 2.5 mg/ml | 0.5 | choline | deionized |
| 91 | | | | | water |
| mRNA- | mRNA | 2.5 mg/mL | 0.5 | choline | PBS |
| 92 | | | | | |

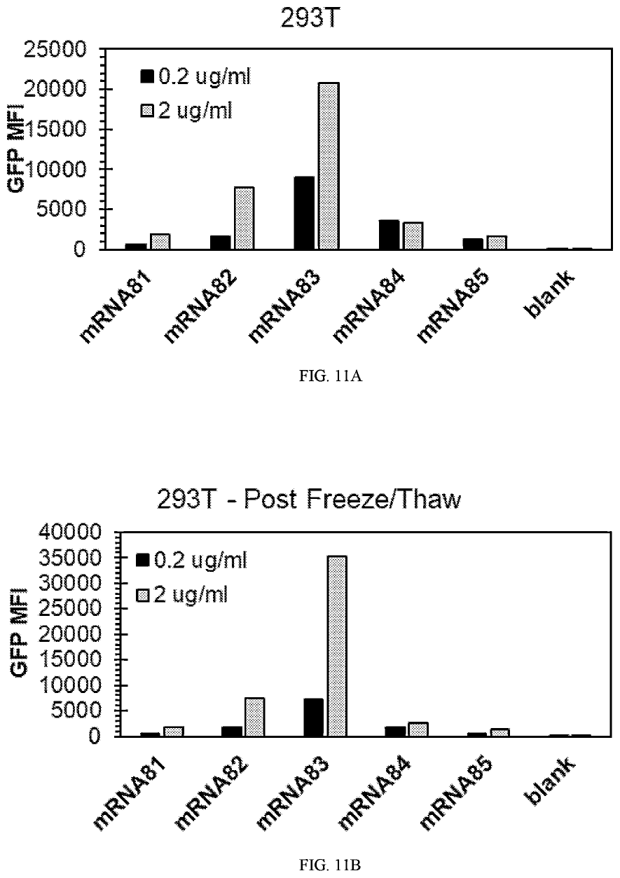

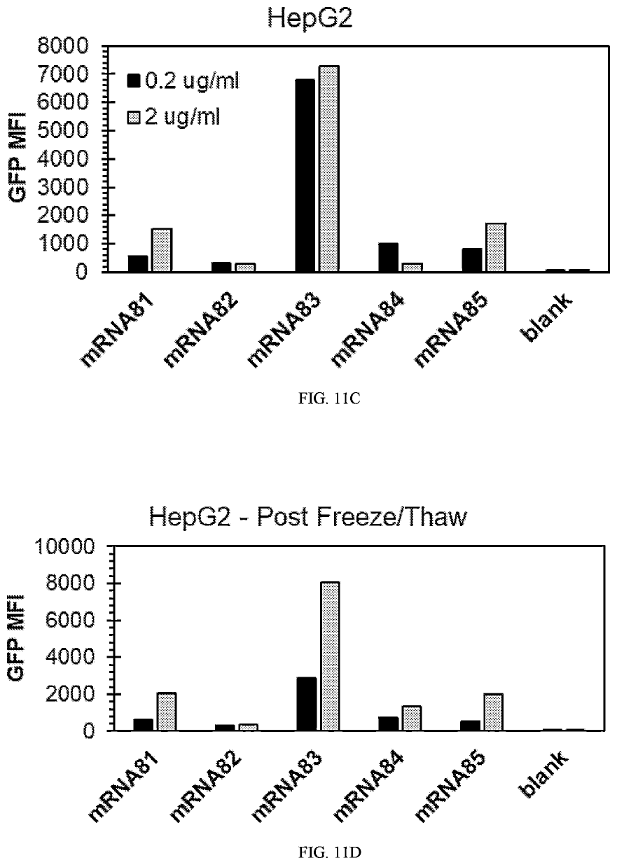

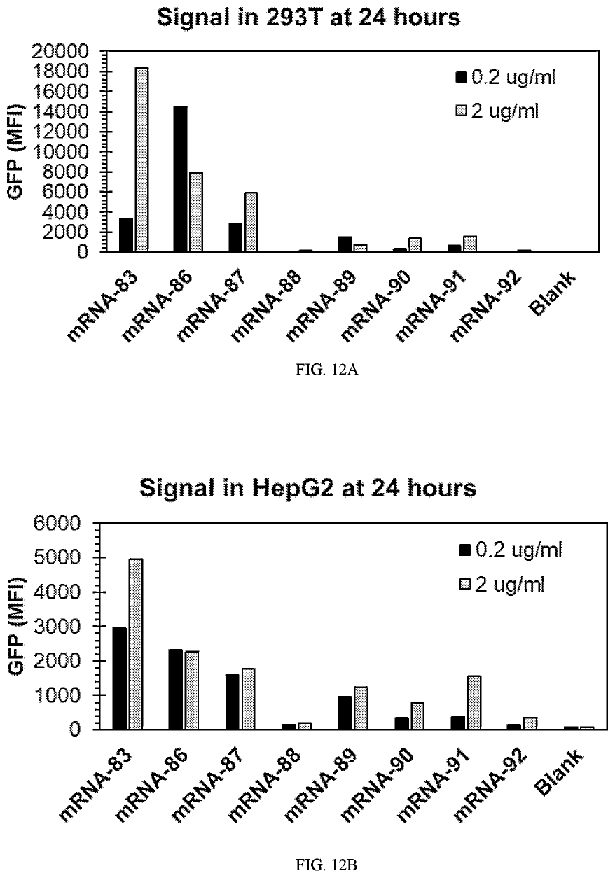

The nanocarriers were then buffer-exchanged by ultrafiltration into PBS. DLS size data were obtained using standard techniques both immediately after coating and after ultrafiltration. The ability of these nanoparticles to transfect cells was tested as given in Example 7, and the GFP signal in 293T cell culture, defined in Example 7, is given in Table 17. Dose variations are summarized in FIGS. 11A, 11B, 11C, and 11D and FIGS. 12A and 12B.

TABLE 17
Nanoparticle characterization results. Size and PDI are
calculated by DLS. Sample size after ultrafiltration was
taken prior to filtering with a 0.45 μm filter. GFP
signal calculations are summarized in the text in Example 7.

| | | | | | Zeta | |
|---|---|---|---|---|---|---|
| | Size after Coating | | Size after Ultrafiltration | | Potential | GFP |
| ID | Size (nm) | PDI | Size (nm) | PDI | (mV) | signal |
| 1 | 139 | 0.103 | 163 | 0.148 | −2.41 | n/a |
| 2 | 138 | 0.207 | 136 | 0.188 | −4.16 | n/a |
| mRNA- | 170 | 0.181 | 181 | 0.199 | −23.2 | 0.39 |
| 84 | | | | | | |
| mRNA- | 167 | 0.412 | 162 | 0.367 | −28.4 | 0.13 |
| 85 | | | | | | |
| mRNA- | 196 | 0.142 | 195 | 0.171 | −23.6 | 0.43 |
| 89 | | | | | | |

### Example 11: Effect of Lipid Ratio Variations on Transfection

mRNA encoding GFP was mixed with a Dex-PLGA (503-80) in DMSO containing 10 vol % deionized water. The RNA in the TEA salt form was present at 2.5 mg/mL and the polymer stabilizer was present at 5 mg/mL or 2.5 mg/mL, as indicated. This solution (0.5 mL) was rapidly mixed with an antisolvent stream of DCM in a multi-inlet vortex mixer (MIVM). The DCM entered the mixer in three separate streams of 0.5 mL volume each. CaCl₂) at a 0.75 or 0.5 charge equivalent ratio with respect to the total acid content of the formulation was included in one DCM stream. Additional DCM, referred to as the “iFNP quench,” was included in the collection vial so that DMSO content after collection was <10 vol %. Accounting for hold-up, the mRNA mass was 0.75 mg (free acid basis) and the Dex-PLGA mass was 1.875 mg.

Prior to the solvent exchange process, a cationic lipid, SM-102 (CAS #2089251-47-6), was added to the dispersion (3.1 mg or 4.85 mg). After 5 minutes age time, POPC (0.7 mg or 1.1 mg or 0.43 mg) and cholesterol (1.3 mg or 2.2 mg) were added. After an additional 5 minutes age time, PEG-DMG (1.5 mg) was added. These components were all added in ethanol. They were in a 47:10:39:4 or 60:5:30:5 (SM-102:POPC:cholesterol:PEG-DMG) mol ratio with respect to each other. The SM-102 was added at approximately 2 or 3 charge equivalents with respect to the RNA acid groups. The DCM solution was concentrated by rotary evaporation about 4-fold to 1-1.5 mL. Then, acetonitrile (8 mL) was slowly added to the vial. The dispersion was concentrated to 1-1.5 mL at 70 torr with the heat bath set to 35° C. Then, acetonitrile (8 mL) was added to the vial. The dispersion was concentrated to 1-1.5 mL at 65 torr with the heat bath set to 35° C. Then, acetonitrile (8 mL) was added to the vial. The dispersion was concentrated to 1 mL at 60 torr with the heat bath set to 35° C.

The inverse nanocarrier dispersion in acetonitrile was then mixed against an equal volume of deionized water in a CIJ mixer, and a collection bath of additional deionized water or phosphate-buffered saline was used to reduce the MeCN volume content to less than 10 vol %. This process produced the PEG-coated nanocarrier. These formulations are summarized in Table 18.

TABLE 18
Parameter variations of mRNA formulations. The mRNA loading represents the fraction of the
inverse nanocarrier mass that is mRNA (the remainder is the stabilizing polymer). Ca Eq is the charge
equivalents of calcium relative to the RNA backbone charges (phosphate groups) on a mole basis. The
N:P ratio is the molar ratio of SM-102 to phosphate groups on the RNA backbone. The lipid ratio
represents the internal molar ratio of SM-102:POPC:cholesterol:PEG-DMG. The coating buffer is the
composition of the collection bath used after coating in the CIJ.

| | mRNA loading (%) | Ca Eq | N:P ratio | Lipid ratio | Coating Buffer |
|---|---|---|---|---|---|
| mRNA-81 | 33 | 0.75 | 1.9 | 47:10:39:4 | PBS |
| mRNA-82 | 33 | 0.75 | 3 | 47:10:39:4 | PBS |
| mRNA-86 | 50 | 0.5 | 3 | 47:10:39:4 | Deionized |
| mRNA-87 | 50 | 0.5 | 3 | 47:10:39:4 | PBS |
| mRNA-88 | 50 | 0.5 | 3 | 60:5:30:5 | PBS |
| mRNA-89 | 50 | 0.5 | 2 | 47:10:39:4 | Deionized |
| mRNA-90 | 50 | 0.5 | 2 | 47:10:39:4 | PBS |

The formulations were processed into a final buffer as follows. The nanocarrier was concentrated by ultrafiltration on a 50 or 100 kDa molecular-weight-cut-off filter at 10° C. PBS at 2× the total nanocarrier input volume was added to the filter and the concentration step was repeated. A second PBS addition and concentration to the desired RNA range afforded the nanocarrier in the final buffer. The final form was then supplemented with 5 wt % trehalose dihydrate as a cryoprotectant and a portion was flash frozen at −196° C. DLS size and zeta potential were measured using standard techniques throughout processing.

Nanoparticles were added to adherent cell cultures of HEK-293T or HepG2 with a starting confluence of 30-50% and were incubated for 24 hours at standard cell culture conditions. Cells were washed and resuspended in PBS+500 ng/mL 4′,6-diamidino-2-phenylindole (DAPI) followed by analysis using the Attune NxT flow cytometer (ThermoFisher). Singlet cells were gated using SSC-A×SSC-H, and live cells were gated from the DAPI-negative population. Fluorescent intensities for GFP were captured on the BL-1 channel without compensation.

The characterization results of each formulation are summarized in Table 19. As a summarizing value, GFP signal is reported in Table 19. This is calculated as [nanoparticle signal−negative control]−[positive control−negative control] at an mRNA dose of 200 ng/mL. The positive control is the standard lipid nanoparticle run in that cohort. The negative control is the cell background signal (a cell blank). Dose variations are summarized in FIGS. 11A, 11B, 11C, and 11D and FIGS. 12A and 12B.

TABLE 19
Characterization of mRNA formulations. Size, PDI, and zeta potential
were measured by dynamic light scattering after ultrafiltration
and filtering with a 0.45 μm filter. Samples were re-analyzed
after one freeze/thaw cycle in 5 wt % trehalose. The GFP signal
represents the GFP signal from the formulation in the indicated
cell line at a 200 ng/mL mRNA dose after 24 hour incubation
relative to a lipid nanoparticle control (mRNA-83)

| | Coated Nanocarrier | | | Post Freeze/Thaw | | | |
|---|---|---|---|---|---|---|---|
| | Size | | Zeta | Size | | GFP Signal | |
| | (nm) | PDI | (mV) | (nm | PDI | 293T | HepG2 |
| mRNA-81 | 121 | 0.15 | −10.8 | 137 | 0.19 | 0.07 | 0.20 |
| mRNA-82 | 126 | 0.19 | −14.3 | 159 | 0.42 | 0.24 | 0.08 |
| mRNA-86 | 212 | 0.15 | −13.0 | 827 | 0.46 | 4.35 | 0.78 |
| mRNA-87 | 138 | 0.17 | −8.6 | 1158 | 0.53 | 0.86 | 0.52 |
| mRNA-88 | 89 | 0.13 | −10.0 | 127 | 0.24 | 0.02 | 0.03 |
| mRNA-89 | 216 | 0.23 | −23.6 | 236 | 0.18 | 0.43 | 0.31 |
| mRNA-90 | 138 | 0.18 | −12.9 | 157 | 0.37 | 0.09 | 0.09 |

### Example 12: iFNP Formulations of SARS RBD mRNA

mRNA encoding SARS-CoV-2 spike protein receptor binding domain (RBD) with pseudo-uridine (C5-glycoside isomer of uridine, 5-ribosyluracil) modification was mixed with Dex-PLGA (503-80) in DMSO containing 10 vol % deionized water. The RNA in the TEA salt form was present at 2 mg/mL and the polymer stabilizer was present at 2 mg/mL. This solution (0.5 mL) was rapidly mixed with an antisolvent stream of DCM in a multi-inlet vortex mixer (MIVM). The DCM entered the mixer in three separate streams of 0.5 mL volume each. CaCl₂) at a 0.5 charge equivalent ratio with respect to the total acid content of the formulation was included in one DCM stream. Additional DCM, referred to as the “iFNP quench,” was included in the collection vial so that DMSO content after collection was <10 vol %. Accounting for hold-up, the mRNA mass was 0.75 mg (free acid basis) and the Dex-PLGA mass was 1.875 mg.

Prior to the solvent exchange process, a cationic lipid, SM-102 (CAS #2089251-47-6, 8-[(2-hydroxyethyl)[6-oxo-6-(undecyloxy)hexyl]amino]-octanoic acid, 1-octylnonyl ester), was added to the dispersion (4.85 mg). After 5 minutes age time, POPC (1-palmitoyl-2-oleoyl-glycero-3-phosphocholine, 1.1 mg) and cholesterol (chol, 2.2 mg) were added. After an additional 5 minutes age time, PEG-DMG (1,2-dimyristoyl-rac-glycero-3-methoxypolyethylene glycol-2000, 1.5 mg) was added. These components were all added in ethanol. They were in a 47:10:39:4 (SM-102:POPC:cholesterol:PEG-DMG) mol ratio with respect to each other. The SM-102 was added at approximately 3 charge equivalents with respect to the RNA acid groups. The DCM solution was concentrated by rotary evaporation about 4-fold to 1-1.5 mL. Then, acetonitrile (8 mL) was slowly added to the vial. The dispersion was concentrated to 1-1.5 mL at 70 torr with the heat bath set to 35° C. Then, acetonitrile (8 mL) was added to the vial. The dispersion was concentrated to 1-1.5 mL at 65 torr with the heat bath set to 35° C. Then, acetonitrile (8 mL) was added to the vial. The dispersion was concentrated to 1 mL at 60 torr with the heat bath set to 35° C.

The inverse nanocarrier dispersion in acetonitrile was then mixed against an equal volume of deionized water in a CIJ mixer, and a collection bath of additional 0.5× phosphate-buffered saline (PBS) was used to reduce the MeCN volume content to less than 10 vol %. This process produced a PEG-coated nanocarrier.

As a control, a standard lipid nanoparticle (LNP) was prepared using a 6:1 N:P (nitrogen:phosphorous) charge ratio and a lipid composition of 50:10:38.5:1.5 (SM-102:DSPC:Chol:PEG-DMG) mol ratio. The ethanol stream containing these lipids at 15 mM was rapidly mixed in a MIVM with three citrate streams (pH 4, 50 mM) containing the mRNA. The LNP was then dialyzed against 0.5×PBS at 4° C. overnight. Samples were then concentrated to the desired range (400 g/mL) and flash frozen with 10 wt % trehalose. They were stored at −80° C. until thawed for use. These formulations are summarized in Table 20.

Particle size, for each of the examples described herein, was characterized by Dynamic Light Scattering (DLS) using a Zetasizer Nano ZS (Malvern, Worcestershire, UK) at 25° C. by diluting each sample ten-fold in water. Size distributions were determined from a CONTIN analysis implemented by the Zetasizer software. The polydispersity index (PDI) is obtained from the Taylor series expansion of the autocorrelation function which is implemented by the Zetasizer software. A ratio of the second to the first moment is defined as the PDI. Zeta potential was measured in 0.1×PBS using the Zetasizer Nano ZS.

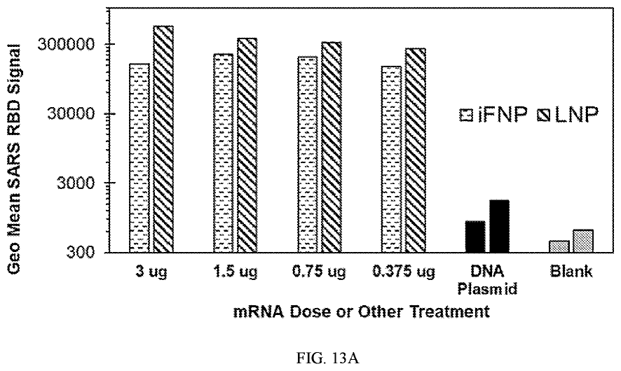

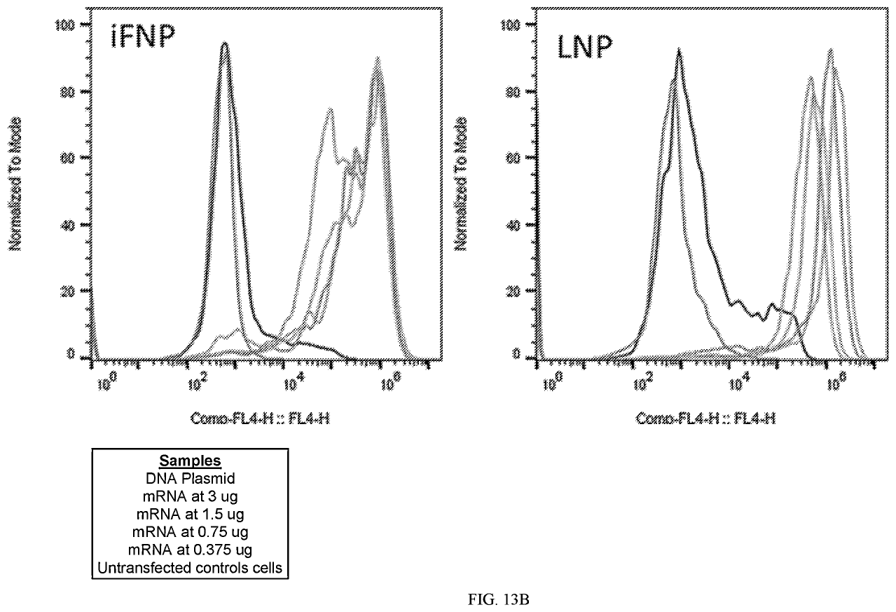

Testing of the formulations for transfection of 293T cells (an adherent human embryonic kidney 293 cell line that contains the SV40 T-antigen) was conducted in 6 well plates with mRNA doses of 3 μg, 1.5 μg, 0.75 μg, and 0.375 g per well. After 18 hours, staining for SARS-CoV2 RBD was carried out using an anti-RBD primary antibody and a fluorescent secondary goat anti-Human IgG Fey Fragment-APC (Jackson ImmunoResearch; Cat #109-135-098). These results are summarized in FIGS. 13A and 13B. As an additional confirmation of expression, treated cells were fixed with 4% paraformaldehyde before and after staining with a fluorescent antibody against SARS-CoV2 RBD. Epifluorescence images confirmed uniform expression across the cell population with significant signal at the cell membrane.

TABLE 20
Characterization of mRNA formulations. Size, PDI, and zeta
potential were measured by dynamic light scattering after
ultrafiltration and filtering with a 0.45 μm filter.
Samples were re-analyzed after one freeze/thaw cycle in
10 wt % trehalose. The RBD signal was the geometric mean
at 0.3 ug/ml RNA dose after 24 hr incubation with 293T cells.

| | Nanocarrier | | | Post Freeze/Thaw | | RBD |
|---|---|---|---|---|---|---|
| | Size | | Zeta | Size | | Signal |
| | (nm) | PDI | (mV) | (nm | PDI | (293T) |
| iFNP-SARS | 216 | 0.27 | −10 | 193 | 0.20 | 195203 |
| LNP-SARS | 112 | 0.16 | −1.1 | 130 | 0.16 | 317788 |

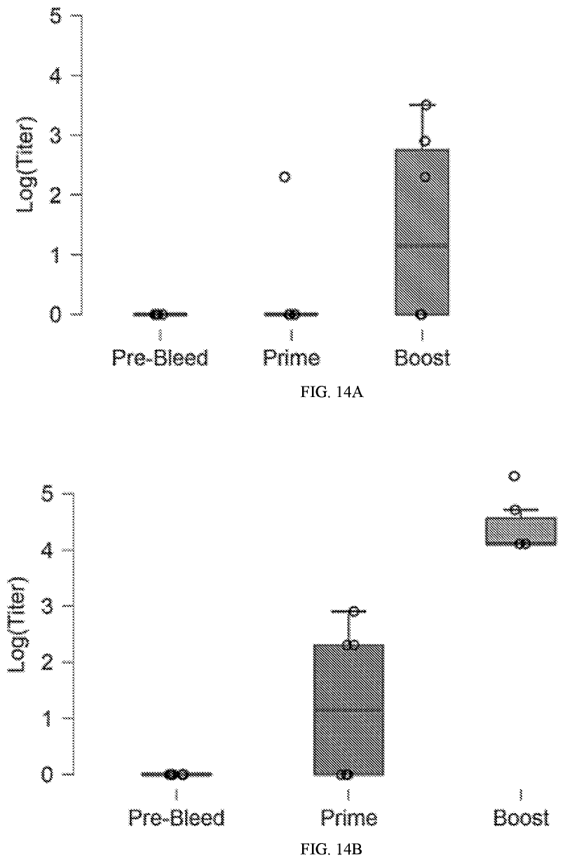

The LNP and iFNP formulations were dosed at 20 g mRNA to BALB/c mice via two 25 μL injections in each hind limb thigh muscle. At day 21, a booster dose was administered in the same fashion. Sera were collected before initial injection (pre-bleed), at day 7 (prime), and day 28 (boost). Antibody titers were analyzed by ELISA (enzyme-linked immunosorbent assay) using standard techniques with plates coated by SARS-CoV2 RBD and detected using a standard chemiluminescent assay. Both iFNP (FIG. 14A) and LNP (FIG. 14B) produced antibody titer responses. On FIGS. 14A and 14B the circles are data points; the grey boxes designate the inner quartiles range; the horizontal line segments within the grey boxes are the median value; and the horizontal line segments above the grey boxes are 1.5 times the inner quartiles range.

These results demonstrate the suitability of iFNP for the encapsulation of an mRNA sequence containing modified uridine residues (pseudo-uridine) and the subsequent expression of said mRNA sequence as a protein in mammalian cells. These results also demonstrate the suitability of iFNP for production of a pharmaceutical composition including an iFNP nanoparticle encapsulating an mRNA sequence that encodes an infectious disease antigen and administration of this to a mammalian subject and, further, the use of iFNP to formulate a vaccine that causes an immune response to be mounted against an antigen encoded by the mRNA sequence.

### Example 13: In Vitro Testing of iFNP Formulations with Parameter Variation

mRNA encoding green fluorescent protein (GFP, DasherGFP from Aldevron, Cat #3870) was mixed with either Dex-PLGA (503-80) or PAsp_{2k Da}-b-PLGA_{15 kDa} in DMSO containing 10 vol % deionized water. The RNA in the TEA salt form was present at 2 mg/mL (PAsp formulation) or 2.5 mg/mL (Dex formulation). The polymer stabilizer was present at 5 mg/mL (Dex) or 3 mg/mL (PAsp). This solution (0.5 mL) was rapidly mixed with an antisolvent stream of DCM in a multi-inlet vortex mixer (MIVM). The DCM entered the mixer in three separate streams of 0.5 mL volume. CaCl₂) at a 0.5 charge equivalent ratio with respect to the total acid content of the formulation was included in one DCM stream. Additional DCM, referred to as the “iFNP quench,” was included in the collection vial so that DMSO content after collection was <10 vol %.

Prior to the solvent exchange process, a cationic lipid, SM-102 (CAS #2089251-47-6), was added to the dispersion. After 5 minutes age time, POPC and cholesterol were added. After an additional 5 minutes age time, PEG-DMG was added. These components were all added in ethanol. The SM-102 was added at either 2 or 4 charge equivalents with respect to the RNA acid groups. The lipid components were maintained in a 47:10:39:4 (SM-102:POPC:Chol:PEG-DMG) mol ratio with respect to each other. The DCM solution was concentrated by rotary evaporation about 4-fold to 1-1.5 mL. Then, acetonitrile (8 mL) was slowly added to the vial. The dispersion was concentrated to 1-1.5 mL at 70 torr with the heat bath set to 35° C. Then, acetonitrile (8 mL) was added to the vial. The dispersion was concentrated to 1-1.5 mL at 65 torr with the heat bath set to 35° C. Then, acetonitrile (8 mL) was added to the vial. The dispersion was concentrated to 1 mL at 60 torr with the heat bath set to 35° C.

The inverse nanocarrier dispersion in acetonitrile was then mixed against an equal volume of deionized water in a CIJ mixer. A collection bath containing different buffer compositions was used to reduce the acetonitrile (MeCN) volume content to less than 10 vol %. This process produced a PEG-coated nanocarrier. The collection bath was selected from the following: deionized water, HEPES (4-(2-hydroxyethyl)-1-piperazineethanesulfonic acid, 30 mM), HEPES+NaCl (135 mM), HEPES+Trehalose (10 wt %), Tris/Acetate (30 mM), Tris/Acetate+NaCl (120 mM), or Tris/Acetate+Trehalose (10 wt %). Nanoparticles with a PAsp core were produced with deionized water for the collection bath. All buffered components were at pH 7.4. After 2-5 minutes, 1×PBS was added at equal volume to each dispersion. The formulations were processed into a final buffer as follows. The nanocarrier was concentrated by ultrafiltration on a 50 or 100 kDa molecular-weight-cut-off filter at 10° C. PBS was added twice to the filter and the concentration step was repeated each time. The concentration was adjusted to 0.15 mg/mL RNA and tested in cell culture. DLS size and zeta potential were measured using standard techniques (Table 21).

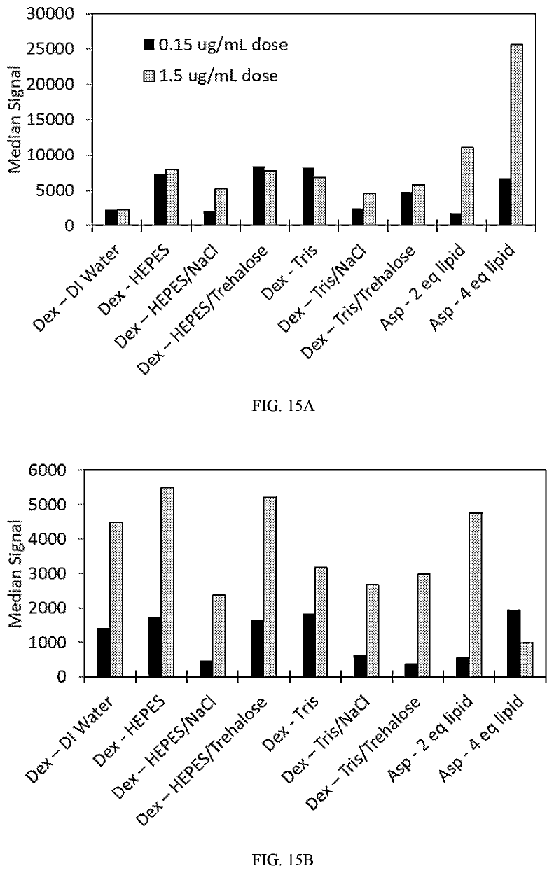

Nanoparticles were added to adherent cell cultures with a starting confluence of 30-50% and were incubated for 24 hours at standard cell culture conditions. Cells were washed and resuspended in PBS+500 ng/mL 4′,6-diamidino-2-phenylindole (DAPI) followed by analysis using the Attune NxT flow cytometer (ThermoFisher). Singlet cells were gated using SSC-A×SSC-H, and live cells were gated from the DAPI-negative population. Fluorescent intensities for GFP were captured on the BL-1 channel without compensation. Cell cultures were HEK-293T and HepG2. HepG2 is a human liver-derived hepatocellular carcinoma cell line. FIG. 15A shows the green fluorescent protein (GFP) signal measured by flow cytometry after 24 hour culture in HEK-293T culture; the median value is reported for two mRNA doses in each well. FIG. 15B shows the green fluorescent protein (GFP) signal measured by flow cytometry after 24 hour culture in HepG2 culture; the median value is reported for two mRNA doses in each well.

TABLE 21
Characterization of mRNA formulations. Size, PDI, and zeta potential
were measured by dynamic light scattering after ultrafiltration.
Formulation naming is [first stabilizing polymer] - [buffer]
except for the PAsp-b-PLGA formulations, which indicate the ratio of
ionizable lipid instead. Dex is Dex-PLGA (503-80) and Asp is PAsp-PLGA.

| Formulation | Size, z-avg (nm) | PDI | Zeta (mV) |
|---|---|---|---|
| Dex - DI Water | 140 | 0.21 | −17 |
| Dex - HEPES | 155 | 0.16 | −15 |
| Dex - HEPES/NaCl | 110 | 0.14 | −14 |
| Dex - HEPES/Trehalose | 150 | 0.15 | −16 |
| Dex - Tris | 150 | 0.17 | −17 |
| Dex - Tris/NaCl | 115 | 0.18 | −14 |
| Dex - Tris/Trehalose | 145 | 0.17 | −16 |
| Asp - 2 eq lipid | 140 | 0.21 | −22 |
| Asp - 4 eq lipid | 120 | 0.34 | −11 |

For example, the results with the PAsp-PLGA NCs with either 2 eq or 4 eq ionizable lipid showed large and dose-dependent expression of GFP. This demonstrated transfection of the cells.

These results demonstrate additional suitable iFNP nanoparticle compositions and process conditions for producing the same. The process and compositions produce nanoparticles with suitable size, polydispersity and surface charge. The nanoparticles have been shown to effectively transfect mammalian cells in culture.

### Example 14: In Vitro Evaluation of iFNP Formulations of Firefly Luciferase mRNA

mRNA encoding Firefly Luciferase modified with 5-methoxyuridine (Cat #L-7202 from TriLink Biotech) was mixed with either Dex-PLGA (503-80) or PAsp_{2 kDa}-b-PLGA_{15 kDa} in DMSO containing 10 vol % deionized water. The RNA in the TEA salt form was present at 2 mg/mL (PAsp formulation) or 2.5 mg/mL (Dex formulation). The polymer stabilizer was present at 5 mg/mL (Dex) or 3 mg/mL (PAsp). This solution was rapidly mixed with an antisolvent stream of DCM in a multi-inlet vortex mixer (MIVM). The DCM entered the mixer in three separate streams of equivalent volume. CaCl₂) at a 0.5 charge equivalent ratio with respect to the total acid content of the formulation was included in one DCM stream. Additional DCM, referred to as the “iFNP quench,” was included in the collection vial so that DMSO content after collection was <10 vol %.

Prior to the solvent exchange process, a cationic lipid, SM-102 (CAS #2089251-47-6), was added to the dispersion. After 5 minutes age time, POPC and cholesterol were added. After an additional 5 minutes age time, PEG-DMG was added. For one sample, PLA-PEG was added in lieu of PEG-DMG at 27 wt % with respect to the total mass of the formulation. These components were all added in ethanol. The SM-102 was added at either 2 or 3 charge equivalents with respect to the RNA acid groups. The lipid components were maintained in a 47:10:39:4 (SM-102:POPC:Chol:PEG-DMG) mol ratio with respect to each other. The DCM solution was concentrated by rotary evaporation about 4-fold. Then acetonitrile was slowly added to the vial. The dispersion was concentrated at 70 torr with the heat bath set to 35° C. Then acetonitrile was added to the vial. The dispersion was concentrated at 65 torr with the heat bath set to 35° C. Then acetonitrile was added to the vial. The dispersion was concentrated at 60 torr with the heat bath set to 35° C.

The inverse nanocarrier dispersion in acetonitrile was then mixed against an equal volume of deionized water in a CIJ mixer. A collection bath containing different buffer compositions was used to reduce the MeCN volume content to less than 10 vol %. This process produced a PEG-coated nanocarrier. The collection bath was selected from the following: deionized water, Tris/Acetate (30 mM), or Tris/Acetate+NaCl (120 mM). All buffered components were at pH 7.4. After 2-5 minutes, 1×PBS was added at equal volume to each dispersion. The formulations were processed into a final buffer as follows. The nanocarrier was concentrated by ultrafiltration on a 50 or 100 kDa molecular-weight-cut-off filter at 10° C. PBS was added three times to the filter and the concentration step was repeated each time. Sucrose was added at 5 wt %, the samples were filtered with a 0.45 m filter, and the concentration was adjusted to 0.25 mg/mL RNA. The samples were then flash frozen using liquid nitrogen until thawed for use. DLS size and zeta potential were measured using standard techniques throughout handling (Table 22).

As a control, two lipid nanoparticles were prepared using a 6:1 N:P ratio and a lipid composition of 50:10:38.5:1.5 (SM-102:DSPC:Chol:PEG-DMG) mol ratio for LNP1 and 50:10:39.6:0.4 (SM-102:DSPC:Chol:PEG-DMG) mol ratio for LNP2. The ethanol stream containing these lipids was rapidly mixed in a MIVM with three citrate streams (pH 4, 50 mM) containing the mRNA. The LNP was then dialyzed against 1×PBS at 4° C. overnight.

TABLE 22
Characterization of mRNA formulations. Size, PDI, and zeta potential were measured
by dynamic light scattering at the indicated point in processing. Formulation
details assume a PEG-DMG coating except where noted and PLA-PEG was used.

| | After Washing | | | After Freeze-Thaw | | Formulation Details | |
|---|---|---|---|---|---|---|---|
| | Size | | Zeta | Size | | Stabilizing | |
| Name | (nm) | PDI | (mV) | (nm) | PDI | polymer | Formulation details |
| fL1 | 263 | 0.23 | −17.2 | 219 | 0.23 | Dex-PLGA | 2 eq SM-102, Tris bath |
| fL2 | 138 | 0.2 | −9.1 | 176 | 0.19 | Dex-PLGA | 2 eq SM-102, Tris/NaCl bath |
| fL3 | 165 | 0.18 | −11.3 | 187 | 0.19 | Dex-PLGA | 3 eq SM-102, Tris/NaCl bath |
| fL4 | 397 | 0.25 | −26 | 255 | 0.16 | Dex-PLGA | PLA-PEG, 2 eq SM-102, |
| | | | | | | | Tris/NaCl bath |
| fL5 | 326 | 0.29 | −27.1 | 418 | 0.45 | PAsp-PLGA | 2 eq SM-102, DI Water bath |
| fL6 | 176 | 0.2 | −14 | 258 | 0.46 | PAsp-PLGA | 2 eq SM-102, Tris/NaCl bath |
| fL7 | 184 | 0.27 | −8.3 | 320 | 0.47 | PAsp-PLGA | 3 eq SM-102, Tris/NaCl bath |
| LNP1 | 96 | 0.12 | −1.9 | 101 | 0.19 | n/a | 1.5 mol % PEG-DMG |
| LNP2 | 206 | 0.19 | −3 | 383 | 0.11 | n/a | 0.4 mol % PEG-DMG |

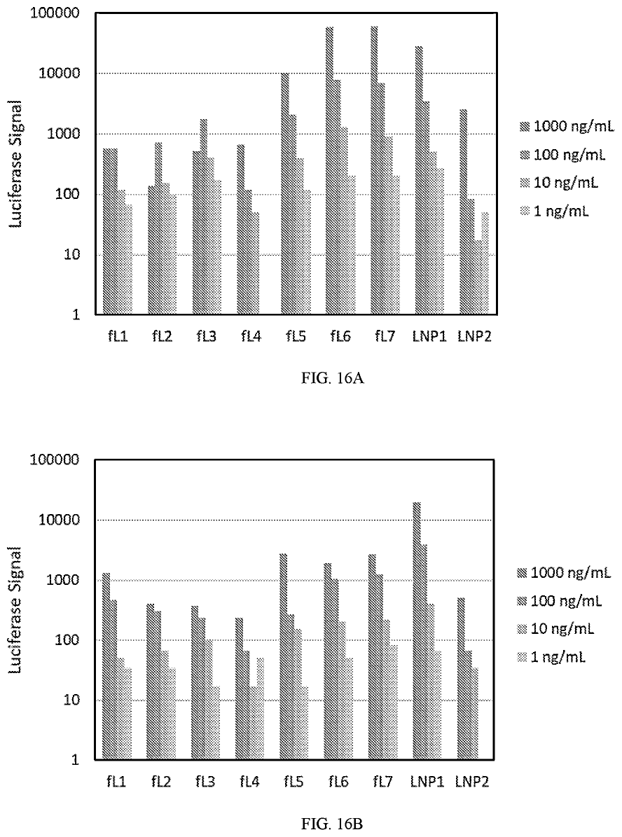

Nanoparticles were added to adherent cell cultures with a starting confluence of 30-50% and were incubated for 12-24 hours with standard cell culture conditions using a serial dilution of nanoparticles as compared to PBS-only controls. At endpoint, cells were lysed and luminescence was quantified using the Pierce Firefly Luciferase Glow Assay Kit according to the manufacturer's instructions. Samples were tested for cell transfection immediately after thawing. FIG. 16A shows the Firefly Luciferase signal after 24 hour incubation of nanoparticles with 293T cells; dose values are of mRNA in the culture media and detection used standard manufacturer recommendations as described in the text. FIG. 16B shows the Firefly Luciferase signal after 24 hour incubation of nanoparticles with HepG2 cells; dose values are of mRNA in the culture media and detection used standard manufacturer recommendations as described in the text.

The iFNP NCs induced large and dose-dependent expression of the encapsulated mRNA. This demonstrated transfection of the cells.

These results demonstrate that iFNP is suitable for the encapsulation of an mRNA sequence with 5-methoxyuridine modification, Cap 1 structure, and polyadenylation. The iFNP nanoparticles are suitable for expression of an mRNA encoded sequence in mammalian cell culture and exhibit a well-defined dose-response curve. These results demonstrate expression in cell culture using iFNP nanoparticles bearing a PLA-PEG coating that is stably attached, unlike PEG-DMG, which is known to partition off and expose the underlying lipid surface.

### Example 15: In Vivo Screening of Luciferase mRNA Formulations by Whole Animal Imaging

The following samples from Example 14 (Table 22) were tested for expression kinetics and biodistribution in BALB/c mice (6-8 week females): LNP1, fL2, fL3, fL5, fL6, fL7. Aliquots were prepared for intravenous (IV) injection (tail vein) and intramuscular (IM) injection (thigh muscle) at 125 g/mL and 250 g/mL RNA respectively. Each mouse was dosed with 12.5 g of mRNA. Bioluminescence imaging was conducted at 9, 24, 48, 96, and 144 hours post injection. A fresh stock solution of luciferin was prepared at 15 mg/ml in Dulbecco's phosphate-buffered saline and filter sterilized through a 0.22 μm filter. Mice were injected via intraperitoneal injection with a 6 mg (compound)/kg (body weight) dose of luciferin. Mice were anesthetized by isoflurane and imaged 15 minutes after luciferin injection for a 60 second exposure time (unless saturated) at each of ventral and dorsal sides.

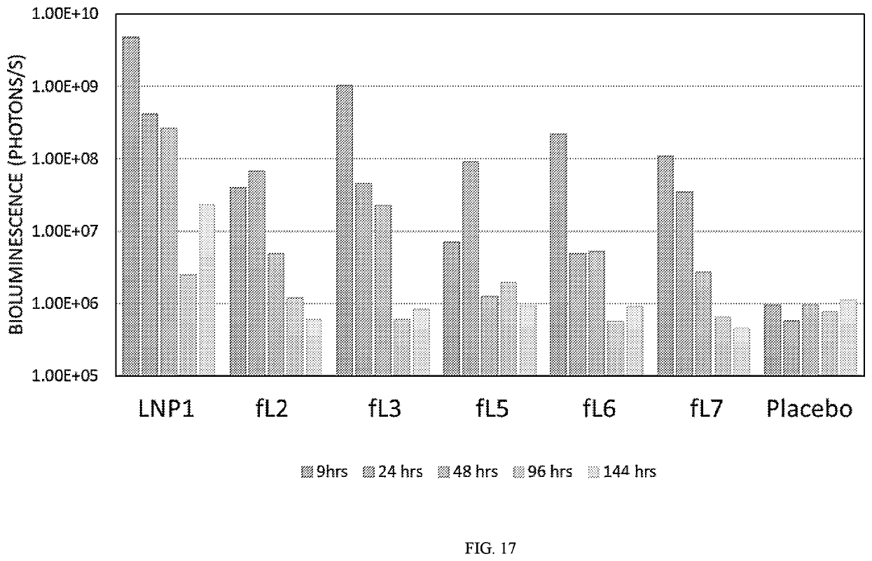

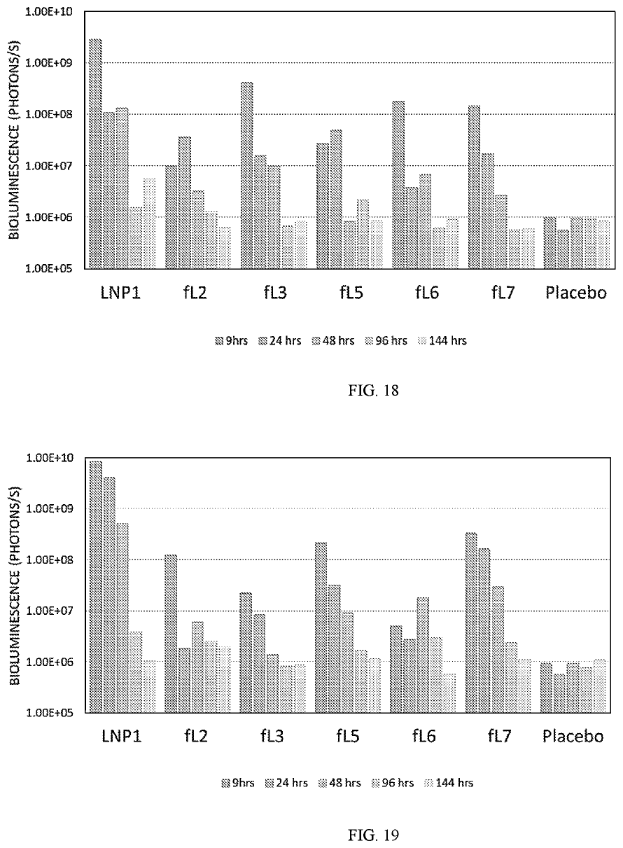

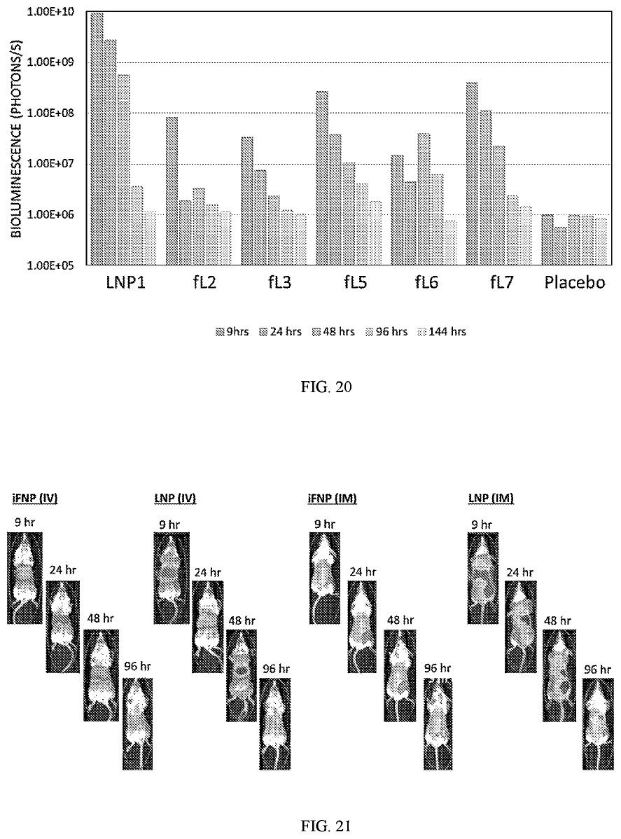

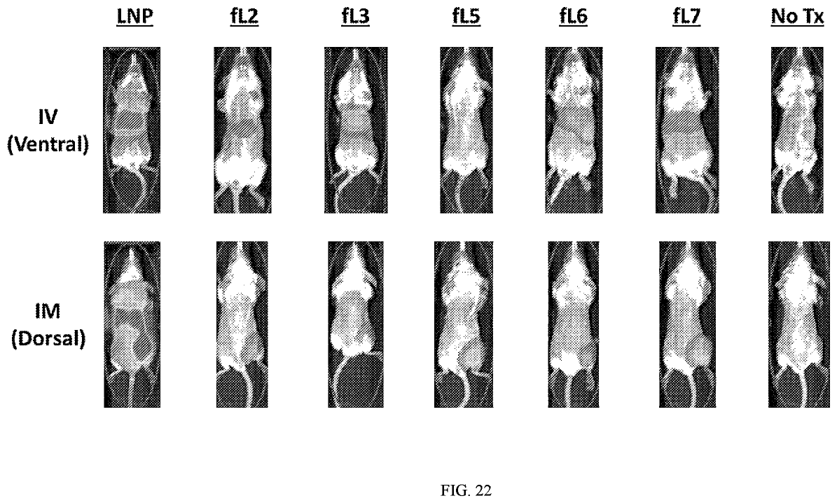

Whole animal luciferase bioluminescence (total emission) for the timepoints is summarized in FIGS. 17-20. Representative imaging results may be found in FIGS. 21 and 22 showing differential biodistribution of expression for the formulations.

These results demonstrate the suitability of iFNP for encapsulation of an mRNA sequence into a nanoparticle and processing into a pharmaceutical composition. These results also demonstrate that said pharmaceutical composition can be administered to a mammalian subject by various routes to achieve expression of the desired encoded enzyme (luciferase in this instance), which may be used to treat a loss-of-function disease such as an inborn error of metabolism. The results show that iFNP formulations may result in hepatic expression or spleen expression or both following IV administration. The results also show that iFNP formulations may result in local muscle/lymph expression following IM injection.

### Example 16: Impact of RNA Salt Form on Carrier RNA Formulation

Carrier RNA (>200 base pairs in size with unmodified bases, Qiagen Carrier RNA, Cat #1017647) was prepared in salt forms other than TEA. The guanidine and choline salt forms were prepared by adding 1.1 molar equivalents of guanidine*HCl or choline*HCl to the carrier RNA (with respect to the phosphate groups, RNA in the sodium salt form) in water. The solution was incubated at 37° C. for 10 min, and then lyophilized to dryness. The diphenhydramine (Benadryl) salt form was prepared by adding 10 equivalents of diphenhydramine*HCl to the carrier RNA (with respect to the phosphate groups, RNA in the sodium salt form) in water, resulting in the precipitation of the RNA in the new salt form. The mixture was incubated at 37° C. for 10 min, and the RNA precipitate was removed from dispersion by centrifuging at 20 k rcf for 10 min. The supernatant was removed from the RNA pellet, fresh deionized water was added to the RNA and then lyophilized to dryness.

The carrier RNA in the guanidine, choline, or Benadryl salt forms were mixed with Dex-PLGA (503-80) in DMSO containing 10 vol % deionized water. The RNA was present at 1.6 mg/mL (on a free-acid basis), and the polymer stabilizer was present at 4 mg/mL. This solution (0.5 mL) was rapidly mixed with an antisolvent stream of DCM in a multi-inlet vortex mixer (MIVM). The DCM entered the mixer in three separate streams of 0.5 mL volume each. CaCl₂) at a 0.5 charge equivalent ratio with respect to the total acid content of the formulation was included in one DCM stream. Additional DCM, referred to as the “iFNP quench,” was included in the collection vial, so that DMSO content after collection was <10 vol %.

Prior to the solvent exchange process, a cationic lipid, SM-102, was added to the dispersion. After 5 minutes age time, POPC and cholesterol were added. After an additional 5 minutes age time, PEG-DMG was added. These components were all added in ethanol. The SM-102 was added at 2 charge equivalents with respect to the RNA acid groups. The lipid components were maintained in a 47:10:39:4 (SM-102:POPC:Chol:PEG-DMG) mol ratio with respect to each other. The DCM solution was concentrated by rotary evaporation about 4-fold to 1-1.5 mL. Then, acetonitrile (8 mL) was slowly added to the vial. The dispersion was concentrated to 1-1.5 mL at 70 torr with the heat bath set to 35° C. Then, acetonitrile (8 mL) was added to the vial. The dispersion was concentrated to 1-1.5 mL at 65 torr with the heat bath set to 35° C. Then, acetonitrile (8 mL) was added to the vial. The dispersion was concentrated to 1 mL at 60 torr with the heat bath set to 35° C.

The inverse nanocarrier dispersion in acetonitrile was then mixed against an equal volume of deionized water in a CIJ mixer. A collection bath containing 30 mM HEPES and 135 mM NaCl at a pH of 7.4 was used to reduce the MeCN volume content to less than 10 vol %. This process produced a PEG-coated nanocarrier. DLS size and zeta potential were measured using standard techniques (Table 23).

TABLE 23
Characterization of mRNA formulations. Size, PDI, and zeta
potential were measured by dynamic light scattering after coating.

| Salt Form | Size, z-avg (nm) | PDI | Zeta (mV) |
|---|---|---|---|
| Benadryl | 141 | 0.16 | −2 |
| Guanidine | 103 | 0.14 | −2 |
| Choline | 106 | 0.14 | −2 |

These results demonstrate the formation of alternative RNA salt forms and their use in the iFNP process to produce nanoparticles with suitable sizes, PDI values that indicate low polydispersity, and near-neutral zeta potential values.

### Example 17: Impact of RNA Salt Form on GFP mRNA Formulations

mRNA encoding GFP (DasherGFP, Aldevron) was prepared in salt forms other than TEA. The guanidine salt form was prepared by adding 1.1 molar equivalents of guanidine*HCl to the mRNA (with respect to the phosphate groups, mRNA in the sodium salt form) in water. The solution was incubated at 37° C. for 10 min, and then lyophilized to dryness. The diphenhydramine (Benadryl) salt form was prepared by adding 10 equivalents of diphenhydramine*HCl to the mRNA (with respect to the phosphate groups, mRNA in the sodium salt form) in water, resulting in the precipitation of the mRNA in the new salt form. The mixture was incubated at 37° C. for 10 min, and the mRNA precipitate was removed from dispersion by centrifuging at 20 k rcf for 10 min. The supernatant was removed from the mRNA pellet, fresh deionized water was added to the mRNA; and the material then lyophilized to dryness.

The mRNA in the Benadryl or guanidine salt forms were mixed with Dex-PLGA (503-80) in DMSO containing 10 vol % deionized water. The RNA was present at 1.6 mg/mL (on a free-acid basis), and the polymer stabilizer was present at 4 mg/mL. This solution (0.5 mL) was rapidly mixed with an antisolvent stream of DCM in a multi-inlet vortex mixer (MIVM). The DCM entered the mixer in three separate streams of 0.5 mL volume each. CaCl₂) at a 0.5 charge equivalent ratio with respect to the total acid content of the formulation was included in one DCM stream. Additional DCM, referred to as the “iFNP quench,” was included in the collection vial so that DMSO content after collection was <10 vol %.

Prior to the solvent exchange process, a cationic lipid, SM-102, was added to the dispersion. After 5 minutes age time, POPC and cholesterol were added. For some formulations, after an additional 5 minutes age time, PEG-DMG was added. These components were all added in ethanol. The SM-102 was added at either 2 charge equivalents with respect to the RNA acid groups. The lipid components were maintained in a 47:10:39:4 (SM-102:POPC:Chol:PEG-DMG) mol ratio with respect to each other. For the formulations that did not contain PEG-DMG, poly(caprolactone)_{5 kDa}-b-PEG_{5 kDa} (PCL-PEG) was added as the coating material such that the final coated particle would be 28% PCL-PEG by mass. The DCM solution was concentrated by rotary evaporation about 4-fold to 1-1.5 mL. Then, acetonitrile (8 mL) was slowly added to the vial. The dispersion was concentrated to 1-1.5 mL at 70 torr with the heat bath set to 35° C. Then, acetonitrile (8 mL) was added to the vial. The dispersion was concentrated to 1-1.5 mL at 65 torr with the heat bath set to 35° C. Then, acetonitrile (8 mL) was added to the vial. The dispersion was concentrated to 1 mL at 60 torr with the heat bath set to 35° C.

The inverse nanocarrier dispersion in acetonitrile was then mixed against an equal volume of deionized water in a CIJ mixer. A collection bath containing 30 mM Tris/Acetate and 120 mM NaCl at a pH of 7.4 was used to reduce the acetonitrile volume content to less than 10 vol %. This process produced a PEG-coated nanocarrier. After 2-5 minutes, 1×PBS was added at equal volume to each dispersion. The formulations were processed into a final buffer as follows. For the PEG-DMG coated formulations, the nanocarrier was concentrated by ultrafiltration on a 50 or 100 kDa molecular-weight-cut-off filter at 10° C. PBS was added twice to the filter and the concentration step was repeated each time. For the PCL-PEG coated formulation, the nanocarrier was dialyzed in an excess of PBS at 4° C. for 12 hrs. The PCL-PEG formulations were then concentrated by ultrafiltration on a 100 kDa molecular-weight-cut-off filter at 10° C. a single time. The concentrations of all formulations were adjusted to 0.15 mg/mL RNA and tested in cell culture. DLS size and zeta potential were measured using standard techniques (Table 24).

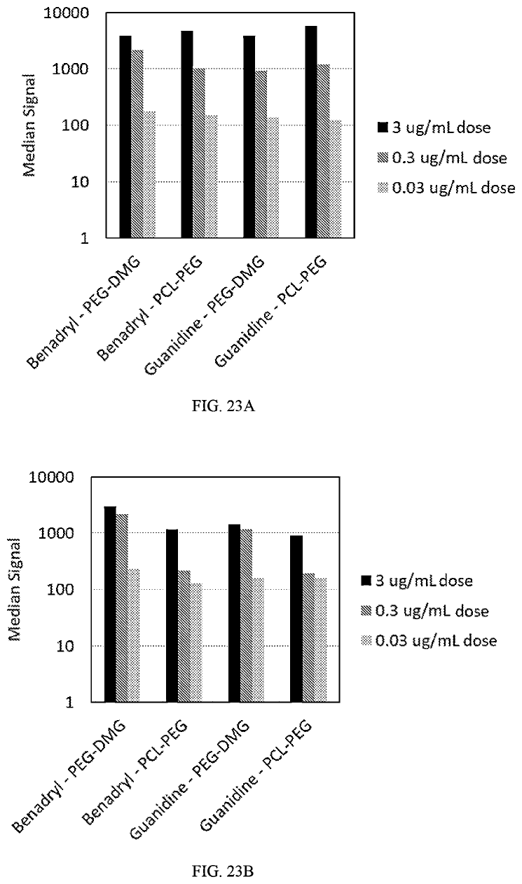

Nanoparticles were added to adherent cell cultures with a starting confluence of 30-50% and were incubated for 24 hours at standard cell culture conditions. Cells were washed and resuspended in PBS+500 ng/mL 4′,6-diamidino-2-phenylindole (DAPI) followed by analysis using the Attune NxT flow cytometer (ThermoFisher). Singlet cells were gated using SSC-A×SSC-H, and live cells were gated from the DAPI-negative population. Fluorescent intensities for GFP were captured on the BL-1 channel without compensation. Cell cultures were HEK-293T and HepG2 (FIG. 23). FIG. 23A shows GFP signal measured by flow cytometry after 24 hour incubation in 293T culture; the median value is reported for two mRNA doses in each well. FIG. 23B shows GFP signal measured by flow cytometry after 24 hour incubation in HepG2 culture; the median value is reported for two mRNA doses in each well.

TABLE 24
Characterization of mRNA formulations. Size, PDI, and zeta potential
were measured by dynamic light scattering after ultrafiltration.
Formulation naming is [mRNA salt form] - [coating material].

| Formulation | Size, z-avg (nm) | PDI | Zeta (mV) |
|---|---|---|---|
| Benadryl - PEG-DMG | 117 | 0.18 | −10 |
| Benadryl - PCL-PEG | 196 | 0.18 | −12 |
| Guanidine - PEG-DMG | 107 | 0.18 | −10 |
| Guanidine - PCL-PEG | 226 | 0.17 | −14 |

These results demonstrate that alternative salt forms of RNA retain the ability to transfect mammalian cells after encapsulation in the iFNP process. These results demonstrate transfection of mammalian cells in a dose dependent manner by iFNP nanocarriers with a stably attached PCL-PEG surface coating for both tested RNA salt forms (diphenhydramine and guanidine salt forms).

### Example 18: Generation of the TEA and Guanidine Salts of a Plasmid

To generate the TEA salt, plasmid in water (pEPito-EF1-EGFPLuc from PlasmidFactory) was concentrated on a centrifugal filter. TEA carbonate buffer (0.5 M) was added to the plasmid and concentrated. This step was repeated two more times. The plasmid was then washed on the centrifugal filter with deionized water five times to remove excess TEA and then concentrated. DMSO was added to the plasmid such that the final solvent was 65 vol % DMSO, 35 vol % water. Plasmid concentration was quantified by UV-Vis spectrometry, and this stock solution was used for subsequent iFNP formulations.

To generate the guanidine salt, plasmid in water (pEPito-EF1-EGFPLuc from PlasmidFactory) was concentrated on a centrifugal filter as much as possible. Guanidine*HCl in water (20 mg/mL) was added to the plasmid and incubated at 37° C. for 10 min. DMSO was added to the plasmid such that the final solvent was 65 vol % DMSO, 35 vol % water. Plasmid concentration was quantified by UV-Vis spectrometry, and this stock solution was used for subsequent iFNP formulations.

### Example 19: iFNP Formulations of Plasmid DNA Stabilized with Dex-PLGA

Plasmid (pEPito-EF1-EGFPLuc from PlasmidFactory) in the TEA salt form was mixed with a Dex-PLGA (503-80) in DMSO containing 10 vol % deionized water. The DNA-TEA was present at 2 mg/mL and the polymer stabilizer was present at 4 mg/mL. This solution (0.5 mL) was rapidly mixed with an antisolvent stream of DCM in a multi-inlet vortex mixer (MIVM). The DCM entered the mixer in three separate streams of 0.5 mL volume each. CaCl₂) at a 0.5 charge equivalent ratio with respect to the total acid content of the formulation was included in one DCM stream. Additional DCM, referred to as the “iFNP quench,” was included in the collection vial, so that DMSO content after collection was <10 vol %.

Prior to the solvent exchange process, a cationic lipid, SM-102 (CAS #2089251-47-6) or D-Lin-MC3-DMA (MC3, [(6Z,9Z,28Z,31Z)-heptatriaconta-6,9,28,31-tetraen-19-yl]-4-(dimethylamino)butanoate) was added to the dispersion at either 2 or 3 charge equivalents with respect to the phosphate groups in the plasmid. After 5 minutes age time, POPC and cholesterol were added. After an additional 5 minutes age time, PEG-DMG was added. These components were all added in ethanol. They were in a 47:10:39:4 (SM-102 or D-Lin-MC3-DMA:POPC:Chol:PEG-DMG) mol ratio with respect to each other. The DCM solution was concentrated by rotary evaporation about 4-fold to 1-1.5 mL. Then, acetonitrile (8 mL) was slowly added to the vial. The dispersion was concentrated to 1-1.5 mL at 70 torr with the heat bath set to 35° C. Then, acetonitrile (8 mL) was added to the vial. The dispersion was concentrated to 1-1.5 mL at 65 torr with the heat bath set to 35° C. Then, acetonitrile (8 mL) was added to the vial. The dispersion was concentrated to 1 mL at 60 torr with the heat bath set to 35° C. The inverse nanocarrier dispersion in acetonitrile was then mixed against an equal volume of deionized water in a CIJ mixer and a collection bath of additional Tris buffered saline was used to reduce the MeCN volume content to less than 10 vol %. This process produced a PEG-coated nanocarrier. The formulations were then dialyzed in Tris buffer overnight at 4° C., filtered with a 0.45 μm filter, and concentrated on a centrifugal filter. The nanocarrier size, polydispersity, and zeta potentials were measured by dynamic light scattering and are given below in Table 25.

TABLE 25
The size characteristics and zeta potentials of iFNP-formulated
plasmid DNA stabilized with Dex-PLGA.

| Ionizable Lipid | Lipid Amount | D, nm | PDI | Zeta Potential, mV |
|---|---|---|---|---|
| SM-102 | 2 eq | 135 | 0.16 | 1.9 |
| SM-102 | 3 eg | 138 | 0.16 | 1.7 |
| MC3 | 2 eq | 223 | 0.17 | 0.7 |
| MC3 | 3 eq | 210 | 0.19 | 1.4 |

### Example 20: iFNP Formulations of Plasmid DNA Stabilized with PAsp-PLGA

Plasmid (pEPito-EF1-EGFPLuc from PlasmidFactory) in the TEA or guanidine salt form was mixed with a PAsp-PLGA in DMSO containing 10 vol % deionized water. The plasmid was present at 1.2 mg/mL on a free acid basis, and the polymer stabilizer was present at 3 mg/mL. This solution (0.5 mL) was rapidly mixed with an antisolvent stream of DCM in a multi-inlet vortex mixer (MIVM). The DCM entered the mixer in three separate streams of 0.5 mL volume each. CaCl₂) at a 0.5 charge equivalent ratio with respect to the total acid content of the formulation (phosphates in the plasmid and carboxylic acids in the PAsp) was included in one DCM stream. Additional DCM, referred to as the “iFNP quench,” was included in the collection vial so that DMSO content after collection was <10 vol %.

Prior to the solvent exchange process, a cationic lipid, SM-102 (CAS #2089251-47-6), was added to the dispersion at 3 charge equivalents with respect to the phosphate groups in the plasmid. After 5 minutes age time, POPC and cholesterol were added. After an additional 5 minutes age time, PEG-DMG was added. These components were all added in ethanol. They were in a 47:10:39:4 (SM-102:POPC:Chol:PEG-DMG) mol ratio with respect to each other. The DCM solution was concentrated by rotary evaporation about 4-fold to 1-1.5 mL. Then, acetonitrile (8 mL) was slowly added to the vial. The dispersion was concentrated to 1-1.5 mL at 70 torr with the heat bath set to 35° C. Then, acetonitrile (8 mL) was added to the vial. The dispersion was concentrated to 1-1.5 mL at 65 torr with the heat bath set to 35° C. Then, acetonitrile (8 mL) was added to the vial. The dispersion was concentrated to 1 mL at 60 torr with the heat bath set to 35° C. The inverse nanocarrier dispersion in acetonitrile was then mixed against an equal volume of deionized water in a CIJ mixer and a collection bath of additional Tris buffered saline was used to reduce the MeCN volume content to less than 10 vol %. This process produced a PEG-coated nanocarrier. The formulations were then dialyzed in Tris buffer overnight at 4° C., filtered with a 0.45 μm filter, and concentrated on a centrifugal filter. The nanocarrier size, polydispersity, and zeta potentials were measured by dynamic light scattering and are given below in Table 26.

TABLE 26
The size characteristics and zeta potentials of iFNP-
formulated plasmid DNA stabilized with PAsp-PLGA.

| Plasmid | D, | | Zeta |
|---|---|---|---|
| Salt Form | nm | PDI | Potential, mV |
| Guanidine | 111 | 0.19 | −0.3 |
| TEA | 109 | 0.19 | 0.1 |

### Example 21: iFNP Formulation Variations of Plasmid DNA in the Guanidine Salt Form Stabilized with PAsp-PLGA

Plasmid (pEPito-EF1-EGFPLuc from PlasmidFactory) in the TEA or guanidine salt form was mixed with a PAsp-PLGA in DMSO containing 10 or 17.5 vol % deionized water. The plasmid was present at 1.2 mg/mL on a free acid basis, and the polymer stabilizer was present at 3 mg/mL. This solution (0.5 mL) was rapidly mixed with an antisolvent stream of DCM or chloroform (CHCl₃) in a multi-inlet vortex mixer (MIVM). The antisolvent stream entered the mixer in three separate streams of 0.5 mL volume each. CaCl₂), ZnCl₂, or MgCl₂ at a 0.5 or 1 charge equivalent ratio with respect to the total acid content of the formulation (phosphates in the plasmid and carboxylic acids in the PAsp) was included in one antisolvent stream. Additional antisolvent, referred to as the “iFNP quench,” was included in the collection vial so that DMSO content after collection was <10 vol %. The iFNP variations are given below in Table 27.

TABLE 27
The iFNP solvent and metal cation conditions for the
encapsulation of plasmid DNA stabilized PAsp-PLGA.

| Formu- | % MQ | Metal | Eq. Metal | |
|---|---|---|---|---|
| lation | in DMSO | Salt | Cation | Antisolvent |
| AG-1 | 10 | CaCl₂ | 0.5 | DCM |
| AG-2 | 10 | CaCl₂ | 0.5 | CHCl₃ |
| AG-3 | 10 | CaCl₂ | 1 | DCM |
| AG-4 | 10 | ZnCl₂ | 0.5 | DCM |
| AG-5 | 10 | MgCl₂ | 0.5 | DCM |
| AG-6 | 17.5 | CaCl₂ | 0.5 | DCM |

Prior to the solvent exchange process, a cationic lipid, SM-102 (CAS #2089251-47-6) was added to the dispersion at 3 charge equivalents with respect to the phosphate groups in the plasmid. After 5 minutes age time, POPC and cholesterol were added. After an additional 5 minutes age time, PEG-DMG was added. These components were all added in ethanol. They were in a 47:10:39:4 (SM-102:POPC:Chol:PEG-DMG) mol ratio with respect to each other. The DCM solution was concentrated by rotary evaporation about 4-fold to 1-1.5 mL. Then, acetonitrile (8 mL) was slowly added to the vial. The dispersion was concentrated to 1-1.5 mL at 70 torr with the heat bath set to 35° C. Then, acetonitrile (8 mL) was added to the vial. The dispersion was concentrated to 1-1.5 mL at 65 torr with the heat bath set to 35° C. Then, acetonitrile (8 mL) was added to the vial. The dispersion was concentrated to 1 mL at 60 torr with the heat bath set to 35° C. The inverse nanocarrier dispersion in acetonitrile was then mixed against an equal volume of deionized water in a CIJ mixer and a collection bath of additional Tris buffered saline was used to reduce the MeCN volume content to less than 10 vol %. This process produced a PEG-coated nanocarrier. The formulations were then dialyzed in Tris buffer overnight at 4° C., filtered with a 0.45 μm filter, and concentrated on a centrifugal filter. The nanocarrier size and polydispersity (PDI) were measured by dynamic light scattering, and are given below in Table 28.

TABLE 28
The size characteristics and zeta potentials of
iFNP-formulated plasmid DNA stabilized with
PAsp-PLGA. The formulation details
are given previously in Table 27.

| Formulation | D, nm | PDI |
|---|---|---|
| AG-1 | 121 | 0.15 |
| AG-2 | 117 | 0.16 |
| AG-3 | 121 | 0.15 |
| AG-4 | 124 | 0.17 |
| AG-5 | 118 | 0.15 |
| AG-6 | 119 | 0.16 |

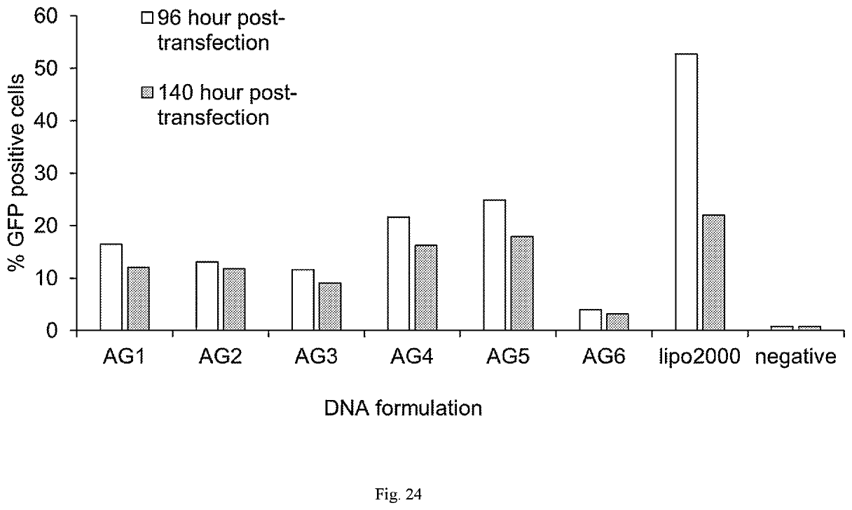

DNA formulations listed in Table 27 were added to adherent HEK-293T cell cultures with a starting confluence of 30-50% at 1.5 ug DNA per mL media. Cells were also transfected with a Lipofectamine 2000-pEPpito complex (lipo2000) as a positive control. After routine cell culture maintenance, GFP signal was measured by flow cytometry over multiple days. To perform flow cytometry, cells were washed and resuspended in PBS+500 ng/mL 4′,6-diamidino-2-phenylindole (DAPI) followed by analysis using the Attune NxT flow cytometer (ThermoFisher). Singlet cells were gated using SSC-A×SSC-H, and live cells were gated from the DAPI-negative population. Fluorescent intensities and percent positivities for GFP were captured on the BL-1 channel without compensation. Results are reported in FIG. 24.

### Example 22: Effect of Salt Form and Stabilizer Selection

mRNA encoding GFP was concentrated in deionized water and 1-1.1 equivalents (charge basis) of guanidine HCl or arginine was added. After 10 minutes incubation at 37° C., the mRNA was mixed with the stabilizing polymer in DMSO containing 10 vol % deionized water. The polymer was varied according to Table 29 and the ratio was 29% RNA on a free acid basis. This solution (0.5 mL) was rapidly mixed with an antisolvent stream of DCM in a multi-inlet vortex mixer (MIVM). The DCM entered the mixer in three separate streams of 0.5 mL volume each. CaCl₂) at 2 or 0.5 charge equivalent ratio with respect to the total acid content of the formulation was included in one DCM stream. Additional DCM was included in the collection vial so that DMSO content after collection was <10 vol %. Accounting for hold-up, the mRNA mass was 0.75 mg (free acid basis), and the polymer mass was 1.86 mg.

Prior to the solvent exchange, a cationic lipid, SM-102 (CAS #2089251-47-6), was added to the dispersion (3.1 mg or 4.7 mg). After 5 minutes age time, POPC (0.7 mg or 1 mg) and cholesterol (1.3 mg or 1.97 mg) were added. After an additional 5 minutes age time, PEG-DMG (1.5 mg or 2.25 mg) was added. These components were all added in ethanol. They were in a 47:10:36:6 (SM-102:POPC:Chol:PEG-DMG) mol ratio with respect to each other. The SM-102 was added at approximately 2 or 3 charge equivalents with respect to the RNA acid groups. The DCM solution was concentrated by rotary evaporation about 4-fold to 1-1.5 mL. Then, acetonitrile (8 mL) was slowly added to the vial. The dispersion was concentrated to 1-1.5 mL at 70 torr with the heat bath set to 35° C. Then, acetonitrile (8 mL) was added to the vial. The dispersion was concentrated to 1-1.5 mL at 65 torr with the heat bath set to 35° C. Then, acetonitrile (8 mL) was added to the vial. The dispersion was concentrated to 1 mL at 60 torr with the heat bath set to 35° C.

The inverse nanocarrier dispersion in acetonitrile was then mixed against an equal volume of deionized water in a CIJ mixer, and a collection bath of additional deionized water or tris-buffered saline was used to reduce the MeCN volume content to less than 10 vol %. This process produced the PEG-coated nanocarrier.

The formulations were processed into a final buffer as follows. The nanocarrier was dialyzed into Tris-buffered saline (pH 7.3) at 4° C. overnight. Then, the concentration was adjusted to 200 g/mL via ultrafiltration with 100 kDa molecular-weight-cut-off membranes as determined by UV-VIS measurement of RNA concentration and by RiboGreen assay using the manufacturer's protocol. 5 wt % sucrose was added, and then samples were flash frozen with liquid nitrogen and stored at −80° C. until use.

Nanoparticles were added to adherent cell cultures of HEK-293T with a starting confluence of 30-50% and were incubated for 24 hours at standard cell culture conditions. Cells were washed and resuspended in PBS+500 ng/mL 4′,6-diamidino-2-phenylindole (DAPI) followed by analysis using the Attune NxT flow cytometer (ThermoFisher). Singlet cells were gated using SSC-A×SSC-H, and live cells were gated from the DAPI-negative population. Fluorescent intensities for GFP were captured on the BL-1 channel without compensation.

The characterization results of each formulation are summarized in Table 29. As a summarizing value, GFP signal is reported in Table 29 as a percentage of the positive control at the same RNA dose (1 ug/mL). The positive control is the standard lipid nanoparticle using the same ionizable lipid at a NP ratio of 6.

TABLE 29
Parameter variations of mRNA formulations and resulting transfection performance. Size
is measured by DLS using either z-avg values or intensity distributions. Zeta potential
measures the surface charge and was also determined using the Zetasizer DLS instrument.
GFP signal is reported as a percentage of the signal from the LNP positive control.

| | | Ca | | Size, z-avg | Size | | Zeta | GFP |
|---|---|---|---|---|---|---|---|---|
| Stabilizer | Salt | (eq) | Process Notes | (nm) | (nm) | PDI | (mV) | (%) |
| 503-80 | Arginine | 0.5 | — | 118 | 141 | 0.15 | 0.1 | 146% |
| 503-Pasp | Guanidine | 2 | Tris quench | 132 | 141 | 0.15 | 0.5 | 72% |
| 503-PAsp | Guanidine | 2 | Water quench | 112 | 127 | 0.14 | −1.5 | 66% |
| 503-85 | Guanidine | 2 | — | 117 | 153 | 0.21 | −6.4 | 14% |
| 503-85 | Guanidine | 2 | 3 eq of CIL | 109 | 139 | 0.23 | −6.6 | 26% |

### Pharmaceutical Compositions and Administration

In an embodiment of the invention, nanoparticles are useful in pharmaceutical compositions prepared with a therapeutically effective amount of a compound and a pharmaceutically acceptable carrier or diluent.

Nanoparticles of an embodiment of the invention can be formulated as pharmaceutical compositions and administered to a subject in need of treatment, for example a mammal, such as a human patient, in a variety of forms adapted to the chosen route of administration, for example, orally, nasally, intraperitoneally, or parenterally, by intravenous, intramuscular, topical or subcutaneous routes, or by injection into tissue.

Thus, nanoparticles of an embodiment of the invention may be systemically administered, e.g., orally, in combination with a pharmaceutically acceptable vehicle such as an inert diluent or an assimilable edible carrier, or by inhalation or insufflation. They may be enclosed in hard or soft shell gelatin capsules, may be compressed into tablets, or may be incorporated directly with the food of the patient's diet. For oral therapeutic administration, the nanoparticles may be combined with one or more excipients and used in the form of ingestible tablets, buccal tablets, troches, capsules, elixirs, suspensions, syrups, wafers, and the like. The nanoparticles may be combined with a fine inert powdered carrier and inhaled by the subject or insufflated. Such compositions and preparations should contain at least 0.1% nanoparticles. The percentage of the compositions and preparations may, of course, be varied and may conveniently be between about 2% to about 60% of the weight of a given unit dosage form. The amount of nanoparticles in such therapeutically useful compositions is such that an effective dosage level may be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the nanoparticles may be incorporated into sustained-release preparations and devices. For example, the nanoparticles may be incorporated into time release capsules, time release tablets, and time release pills.

The nanoparticles may also be administered intravenously or intraperitoneally by infusion or injection. Solutions of the nanoparticles can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders including the nanoparticles which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium including, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. Isotonic agents, for example, sugars, buffers or sodium chloride can be included. Prolonged absorption of the injectable compositions can be brought about by the use in the compositions of agents delaying absorption, for example, aluminum monostearate and gelatin.

Sterile injectable solutions are prepared by incorporating the nanoparticles in the required amount in the appropriate solvent with various of the other ingredients enumerated above, as required, followed by filter sterilization. In the case of sterile powders for the preparation of sterile injectable solutions, methods of preparation include vacuum drying and freeze drying techniques, which yield a powder of the active ingredient plus any additional desired ingredient present in the previously sterile-filtered solutions.

For topical administration, the nanoparticles may be applied in pure form. However, it may be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Other solid carriers include nontoxic polymeric nanoparticles or microparticles. Useful liquid carriers include water, alcohols or glycols or water/alcohol/glycol blends, in which the nanoparticles can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the nanoparticles to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508), all of which are hereby incorporated by reference.

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949, which is hereby incorporated by reference.

For example, the concentration of the nanoparticles in a liquid composition, such as a lotion, can be from about 0.1-25% by weight, or from about 0.5-10% by weight. The concentration in a semi-solid or solid composition such as a gel or a powder can be about 0.1-5% by weight, or about 0.5-2.5% by weight.

The amount of the nanoparticles required for use in treatment may vary not only with the particular salt selected but also with the route of administration, the nature of the condition being treated, and the age and condition of the patient and will be ultimately at the discretion of the attendant physician or clinician.

Effective dosages and routes of administration of agents of an embodiment of the invention can be conventional. The exact amount (effective dose) of the agent may vary from subject to subject, depending on, for example, the species, age, weight and general or clinical condition of the subject, the severity or mechanism of any disorder being treated, the particular agent or vehicle used, the method and scheduling of administration, and the like. A therapeutically effective dose can be determined empirically, by conventional procedures known to those of skill in the art. See, e.g., The Pharmacological Basis of Therapeutics, Goodman and Gilman, eds., Macmillan Publishing Co., New York. For example, an effective dose can be estimated initially either in cell culture assays or in suitable animal models. The animal model may also be used to determine the appropriate concentration ranges and routes of administration. Such information can then be used to determine useful doses and routes for administration in humans. A therapeutic dose can also be selected by analogy to dosages for comparable therapeutic agents.

The particular mode of administration and the dosage regimen may be selected by the attending clinician, taking into account the particulars of the case (e.g., the subject, the disease, the disease state involved, and whether the treatment is prophylactic). Treatment may involve daily or multi-daily doses of compound(s) over a period of a few days to months, or even years.

In general, however, a suitable dose can be in the range of from about 0.001 to about 100 mg/kg, e.g., from about 0.01 to about 100 mg/kg of body weight per day, such as above about 0.1 mg per kilogram, or in a range of from about 1 to about 10 mg per kilogram body weight of the recipient per day. For example, a suitable dose may be about 1 mg/kg, 10 mg/kg, or 50 mg/kg of body weight per day.

The nanoparticles are conveniently administered in unit dosage form; for example, containing 0.05 to 10000 mg, 0.5 to 10000 mg, 5 to 1000 mg, or about 100 mg of active ingredient per unit dosage form.

The nanoparticles can be administered to achieve peak plasma concentrations of, for example, from about 0.5 to about 75 μM, about 1 to 50 μM, about 2 to about 30 μM, or about 5 to about 25 μM. Exemplary desirable plasma concentrations include at least or no more than 0.25, 0.5, 1, 5, 10, 25, 50, 75, 100 or 200 μM. For example, plasma levels may be from about 1 to 100 micromolar or from about 10 to about 25 micromolar. This may be achieved, for example, by the intravenous injection of a 0.05 to 5% solution of the nanoparticles, optionally in saline, or orally administered as a bolus containing about 1-100 mg of the nanoparticles. Desirable blood levels may be maintained by continuous infusion to provide about 0.00005-5 mg per kg body weight per hour, for example at least or no more than 0.00005, 0.0005, 0.005, 0.05, 0.5, or 5 mg/kg/hr. Alternatively, such levels can be obtained by intermittent infusions containing about 0.0002-20 mg per kg body weight, for example, at least or no more than 0.0002, 0.002, 0.02, 0.2, 2, 20, or 50 mg of the nanoparticles per kg of body weight.

The nanoparticles may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator.

### Aspects of the Invention

Aspect 1. A nanoparticle comprising:
a core comprising a more polar region of a first stabilizing amphiphilic copolymer and at least one water soluble agent; and
a shell comprising a less polar region of the first stabilizing amphiphilic copolymer, at least one lipid, and a second stabilizing amphiphilic agent,
wherein the shell surrounds the core.
   Aspect 2. The nanoparticle of aspect 1,
wherein the shell comprises an interior surface and an exterior surface,
wherein the interior surface of the shell is in contact with the core,
wherein the second stabilizing amphiphilic agent comprises a more polar region and a less polar region, and
wherein the more polar region of the second stabilizing amphiphilic agent is at the exterior surface of the shell.
   Aspect 3. The nanoparticle of aspect 2,
wherein the shell comprises the less polar region of the second stabilizing amphiphilic agent.
   Aspect 4. The nanoparticle of any one of aspects 2 through 3,
wherein the at least one water soluble agent is not at the exterior surface of the shell.
   Aspect 5. The nanoparticle of any one of aspects 2 through 4, further comprising a corona,
wherein the corona surrounds the shell and
wherein the corona comprises the more polar region of the second stabilizing amphiphilic agent.
   Aspect 6. The nanoparticle of aspect 5,
wherein the at least one water soluble agent is not in contact with the more polar region of the second stabilizing amphiphilic agent and
wherein the corona does not comprise the at least one water soluble agent.
   Aspect 7. The nanoparticle of any one of aspects 1 through 6, wherein the at least one water soluble agent is selected from the group consisting of a nucleic acid, a polynucleic acid, ribonucleic acid (RNA), messenger ribonucleic acid (mRNA), self-amplifying messenger ribonucleic acid (saRNA), small interfering ribonucleic acid (siRNA), micro ribonucleic acid (microRNA), circular ribonucleic acid (circular RNA), transfer ribonucleic acid (tRNA), small guide ribonucleic acid (sgRNA), deoxyribonucleic acid (DNA), an antisense oligonucleotide (ASO), a plasmid, an episome, and combinations.
   Aspect 8. The nanoparticle of any one of aspects 1 through 6, wherein the at least one water soluble agent is selected from the group consisting of DNA, mRNA, and saRNA.
   Aspect 9. The nanoparticle of any one of aspects 1 through 6, wherein the at least one water soluble agent comprises mRNA encoding SARS-CoV-2 spike protein receptor binding domain (RBD).
   Aspect 10. The nanoparticle of any one of aspects 1 through 6, wherein the at least one water soluble agent comprises mRNA encoding SARS-CoV-2 spike protein receptor binding domain (RBD) with pseudo-uridine (C5-glycoside isomer of uridine, 5-ribosyluracil) modification.
   Aspect 11. The nanoparticle of any one of aspects 1 through 10, wherein the first stabilizing amphiphilic copolymer is selected from the group consisting of poly(aspartic acid)-block-poly(lactic acid) (PAsp-b-PLA), poly(aspartic acid)-block-poly(lactic-co-glycolic acid) (PAsp-b-PLGA), dextran-poly(lactic acid) (Dex-PLA), and dextran-poly(lactic-co-glycolic acid) (Dex-PLGA).
   Aspect 12. The nanoparticle of aspect 11,
wherein the dextran and poly(aspartic acid) each have a molecular weight within a range of from about 500 to about 500,000 Da, from about 500 to about 50,000 Da, or from about 750 Da to about 20,000 Da and
wherein the poly(lactic acid) and poly(lactic-co-glycolic acid) each have a molecular weight within a range of from about 500 to about 500,000 Da, from about 500 to about 50,000 Da, or from about 750 Da to about 20,000 Da.
   Aspect 13. The nanoparticle of any one of aspects 1 through 10, wherein the first stabilizing amphiphilic copolymer is selected from the group consisting of poly(glutamic acid)-block-poly(lactic acid) (PGlu-b-PLA), poly(glutamic acid)-block-poly(lactic-co-glycolic acid) (PGlu-b-PLGA), poly(glutamic acid)-block-poly(caprolactone) (PGlu-b-PCL), poly(aspartic acid)-block-poly(caprolactone) (PAsp-b-PCL), and dextran-poly(caprolactone) (Dex-PCL).
   Aspect 14. The nanoparticle of aspect 13,
wherein the dextran, poly(aspartic acid), and poly(glutamic acid) each have a molecular weight within a range of from about 500 to about 500,000 Da, from about 500 to about 50,000 Da, or from about 750 Da to about 20,000 Da and
wherein the poly(lactic acid), poly(lactic-co-glycolic acid), and poly(caprolactone) each have a molecular weight within a range of from about 500 to about 500,000 Da, from about 500 to about 50,000 Da, or from about 750 Da to about 20,000 Da.
   Aspect 15. The nanoparticle of any one of aspects 1 through 14, wherein the at least one lipid is selected from the group consisting of a phospholipid, a cationic lipid, an anionic lipid, a sterol, a monoglyceride, a triglyceride, a fatty acid methyl ester, a fatty acid ethyl ester, and combinations.
   Aspect 16. The nanoparticle of any one of aspects 1 through 14, wherein the at least one lipid is selected from the group consisting of 1,2-distearoyl-s-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1,2-di-O-octadecenyl-sn-glycero-3-phosphocholine (18:0 Diether PC), 1-oleoyl-2-cholesterylhemisuccinoyl-sn-glycero-3-phosphocholine (OchemsPC), 1-hexadecyl-sn-glycero-3-phosphocholine (Cl 6 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), dipalmitoylphosphatidylglycerol (DPPG), palmitoyloleoylphosphatidylethanolamine (POPE), distearoyl-phosphatidyl-ethanolamine (DSPE), dipalmitoyl phosphatidyl ethanolamine (DPPE), dimyristoylphosphoethanolamine (DMPE), 1-stearoyl-2-oleoyl-phosphatidy ethanolamine (SOPE), 1-stearoyl-2-oleoyl-phosphatidylcholine (SOPC), sphingomyelin, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine (LPE), and combinations.
   Aspect 17. The nanoparticle of any one of aspects 1 through 14, where the at least one lipid comprises 1-palmitoyl-2-oleoyl-sn-glycero-3-phosphocholine (POPC).
   Aspect 18. The nanoparticle of any one of aspects 1 through 14, where the at least one lipid comprises a cationic lipid and/or an ionizable cationic lipid.
   Aspect 19. The nanoparticle of any one of aspects 1 through 14, wherein the at least one lipid is a blend of a cationic lipid, a phospholipid, and a cholesterol or a sterol.
   Aspect 20. The nanoparticle of any one of aspects 1 through 19, wherein the second stabilizing agent is selected from the group consisting of 1-(monomethoxy-polyethyleneglycol)-2,3-dimyristoylglycerol (PEG-DMG), pegylated distearoyl-phosphatidyl-ethanolamine (PEG-DSPE), polyethyleneglycol-block-poly(lactic acid) (PEG-b-PLA), polyethyleneglycol-block-poly(lactic-co-glycolic acid) (PEG-b-PLGA), and polyethyleneglycol-block-poly(caprolactone) (PEG-b-PCL).
   Aspect 21. The nanoparticle of aspect 20, wherein the poly(lactic acid), poly(lactic-co-glycolic acid), and poly(caprolactone) each have a molecular weight within a range of from about 500 to about 500,000 Da, from about 500 to about 50,000 Da, or from about 750 Da to about 20,000 Da.
   Aspect 22. The nanoparticle of any one of aspects 1 through 21, further comprising at least one hydrophobic polymer.
   Aspect 23. The nanoparticle of aspect 22, wherein the at least one hydrophobic polymer is selected from the group consisting of poly(lactic acid) (PLA), poly(lactic-co-glycolic acid) (PLGA), polycaprolactone (PCL), and combinations.
   Aspect 24. The nanoparticle of aspect 23, wherein the poly(lactic acid), poly(lactic-co-glycolic acid), and poly(caprolactone) each have a molecular weight within a range of from about 500 to about 500,000 Da, from about 500 to about 50,000 Da, or from about 750 Da to about 20,000 Da.
   Aspect 25. The nanoparticle of any one of aspects 1 through 6 and 11 through 24,
wherein the at least one water soluble agent comprises mRNA encoding SARS-CoV-2 spike protein receptor binding domain (RBD) and/or mRNA encoding SARS-CoV-2 spike protein receptor binding domain (RBD) with pseudo-uridine (C5-glycoside isomer of uridine, 5-ribosyluracil) modification,
wherein the first stabilizing amphiphilic copolymer comprises dextran-poly(lactic acid) (Dex-PLA), dextran-poly(lactic-co-glycolic acid) (Dex-PLGA) and/or dextran-poly(caprolactone) (Dex-PCL),
wherein the at least one lipid comprises a cationic lipid, and
wherein the second stabilizing amphiphilic agent comprises a polyethylene glycol (PEG) copolymer and/or a polyethylene glycol (PEG) lipid.
   Aspect 26. The nanoparticle of aspect 25,
wherein the at least one water soluble agent comprises mRNA encoding SARS-CoV-2 spike protein receptor binding domain (RBD) with pseudo-uridine (C5-glycoside isomer of uridine, 5-ribosyluracil) modification,
wherein the first stabilizing amphiphilic copolymer comprises dextran-poly(lactic-co-glycolic acid) (Dex-PLGA),
wherein the at least one lipid comprises 8-[(2-hydroxyethyl)[6-oxo-6-(undecyloxy)hexyl]amino]-octanoic acid, 1-octylnonyl ester (SM-102), 1-palmitoyl-2-oleoyl-glycero-3-phosphocholine (POPC), and cholesterol, and
wherein the second stabilizing amphiphilic agent comprises 1,2-dimyristoyl-rac-glycero-3-methoxypolyethylene glycol-2000 (PEG-DMG).
   Aspect 27. A pharmaceutical composition comprising a therapeutically effective amount of the nanoparticle of any one of aspects 1 through 26 and a pharmaceutical acceptable carrier or diluent.
   Aspect 28. A method of administration to a subject, comprising administering to the subject a therapeutically effective amount of the nanoparticle of any one of aspects 1 through 26 or the pharmaceutical composition of aspect 27.
   Aspect 29. A method of administration to a cell, comprising contacting the cell with the nanoparticle of any one of aspects 1 through 26 or the pharmaceutical composition of aspect 27.
   Aspect 30. The method of aspect 29, wherein the cell is selected from the group consisting of a mammalian cell and a human cell.
   Aspect 31. The method of any one of aspects 29 and 30, wherein the administration to the cell is performed in vitro.
   Aspect 32. A method for preventing or treating an infectious disease, comprising administering a therapeutically effective amount of the nanoparticle of any one of aspects 1 through 26 to a subject suffering from the infectious disease,
wherein the at least one water soluble agent induces production of an antigen associated with the infectious disease by a cell of the subject and
wherein the antigen induces an immune response by the subject to the infectious disease.
   Aspect 33. The nanoparticle of any one of aspects 1 through 26 for use in the prevention or treatment of an infectious disease.
   Aspect 34. Use of the nanoparticle of any one of aspects 1 through 26 in the manufacture of a medicament for the prevention or treatment of an infectious disease.
   Aspect 35. The method of aspect 32, the nanoparticle of aspect 33, or the use of aspect 34,
wherein the infectious disease is a virus.
   Aspect 36. The method of aspect 32, the nanoparticle of aspect 33, or the use of aspect 34, wherein the infectious disease is selected from the group consisting of adenovirus, Herpes simplex type 1, Herpes simplex type 2; encephalitis virus, papillomavirus, Varicella-zoster virus, Epstein-barr virus, Human cytomegalovirus, Human herpes virus type 8, Human papillomavirus, BK virus, JC virus, Smallpox, polio virus, Hepatitis B virus, Human bocavirus, Parvovirus B19, Human astrovirus, Norwalk virus, coxsackievirus, hepatitis A virus, poliovirus, rhinovirus, Hepatitis C virus, Yellow Fever virus, Dengue virus, West Nile virus, Rubella virus, Hepatitis E virus, Human Immunodeficiency virus (HIV), Guanarito virus, Junin virus, Lassa virus, Machupo virus, Sabia virus, Crimean-Congo hemorrhagic fever virus, Ebola virus, Marburg virus, Measles virus, Mumps virus, Parainfluenza virus, Respiratory syncytial virus, Human metapneumovirus, Hendra virus, Nipah virus, Hepatitis D, Rotavirus, Orbivirus, Coltivirus, Banna virus, Human Enterovirus, Hanta virus, West Nile virus, Japanese encephalitis virus, Vesicular exanthernavirus, and Eastern equine encephalitis.
   Aspect 37. The method of aspect 32, the nanoparticle of aspect 33, or the use of aspect 34, wherein the infectious disease is selected from the group consisting of a virus, a coronavirus, Middle East Respiratory Syndrome Corona Virus, Severe acute respiratory syndrome virus, SARS-CoV-2, rabies virus, influenza, Zika virus, cytomegalovirus, and Chikungunya virus.
   Aspect 38. A method for preventing or treating a cancer comprising administering a therapeutically effective amount of the nanoparticle of any one of aspects 1 through 26 to a subject suffering from the cancer,
wherein the at least one water soluble agent induces production by a cell of the subject of a tumor antigen associated with cancerous cells of the cancer and
wherein the antigen induces an immune response to the cancer by the subject.
   Aspect 39. The nanoparticle of any one of aspects 1 through 26 for use in the prevention or treatment of a cancer.
   Aspect 40. Use of the nanoparticle of any one of aspects 1 through 26 in the manufacture of a medicament for the prevention or treatment of a cancer.
   Aspect 41. A method for preventing or treating a loss-of-function disease comprising administering a therapeutically effective amount of the nanoparticle of any one of aspects 1 through 26 to a subject suffering from the loss-of-function disease,
wherein the at least one water soluble agent induces production of a protein that restores the lost function.
   Aspect 42. The nanoparticle of any one of aspects 1 through 26 for use in the prevention or treatment of a loss-of-function disease.
   Aspect 43. Use of the nanoparticle of any one of aspects 1 through 26 in the manufacture of a medicament for the prevention or treatment of a loss-of-function disease.
   Aspect 44. The method of aspect 41, the nanoparticle of aspect 42, or the use of aspect 43, wherein the loss-of-function disease is selected from the group consisting of a urea cycle disorder, N-acetylglutamate synthase (NAGS) deficiency, carbamoyl phosphate synthetase (CPS) deficiency, ornithine transcarbamoylase (OTC) deficiency, Citrullinemia Type 1 (CTLN1), Citrullinemia Type 2 (CTLN2), Argininosuccinic aciduria, Argininemia, and Hyperornithinemia-Hyperammonemia-Homocitrullinuria (H1H) syndrome.
   Aspect 45. The method of aspect 41, the nanoparticle of aspect 42, or the use of aspect 43, wherein the loss-of-function disease is selected from the group consisting of a polygenic disorder, a monogenic disorder, a polygenic liver disorder, and a monogenic liver disorder.
   Aspect 46. A method for preventing or treating a disease associated with a premature stop codon, comprising administering a therapeutically effective amount of the nanoparticle of any one of aspects 1 through 26 to a subject suffering from the disease associated with the premature stop codon,
wherein the at least one water soluble agent comprises tRNA and
wherein the tRNA enables translation through a premature stop codon.
   Aspect 47. The nanoparticle of any one of aspects 1 through 26 for use in the prevention or treatment of a disease associated with a premature stop codon.
   Aspect 48. Use of the nanoparticle of any one of aspects 1 through 26 in the manufacture of a medicament for the prevention or treatment of a disease associated with a premature stop codon.
   Aspect 49. The method of aspect 46, the nanoparticle of aspect 47, or the use of aspect 48, wherein the disease associated with a premature stop codon is selected from the group consisting of beta-thalassemia and Charcot-Marie-Tooth disease.
   Aspect 50. A method for gene editing, comprising
contacting the nanoparticle of any one of aspects 1 through 26 with a cell, so that a DNA sequence is inserted into a genome of the cell,
wherein the at least one water soluble agent comprises an endonuclease and/or an mRNA encoding an endonuclease, a small guide RNA (sgRNA), and the DNA sequence.
   Aspect 51. The method of aspect 50, wherein the nanoparticle is contacted with the cell in vitro.
   Aspect 52. The method of any one of aspects 50 and 51, wherein the endonuclease is selected from the group consisting of a Cas protein, Cas9, and a TALEN.
   Aspect 53. The nanoparticle of any one of aspects 1 through 26 for use in gene editing.
   Aspect 54. Use of the nanoparticle of any one of aspects 1 through 26 in the manufacture of a medicament for gene editing.
   Aspect 55. A method for producing a nanoparticle comprising
dissolving at least one water soluble agent in a first polar process solvent to form a water soluble agent solution;
dissolving a first stabilizing amphiphilic copolymer in a second polar process solvent to form a copolymer solution;
continuously mixing the water soluble agent solution and the copolymer solution with an antisolvent to form a mixed solution from which nanoparticles assemble to form an inverse nanoparticle dispersion;
adding at least one lipid to the inverse nanoparticle dispersion;
adding a second stabilizing amphiphilic agent to the inverse nanoparticle dispersion;
combining the inverse nanoparticle dispersion with a reforming solvent to form a reforming dispersion; and
continuously mixing the reforming dispersion with an aqueous solvent to form the nanoparticle,
wherein the first stabilizing amphiphilic copolymer comprises at least one region that is more polar and at least one region that is less polar,
wherein the second polar process solvent can be the same as or different from the first polar process solvent,
wherein the antisolvent is less polar than the first polar process solvent,
wherein the antisolvent is less polar than the second polar process solvent,
wherein the nanoparticle comprises a core and a shell,
wherein the core comprises the more polar region of the first stabilizing amphiphilic copolymer and the at least one water soluble agent, and
wherein the shell comprises the less polar region of the first stabilizing amphiphilic copolymer.
   Aspect 56. The method aspect 55, wherein the aqueous solvent is an aqueous buffer.
   Aspect 57. The method of any one of aspects 55 through 56, wherein the second polar process solvent is the same as the first polar process solvent.
   Aspect 58. The method of any one of aspects 55 through 57, wherein the water soluble agent solution and the copolymer solution are a single mixed solution.
   Aspect 59. The method of any one of aspects 55 through 56, wherein the second polar process solvent is different from the first polar process solvent.
   Aspect 60. The method of aspects 55 through 59,
wherein the shell comprises an interior surface and an exterior surface,
wherein the interior surface of the shell is in contact with the core,
wherein the second stabilizing amphiphilic agent comprises a more polar region and a less polar region, and
wherein the more polar region of the second stabilizing amphiphilic agent is at the exterior surface of the shell.
   Aspect 61. The method of aspect 60,
wherein the shell comprises the less polar region of the second stabilizing amphiphilic agent.
   Aspect 62. The method of any one of aspects 60 through 61,
wherein the at least one water soluble agent is not at the exterior surface of the shell.
   Aspect 63. The method of any one of aspects 60 through 62,
wherein a corona surrounds the shell and
wherein the corona comprises the more polar region of the second stabilizing amphiphilic agent.
   Aspect 64. The method of aspect 63,
wherein the at least one water soluble agent is not in contact with the more polar region of the second stabilizing amphiphilic agent and
wherein the corona does not comprise the at least one water soluble agent.
   Aspect 65. The method of any one of aspects 55 and 64, wherein the reforming solvent is acetonitrile.
   Aspect 66. The method of any one of aspects 55 through 65, wherein the at least one water soluble agent is selected from the group consisting of a nucleic acid, a polynucleic acid, ribonucleic acid (RNA), messenger ribonucleic acid (mRNA), self-amplifying messenger ribonucleic acid (saRNA), small interfering ribonucleic acid (siRNA), micro ribonucleic acid (microRNA), circular ribonucleic acid (circular RNA), transfer ribonucleic acid (tRNA), small guide ribonucleic acid (sgRNA), deoxyribonucleic acid (DNA), an antisense oligonucleotide (ASO), a plasmid, an episome, and combinations.
   Aspect 67. The method of any one of aspects 55 through 65, wherein the at least one water soluble agent comprises mRNA encoding SARS-CoV-2 spike protein receptor binding domain (RBD).
   Aspect 68. The method of any one of aspects 55 through 65, wherein the at least one water soluble agent comprises mRNA encoding SARS-CoV-2 spike protein receptor binding domain (RBD) with pseudo-uridine (C5-glycoside isomer of uridine, 5-ribosyluracil) modification.
   Aspect 69. The method of any one of aspects 55 through 68, wherein the first stabilizing amphiphilic copolymer is selected from the group consisting of poly(aspartic acid)-block-poly(lactic acid) (PAsp-b-PLA), poly(aspartic acid)-block-poly(lactic-co-glycolic acid) (PAsp-b-PLGA), dextran-poly(lactic acid) (Dex-PLA), dextran-poly(lactic-co-glycolic acid) (Dex-PLGA), poly(glutamic acid)-block-poly(lactic acid) (PGlu-b-PLA), poly(glutamic acid)-block-poly(lactic-co-glycolic acid) (PGlu-b-PLGA), poly(glutamic acid)-block-poly(caprolactone) (PGlu-b-PCL), poly(aspartic acid)-block-poly(caprolactone) (PAsp-b-PCL), and dextran-poly(caprolactone) (Dex-PCL).
   Aspect 70. The method of aspect 69,
wherein the dextran, poly(aspartic acid), and poly(glutamic acid) each have a molecular weight within a range of from about 500 to about 500,000 Da, from about 500 to about 50,000 Da, or from about 750 Da to about 20,000 Da and
wherein the poly(lactic acid), poly(lactic-co-glycolic acid), and poly(caprolactone) each have a molecular weight within a range of from about 500 to about 500,000 Da, from about 500 to about 50,000 Da, or from about 750 Da to about 20,000 Da.
   Aspect 71. The method of any one of aspects 55 through 68, wherein the first stabilizing amphiphilic copolymer is selected from the group consisting of poly(glutamic acid)-block-poly(lactic acid) (PGlu-b-PLA) and poly(glutamic acid)-block-poly(lactic-co-glycolic acid) (PGlu-b-PLGA).
   Aspect 72. The method of aspect 71,
wherein the poly(glutamic acid) has a molecular weight within a range of from about 500 to about 500,000 Da, from about 500 to about 50,000 Da, or from about 750 Da to about 20,000 Da and
wherein the poly(lactic acid) and poly(lactic-co-glycolic acid) each have a molecular weight within a range of from about 500 to about 500,000 Da, from about 500 to about 50,000 Da, or from about 750 Da to about 20,000 Da.
   Aspect 73. The method of any one of aspects 55 through 72, wherein the at least one lipid is selected from the group consisting of a phospholipid, a cationic lipid, an anionic lipid, a sterol, a monoglyceride, a triglyceride, a fatty acid methyl ester, a fatty acid ethyl ester, and combinations.
   Aspect 74. The method of any one of aspects 55 through 72, where the at least one lipid comprises a cationic lipid and/or 1-palmitoyl-2-oleoyl-sn-glycero-3-phosphocholine (POPC).
   Aspect 75. The method of any one of aspects 55 through 72, wherein the at least one lipid is a blend of a cationic lipid, a phospholipid, and a cholesterol or a sterol.
   Aspect 76. The method of any one of aspects 55 through 75, wherein the second stabilizing amphiphilic agent is selected from the group consisting of pegylated 1-(monomethoxy-polyethyleneglycol)-2,3-dimyristoylglycerol (PEG-DMG), pegylated distearoyl-phosphatidyl-ethanolamine (PEG-DSPE), polyethyleneglycol-block-poly(lactic acid) (PEG-b-PLA), polyethyleneglycol-block-poly(lactic-co-glycolic acid) (PEG-b-PLGA), and polyethyleneglycol-block-poly(caprolactone) (PEG-b-PCL).
   Aspect 77. The method of aspect 76, wherein the poly(lactic acid), poly(lactic-co-glycolic acid), and poly(caprolactone) each have a molecular weight within a range of from about 500 to about 500,000 Da, from about 500 to about 50,000 Da, or from about 750 Da to about 20,000 Da.
   Aspect 78. The method of any one of aspects 55 through 65,
wherein the at least one water soluble agent comprises mRNA encoding SARS-CoV-2 spike protein receptor binding domain (RBD) and/or mRNA encoding SARS-CoV-2 spike protein receptor binding domain (RBD) with pseudo-uridine (C5-glycoside isomer of uridine, 5-ribosyluracil) modification,
wherein the first stabilizing amphiphilic copolymer comprises dextran-poly(lactic acid) (Dex-PLA), dextran-poly(lactic-co-glycolic acid) (Dex-PLGA) and/or dextran-poly(caprolactone) (Dex-PCL),
wherein the at least one lipid comprises a cationic lipid, and
wherein the second stabilizing amphiphilic agent comprises a polyethylene glycol (PEG) copolymer and/or a polyethylene glycol (PEG) lipid.
   Aspect 79. The method of aspect 78,
wherein the at least one water soluble agent comprises mRNA encoding SARS-CoV-2 spike protein receptor binding domain (RBD) with pseudo-uridine (C5-glycoside isomer of uridine, 5-ribosyluracil) modification,
wherein the first stabilizing amphiphilic copolymer comprises dextran-poly(lactic-co-glycolic acid) (Dex-PLGA),
wherein the at least one lipid comprises 8-[(2-hydroxyethyl)[6-oxo-6-(undecyloxy)hexyl]amino]-octanoic acid, 1-octylnonyl ester (SM-102), 1-palmitoyl-2-oleoyl-glycero-3-phosphocholine (POPC), and cholesterol, and
wherein the second stabilizing amphiphilic agent comprises 1,2-dimyristoyl-rac-glycero-3-methoxypolyethylene glycol-2000 (PEG-DMG).
   Aspect 80. The nanoparticle of any one of aspects 1 through 26 and 33, 35, 36, 37, 39, 42, 44, 45, 47, 49, and 53, wherein the at least one water soluble agent comprises a ribonucleic acid (RNA) of which at least one uridine is replaced with a modified uridine.
   Aspect 81. The nanoparticle of aspect 80,
wherein at least 50% of the uridines of the ribonucleic acid (RNA) are each replaced with a modified uridine and
wherein the modified uridines may be the same or different.
   Aspect 82. The nanoparticle of aspect 80,
wherein all of the uridines of the ribonucleic acid (RNA) are each replaced with a modified uridine and
wherein the modified uridines may be the same or different.
   Aspect 83. The nanoparticle of any one of aspects 80 through 82, wherein each modified uridine is independently selected from the group consisting of pseudouridine, 5-methoxyuridine, and N1-methylpseudouridine.
   Aspect 84. The nanoparticle of aspect 83, wherein each modified uridine is independently selected from the group consisting of pseudouridine and 5-methoxyuridine.
   Aspect 85. The nanoparticle of any one of aspects 80 through 84, wherein the ribonucleic acid (RNA) is selected from the group consisting of messenger ribonucleic acid (mRNA), self-amplifying messenger ribonucleic acid (saRNA), small interfering ribonucleic acid (siRNA), micro ribonucleic acid (microRNA), circular ribonucleic acid (circular RNA), transfer ribonucleic acid (tRNA), small guide ribonucleic acid (sgRNA), and an antisense oligonucleotide ribonucleic acid (ASO RNA).
   Aspect 86. The nanoparticle of any one of aspects 1 through 26 and 33, 35, 36, 37, 39, 42, 44, 45, 47, 49, and 53 and 80 through 85, wherein the at least one water soluble agent is a nucleic acid that is at least partially neutralized with a base.
   Aspect 87. The nanoparticle of aspect 86, wherein the nucleic acid is 50% neutralized with a base.
   Aspect 88. The nanoparticle of aspect 86, wherein the nucleic acid is fully neutralized with a base.
   Aspect 89. The nanoparticle of any one of aspects 86 through 88, wherein the nucleic acid is a ribonucleic acid (RNA).
   Aspect 90. The nanoparticle of any one of aspects 86 through 89, wherein the nucleic acid is selected from the group consisting of messenger ribonucleic acid (mRNA), self-amplifying messenger ribonucleic acid (saRNA), small interfering ribonucleic acid (siRNA), micro ribonucleic acid (microRNA), circular ribonucleic acid (circular RNA), transfer ribonucleic acid (tRNA), small guide ribonucleic acid (sgRNA), and an antisense oligonucleotide ribonucleic acid (ASO RNA).
   Aspect 91. The nanoparticle of any one of aspects 86 through 90, wherein the base is an amine.
   Aspect 92. The nanoparticle of any one of aspects 86 through 90, wherein the base is guanidine.
   Aspect 93. The nanoparticle of any one of aspects 86 through 90, wherein the base is a guanidine derivative or a guanidinium salt.
   Aspect 94. The nanoparticle of any one of aspects 86 through 90, wherein the base is arginine.
   Aspect 95. The nanoparticle of any one of aspects 86 through 90, wherein the base is a tertiary amine.
   Aspect 96. The nanoparticle of any one of aspects 86 through 90, wherein the base is selected from the group consisting of triethylamine and diphenhydramine.
   Aspect 97. The method of any one of aspects 55 through 77, wherein the at least one water soluble agent comprises a ribonucleic acid (RNA) of which at least one uridine is replaced with a modified uridine.
   Aspect 98. The method of aspect 97,
wherein at least 50% of the uridines of the ribonucleic acid (RNA) are each replaced with a modified uridine and
wherein the modified uridines may be the same or different.
   Aspect 99. The method of aspect 97,
wherein all of the uridines of the ribonucleic acid (RNA) are each replaced with a modified uridine and
wherein the modified uridines may be the same or different.
   Aspect 100. The method of any one of aspects 97 through 99, wherein each modified uridine is independently selected from the group consisting of pseudouridine, 5-methoxyuridine, and N1-methylpseudouridine.
   Aspect 101. The method of aspect 100, wherein each modified uridine is independently selected from the group consisting of pseudouridine and 5-methoxyuridine.
   Aspect 102. The method of any one of aspects 97 through 101, wherein the ribonucleic acid (RNA) is selected from the group consisting of messenger ribonucleic acid (mRNA), self-amplifying messenger ribonucleic acid (saRNA), small interfering ribonucleic acid (siRNA), micro ribonucleic acid (microRNA), circular ribonucleic acid (circular RNA), transfer ribonucleic acid (tRNA), small guide ribonucleic acid (sgRNA), and an antisense oligonucleotide ribonucleic acid (ASO RNA).
   Aspect 103. The method of any one of aspects 55 through 77 and 97 through 102, wherein the at least one water soluble agent is a nucleic acid that is at least partially neutralized with a base prior to dissolving the at least one water soluble agent in the first polar process solvent.
   Aspect 104. The method of aspect 103, wherein the nucleic acid is 50% neutralized with a base.
   Aspect 105. The method of aspect 103, wherein the nucleic acid is fully neutralized with a base.
   Aspect 106. The method of any one of aspects 103 through 105, wherein the nucleic acid is a ribonucleic acid (RNA).
   Aspect 107. The method of any one of aspects 103 through 106, wherein the nucleic acid is selected from the group consisting of messenger ribonucleic acid (mRNA), self-amplifying messenger ribonucleic acid (saRNA), small interfering ribonucleic acid (siRNA), micro ribonucleic acid (microRNA), circular ribonucleic acid (circular RNA), transfer ribonucleic acid (tRNA), small guide ribonucleic acid (sgRNA), and an antisense oligonucleotide ribonucleic acid (ASO RNA).
   Aspect 108. The method of any one of aspects 103 through 107, wherein the base is an amine.
   Aspect 109. The method of any one of aspects 103 through 107, wherein the base is guanidine.
   Aspect 110. The method of any one of aspects 103 through 107, wherein the base is a guanidine derivative or a guanidinium salt.
   Aspect 111. The method of any one of aspects 103 through 107, wherein the base is arginine.
   Aspect 112. The method of any one of aspects 103 through 107, wherein the base is a tertiary amine.
   Aspect 113. The method of any one of aspects 103 through 107, wherein the base is selected from the group consisting of triethylamine and diphenhydramine.
   Aspect 114. The method of any one of aspects 55 through 77, wherein the at least one water soluble agent is a salt of a plasmid.
   Aspect 115. The method of aspect 114, wherein the at least one water soluble agent is a triethylamine (TEA) salt of the plasmid.
   Aspect 116. The method of aspect 114, wherein the at least one water soluble agent is a guanidine salt of the plasmid.
   Aspect 117. The method of any one of aspects 114 through 116, wherein the plasmid is an episome.
   Aspect 118. The method of any one of aspects 114 through 117, wherein the first stabilizing amphiphilic copolymer is a polysaccharide copolymer.
   Aspect 119. The method of any one of aspects 114 through 117, wherein the first stabilizing amphiphilic copolymer is dextran-poly(lactic-co-glycolic acid) (Dex-PLGA).
   Aspect 120. The method of aspect 119,
wherein the dextran has a molecular weight within a range of from about 500 to about 500,000 Da, from about 500 to about 50,000 Da, or from about 750 Da to about 20,000 Da and
wherein the poly(lactic-co-glycolic acid) has a molecular weight within a range of from about 500 to about 500,000 Da, from about 500 to about 50,000 Da, or from about 750 Da to about 20,000 Da.
   Aspect 121. The method of any one of aspects 114 through 117, wherein the first stabilizing amphiphilic copolymer is a polypeptide copolymer.
   Aspect 122. The method of any one of aspects 114 through 117, wherein the first stabilizing amphiphilic copolymer is poly(aspartic acid)-poly(lactic-co-glycolic acid) (PAsp-PLGA).
   Aspect 123. The method of aspect 122,
wherein the poly(aspartic acid) has a molecular weight within a range of from about 500 to about 500,000 Da, from about 500 to about 50,000 Da, or from about 750 Da to about 20,000 Da and
wherein the poly(lactic-co-glycolic acid) has a molecular weight within a range of from about 500 to about 500,000 Da, from about 500 to about 50,000 Da, or from about 750 Da to about 20,000 Da.
   Aspect 124. The method of any one of aspects 114 through 123, wherein the at least one lipid is selected from the group consisting of a cationic lipid, 1-palmitoyl-2-oleoyl-sn-glycero-3-phosphocholine (POPC), cholesterol, and combinations.
   Aspect 125. The method of any one of aspects 114 through 124, wherein the second amphiphilic stabilizing copolymer is a pegylated copolymer.
   Aspect 126. The method of any one of aspects 114 through 124, wherein the second amphiphilic stabilizing copolymer is 1-(monomethoxy-polyethyleneglycol)-2,3-dimyristoylglycerol (PEG-DMG).
   Aspect 127. The method of any one of aspects 55 through 79 and 97 through 126, wherein the antisolvent comprises a metal salt.
   Aspect 128. The method of aspect 127, wherein the metal salt is selected from the group consisting of MgCl₂, CaCl₂), and ZnCl₂.

The embodiments illustrated and discussed in this specification are intended only to teach those skilled in the art the best way known to the inventors to make and use the invention. Nothing in this specification should be considered as limiting the scope of the invention. All examples presented are representative and non-limiting. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

1. A nanoparticle comprising:
a core comprising a more polar region of a first stabilizing amphiphilic copolymer and at least one water soluble agent; and
a shell comprising a less polar region of the first stabilizing amphiphilic copolymer, at least one lipid, and a second stabilizing amphiphilic agent,
wherein the shell surrounds the core.

2. The nanoparticle of claim 1,
wherein the shell comprises an interior surface and an exterior surface,
wherein the interior surface of the shell is in contact with the core,
wherein the second stabilizing amphiphilic agent comprises a more polar region and a less polar region, and
wherein the more polar region of the second stabilizing amphiphilic agent is at the exterior surface of the shell.

3. The nanoparticle of claim 2, wherein the shell comprises the less polar region of the second stabilizing amphiphilic agent.

4. The nanoparticle of any one of claim 3, wherein the at least one water soluble agent is not at the exterior surface of the shell.

5. The nanoparticle of claim 3, further comprising a corona,
wherein the corona surrounds the shell and
wherein the corona comprises the more polar region of the second stabilizing amphiphilic agent.

6. The nanoparticle of claim 5,
wherein the at least one water soluble agent is not in contact with the more polar region of the second stabilizing amphiphilic agent and
wherein the corona does not comprise the at least one water soluble agent.

7. The nanoparticle of claim 1, wherein the at least one water soluble agent is selected from the group consisting of a nucleic acid, a polynucleic acid, ribonucleic acid (RNA), messenger ribonucleic acid (mRNA), self-amplifying messenger ribonucleic acid (saRNA), small interfering ribonucleic acid (siRNA), micro ribonucleic acid (microRNA), circular ribonucleic acid (circular RNA), transfer ribonucleic acid (tRNA), small guide ribonucleic acid (sgRNA), deoxyribonucleic acid (DNA), an antisense oligonucleotide (ASO), a plasmid, an episome, and combinations.

8. The nanoparticle of claim 1, wherein the at least one water soluble agent is selected from the group consisting of DNA, mRNA, and saRNA.

9. The nanoparticle of claim 1, wherein the at least one water soluble agent comprises mRNA encoding SARS-CoV-2 spike protein receptor binding domain (RBD).

10. The nanoparticle of claim 1, wherein the at least one water soluble agent comprises mRNA encoding SARS-CoV-2 spike protein receptor binding domain (RBD) with pseudo-uridine (C5-glycoside isomer of uridine, 5-ribosyluracil) modification.

11. The nanoparticle of claim 1, wherein the first stabilizing amphiphilic copolymer is selected from the group consisting of poly(aspartic acid)-block-poly(lactic acid) (PAsp-b-PLA), poly(aspartic acid)-block-poly(lactic-co-glycolic acid) (PAsp-b-PLGA), dextran-poly(lactic acid) (Dex-PLA), and dextran-poly(lactic-co-glycolic acid) (Dex-PLGA).

12. The nanoparticle of claim 11,
wherein the dextran and poly(aspartic acid) each have a molecular weight within a range of from about 500 to about 500,000 Da, from about 500 to about 50,000 Da, or from about 750 Da to about 20,000 Da and
wherein the poly(lactic acid) and poly(lactic-co-glycolic acid) each have a molecular weight within a range of from about 500 to about 500,000 Da, from about 500 to about 50,000 Da, or from about 750 Da to about 20,000 Da.

13. The nanoparticle of claim 1, wherein the first stabilizing amphiphilic copolymer is selected from the group consisting of poly(glutamic acid)-block-poly(lactic acid) (Pglu-b-PLA), poly(glutamic acid)-block-poly(lactic-co-glycolic acid) (Pglu-b-PLGA), poly(glutamic acid)-block-poly(caprolactone) (Pglu-b-PCL), poly(aspartic acid)-block-poly(caprolactone) (PAsp-b-PCL), and dextran-poly(caprolactone) (Dex-PCL).

14. The nanoparticle of claim 13,
wherein the dextran, poly(aspartic acid), and poly(glutamic acid) each have a molecular weight within a range of from about 500 to about 500,000 Da, from about 500 to about 50,000 Da, or from about 750 Da to about 20,000 Da and
wherein the poly(lactic acid), poly(lactic-co-glycolic acid), and poly(caprolactone) each have a molecular weight within a range of from about 500 to about 500,000 Da, from about 500 to about 50,000 Da, or from about 750 Da to about 20,000 Da.

15. The nanoparticle of claim 1, wherein the at least one lipid is selected from the group consisting of a phospholipid, a cationic lipid, an anionic lipid, a sterol, a monoglyceride, a triglyceride, a fatty acid methyl ester, a fatty acid ethyl ester, and combinations.

16. The nanoparticle of claim 1, wherein the at least one lipid is selected from the group consisting of 1,2-distearoyl-s-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1,2-di-O-octadecenyl-sn-glycero-3-phosphocholine (18:0 Diether PC), 1-oleoyl-2-cholesterylhemisuccinoyl-sn-glycero-3-phosphocholine (OchemsPC), 1-hexadecyl-sn-glycero-3-phosphocholine (Cl 6 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), dipalmitoylphosphatidylglycerol (DPPG), palmitoyloleoylphosphatidylethanolamine (POPE), distearoyl-phosphatidyl-ethanolamine (DSPE), dipalmitoyl phosphatidyl ethanolamine (DPPE), dimyristoylphosphoethanolamine (DMPE), 1-stearoyl-2-oleoyl-phosphatidy ethanolamine (SOPE), 1-stearoyl-2-oleoyl-phosphatidylcholine (SOPC), sphingomyelin, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine (LPE), and combinations.

17. The nanoparticle of claim 1, where the at least one lipid comprises 1-palmitoyl-2-oleoyl-sn-glycero-3-phosphocholine (POPC).

18. The nanoparticle of claim 1, where the at least one lipid comprises a cationic lipid and/or an ionizable cationic lipid.

19. The nanoparticle of claim 1, wherein the at least one lipid is a blend of a cationic lipid, a phospholipid, and a cholesterol or a sterol.

20. The nanoparticle of claim 1, wherein the second stabilizing agent is selected from the group consisting of 1-(monomethoxy-polyethyleneglycol)-2,3-dimyristoylglycerol (PEG-DMG), pegylated distearoyl-phosphatidyl-ethanolamine (PEG-DSPE), polyethyleneglycol-block-poly(lactic acid) (PEG-b-PLA), polyethyleneglycol-block-poly(lactic-co-glycolic acid) (PEG-b-PLGA), and polyethyleneglycol-block-poly(caprolactone) (PEG-b-PCL).

21. The nanoparticle of claim 20, wherein the poly(lactic acid), poly(lactic-co-glycolic acid), and poly(caprolactone) each have a molecular weight within a range of from about 500 to about 500,000 Da, from about 500 to about 50,000 Da, or from about 750 Da to about 20,000 Da.

22. The nanoparticle of claim 1, further comprising at least one hydrophobic polymer.

23. The nanoparticle of claim 22, wherein the at least one hydrophobic polymer is selected from the group consisting of poly(lactic acid) (PLA), poly(lactic-co-glycolic acid) (PLGA), polycaprolactone (PCL), and combinations.

24. The nanoparticle of claim 23, wherein the poly(lactic acid), poly(lactic-co-glycolic acid), and poly(caprolactone) each have a molecular weight within a range of from about 500 to about 500,000 Da, from about 500 to about 50,000 Da, or from about 750 Da to about 20,000 Da.

25. The nanoparticle of claim 1,
wherein the at least one water soluble agent comprises mRNA encoding SARS-CoV-2 spike protein receptor binding domain (RBD) and/or mRNA encoding SARS-CoV-2 spike protein receptor binding domain (RBD) with pseudo-uridine (C5-glycoside isomer of uridine, 5-ribosyluracil) modification,
wherein the first stabilizing amphiphilic copolymer comprises dextran-poly(lactic acid) (Dex-PLA), dextran-poly(lactic-co-glycolic acid) (Dex-PLGA) and/or dextran-poly(caprolactone) (Dex-PCL),
wherein the at least one lipid comprises a cationic lipid, and
wherein the second stabilizing amphiphilic agent comprises a polyethylene glycol (PEG) copolymer and/or a polyethylene glycol (PEG) lipid.

26. The nanoparticle of claim 25,
wherein the at least one water soluble agent comprises mRNA encoding SARS-CoV-2 spike protein receptor binding domain (RBD) with pseudo-uridine (C5-glycoside isomer of uridine, 5-ribosyluracil) modification,
wherein the first stabilizing amphiphilic copolymer comprises dextran-poly(lactic-co-glycolic acid) (Dex-PLGA),
wherein the at least one lipid comprises 8-[(2-hydroxyethyl)[6-oxo-6-(undecyloxy)hexyl]amino]-octanoic acid, 1-octylnonyl ester (SM-102), 1-palmitoyl-2-oleoyl-glycero-3-phosphocholine (POPC), and cholesterol, and
wherein the second stabilizing amphiphilic agent comprises 1,2-dimyristoyl-rac-glycero-3-methoxypolyethylene glycol-2000 (PEG-DMG).

27. A pharmaceutical composition comprising a therapeutically effective amount of the nanoparticle of claim 1 and a pharmaceutical acceptable carrier or diluent.

28. A method of administration to a subject, comprising administering to the subject a therapeutically effective amount of the nanoparticle of claim 1.

29. A method of administration to a cell, comprising contacting the cell with the nanoparticle of claim 1.

30. The method of claim 29, wherein the cell is selected from the group consisting of a mammalian cell and a human cell.

31. The method of claim 29, wherein the administration to the cell is performed in vitro.

32. A method for preventing or treating an infectious disease, comprising administering a therapeutically effective amount of the nanoparticle of claim 1 to a subject suffering from the infectious disease,
wherein the at least one water soluble agent induces production of an antigen associated with the infectious disease by a cell of the subject and
wherein the antigen induces an immune response by the subject to the infectious disease.

33. The nanoparticle of claim 1 for use in the prevention or treatment of an infectious disease.

34. Use of the nanoparticle of claim 1 in the manufacture of a medicament for the prevention or treatment of an infectious disease.

35. The method of claim 32, wherein the infectious disease is a virus.

36. The method of claim 32, wherein the infectious disease is selected from the group consisting of adenovirus, Herpes simplex type 1, Herpes simplex type 2; encephalitis virus, papillomavirus, Varicella-zoster virus, Epstein-barr virus, Human cytomegalovirus, Human herpes virus type 8, Human papillomavirus, BK virus, JC virus, Smallpox, polio virus, Hepatitis B virus, Human bocavirus, Parvovirus B19, Human astrovirus, Norwalk virus, coxsackievirus, hepatitis A virus, poliovirus, rhinovirus, Hepatitis C virus, Yellow Fever virus, Dengue virus, West Nile virus, Rubella virus, Hepatitis E virus, Human Immunodeficiency virus (HIV), Guanarito virus, Junin virus, Lassa virus, Machupo virus, Sabia virus, Crimean-Congo hemorrhagic fever virus, Ebola virus, Marburg virus, Measles virus, Mumps virus, Parainfluenza virus, Respiratory syncytial virus, Human metapneumovirus, Hendra virus, Nipah virus, Hepatitis D, Rotavirus, Orbivirus, Coltivirus, Banna virus, Human Enterovirus, Hanta virus, West Nile virus, Japanese encephalitis virus, Vesicular exanthernavirus, and Eastern equine encephalitis.

37. The method of claim 32, wherein the infectious disease is selected from the group consisting of a virus, a coronavirus, Middle East Respiratory Syndrome Corona Virus, Severe acute respiratory syndrome virus, SARS-CoV-2, rabies virus, influenza, Zika virus, cytomegalovirus, and Chikungunya virus.

38. A method for preventing or treating a cancer comprising administering a therapeutically effective amount of the nanoparticle of claim 1 to a subject suffering from the cancer,
wherein the at least one water soluble agent induces production by a cell of the subject of a tumor antigen associated with cancerous cells of the cancer and
wherein the antigen induces an immune response to the cancer by the subject.

39. The nanoparticle of claim 1 for use in the prevention or treatment of a cancer.

40. Use of the nanoparticle of claim 1 in the manufacture of a medicament for the prevention or treatment of a cancer.

41. A method for preventing or treating a loss-of-function disease comprising administering a therapeutically effective amount of the nanoparticle of claim 1 to a subject suffering from the loss-of-function disease,
wherein the at least one water soluble agent induces production of a protein that restores the lost function.

42. The nanoparticle of claim 1 for use in the prevention or treatment of a loss-of-function disease.

43. Use of the nanoparticle of claim 1 in the manufacture of a medicament for the prevention or treatment of a loss-of-function disease.

44. The method of claim 41, wherein the loss-of-function disease is selected from the group consisting of a urea cycle disorder, N-acetylglutamate synthase (NAGS) deficiency, carbamoyl phosphate synthetase (CPS) deficiency, ornithine transcarbamoylase (OTC) deficiency, Citrullinemia Type 1 (CTLN1), Citrullinemia Type 2 (CTLN2), Argininosuccinic aciduria, Argininemia, and Hyperornithinemia-Hyperammonemia-Homocitrullinuria (HHH) syndrome.

45. The method of claim 41, wherein the loss-of-function disease is selected from the group consisting of a polygenic disorder, a monogenic disorder, a polygenic liver disorder, and a monogenic liver disorder.

46. A method for preventing or treating a disease associated with a premature stop codon, comprising administering a therapeutically effective amount of the nanoparticle of claim 1 to a subject suffering from the disease associated with the premature stop codon,
wherein the at least one water soluble agent comprises tRNA and
wherein the tRNA enables translation through a premature stop codon.

47. The nanoparticle of claim 1 for use in the prevention or treatment of a disease associated with a premature stop codon.

48. Use of the nanoparticle of claim 1 in the manufacture of a medicament for the prevention or treatment of a disease associated with a premature stop codon.

49. The method of claim 46, wherein the disease associated with a premature stop codon is selected from the group consisting of beta-thalassemia and Charcot-Marie-Tooth disease.

50. A method for gene editing, comprising
contacting the nanoparticle of claim 1 with a cell, so that a DNA sequence is inserted into a genome of the cell,
wherein the at least one water soluble agent comprises an endonuclease and/or an mRNA encoding an endonuclease, a small guide RNA (sgRNA), and the DNA sequence.

51. The method of claim 50, wherein the nanoparticle is contacted with the cell in vitro.

52. The method of claim 50, wherein the endonuclease is selected from the group consisting of a Cas protein, Cas9, and a TALEN.

53. The nanoparticle of claim 1 for use in gene editing.

54. Use of the nanoparticle of claim 1 in the manufacture of a medicament for gene editing.

55. A method for producing a nanoparticle comprising
dissolving at least one water soluble agent in a first polar process solvent to form a water soluble agent solution;
dissolving a first stabilizing amphiphilic copolymer in a second polar process solvent to form a copolymer solution;
continuously mixing the water soluble agent solution and the copolymer solution with an antisolvent to form a mixed solution from which nanoparticles assemble to form an inverse nanoparticle dispersion;
adding at least one lipid to the inverse nanoparticle dispersion;
adding a second stabilizing amphiphilic agent to the inverse nanoparticle dispersion;
combining the inverse nanoparticle dispersion with a reforming solvent to form a reforming dispersion; and
continuously mixing the reforming dispersion with an aqueous solvent to form the nanoparticle,
wherein the first stabilizing amphiphilic copolymer comprises at least one region that is more polar and at least one region that is less polar,
wherein the second polar process solvent can be the same as or different from the first polar process solvent,
wherein the antisolvent is less polar than the first polar process solvent,
wherein the antisolvent is less polar than the second polar process solvent,
wherein the nanoparticle comprises a core and a shell,
wherein the core comprises the more polar region of the first stabilizing amphiphilic copolymer and the at least one water soluble agent, and
wherein the shell comprises the less polar region of the first stabilizing amphiphilic copolymer.

56. The method claim 55, wherein the aqueous solvent is an aqueous buffer.

57. The method of claim 55, wherein the second polar process solvent is the same as the first polar process solvent.

58. The method of claim 55, wherein the water soluble agent solution and the copolymer solution are a single mixed solution.

59. The method of claim 55, wherein the second polar process solvent is different from the first polar process solvent.

60. The method of claim 55,
wherein the shell comprises an interior surface and an exterior surface,
wherein the interior surface of the shell is in contact with the core,
wherein the second stabilizing amphiphilic agent comprises a more polar region and a less polar region, and
wherein the more polar region of the second stabilizing amphiphilic agent is at the exterior surface of the shell.

61. The method of claim 60, wherein the shell comprises the less polar region of the second stabilizing amphiphilic agent.

62. The method of claim 61, wherein the at least one water soluble agent is not at the exterior surface of the shell.

63. The method of claim 61,
wherein a corona surrounds the shell and
wherein the corona comprises the more polar region of the second stabilizing amphiphilic agent.

64. The method of claim 63,
wherein the at least one water soluble agent is not in contact with the more polar region of the second stabilizing amphiphilic agent and
wherein the corona does not comprise the at least one water soluble agent.

65. The method of claim 55, wherein the reforming solvent is acetonitrile.

66. The method of claim 55, wherein the at least one water soluble agent is selected from the group consisting of a nucleic acid, a polynucleic acid, ribonucleic acid (RNA), messenger ribonucleic acid (mRNA), self-amplifying messenger ribonucleic acid (saRNA), small interfering ribonucleic acid (siRNA), micro ribonucleic acid (microRNA), circular ribonucleic acid (circular RNA), transfer ribonucleic acid (tRNA), small guide ribonucleic acid (sgRNA), deoxyribonucleic acid (DNA), an antisense oligonucleotide (ASO), a plasmid, an episome, and combinations.

67. The method of claim 55, wherein the at least one water soluble agent comprises mRNA encoding SARS-CoV-2 spike protein receptor binding domain (RBD).

68. The method of claim 55, wherein the at least one water soluble agent comprises mRNA encoding SARS-CoV-2 spike protein receptor binding domain (RBD) with pseudo-uridine (C5-glycoside isomer of uridine, 5-ribosyluracil) modification.

69. The method of claim 55, wherein the first stabilizing amphiphilic copolymer is selected from the group consisting of poly(aspartic acid)-block-poly(lactic acid) (PAsp-b-PLA), poly(aspartic acid)-block-poly(lactic-co-glycolic acid) (PAsp-b-PLGA), dextran-poly(lactic acid) (Dex-PLA), dextran-poly(lactic-co-glycolic acid) (Dex-PLGA), poly(glutamic acid)-block-poly(lactic acid) (Pglu-b-PLA), poly(glutamic acid)-block-poly(lactic-co-glycolic acid) (Pglu-b-PLGA), poly(glutamic acid)-block-poly(caprolactone) (Pglu-b-PCL), poly(aspartic acid)-block-poly(caprolactone) (PAsp-b-PCL), and dextran-poly(caprolactone) (Dex-PCL).

70. The method of claim 69,
wherein the dextran, poly(aspartic acid), and poly(glutamic acid) each have a molecular weight within a range of from about 500 to about 500,000 Da, from about 500 to about 50,000 Da, or from about 750 Da to about 20,000 Da and
wherein the poly(lactic acid), poly(lactic-co-glycolic acid), and poly(caprolactone) each have a molecular weight within a range of from about 500 to about 500,000 Da, from about 500 to about 50,000 Da, or from about 750 Da to about 20,000 Da.

71. The method of claim 55, wherein the first stabilizing amphiphilic copolymer is selected from the group consisting of poly(glutamic acid)-block-poly(lactic acid) (Pglu-b-PLA) and poly(glutamic acid)-block-poly(lactic-co-glycolic acid) (Pglu-b-PLGA).

72. The method of claim 71,
wherein the poly(glutamic acid) has a molecular weight within a range of from about 500 to about 500,000 Da, from about 500 to about 50,000 Da, or from about 750 Da to about 20,000 Da and
wherein the poly(lactic acid) and poly(lactic-co-glycolic acid) each have a molecular weight within a range of from about 500 to about 500,000 Da, from about 500 to about 50,000 Da, or from about 750 Da to about 20,000 Da.

73. The method of claim 55, wherein the at least one lipid is selected from the group consisting of a phospholipid, a cationic lipid, an anionic lipid, a sterol, a monoglyceride, a triglyceride, a fatty acid methyl ester, a fatty acid ethyl ester, and combinations.

74. The method of claim 55, where the at least one lipid comprises a cationic lipid and/or 1-palmitoyl-2-oleoyl-sn-glycero-3-phosphocholine (POPC).

75. The method of claim 55, wherein the at least one lipid is a blend of a cationic lipid, a phospholipid, and a cholesterol or a sterol.

76. The method of claim 55, wherein the second stabilizing amphiphilic agent is selected from the group consisting of pegylated 1-(monomethoxy-polyethyleneglycol)-2,3-dimyristoylglycerol (PEG-DMG), pegylated distearoyl-phosphatidyl-ethanolamine (PEG-DSPE), polyethyleneglycol-block-poly(lactic acid) (PEG-b-PLA), polyethyleneglycol-block-poly(lactic-co-glycolic acid) (PEG-b-PLGA), and polyethyleneglycol-block-poly(caprolactone) (PEG-b-PCL).

77. The method of claim 76, wherein the poly(lactic acid), poly(lactic-co-glycolic acid), and poly(caprolactone) each have a molecular weight within a range of from about 500 to about 500,000 Da, from about 500 to about 50,000 Da, or from about 750 Da to about 20,000 Da.

78. The method of claim 55,
wherein the at least one water soluble agent comprises mRNA encoding SARS-CoV-2 spike protein receptor binding domain (RBD) and/or mRNA encoding SARS-CoV-2 spike protein receptor binding domain (RBD) with pseudo-uridine (C5-glycoside isomer of uridine, 5-ribosyluracil) modification,
wherein the first stabilizing amphiphilic copolymer comprises dextran-poly(lactic acid) (Dex-PLA), dextran-poly(lactic-co-glycolic acid) (Dex-PLGA) and/or dextran-poly(caprolactone) (Dex-PCL),
wherein the at least one lipid comprises a cationic lipid, and
wherein the second stabilizing amphiphilic agent comprises a polyethylene glycol (PEG) copolymer and/or a polyethylene glycol (PEG) lipid.

79. The method of claim 78,
wherein the at least one water soluble agent comprises mRNA encoding SARS-CoV-2 spike protein receptor binding domain (RBD) with pseudo-uridine (C5-glycoside isomer of uridine, 5-ribosyluracil) modification,
wherein the first stabilizing amphiphilic copolymer comprises dextran-poly(lactic-co-glycolic acid) (Dex-PLGA),
wherein the at least one lipid comprises 8-[(2-hydroxyethyl)[6-oxo-6-(undecyloxy)hexyl]amino]-octanoic acid, 1-octylnonyl ester (SM-102), 1-palmitoyl-2-oleoyl-glycero-3-phosphocholine (POPC), and cholesterol, and
wherein the second stabilizing amphiphilic agent comprises 1,2-dimyristoyl-rac-glycero-3-methoxypolyethylene glycol-2000 (PEG-DMG).

80. The nanoparticle of claim 1, wherein the at least one water soluble agent comprises a ribonucleic acid (RNA) of which at least one uridine is replaced with a modified uridine.

81. The nanoparticle of claim 80,
wherein at least 50% of the uridines of the ribonucleic acid (RNA) are each replaced with a modified uridine and
wherein the modified uridines may be the same or different.

82. The nanoparticle of claim 80,
wherein all of the uridines of the ribonucleic acid (RNA) are each replaced with a modified uridine and
wherein the modified uridines may be the same or different.

83. The nanoparticle of claim 80, wherein each modified uridine is independently selected from the group consisting of pseudouridine, 5-methoxyuridine, and N1-methylpseudouridine.

84. The nanoparticle of claim 83, wherein each modified uridine is independently selected from the group consisting of pseudouridine and 5-methoxyuridine.

85. The nanoparticle of claim 80, wherein the ribonucleic acid (RNA) is selected from the group consisting of messenger ribonucleic acid (mRNA), self-amplifying messenger ribonucleic acid (saRNA), small interfering ribonucleic acid (siRNA), micro ribonucleic acid (microRNA), circular ribonucleic acid (circular RNA), transfer ribonucleic acid (tRNA), small guide ribonucleic acid (sgRNA), and an antisense oligonucleotide ribonucleic acid (ASO RNA).

86. The nanoparticle of claim 1, wherein the at least one water soluble agent is a nucleic acid that is at least partially neutralized with a base.

87. The nanoparticle of claim 86, wherein the nucleic acid is 50% neutralized with a base.

88. The nanoparticle of claim 86, wherein the nucleic acid is fully neutralized with a base.

89. The nanoparticle of claim 86, wherein the nucleic acid is a ribonucleic acid (RNA).

90. The nanoparticle of claim 86, wherein the nucleic acid is selected from the group consisting of messenger ribonucleic acid (mRNA), self-amplifying messenger ribonucleic acid (saRNA), small interfering ribonucleic acid (siRNA), micro ribonucleic acid (microRNA), circular ribonucleic acid (circular RNA), transfer ribonucleic acid (tRNA), small guide ribonucleic acid (sgRNA), and an antisense oligonucleotide ribonucleic acid (ASO RNA).

91. The nanoparticle of claim 86, wherein the base is an amine.

92. The nanoparticle of claim 86, wherein the base is guanidine.

93. The nanoparticle of claim 86, wherein the base is a guanidine derivative or a guanidinium salt.

94. The nanoparticle of claim 86, wherein the base is arginine.

95. The nanoparticle of claim 86, wherein the base is a tertiary amine.

96. The nanoparticle of claim 86, wherein the base is selected from the group consisting of triethylamine and diphenhydramine.

97. The method of claim 55, wherein the at least one water soluble agent comprises a ribonucleic acid (RNA) of which at least one uridine is replaced with a modified uridine.

98. The method of claim 97,
wherein at least 50% of the uridines of the ribonucleic acid (RNA) are each replaced with a modified uridine and
wherein the modified uridines may be the same or different.

99. The method of claim 97,
wherein all of the uridines of the ribonucleic acid (RNA) are each replaced with a modified uridine and
wherein the modified uridines may be the same or different.

100. The method of claim 97, wherein each modified uridine is independently selected from the group consisting of pseudouridine, 5-methoxyuridine, and N1-methylpseudouridine.

101. The method of claim 100, wherein each modified uridine is independently selected from the group consisting of pseudouridine and 5-methoxyuridine.

102. The method of claim 97, wherein the ribonucleic acid (RNA) is selected from the group consisting of messenger ribonucleic acid (mRNA), self-amplifying messenger ribonucleic acid (saRNA), small interfering ribonucleic acid (siRNA), micro ribonucleic acid (microRNA), circular ribonucleic acid (circular RNA), transfer ribonucleic acid (tRNA), small guide ribonucleic acid (sgRNA), and an antisense oligonucleotide ribonucleic acid (ASO RNA).

103. The method of claim 55, wherein the at least one water soluble agent is a nucleic acid that is at least partially neutralized with a base prior to dissolving the at least one water soluble agent in the first polar process solvent.

104. The method of claim 103, wherein the nucleic acid is 50% neutralized with a base.

105. The method of claim 103, wherein the nucleic acid is fully neutralized with a base.

106. The method of claim 103, wherein the nucleic acid is a ribonucleic acid (RNA).

107. The method of claim 103, wherein the nucleic acid is selected from the group consisting of messenger ribonucleic acid (mRNA), self-amplifying messenger ribonucleic acid (saRNA), small interfering ribonucleic acid (siRNA), micro ribonucleic acid (microRNA), circular ribonucleic acid (circular RNA), transfer ribonucleic acid (tRNA), small guide ribonucleic acid (sgRNA), and an antisense oligonucleotide ribonucleic acid (ASO RNA).

108. The method of claim 103, wherein the base is an amine.

109. The method of claim 103, wherein the base is guanidine.

110. The method of claim 103, wherein the base is a guanidine derivative or a guanidinium salt.

111. The method of claim 103, wherein the base is arginine.

112. The method of claim 103, wherein the base is a tertiary amine.

113. The method of claim 103, wherein the base is selected from the group consisting of triethylamine and diphenhydramine.

114. The method of claim 55, wherein the at least one water soluble agent is a salt of a plasmid.

115. The method of claim 114, wherein the at least one water soluble agent is a triethylamine (TEA) salt of the plasmid.

116. The method of claim 114, wherein the at least one water soluble agent is a guanidine salt of the plasmid.

117. The method of claim 114, wherein the plasmid is an episome.

118. The method of claim 114, wherein the first stabilizing amphiphilic copolymer is a polysaccharide copolymer.

119. The method of claim 114, wherein the first stabilizing amphiphilic copolymer is dextran-poly(lactic-co-glycolic acid) (Dex-PLGA).

120. The method of claim 119,
wherein the dextran has a molecular weight within a range of from about 500 to about 500,000 Da, from about 500 to about 50,000 Da, or from about 750 Da to about 20,000 Da and
wherein the poly(lactic-co-glycolic acid) has a molecular weight within a range of from about 500 to about 500,000 Da, from about 500 to about 50,000 Da, or from about 750 Da to about 20,000 Da.

121. The method of claim 114, wherein the first stabilizing amphiphilic copolymer is a polypeptide copolymer.

122. The method of claim 114, wherein the first stabilizing amphiphilic copolymer is poly(aspartic acid)-poly(lactic-co-glycolic acid) (PAsp-PLGA).

123. The method of claim 122,
wherein the poly(aspartic acid) has a molecular weight within a range of from about 500 to about 500,000 Da, from about 500 to about 50,000 Da, or from about 750 Da to about 20,000 Da and
wherein the poly(lactic-co-glycolic acid) has a molecular weight within a range of from about 500 to about 500,000 Da, from about 500 to about 50,000 Da, or from about 750 Da to about 20,000 Da.

124. The method of claim 114, wherein the at least one lipid is selected from the group consisting of a cationic lipid, 1-palmitoyl-2-oleoyl-sn-glycero-3-phosphocholine (POPC), cholesterol, and combinations.

125. The method of claim 114, wherein the second amphiphilic stabilizing copolymer is a pegylated copolymer.

126. The method of claim 114, wherein the second amphiphilic stabilizing copolymer is 1-(monomethoxy-polyethyleneglycol)-2,3-dimyristoylglycerol (PEG-DMG).

127. The method of claim 55, wherein the antisolvent comprises a metal salt.

128. The method of claim 127, wherein the metal salt is selected from the group consisting of MgCl₂, CaCl₂), and ZnCl₂.
